(19) United States
(12) Patent Application Publication
(10) Pub. No.: US 2025/0381168 A1
(43) Pub. Date: Dec. 18, 2025

(54) VLA4 INHIBITORS AND USES THEREOF

(71) Applicant: Washington University, St. Louis, MO (US)

(72) Inventors: Peter RUMINSKI, St. Louis, MO (US); John DIPERSIO, St. Louis, MO (US); Michael RETTIG, St. Louis, MO (US)

(21) Appl. No.: 18/877,795

(22) PCT Filed: Jun. 21, 2023
(86) PCT No.: PCT/US2023/068760

§ 371 Date: Dec. 20, 2024

Related U.S. Application Data
(60) Provisional application No. 63/353,947, filed on Jun. 21, 2022.

(51) Int. Cl. A61K 31/40; A61K 31/198; A61K 31/395; A61K 31/4025; A61K 31/4192; A61K 38/19

(52) U.S. Cl. CPC A61K 31/40; A61K 31/198; A61K 31/395; A61K 31/4025; A61K 31/4192; A61K 38/195

(57) ABSTRACT

The present disclosure provides therapeutic agents comprising VLA-4 (a4b1) and a4b?inhibiting agents defined herein. Methods of using the therapeutic agents are also provided. The present disclosure further provides combinations of a VLA-4 inhibitor and one or more agents which interact with a chemokine receptor or chemokine receptors, and methods of use thereof. In some embodiments, the disclosed combinations may be used in a method of mobilizing hematopoietic stem cells. In some embodiments, the disclosed methods may be used in the treatment of a condition that requires the collection of hematopoietic stem cells for transfusions or in chemotherapy. The present disclosure further provides methods of treating a patient comprising administering an agent which interacts with a chemokine such as G-CSF, plerixafor, BL-8040 (Motixafortide), or Crop and VLA-4 inhibitors.

### CROSS-REFERENCE TO RELATED APPLICATIONS

This application claims priority from U.S. Provisional Application Ser. No. 63/353,947 filed on 21 Jun. 2022, which is incorporated herein by reference in its entirety.

### STATEMENT REGARDING FEDERALLY SPONSORED RESEARCH OR DEVELOPMENT

This invention was made with government support under CA210084 awarded by the National Institutes of Health. The government has certain rights in the invention.

### MATERIAL INCORPORATED-BY-REFERENCE

Not applicable.

### FIELD OF THE TECHNOLOGY

The present disclosure relates to the fields of pharmaceuticals, medicine, and cell biology. More specifically, it relates to pharmaceutical agents which are useful as antagonists (i.e., inhibitors) of one or more integrins, such as the integrin a4b1 (VLA-4), which, when used alone or in combination with other known agents, can mobilize hematopoietic stem cells (HSC) into the peripheral blood to enhance the collection of hematopoietic stem cells from a donor.

### BACKGROUND

Hematopoietic stem cell transplantation (HSCT) is the major curative therapy available for many hematological diseases including hematological cancers and more recently in gene therapy directed at blood borne diseases resulting from genetic mutations, such as sickle cell disease. In this technique, HSCT is used to facilitate repopulation of healthy bone marrow and immune system cells after a high-dose chemotherapy treatment for cancers including but not limited to Hodgkin's and non-Hodgkin's lymphoma, multiple myeloma, and leukemia, or, in the case of gene therapy, to repopulate with the ex vivo genetically modified cells correcting the disease gene defect. In order to facilitate transplantation when the cells are needed, hematopoietic stem/progenitor cells (HSPCs) are collected from the patient's blood, harvested, frozen, and then stored while the patient receives high-dose chemotherapy and/or radiation therapy. In order to achieve a successful transplantation, an intravenous infusion of a minimum number of 2×10⁶ CD34+ stem cells/kg body weight is often needed; however, a dose of 5×10⁶ CD34+ cells/kg is considered preferable for early and long term multi-lineage engraftment.

Currently, the stem cells for hematopoietic stem cell transplants are often harvested from peripheral blood. Due to the low amount of these cells in circulating peripheral blood, the stem cells often must be stimulated to increase the quantity in the peripheral blood, a process that, using current therapeutic agents, can take almost a week. Even then, the collection is still done over several days to achieve sufficient concentrations of the stem cells for transplantation. This greatly increases the cost of the transplant and results in a significant burden on the patient or donor. Currently, cytokines, such as granulocyte-colony forming unit (G-CSF), and immunostimulants, such as plerixafor, are used to increase the amount of hematopoietic stem cells in the peripheral blood, but a single agent often results in insufficient mobilization of stem cells. Additional methods of harvesting hematopoietic stem cells have been developed which involve combining G-CSF with multiple other agents such as plerixafor or another cytokine. Unfortunately, this treatment regimen can take from 5 to 8 days, with daily dosing of G-CSF, and even these combined therapies often fail to increase the concentrations to sufficient levels for transplantation in many patients even with these multiple days of apheresis. This results in excessive cost and burdensome time constraints. Additionally, G-CSF often has undesirable side effects and is contraindicated in some individuals, such as those with sickle cell disease. Therefore, a need remains for better methods to harvest hematopoietic stem cells, preferably using an agent that can rapidly mobilize these stem cells, with the ability to collect a sufficient quantity within a six to eight hour period in a single day after administration of a single dose of a mobilizing agent. Furthermore, gene editing, using techniques such as CRISPR, as a means to treat and potentially cure hematologic diseases caused by genetic mutations, also require ample hematopoietic stem cells with which to perform gene editing prior to infusion back into a patient. A more efficient and timely method for mobilizing and collecting these stem cells for such gene therapy would be desirable. And as stated above, some pathologies that are amenable to gene therapy, such as sickle cell disease, cannot use G-CSG as a mobilizing agent, thereby necessitating the need for new potent and suitable drug to accommodate these procedures.

Small molecule inhibitors of the integrin a4b1 (VLA-4) have been shown to rapidly mobilize HSCs after a single dose in mice (Christopher et al., Blood. 2009; 114(7):1331-9; Ramirez et al. Blood. 2009; 114(7):1340-3). This mobilization effect has also shown to be synergistic when dosed in combination with a CXCR4 inhibitor, such as Plerixafor (Ramirez et al. Blood. 2009; 114(7):1340-3). Previous small molecule inhibitors of VLA-4 reported in the literature were shown to be quite insoluble, lacked sufficient inhibition of VLA-4, and/or had poor pharmacokinetic properties that resulted in a significant, but short lasting effect on HSPC mobilization in mice. Most of the more potent VLA-4 inhibitors provided rapid and significant HSPC mobilization that peaked by 2 hours post dosing, but mobilization returned to baseline by 4 hours, not a sufficient enough amount of time to harvest an adequate number of HSPCs. Therefore, having a potent mobilization agent that could rapidly mobilize and extend this mobilization beyond 4 hours after a single dose would be desirable. Such an agent with these extended mobilization properties would allow for collection of an adequate number of HSPCs from a donor within a single day.

Potent VLA-4 inhibitors which are readily soluble in saline, have desirable pharmacokinetic properties, and that result in significant and extended mobilization of HSPCs lasting at least 6 hours after administration of a single dose have now been identified, and are disclosed herein. Furthermore, this HSPC mobilization effect is synergistic when co-administered with a single dose of a CXCR4 inhibitor such as, but not limited to, Plerixafor or Motixafortide, and/or a CXCR2 chemokine agonist. The ability of these novel inhibitors to achieve an adequate degree of mobilization and collection of HSPCs from a donor within a single day would greatly contribute toward a more efficient and cost-effective way to harvest HSPCs for stem cell transplantation and gene therapy.

### SUMMARY

Among the various aspects of the present disclosure is the provision of novel VLA-4 compositions, combination therapies with chemokine interacting agents, or methods of using the same. The present disclosure provides compounds which are a4b1 (VLA-4) or a4b7 antagonists (i.e., inhibitors), pharmaceutical compositions, methods for their manufacture, or methods for their use. The present disclosure provides methods using a compound that is a VLA-4 antagonist in combination with an agent which inhibits the CXCR4 receptor, and/or a CXCR2 agonist or similar cytokine agent, including methods of use or methods of treatment therewith. Also, provided herein are compositions comprising these novel a4b1 (VLA-4) or a4b7 antagonists. The present disclosure provides methods using a compound that includes VLA-4 antagonists in combination with a first or second agent which interacts with a chemokine (such as CXCR2 agonist or a CXCR4 inhibitor) including methods of use or methods of treatment therewith. Also, provided herein are compositions comprising these compounds.

An aspect of the present disclosure provides for a composition comprising a VLA-4 inhibiting agent of formula:

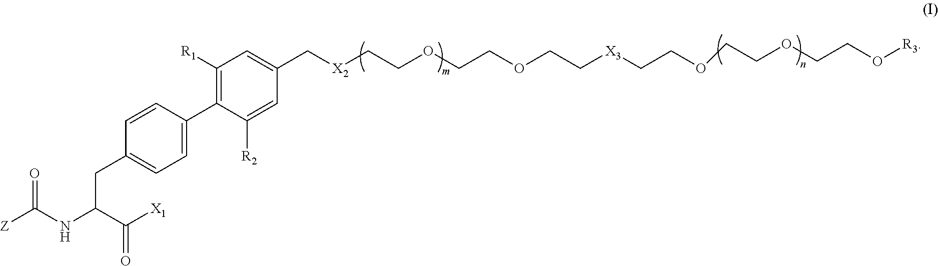

In some embodiments,

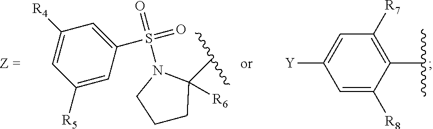

m=1-3; when

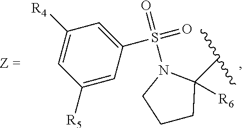

then n=19-1000; or when

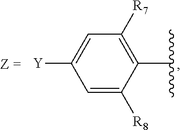

then n=222-1000; R₁ or R₂ are each independently hydroxyl, alkoxy_{(C≤8)} or substituted alkoxy_{(C≤8)}; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), wherein R₉ or R₁₀ are each independently hydrogen, alkyl_{(C≤6)}, substituted alkyl_{(C≤6)}; —(CH₂)_{g}—CH₂—CO₂R₉, wherein g is 0 or 1, and R₉ is hydrogen, alkyl_{(C≤6)}, substituted alkyl_{(C≤6)}; or wherein R₃ may represent a biomarker tag for in vitro or in vivo utility, an antibody targeting a specific protein or receptor, or another entity, which when attached to formula (I) enhances biological effects of combined entities, all attached to formula (I) at R₃; if m=1, or n=19-32, or X₃ is oxygen, then R₃ can also be:

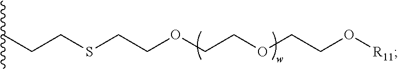

wherein w=100-900; and/or R₁₁ is defined as above for R₃; and/or, if X₃ is oxygen, m=1 or n=19-1000, then R₃ can also be:

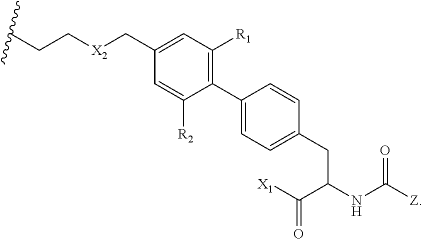

In some embodiments, R₁, R₂, X₁, and/or Z are defined above; R₄ or R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, or a substituent convertible in vivo to hydroxy; or a pharmaceutically acceptable salt thereof; R₆ is hydrogen, alkyl_{(C≤6)}, or substituted alkyl_{(C≤6)}; R₇ or R₈ are each independently hydrogen, halo, haloalkyl_{(C≤8)}; Y is hydrogen, cyano, halo, haloalkyl, hydroxy, or —C(O)X₄, wherein: X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof; X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof; X₂ is oxygen or sulfur; X₃ is oxygen, sulfur, —NH(C═O)—, —(C═O)NH—, —N(R₁₂)—; wherein R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof. Another aspect of the present disclosure provides for a composition comprising a VLA-4 inhibiting agent of formula:

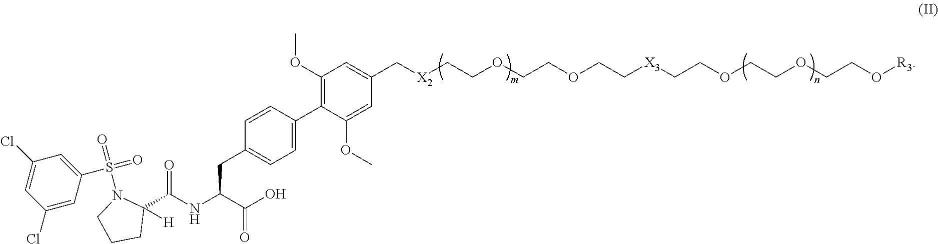

In some embodiments, m=1-3; n=19-1000; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), wherein R₉ or R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)}; or wherein R₃ may represent a biomarker tag for in vitro or in vivo utility, an antibody targeting a specific protein or receptor, or another entity, which when attached to formula (II) enhances biological effects of combined entities, all attached to formula (II) at R₃; or if m=1, or n=19-32, or X₃ is oxygen, then R₃ can also be:

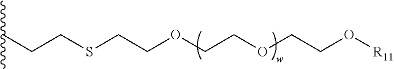

wherein w=100-900;
and/or R₁₁ is defined as above for R₃; and/or, if X₃ is oxygen, m=1 or n=19-1000, then R₃ can also be:

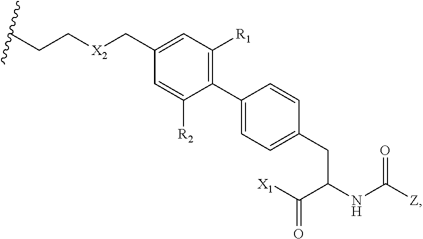

wherein R₁, R₂, X₁, or Z are defined above; X₂ is oxygen or sulfur; or X₃ is oxygen, sulfur, —NH(C═O)—, —(C═O)NH—, —N(R₁₂)—, wherein R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

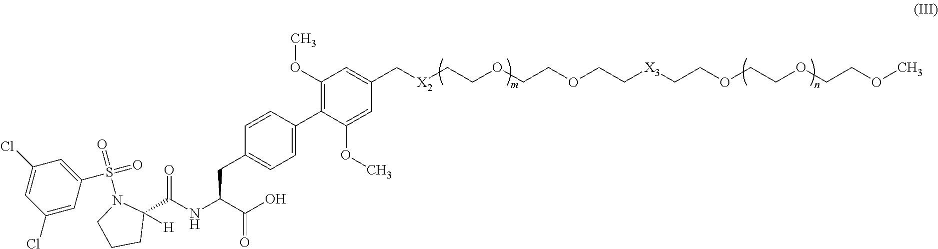

where: m=1-3; n=19-1000; X₂ is oxygen or sulfur; or X₃ is oxygen, sulfur, —NH(C═O)—, —(C═O)NH—, —N(R₁₂)—, wherein R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

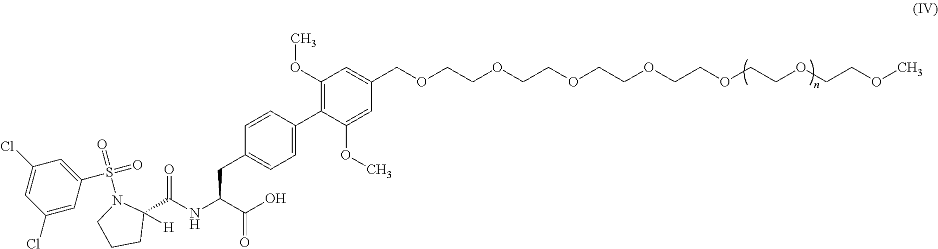

where: n=19; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

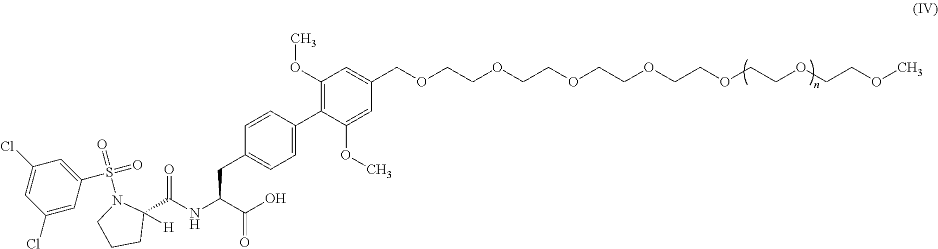

where: n=31; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

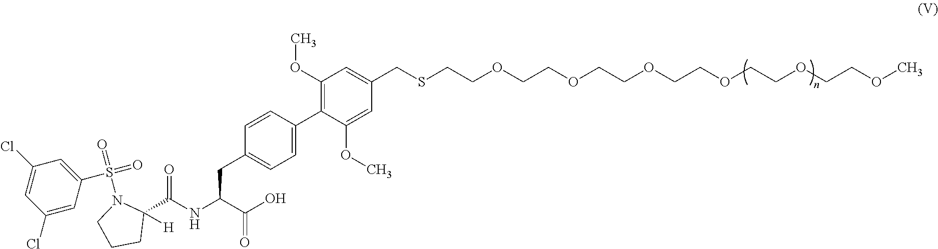

where: n=19-1000; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

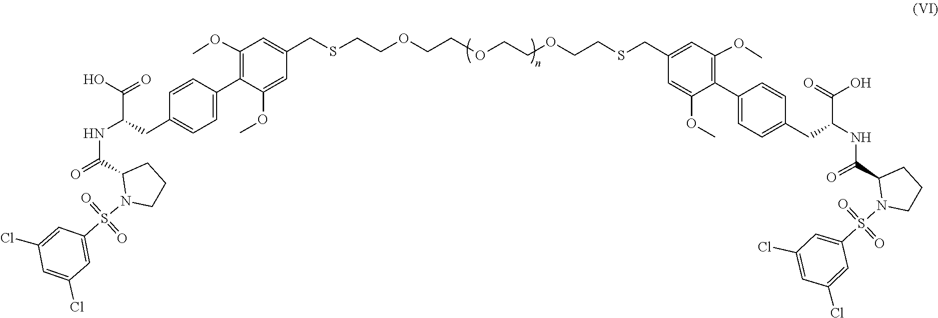

where: n=19-1000; or a pharmaceutically acceptable salt, solvate, polymorph, tautomer, prodrug, analog, or stereoisomer thereof or optionally substituted analog thereof. Yet another aspect of the present disclosure provides for a composition comprising a VLA-4 inhibiting agent of formula:

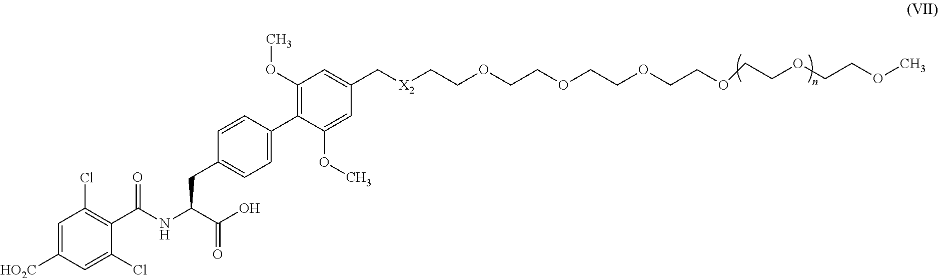

where: n=222-1000; X₂ is oxygen or sulfur; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

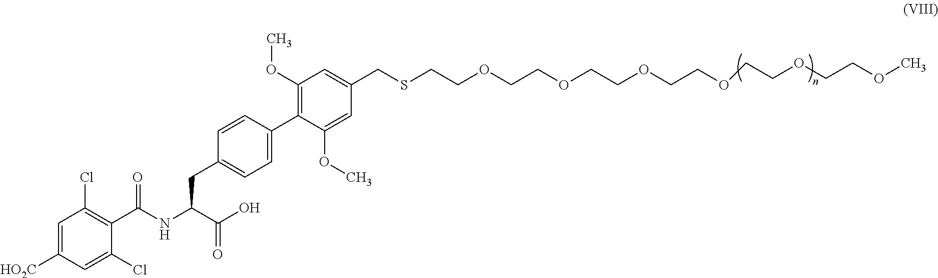

where: n=222-1000; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

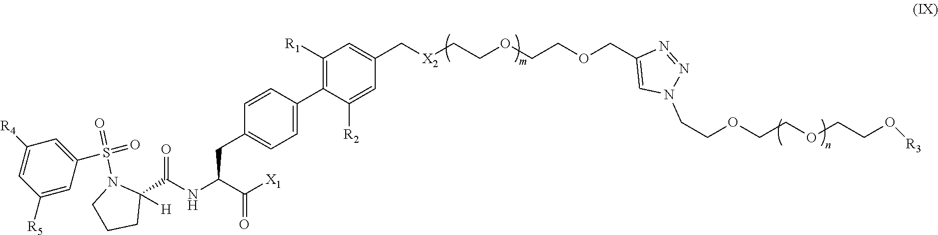

where: m=1-3; n=19-1000; X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof; X₂ is oxygen or sulfur; R₁ or R₂ are each independently hydroxyl, alkoxy_{(C≤8)} or substituted alkoxy_{(C≤8)}; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), wherein R₉ or R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)}; or wherein R₃ may represent a biomarker tag for in vitro or in vivo utility, an antibody targeting a specific protein or receptor, or another entity, which when attached to formula (IX) enhances biological effects of combined entities, all attached to formula (IX) at R₃; or if m=1, or n=19-32, then R₃ can also be:

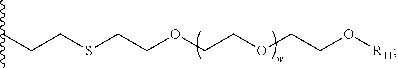

wherein w=100-900; or R₁₁ is defined as above for R₃; or R₄ or R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, or a substituent convertible in vivo to hydroxy; or a pharmaceutically acceptable salt thereof; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

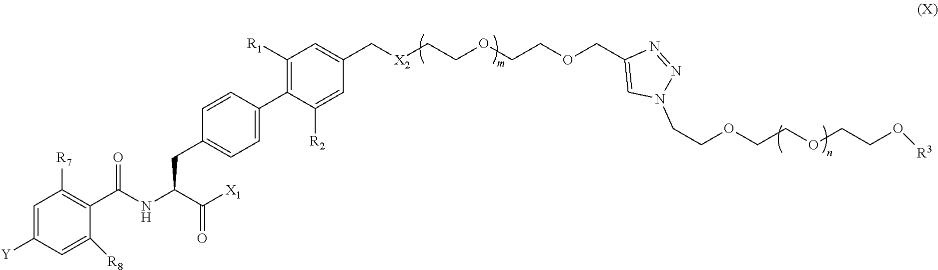

where: m=1-3; n=222-1000; X₁, X₂, R₁, R₂, or R₃ are as defined above, R₇ or R₈ are each independently hydrogen, halo, haloalkyl_{(C≤8)}; Y is hydrogen, cyano, halo, haloalkyl, hydroxy, or —C(O)X₄; wherein, X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

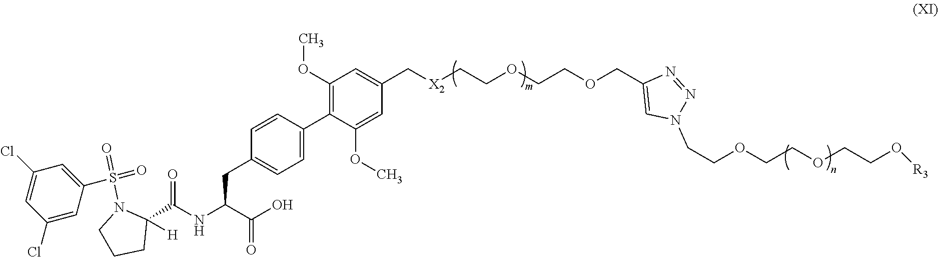

where: m=1-3; n=19-1000; X₂ is oxygen or sulfur; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), wherein R₉ or R₁₀ are each independently hydrogen, alkyl_{(C≤6)}, substituted alkyl_{(C≤6)}; —CH₂—CH₂—CO₂R₉, wherein R₉ is hydrogen, alkyl_{(C≤6)}, substituted alkyl_{(C≤6)}; or wherein R₃ may represent a biomarker tag for in vitro or in vivo utility, an antibody targeting a specific protein or receptor, or another entity, which when attached to formula (XI) enhances biological effects of combined entities, all attached to formula (XI) at R₃; if m=1, or n=19-32, then R₃ can also be:

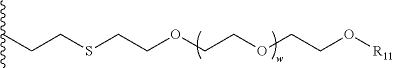

wherein w=100-900; or R₁₁ is defined as above for R₃; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

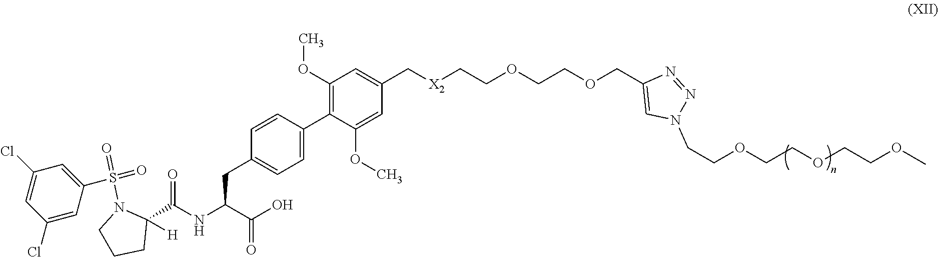

where: n=19-1000; X₂ is oxygen or sulfur; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a VLA-4 inhibiting agent of formula:

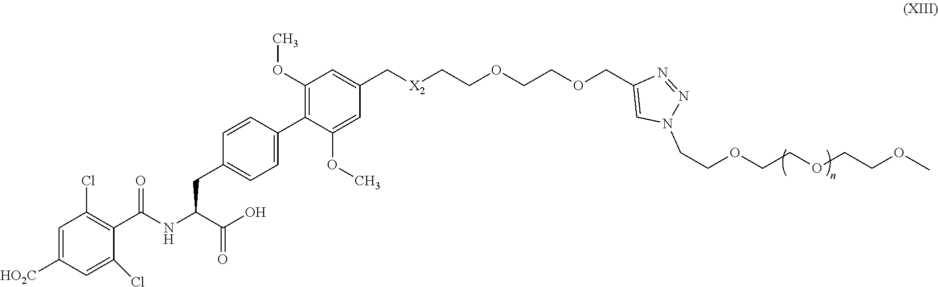

where: n=222-1000; X₂ is oxygen or sulfur; or a pharmaceutically acceptable salt, solvate, polymorph, tautomer, prodrug, analog, or stereoisomer thereof or optionally substituted analog thereof or bis-derivative thereof. Yet another aspect of the present disclosure provides for a composition comprising a formula selected from any one of the formulas:

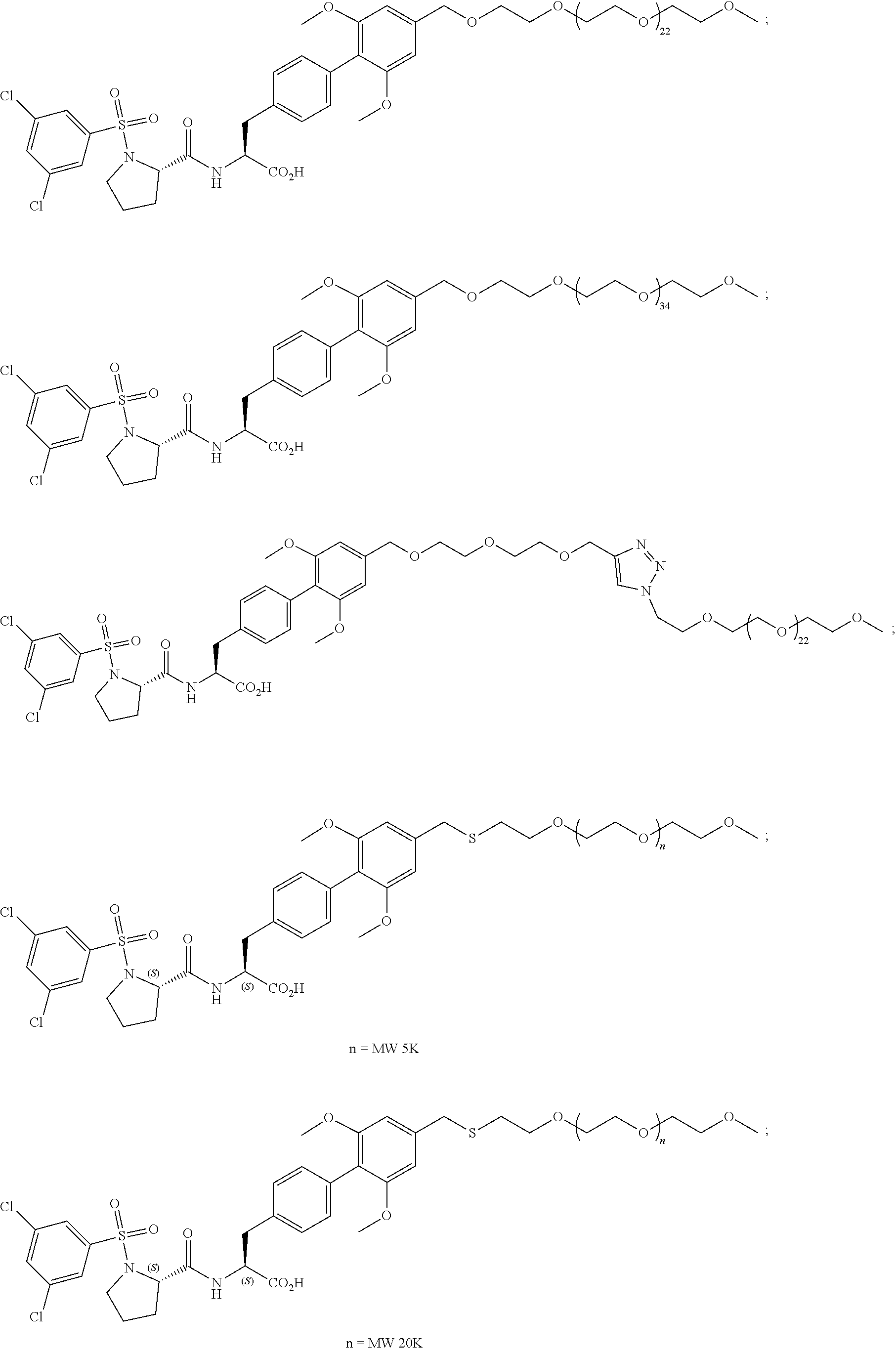

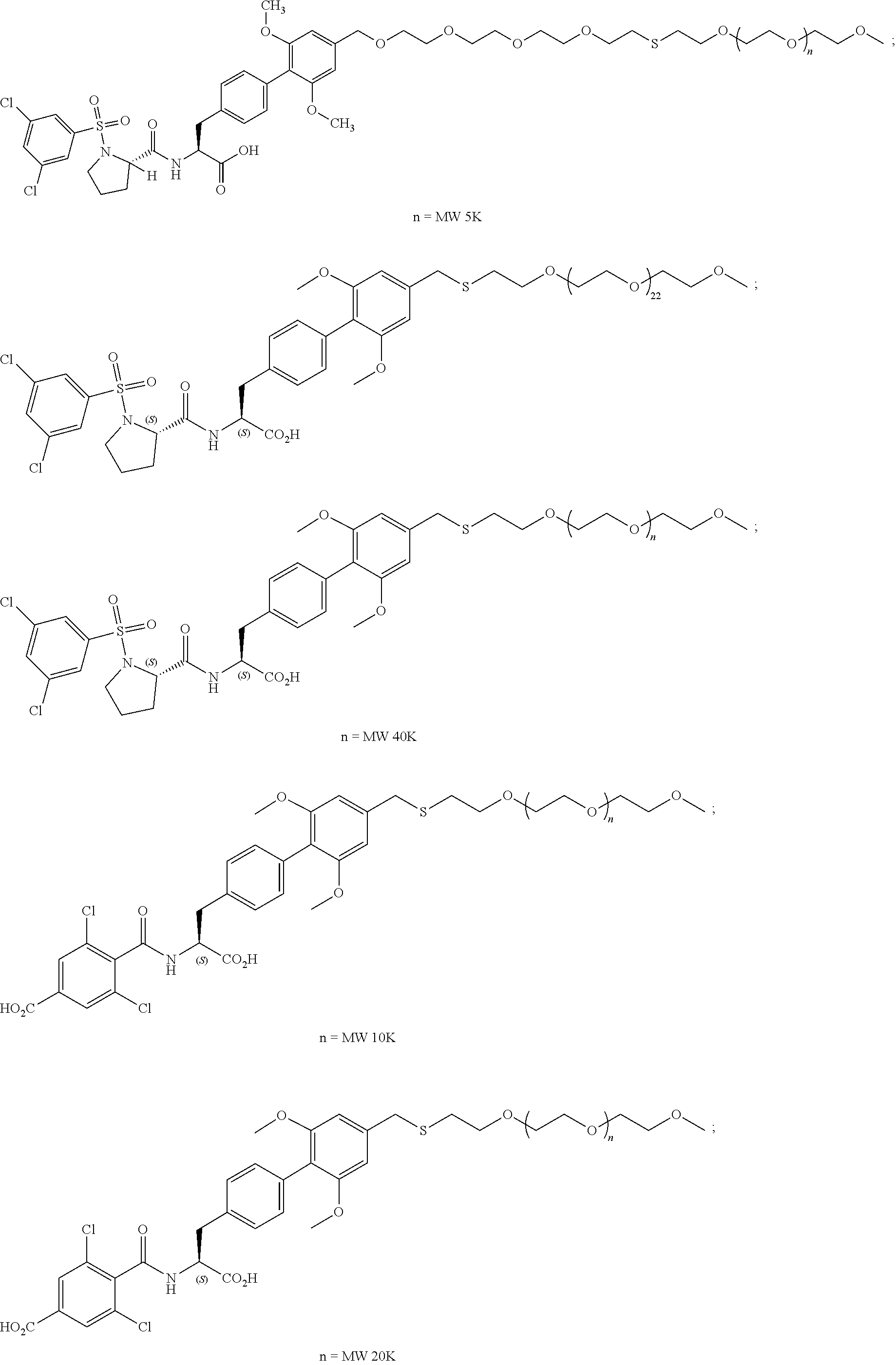

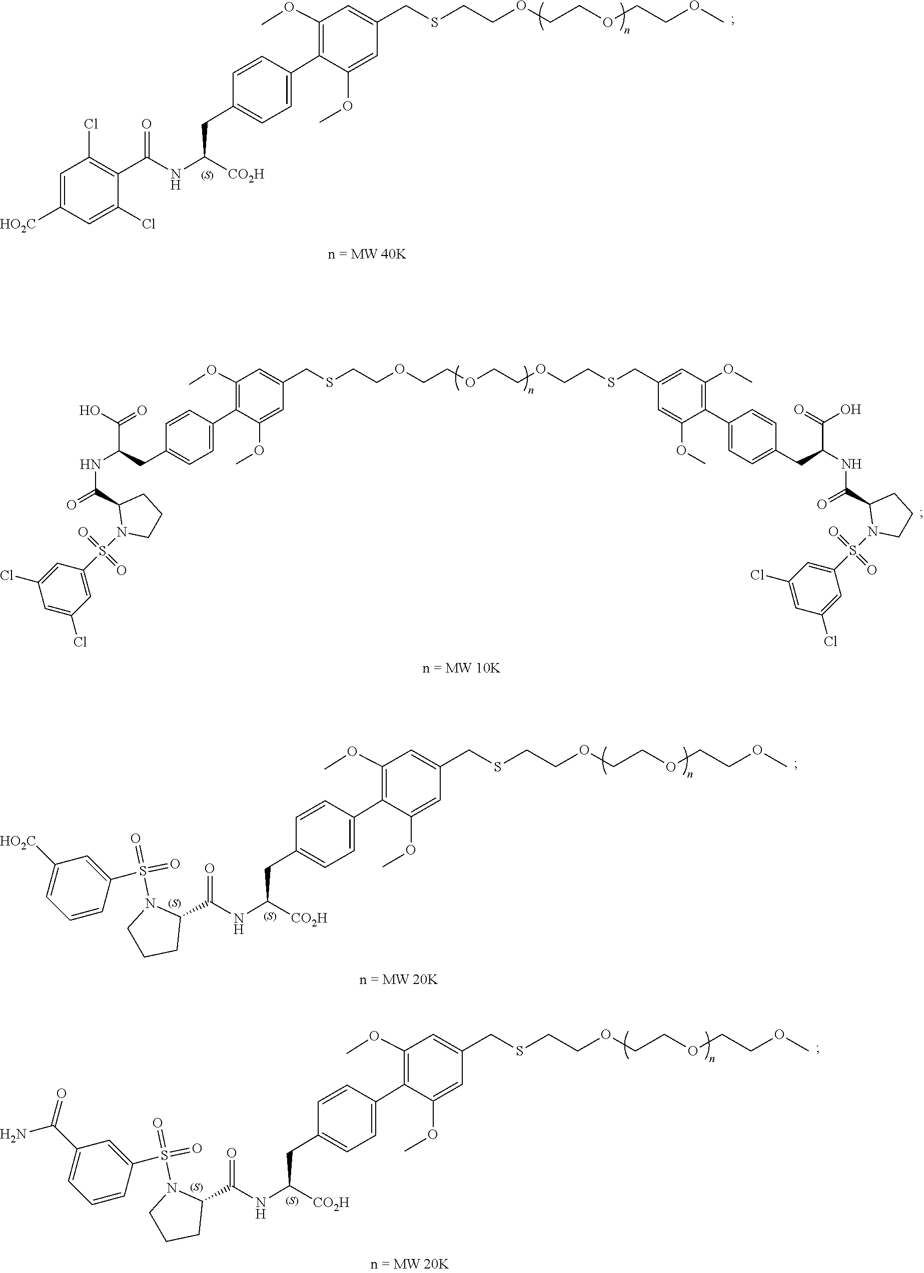

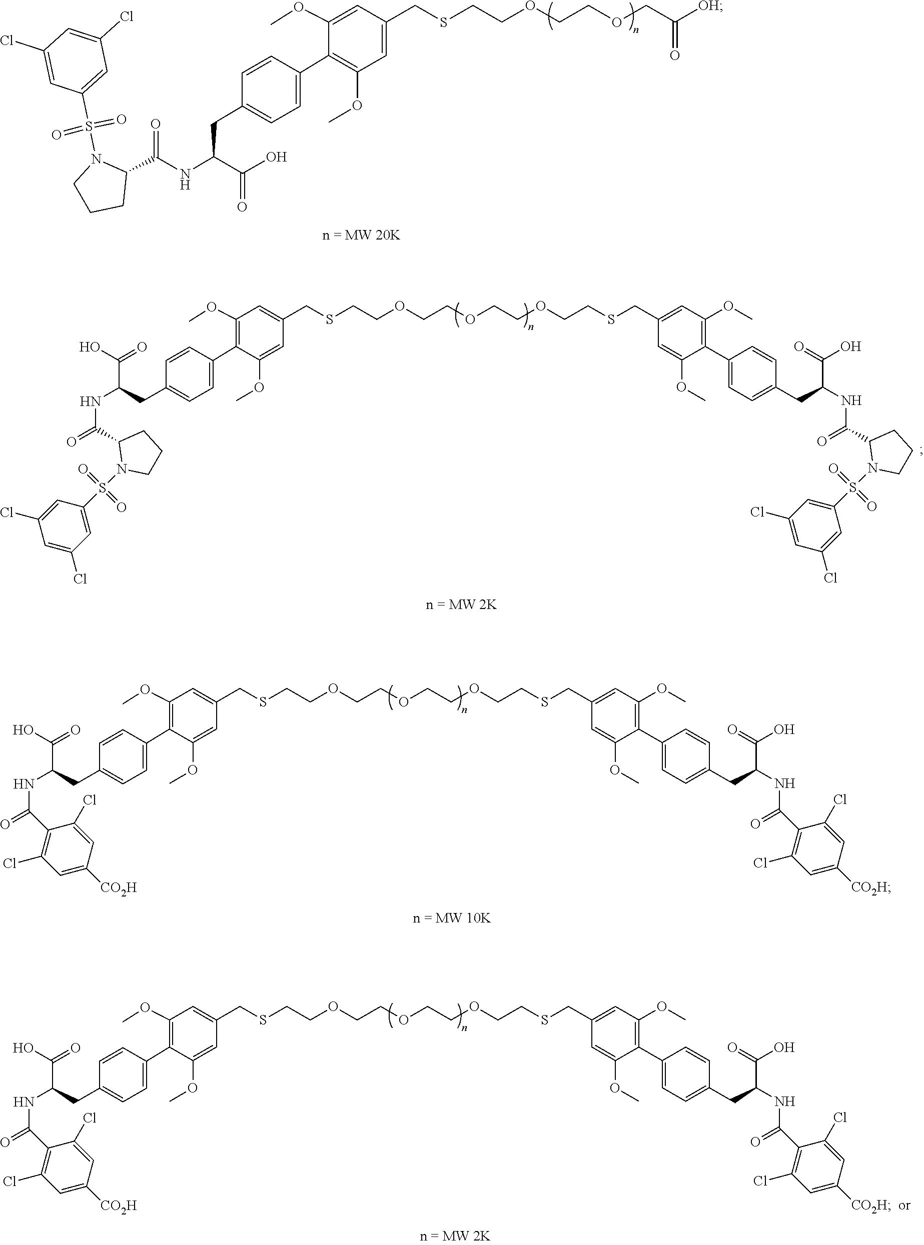

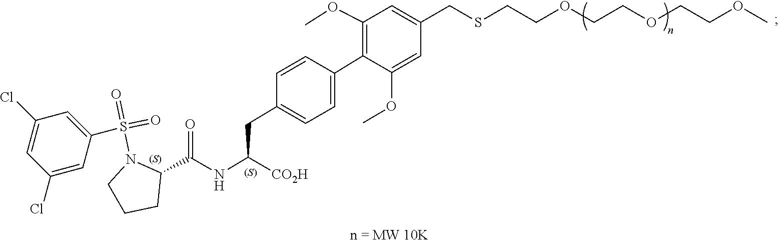

or a pharmaceutically acceptable salt thereof. Yet another aspect of the present disclosure provides for a pharmaceutical composition comprising: a) the composition of any one of the preceding embodiments; b) an excipient; and/or c) saline. In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated for oral, subcutaneous, intravenous, or intraperitoneal administration. Yet another aspect of the present disclosure provides for a composition comprising: a VLA-4 inhibitor of any one of the preceding embodiments; or one or more agents which interact with one or more chemokine receptors. In some embodiments, the one or more agents which interact with a chemokine receptor is an agent which interacts with a C—X—C chemokine receptor. In some embodiments, the one or more agents is a CXCR4 inhibitor or CXCR2 agonist. In some embodiments, the one or more agents is a CXCR4 inhibitor selected from, but not limited to AMD3100 (plerixafor), BL-8040 (Motixafortide), AMD3465, CTCE-0214, CTCE-9908, CP-1221 (linear peptides, cyclic peptides, natural amino-acids, unnatural amino acids, or peptidomimetic compounds), T140 or analogs, 4F-benzoyl-TN24003, KRH-1120, KRH-1636, KRH-2731, polyphemusin analogue, ALX40-4C, or combinations thereof. In some embodiments, the one or more agents is a CXCR2 agonist selected from Groβ or a derivative of Groβ. In some embodiments, the derivative of Groβ is a truncated Groβ. In some embodiments, the truncated Groβ is SB-251353. In some embodiments, the composition further comprises an inhibitor of integrin α9β1, G-CSF, a derivative of G-CSF, or a combination thereof. Yet another aspect of the present disclosure provides for a composition comprising: a VLA-4 inhibitor compound; or an agent which interacts with one or more chemokines. In some embodiments, the agent which interacts with a chemokine is selected from an agent which interacts with a C—X—C chemokine or a C—X—C chemokine receptor. In some embodiments, the agent is a CXCR4 inhibitor. In some embodiments, the agent is a CXCR2 agonist. In some embodiments, the CXCR4 inhibitor is one or more of, but not limited to AMD3100 (plerixafor), BL-8040 (Motixafortide), AMD3465, CTCE-0214, CTCE-9908, CP-1221 (e.g., linear peptides, cyclic peptides, natural amino-acids, unnatural amino acids, peptidomimetic compounds), T140 or analogs, 4F-benzoyl-TN24003, KRH-1120, KRH-1636, KRH-2731, polyphemusin analogue, ALX40-4C, or combinations thereof. In some embodiments, the CXCR2 agonist is Groβ or a derivative of Groβ. In some embodiments, the derivative of Groβ is a truncated Groβ. In some embodiments, the truncated Groβ is SB-251353. In some embodiments, the pharmaceutical composition is formulated as a unit dose. Yet another aspect of the present disclosure provides for a pharmaceutical composition comprising the composition of any one of the preceding embodiments. In some embodiments, the pharmaceutical composition of the combination of a VLA4 inhibitor or an agent which interacts with one or more chemokines of any one of the preceding embodiments is formulated or administered as a unit dose or formulated or administered independently of each other. Yet another aspect of the present disclosure provides for a method of treating a patient or a donor to enhance the mobilization and/or collection of a sufficient quantity of hematopoietic stem/progenitor cells into the peripheral blood of the patient or the donor comprising administering to the patient or donor the composition or pharmaceutical composition of any one of the preceding embodiments in an amount sufficient to mobilize and/or collect a sufficient quantity of hematopoietic stem/progenitor cells into the peripheral blood. In some embodiments, the amount sufficient to mobilize and/or collect a sufficient quantity of hematopoietic stem/progenitor cells is an amount that results in multilineage engraftment in a recipient. In some embodiments, the amount sufficient to mobilize and/or collect a sufficient quantity of hematopoietic stem/progenitor cells is an amount that results in neutrophil or platelet engraftment. In some embodiments, the amount sufficient to mobilize and/or collect a sufficient quantity of hematopoietic stem/progenitor cells is an amount sufficient for use in gene editing or genetic engineering. In some embodiments, the amount sufficient to mobilize and/or collect a sufficient quantity of hematopoietic stem/progenitor cells is an amount sufficient to be therapeutically effective in a subject having a disease, disorder, or condition that is treatable with hematopoietic stem/progenitor cells. In some embodiments, the disease, disorder, or condition is associated with impaired production of hematopoietic progenitor and/or stem cells resulting from a high dose of chemotherapy, radiotherapy, another therapeutic agent, such as for treating blood cancers or a genetic abnormality. In some embodiments, the disease, disorder, or condition is associated with a blood cancer or a genetic abnormality; a blood borne disease (e.g., sickle cell disease); or a hematopoietic malignancy (e.g., leukemia, lymphoma, or myeloma, such as multiple myeloma or acute myeloid leukemia). In some embodiments, the amount sufficient to mobilize and/or collect a sufficient quantity of hematopoietic stem/progenitor cells into the peripheral blood of a human donor is at least about 2 million CD34+ stem cells per kilogram recipient body weight. Yet another aspect of the present disclosure provides for a method of treating a patient, comprising collecting hematopoietic stem/progenitor cells from a patient or the donor, resulting in collected hematopoietic stem/progenitor cells; or infusing the collected hematopoietic stem/progenitor cells of the preceding embodiments. In some embodiments, the patient may have impaired production of hematopoietic progenitor and/or stem cells resulting from a high dose of chemotherapy, radiotherapy, another therapeutic agent, such as for treating blood cancers or a genetic abnormality. In some embodiments, the method further comprises collecting hematopoietic stem/progenitor cells from the patient, resulting in collected hematopoietic stem/progenitor cells; or gene editing the collected hematopoietic stem/progenitor cells of the patient, wherein the gene editing corrects a mutation causing a blood borne disease, resulting in gene edited hematopoietic stem/progenitor cells; or infusing the gene edited hematopoietic stem/progenitor cells into the patient to attenuate or treat the cause or pathology of the blood borne disease. In some embodiments, the blood borne disease is sickle cell disease. Yet another aspect of the present disclosure provides for a method of treating and/or preventing a disease, disorder, or condition in a patient in need thereof, comprising administering to the patient a composition or pharmaceutical composition of any one of the preceding embodiments in an amount sufficient to treat and/or prevent the disease, disorder, or condition. In some embodiments, a compound or composition of any one of the preceding embodiments increases effectiveness of an anti-cancer therapy. In some embodiments, the anti-cancer therapy is used to treat a patient who have or are at risk for a hematopoietic malignancy (e.g., lymphoma, myeloma, leukemia), wherein the compositions of any one of the preceding embodiments are administered or employed prior to, during, or subsequent to an anti-cancer therapy (e.g., chemotherapeutic agents, radiotherapy). In some embodiments, the hematopoietic malignancy is multiple myeloma or acute myeloid leukemia. In some embodiments, a compound or composition of any one of the preceding embodiments is administered or employed in combination with bi-specific antibodies or other immuno-oncology agents for treating patients with a leukemia, lymphoma, or myeloma. In some embodiments, a compound or composition of any one of the preceding embodiments is administered or employed in combination with bi-specific antibodies or other immuno-oncology agents for treating a patient having multiple myeloma or acute myeloid leukemia. Yet another aspect of the present disclosure provides for a method of treating a disease, disorder, or condition associated with cell adhesion-mediated inflammatory pathways with a pharmaceutical composition of any one of the preceding embodiments. In some embodiments, the disease, disorder, or condition is, but not limited to, asthma, multiple sclerosis, rheumatoid arthritis, atherosclerosis, inflammatory bowel disease, Crohn's disease, ulcerative colitis, graft vs host disease, neuroinflammation, neurodegeneration, or spinal cord injury. Yet another aspect of the present disclosure provides for a method of binding inhibition of an integrin comprising contacting the integrin with a composition of any one of the preceding embodiments. In some embodiments, the integrin is VLA4 (a4b1) or a4b7. In some embodiments, the integrin is VLA4 (a4b1). In some embodiments, the integrin is a4b7. In some embodiments, the method is performed in vitro. In some embodiments, the method is performed ex vivo or in vivo. In some embodiments, the binding inhibition is sufficient to treat or prevent a disease, disorder, or condition in a patient or to enhance or extend mobilization and/or collection of sufficient amounts of hematopoietic stem/progenitor cells into the peripheral blood. In some embodiments, the binding inhibition in combination with one or more agents which interact with one or more chemokine receptors is sufficient to treat or prevent a disease, disorder, or condition in a patient or to enhance or extend mobilization and/or collection of sufficient amounts of hematopoietic stem/progenitor cells into the peripheral blood. Yet another aspect of the present disclosure provides for a pharmaceutical composition comprising a composition of any one of the preceding embodiments which provides for significant or extended mobilization of hematopoietic stem/progenitor cells into the peripheral blood of a patient or donor lasting greater than 4 hours after a single administered dose. Yet another aspect of the present disclosure provides for a pharmaceutical composition comprising a composition of any one of the preceding embodiments comprising a PEG MW equal to or greater than 20 KD which provides for significant or extended mobilization of hematopoietic stem/progenitor cells into the peripheral blood of a patient or donor lasting greater than 24 hours after a single administered dose.

Other objects, features, and advantages of the present disclosure will become apparent from the following detailed description, examples, and supporting biological data, and in particular, the unexpected ability of the compounds as described herein to afford extended mobilization of HSPCs after a single dose. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1B is an exemplary embodiment showing a liquid chromatography-mass spectrometry (LC-MS) report for Example 4 in accordance with the present disclosure. FIG. 1A includes chromatograms and FIG. 1B includes mass spectra for Example 4.

FIG. 2A-FIG. 2B is an exemplary embodiment showing an LC-MS report for Example 5 in accordance with the present disclosure. FIG. 2A includes chromatograms and FIG. 2B includes a mass spectrum for Example 5.

FIG. 3A-FIG. 3B is an exemplary embodiment showing an LC-MS report for Example 6 in accordance with the present disclosure. FIG. 3A includes chromatograms and FIG. 3B includes a mass spectrum for Example 6.

FIG. 4A-FIG. 4B is an exemplary embodiment showing an LC-MS report for Comparator Compound 15 in accordance with the present disclosure. FIG. 4A includes chromatograms and FIG. 4B includes a mass spectrum for Comparator Compound 15.

FIG. 5 shows a 1HNMR spectrum for Example 13 in accordance with the present disclosure.

FIG. 6 shows a 1HNMR spectrum for Example 14 in accordance with the present disclosure.

FIG. 7 shows a 1HNMR spectrum for Example 15 in accordance with the present disclosure.

FIG. 8 shows a 1HNMR spectrum for Example 16 in accordance with the present disclosure.

FIG. 9 shows a 1HNMR spectrum for Example 17 in accordance with the present disclosure.

FIG. 10 shows a 1HNMR spectrum for Example 19 in accordance with the present disclosure.

FIG. 11 shows hematopoietic stem/progenitor cell (HSPC) mobilization for Comparator Compounds 1-4 (C1-C4) in accordance with the present disclosure.

FIG. 12 shows HSPC mobilization for Comparator Compounds 5-8 (C5-C8) in accordance with the present disclosure.

FIG. 13 shows HSPC mobilization for Example 1 (Ex 1) and Comparator Compounds 1 and 8 (C1 and C8) in accordance with the present disclosure.

FIG. 14 shows HSPC mobilization for Examples 1 and 3 (Ex 1 and Ex 3) and Comparator Compounds 9, 10, 11, and 18 (C9, C10, C11, and C18) in accordance with the present disclosure.

FIG. 15 shows HSPC mobilization for Examples 1 and 2 (Ex 1 and Ex 2) and Comparator Compounds 7, 12, 13, and 14 (C7, C12, C13, and C14) in accordance with the present disclosure.

FIG. 16 shows HSPC mobilization for Examples 1 and 2 (Ex 1 and Ex 2) and Comparator Compounds 14, 15, and 17 (C14, C15, and C17) in accordance with the present disclosure.

FIG. 17 shows HSPC mobilization for Examples 1, 4, 6, 7, 9 (Ex 1, Ex 4, Ex 6, Ex 7, and Ex 9) and Comparator Compounds 12 and 16 (C12 and C16) in accordance with the present disclosure.

FIG. 18 shows HSPC mobilization for Examples 1, 5, 9 (Ex 1, Ex 5, and Ex 9) and Comparator Compounds 1, 17, 19, and 20 (C1, C17, C19, and C20) in accordance with the present disclosure.

FIG. 19 shows HSPC mobilization for prior art compounds BOP and Firategrast with the CXCR2 agonist truncated Groβ (Groβt, abbreviated Gro) in accordance with the present disclosure.

FIG. 20 shows HSPC mobilization for Example 1 (Ex 1) alone or in combination with a single dose of the CXCR4 inhibitor AMD3100 (Plerixafor, abbreviated AMD) in accordance with the present disclosure.

FIG. 21 shows HSPC mobilization for Examples 1, 4, and 5 (Ex 1, Ex 4, and Ex 5) alone or in combination with a single dose of Plerixafor (Pier) in accordance with the present disclosure.

FIG. 22 shows HSPC mobilization for Examples 1 and 2 (Ex 1 and Ex 2) combined with either plerixafor (Pier), with the CXCR2 agonist truncated Groβ (Gro), or where all 3 are used in combination in accordance with the present disclosure.

FIG. 23 shows HSPC mobilization for Example 1 (Ex 1) or Comparator Compound 17 (C17) combined with plerixafor (pier) and Groβt (Gro), as well as Comparator Compounds 4 and 12 (C4 and C12) in accordance with the present disclosure.

FIG. 24 shows HSPC mobilization for Examples 1, 5, 10, 8 and 11 (Ex 1, Ex 5, Ex 10, Ex 8, and Ex 11) and Comparator Compound 21 (C21) in accordance with the present disclosure.

FIG. 25 shows HSPC mobilization for Examples 9 and 12 (Ex 9 and Ex 12) and Comparator Compound 20 (C20) in accordance with the present disclosure.

FIG. 26 shows HSPC mobilization for Example 9 (Ex 9), Comparator Compound 1 (C1), and prior art small molecule VLA4 inhibitors Firategrast, RO0270608, and Carotegrast in accordance with the present disclosure.

### DETAILED DESCRIPTION

The present disclosure is based, at least in part, on the unexpected and surprising discovery that defined lengths of polyethylene glycol (PEG) units, covalently and specifically attached to the disclosed chemical structures (as depicted in Examples 1-19, see e.g., TABLE 1) provide for superior and significantly extended mobilization of hematopoietic stem cells into the peripheral blood after administration of a single subcutaneous dose, compared to similar chemical structures that do not possess such specific structural attributes (as depicted in Comparator Compounds 1-22, see e.g., TABLE 2). Furthermore, these specific PEG compounds disclosed herein maintain high potency as inhibitors of VLA4 (a4b1) and a4b7 and also provide for superior solubility in straight saline.

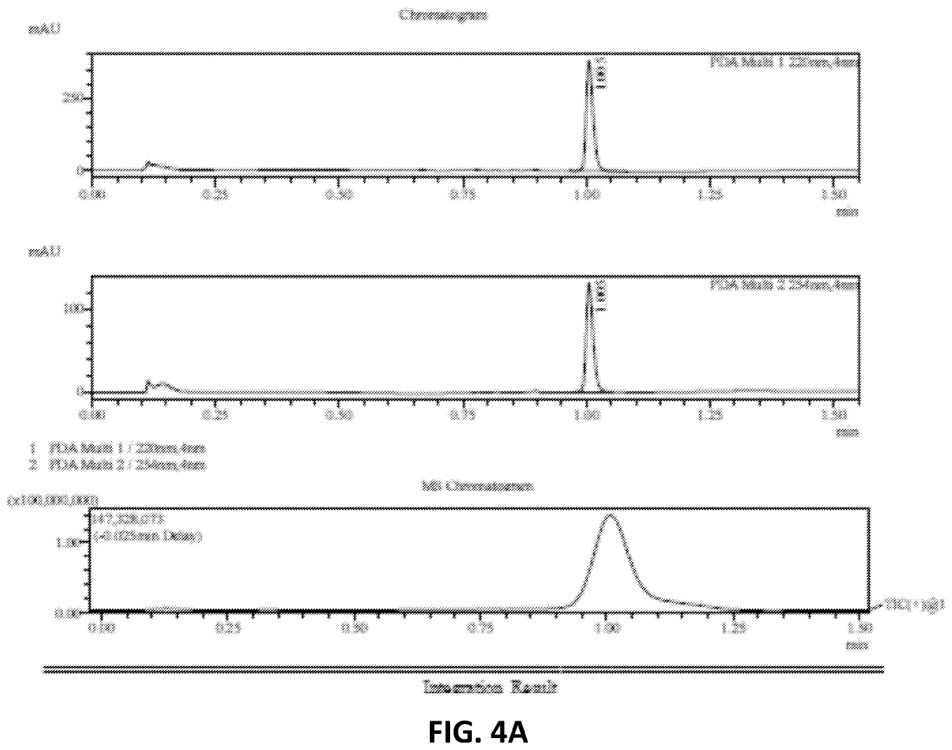

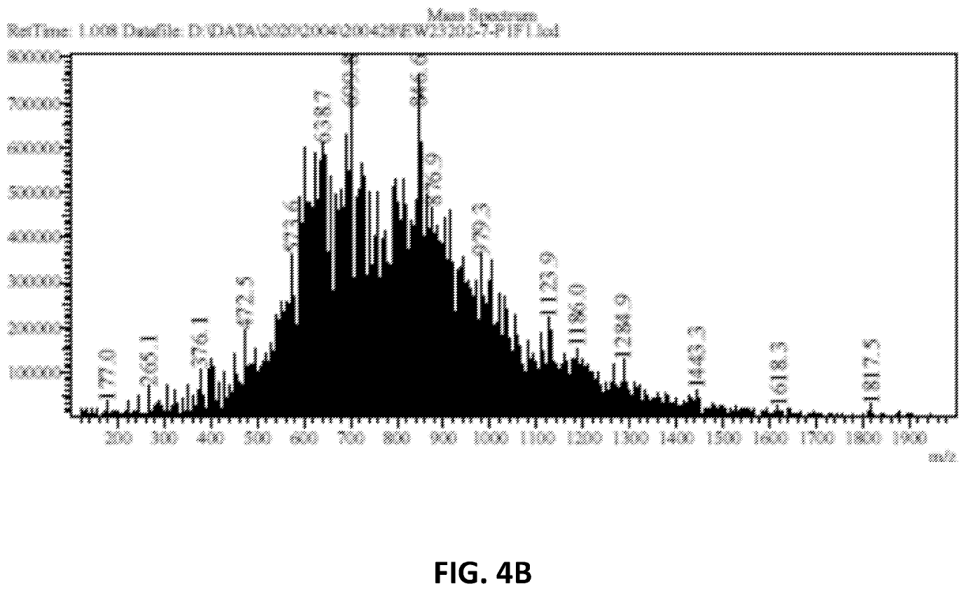

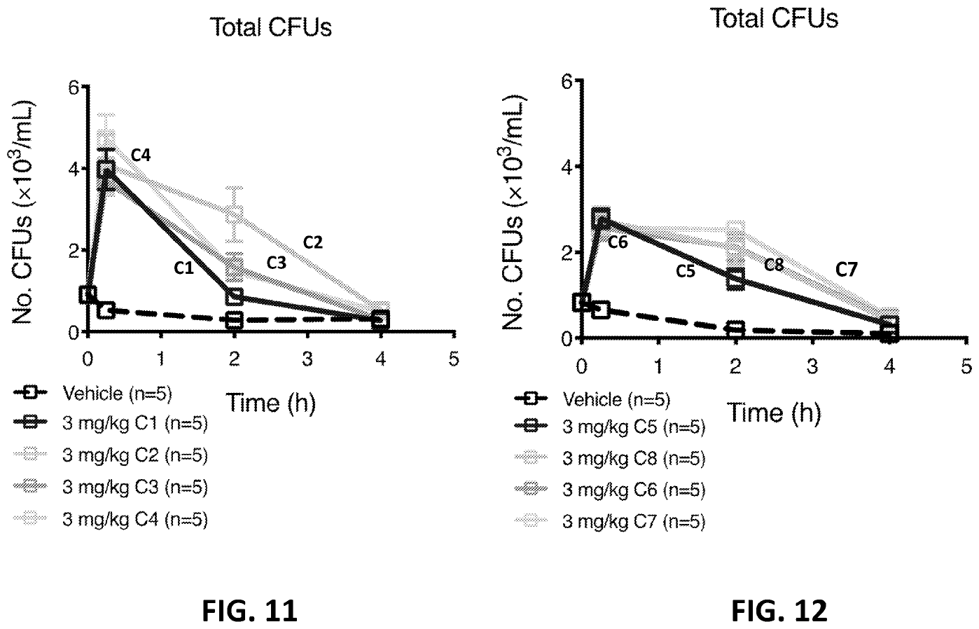

The present disclosure entails two main core structures, a “di-chlorophenyl sulfonamide core” as represented by Example 1 and a “di-chlorobenzoic acid core” as represented by Example 9, with the aforementioned PEG groups of varying lengths covalently attached to each core via a linker at the specific attachment point on the terminal phenyl group as depicted in Examples 1 and 9. As shown and detailed in the HSPC mobilization section of Exemplary Embodiment 2 (see e.g., FIG. 11-FIG. 25), it is not sufficient to attach any type or length of a PEG chain to these core structures in order to achieve the desired extended mobilization of HSPCs at a high level beyond 4 hours. For the “di-chlorophenyl sulfonamide core”, an m-PEG chain of at least 24 PEG units is required for superior rapid and extended mobilization out to at least 6 hours after a single administered dose, as represented by Examples 1, 2, 4, 5, 6, 7 and 8. This is in contrast to non-PEG or PEG chains less than 24 PEG units, as represented by Comparator Compounds C5-8, C12-14, and C16. As demonstrated, PEG lengths of 4, 8, 12, and 16 PEG units do not extend mobilization at 4 hours or longer as compared to the disclosed claimed matter with PEG lengths of 24 PEG units or greater. Based upon this data, it should be evident that a PEG unit greater than 16 PEG units is required for extended mobilization. Due to limited available reagents with specific PEG units needed for the synthesis of these compounds, specific PEG lengths between 17 and 23 PEG units were not readily available. Therefore, clear, extended HSPC mobilization, as directly correlated to specific PEG lengths, lies somewhere between 16 and 24 PEG units, but with the claimed minimum of 24 PEG units as needed for robust and reproducible extended HSPC mobilization for the “di-chlorophenyl sulfonamide core” (represented as n=19-1000 in the formulas section) can be envisioned as a reasonable minimum PEG length to achieve the desired extended HSPC mobilization effect for this core. Furthermore, as demonstrated by Comparator Compounds C15 and C21, attaching any PEG functionality, even if greater than 24 PEG units, does not guarantee extended or even significant mobilization, highlighting the non-obvious nature of the claims and examples, as disclosed herein. This nonobviousness is further exemplified by Example 11, a bis-“di-chlorophenyl sulfonamide core” separated by a 10 KD PEG linker, which quite surprisingly and unexpectedly extends mobilization out to 24 hours. To further highlight the non-obvious nature of the disclosed claims, context of where the PEG chain is attached relative to the core structure is demonstrated by Example 3 and Comparator Compounds C9 and C10. Example 3 utilizes the facile synthesis afforded by “Click” chemistry to attach a 24 PEG unit azide chain to an acetylenic functionality on the core structure to form a 24 PEG unit triazole coupled to the “di-chlorophenyl sulfonamide core” via a 3 unit PEG linker. Example 3 provides for extended mobilization past 4 hours. By contrast C9, which has the same 24 PEG unit triazole, as well as C10, which has a 36 PEG unit triazole, but both attached to the “di-chlorophenyl sulfonamide core” via a shorter linker than Example 3, demonstrate inferior mobilization at 4 hours compared to Example 3, as depicted in FIG. 4 in the mobilization data section. Additionally, C11, which is a 24 PEG unit attached to a truncated “di-chlorophenyl sulfonamide core”, provides no mobilization at any time point, demonstrating that a long PEG chain by itself does not mobilize HSPCs, and that superior and extended mobilization of HSPCs is dependent upon the entirety of the structural matter disclosed herein, comprised of the core structure covalently attached to the PEG groups as defined and depicted in the claims.

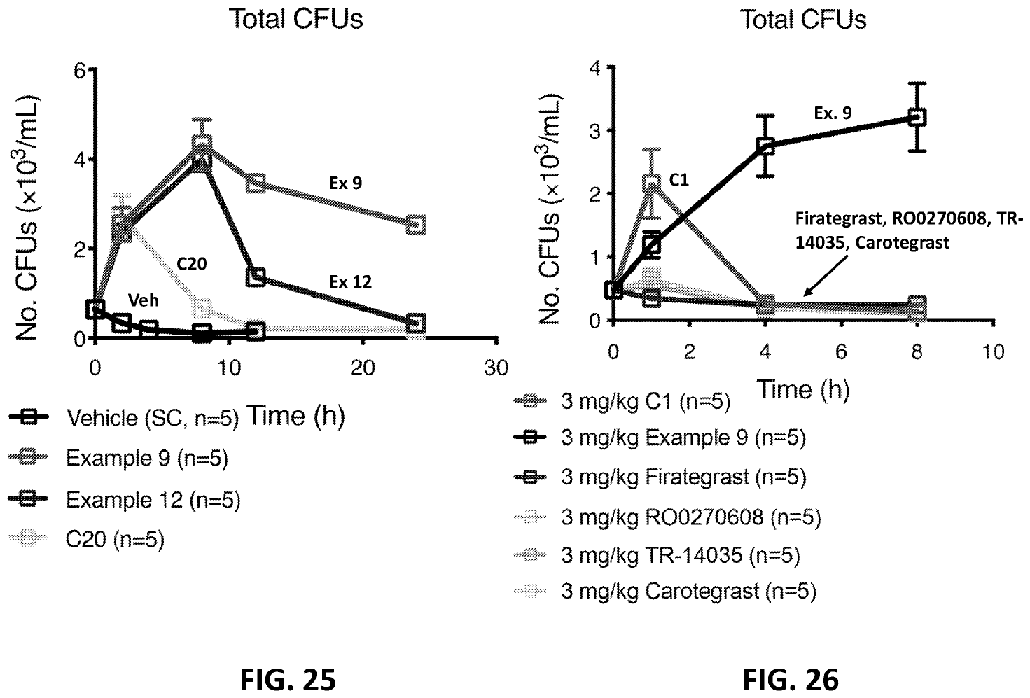

To further exemplify the non-obvious nature of the disclosed claims of the disclosed technology, the minimum PEG chain length required for extended mobilization attached to the “di-chlorophenyl sulfonamide core”, as in Example 1, does not correlate to the minimum PEG chain length required for extended mobilization when attached to the “di-chlorobenzoic acid core”. A 24 PEG unit attached to the “di-chlorobenzoic acid core”, Comparator Compound C17, and even a 2 KD PEG, Comparator Compound C19, and a 5 KD PEG, Comparator Compound C20, do not provide for extended mobilization. For a “di-chlorobenzoic acid core”, a 10 KD PEG, Example 12, a 20 KD PEG, Example 9, and a 40 KD PEG, Example 10 are required for extended mobilization, all providing superior mobilization past 6 hours, and Examples 9 and 10 out to 24 hours as shown in FIG. 18, FIG. 24, and FIG. 25. As with the “di-chlorophenyl sulfonamide core”, and due to the limited availability of specific PEG length reagents, it should be apparent from the disclosed data, represented by the Examples 9, 10 and 12, and Comparator Compounds 17, 19 and 20, that robust and extended HSPC mobilization for the “di-chlorobenzoic acid core” requires a PEG length greater than 5 KD MW (˜110 PEG units, Comparator Compound 20), and reasonably somewhere between 5 and 10 KD, but that a minimum of 10 KD (corresponding to n=222-1000 PEG units in the formulas section for the “di-chlorobenzoic acid core”) can be envisioned as a reasonable minimum PEG length to achieve the desired robust and extended HSPC mobilization effect for this core.

As shown herein, these new VLA-4 inhibitors are superior to previously produced molecules (see e.g., U.S. application Ser. No. 16/401,950, incorporated herein by reference in its entirety) in regards to providing more and extended mobilization of hematopoietic stem cells in mice.

Disclosed herein are new compounds and compositions with integrin receptor antagonist properties, methods for their manufacture, and methods for their use, including for the treatment and/or prevention of disease.

### Compounds and Synthetic Methods

The compounds provided by the present disclosure may be made using the methods outlined below and further described in the Examples section. General synthetic sequences for preparing the compounds useful in the present disclosure are outlined in Schemes I-XVII. Both an explanation of, and the actual procedures for, the various aspects of the present disclosure are described where appropriate. The following Schemes, Methods, Descriptions, and Exemplary Embodiments are intended to be merely illustrative of the present disclosure, and not limiting thereof in either scope or spirit. Those with skill in the art will readily understand that known variations of the conditions and processes described in the Schemes and Exemplary Embodiments can be used to synthesize the compounds of the present disclosure. Starting materials and equipment employed were either commercially available prepared by methods previously reported or readily duplicated by those skilled in the art. For illustrative purposes, it should be understood that X₁, as described herein, will often be depicted as a methyl ester throughout the schemes below, but in practice need not be limited to this ester, as other esters or acid derivatives appropriate to the reaction conditions or availability of reagents or otherwise specific preferences can equally be utilized.

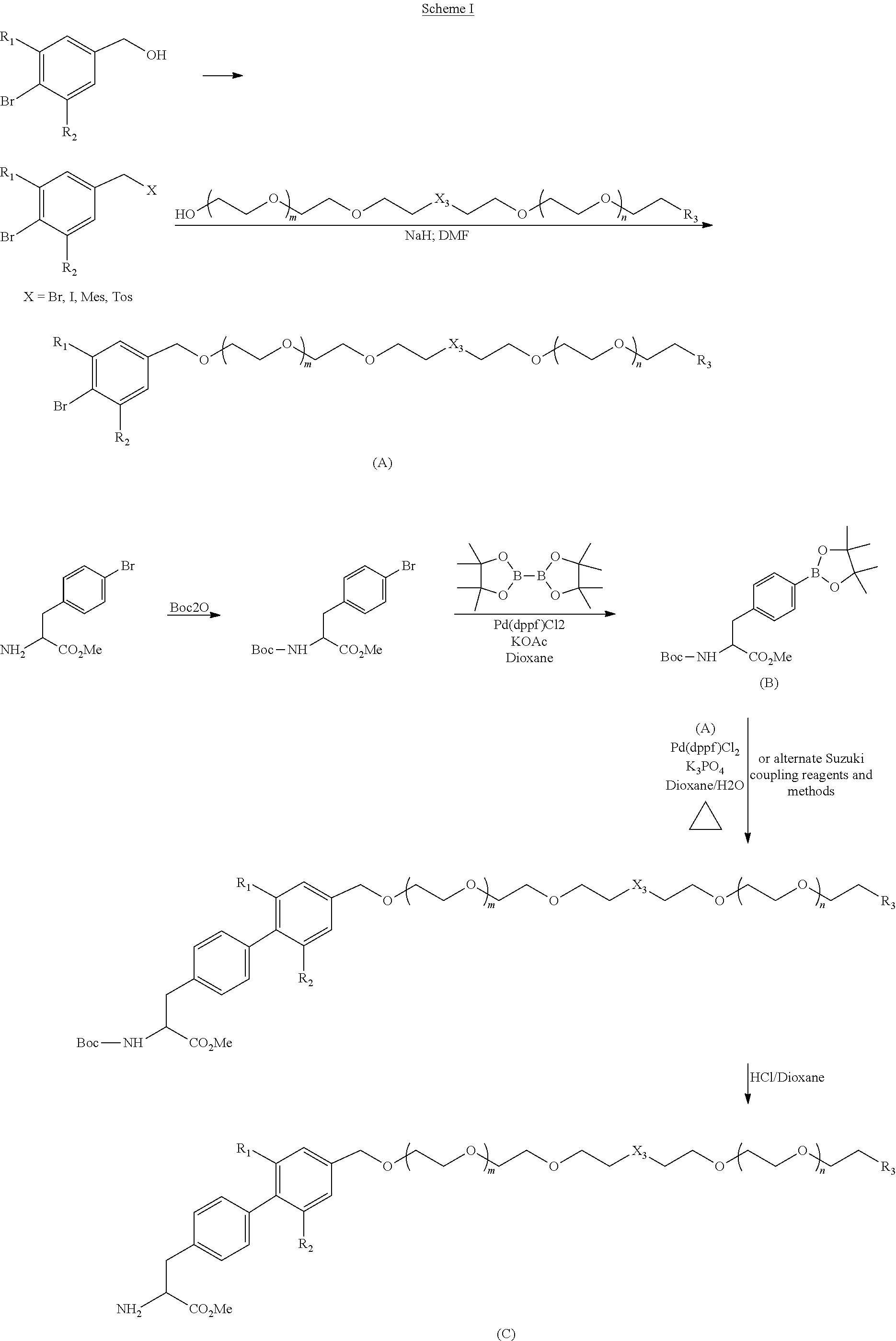

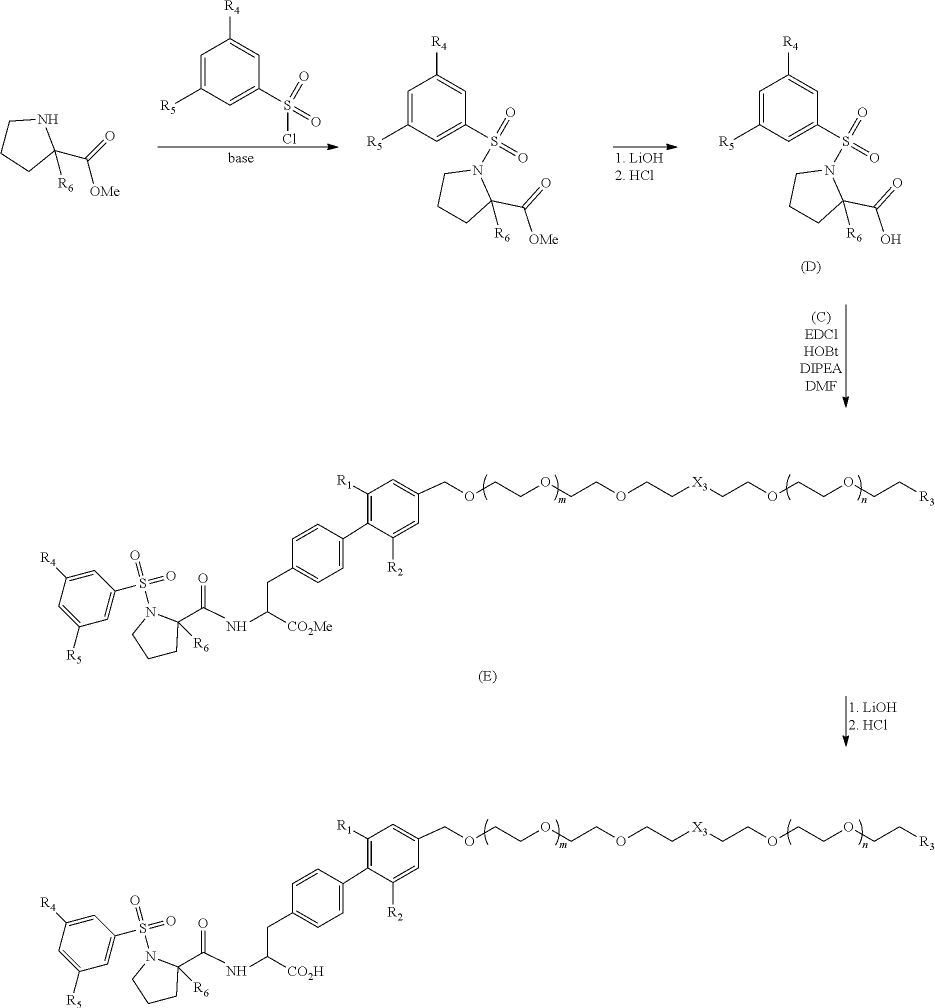

Scheme I represents a general methodology that may be used in the preparation of compounds disclosed herein, where R₁₋₆, X₃, m, and n are as defined herein, and where Z=

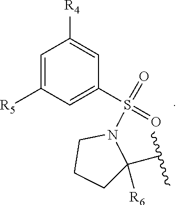

Briefly, the appropriate 4-Bromo, 3,5-di-alkoxy (R₁ and R₂ as defined herein) benzyl alcohol is converted to the corresponding benzyl bromide, or other suitable electrophile leaving group, such as a tosylate, mesylate, or iodine, utilizing reagents commonly known to those skilled in the art. The benzyl bromide is then reacted with the appropriate PEG alcohol under conditions known in the art, such as NaH in anhydrous DMF, or using alternative displacement conditions and reagents, to give intermediate (A).

In the most straightforward and simplest examples, X₃ is oxygen and R₃ is methoxy or another group as defined in the general claims, with m and n also defined as in the general claims. Where X₃ is oxygen and R₃ is not a methoxy, larger alkoxy, or other simple functional group, but consists of a group that requires the addition or coupling of a second functionality to the R₃ terminal end of the PEG alcohol, forming, for example, a PEGylated sulfide or amine at the R₃ position, then such reaction can take place after the now penultimate intermediate (A) is formed. Examples depicting such additional modifications can be illustrated herein by the following:

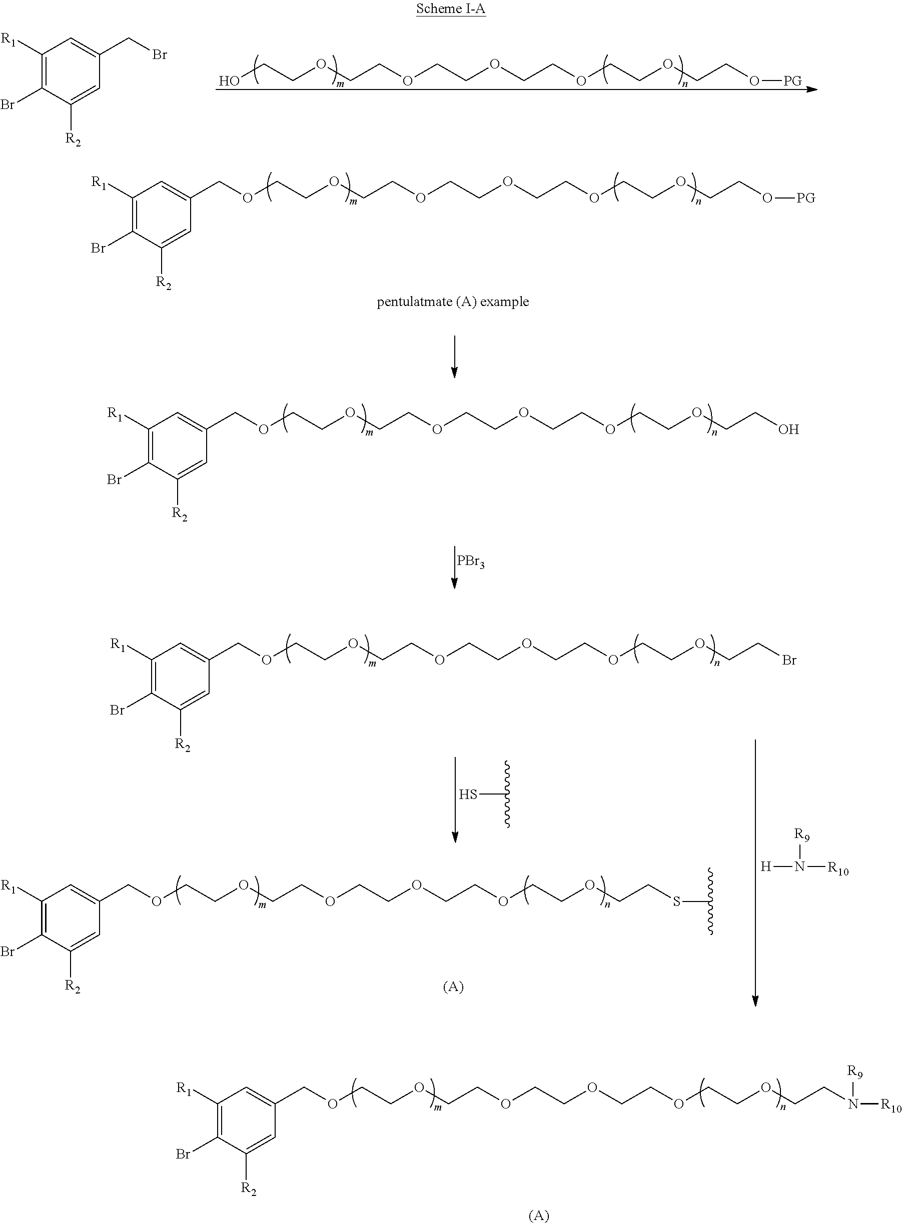

In the generic Scheme I-A, the resultant sulfide or other linking functionality are defined by R₃ in the general claims. Reagents and conditions for forming such coupling groups are standard and known to those skilled in the art. Appropriate protecting groups may be utilized where needed and necessary for efficient reaction outcomes, and in particular in the formation of penultimate Intermediate (A). These more complicated examples for R₃ are not meant to be limiting or constrained by any specific examples depicted in these schemes, and those skilled in the art can appreciate that many potential derivations afforded via the group R₃ can be envisioned, including groups such as long or branched PEG groups conjugated to the terminal end of a penultimate intermediate (A) through various functionalities.

In Scheme I-A, the penultimate (A) example consists of a terminal hydroxyl group protected by an appropriate protecting group, such as a silyl protecting group or similar. The PEG group is attached to the R₁, R₂-bromobenzylic group as described and depicted in the main Scheme I, utilizing the appropriate PEG alcohol with the protected alcohol on the terminal side of the reagent. After removing the hydroxyl protecting group using reagents specific to the removal of the protecting group utilized, the hydroxyl is converted to the bromide or other leaving group, such as a mesylate or tosylate. This can then be reacted with the desired mercaptan or amine, as depicted in the scheme to form Intermediate (A).

These Intermediates (A) with conjugated R₃ functionalities can now be reacted with Intermediate (B) from the Main Scheme I and the reaction sequence continued as depicted in the main Scheme I to form the desired products of this disclosure.

As stated, these example schemes are not meant to be limiting, and those skilled in the art can appreciate similar such reactions using appropriate reagents and reaction conditions to form other such product derivatives at R₃ as defined herein.

Alternatively, further functionalization at R₃, as described and exemplified above, can take place, where feasible, and with appropriate protecting groups, at the end of the entire reaction sequence of Scheme I, as opposed to adding it to penultimate Intermediate (A) and prior to Suzuki coupling of the resultant Intermediate (A) with Intermediate (B).

This alternative method can be depicted as in the generic example of Scheme I-B below:

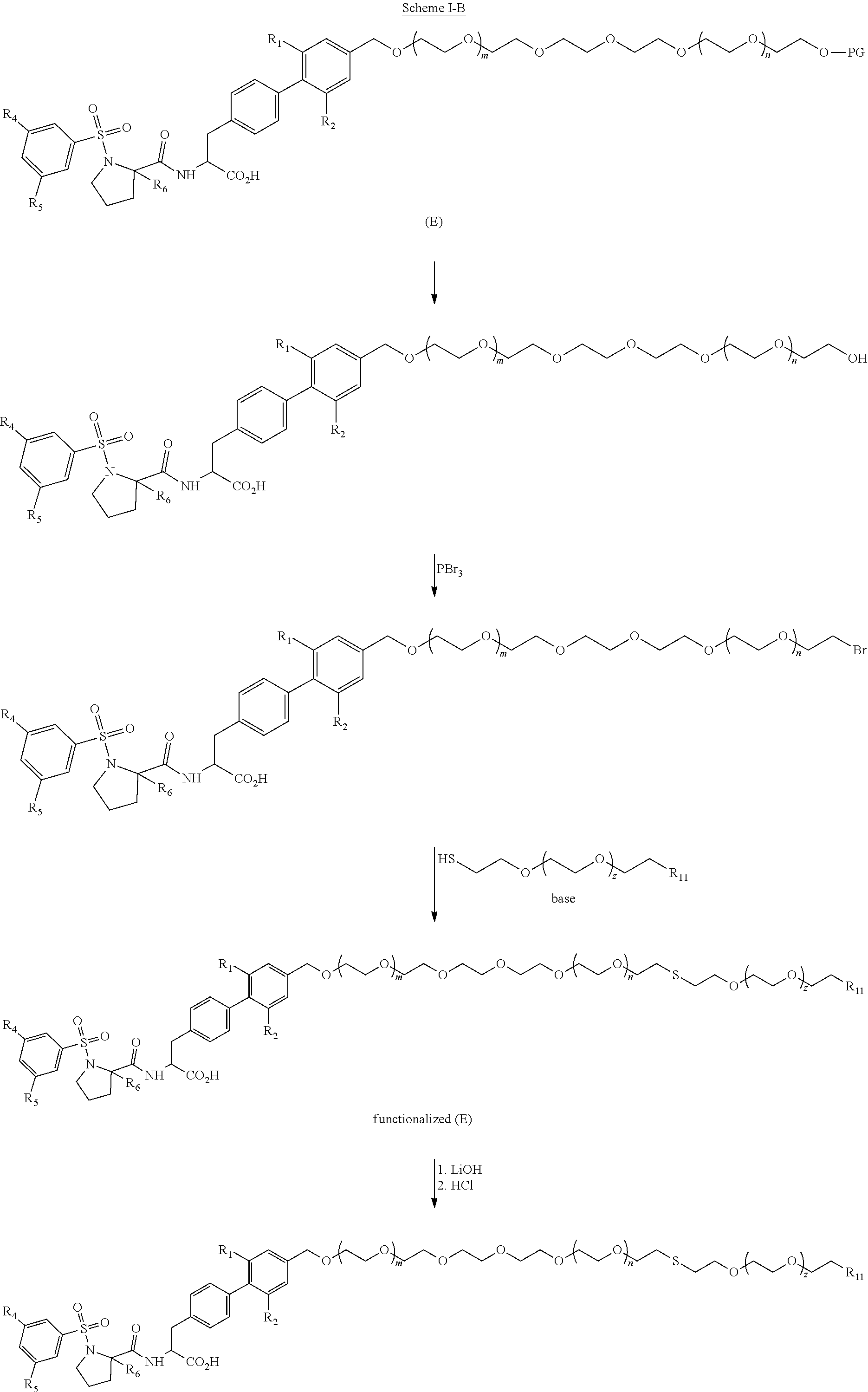

In this example, functionalization at R₃ results in the formation of a sulfide analogue, in this case, a PEG sulfide, knowing that any sulfide, as defined for R₃ as described herein, can be envisioned here.

Briefly, Intermediate (E) is formed via initial reaction of the appropriate PEG diol, with a hydroxyl protecting group at one end, reacted with the appropriate 4-Bromo, 3,5-di-alkoxy (R₁ and R₂) benzyl bromide in the first step of the main Scheme I. Continuing the reaction sequence results in Intermediate (E) in Scheme I-B above, which contains the terminal hydroxyl group, protected with an appropriate protecting group that can withstand all of the reaction conditions encountered throughout the sequence from the main Scheme I outline, such as an appropriate silyl group, or similar. The protecting group is then removed under conditions specific to such protecting group, and that will be known to those skilled in the art. The free alcohol is then converted to a Br, Cl, Iodo, or a mesylate or tosylate, utilizing standard conversion reagents and conditions. In the example above, it is converted to a bromide utilizing PBr₃. This bromide (or other electrophile) is then reacted with the appropriate mercaptan (in this case a PEG mercaptan) to yield the newly functionalized Intermediate (E) as depicted in the scheme above, with z and R₁₁ as defined herein. Hydrolysis of the ester then yields the desired products of this disclosure, in this case, terminal sulfide products.

It can be further envisioned that the example depicted in Scheme I-B is not to be limiting in nature, but can be applied to other functional groups, as defined by R₃, by substituting the appropriately protected terminal group that can then be de-protected and functionalized to provide the desired group defined by R₃, other than the terminal sulfides of Scheme I-B, such as amines, sulfones, etc.

When X₃ is other than oxygen, and is sulfur, amino, or an amide as defined for X₃ in the claims, then the formation of Intermediate (A) can be synthesized stepwise as in the following examples:

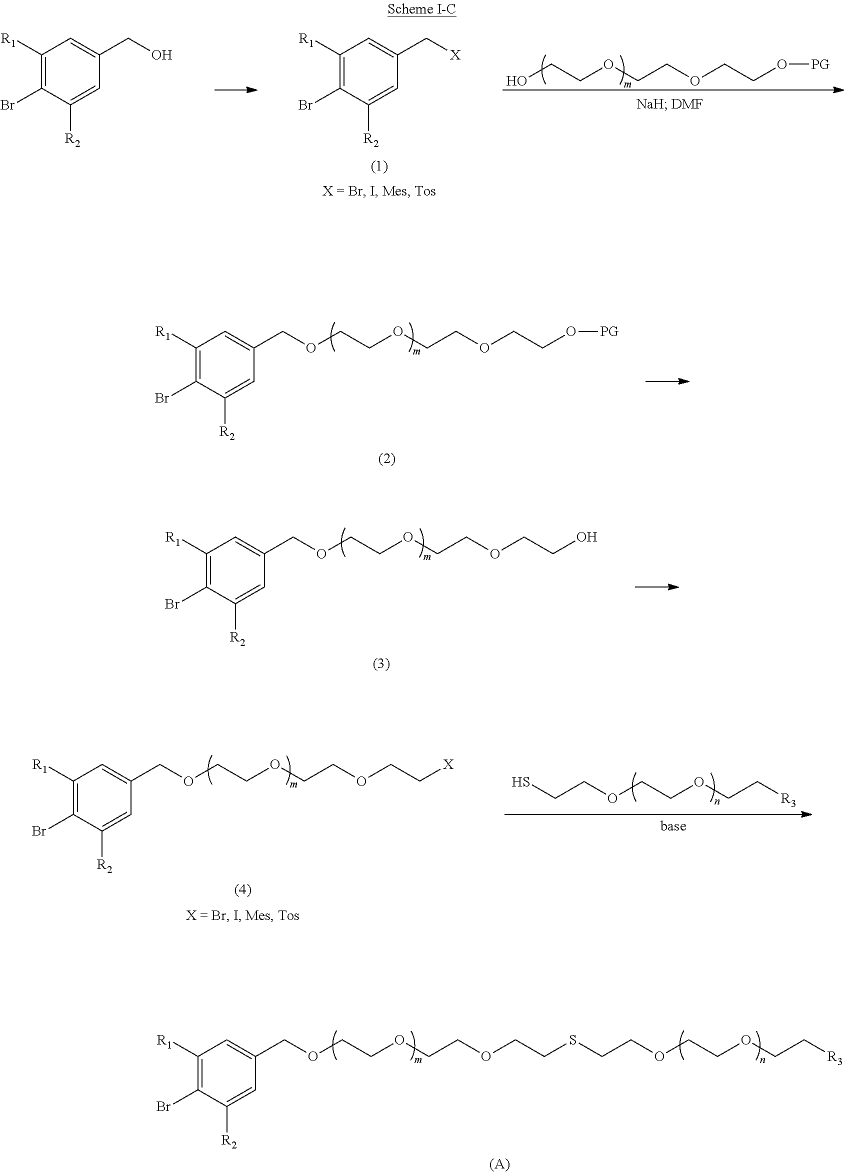

In the general example depicted in Scheme 1-C, X₃ is sulfur, and the initial reaction of the appropriate PEG diol, with m as defined herein, and with one of the terminal hydroxyl groups protected with a suitable protecting group, (such as TMS or other silyl protecting group, or a different suitable protecting group), is reacted with the benzyl halide, mesylate or tosylate (1) to form the terminally protected PEG intermediate (2). After de-protection using standard de-protection reagents and conditions known to those skilled in the art, alcohol intermediate (3) is formed. The terminal alcohol is then converted to a terminal halide, mesylate, or tosylate intermediate (4), using standard reagents and conditions known to those skilled in the art, for example, using PBr₃ if converting to the bromide. This intermediate (4) is then reacted with the appropriate PEG thiol under basic conditions, using reagents common for such nucleophilic displacement reactions, to form Intermediate (A) as above, with R₁, R₂, m, n, and R₃ as defined herein. This Intermediate (A) can then continue on with the next steps in the main Scheme I to yield the products as depicted in this disclosure.

In its simplest form, R₃ is methoxy or a larger alkoxy. Where R₃ is not a methoxy, larger alkoxy, or other simple functional group, but consists of a group that requires the addition or coupling of a second functionality to the R₃ terminal end of the PEG alcohol, forming, for example, an amide, sulfonamide, etc. at the R₃ position, then such reaction can take place as described and depicted above when X₂ is oxygen, as in Schemes I-A and I-B.

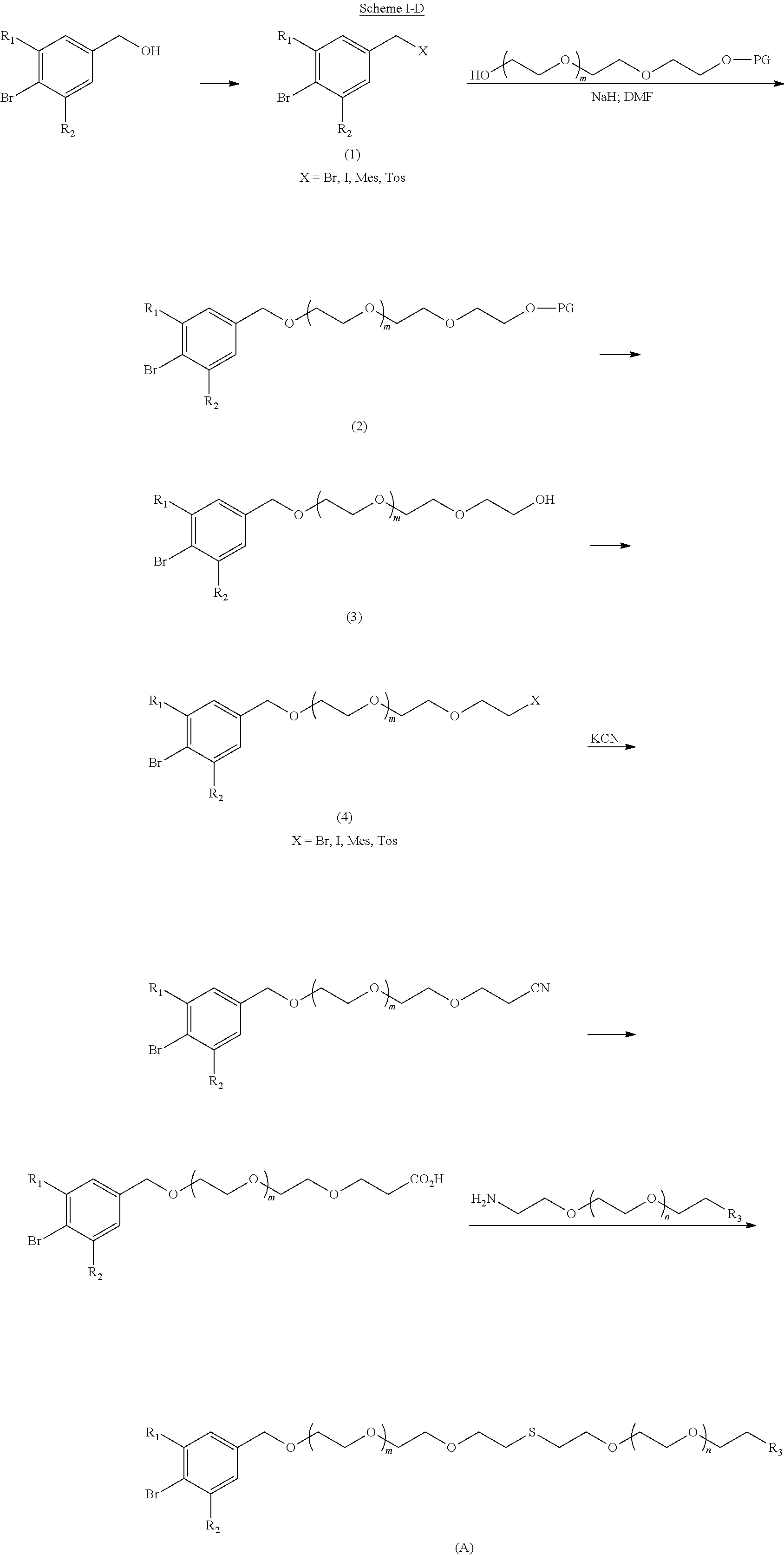

Scheme I-D represents a generic example of a synthetic sequence where X₃ is represented by an amide. As in Scheme I-C, the appropriate PEG diol, with m as defined herein, and with one of the terminal hydroxyl groups protected with a suitable protecting group, (such as TMS or other silyl protecting group, or a different suitable protecting group), is reacted with the benzyl halide, mesylate or tosylate (1) to form the terminally protected PEG intermediate (2). After de-protection using standard de-protection reagents and conditions known to those skilled in the art, alcohol intermediate (3) is formed. The terminal alcohol is then converted to a terminal halide, mesylate, or tosylate intermediate (4), using standard reagents and conditions known to those skilled in the art, for example, using PBr₃ if converting to the bromide. This intermediate (4) is then reacted with potassium or sodium cyanide under appropriate reaction conditions known to those skilled in the art, to form a terminal nitrile. This nitrile is then converted to the corresponding carboxylic acid under conditions known to those skilled in the art, such as an aqueous acid, like HCl, at elevated temperatures. After formation, the carboxylic acid is then coupled with an appropriate amine under standard coupling reaction conditions, such as EDCl and HOBt, or similar coupling reagents known to those skilled in the art, to from the desired amide Intermediate (A), as depicted in Scheme I-D, with m, n, and R₃ as defined herein.

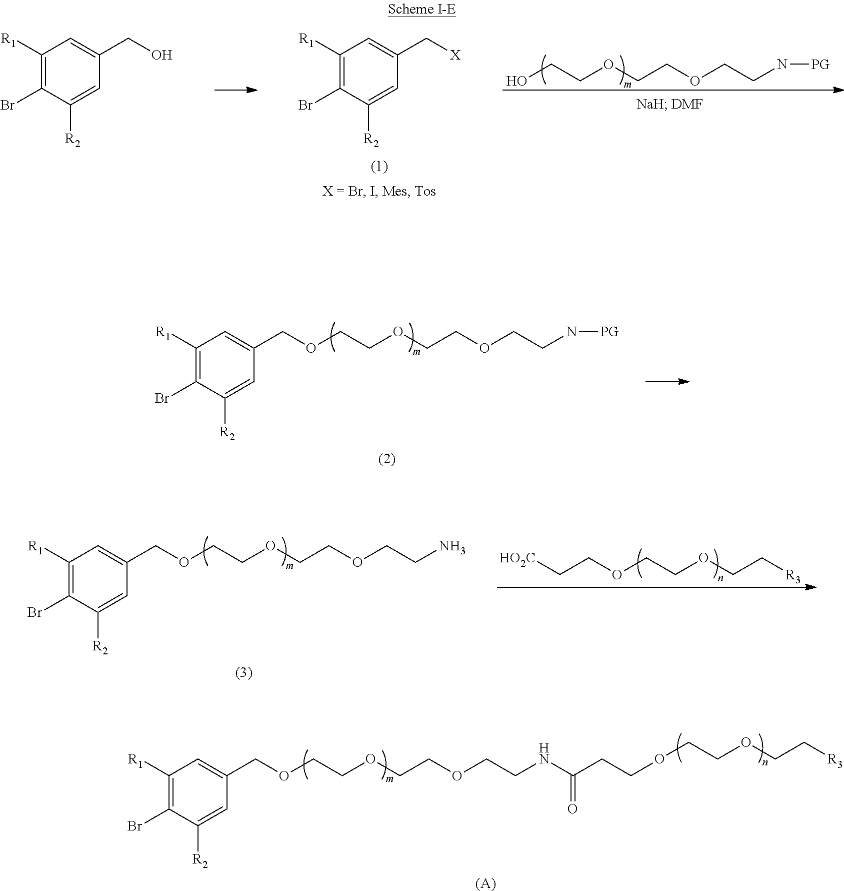

Scheme I-E depicts an example where X₂ is represented by a reverse amide to the X₃ amide of Scheme I-D. In this example, an appropriately N-protected PEG alcohol is reacted with Intermediate (1) to yield the N-protected Intermediate (2). After de-protection of the amine using reagents appropriate for the particular protecting group utilized, the amine Intermediate (3) is then coupled with the appropriate PEG acid under standard coupling reaction conditions, such as EDCl and HOBt, or similar coupling reagents known to those skilled in the art, to from the desired amide Intermediate (A), as depicted in Scheme I-E, with m, n, and R₃ as defined herein.

Separate to the formation of Intermediate (A) in the main Scheme I and in the penultimate intermediates (A) (generic examples of which are depicted in Schemes I-A, B, C, D, and E), boronic pinacol ester, Intermediate B, is formed from 4-bromo phenylalanine methyl ester after addition of a Boc protecting group to the amine. Intermediate B is formed via a Suzuki coupling reaction of the Boc protected 4-bromo phenylalanine methyl ester with Bis(pinacolato)diboron.

Intermediate B is then reacted with Intermediate A from above, via another Suzuki coupling, to form, after acidic de-protection of the Boc amine, Intermediate C. Various reagents and experimental conditions known to those skilled in the art can be utilized to conduct these Suzuki coupling reactions, such as the palladium catalyst and reagents depicted in Scheme I.

Separately, prolyl sulfonamide Intermediate D is formed by reaction of the appropriate R₄ and R₅ (as defined herein), containing sulfonyl chloride with the appropriate prolyl ester, followed by hydrolysis to the free acid.

Intermediate D is then reacted with Intermediate C, using standard amide forming coupling reagents and experimental procedures known to those skilled in the art, such as HOBt, EDCl in DMF, or DMA with a base such as DIEA, or similar. This reaction results in ester Intermediate E.

Finally, hydrolysis of Intermediate E leads to the formation of the desired integrin inhibitors as claimed within this disclosure.

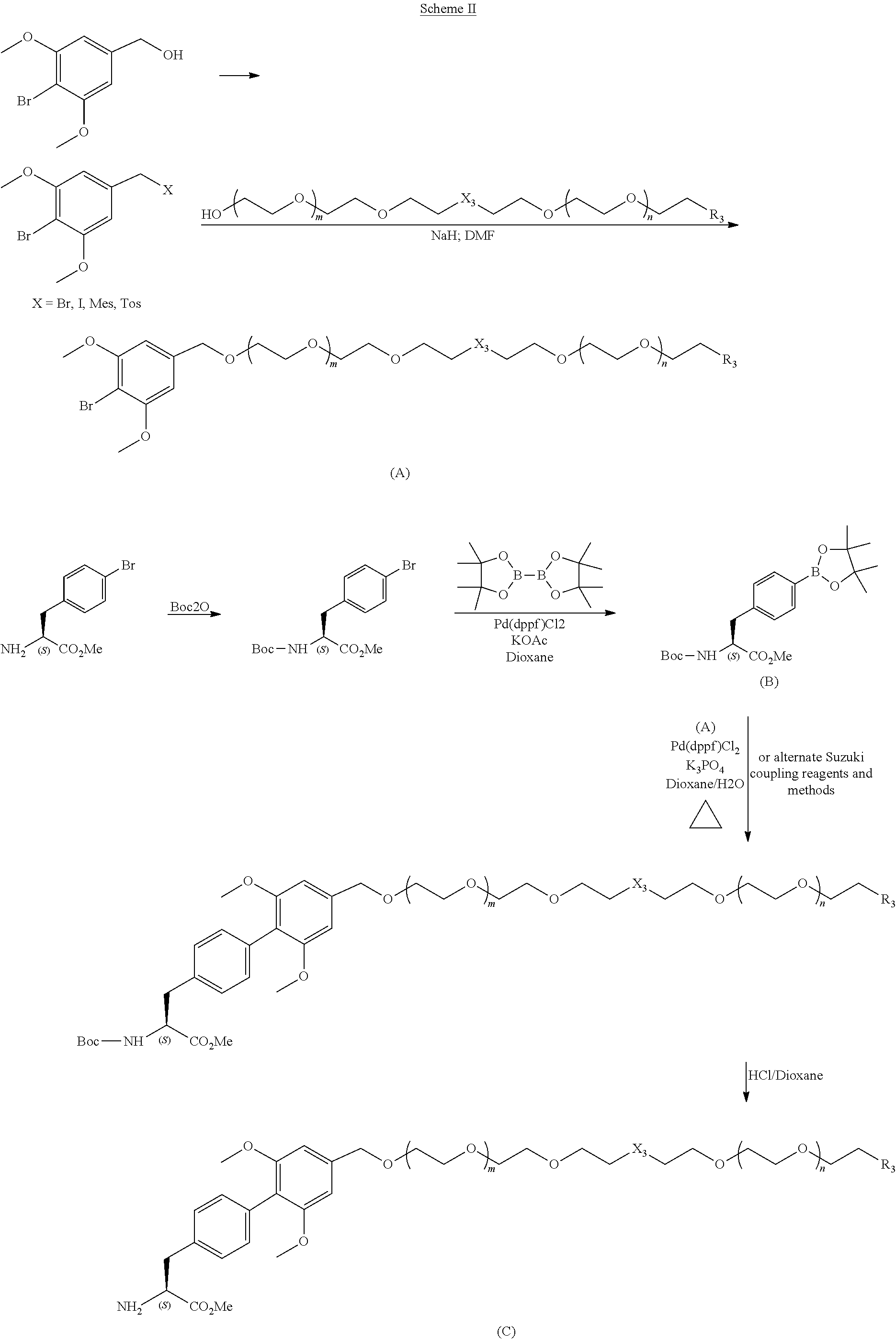

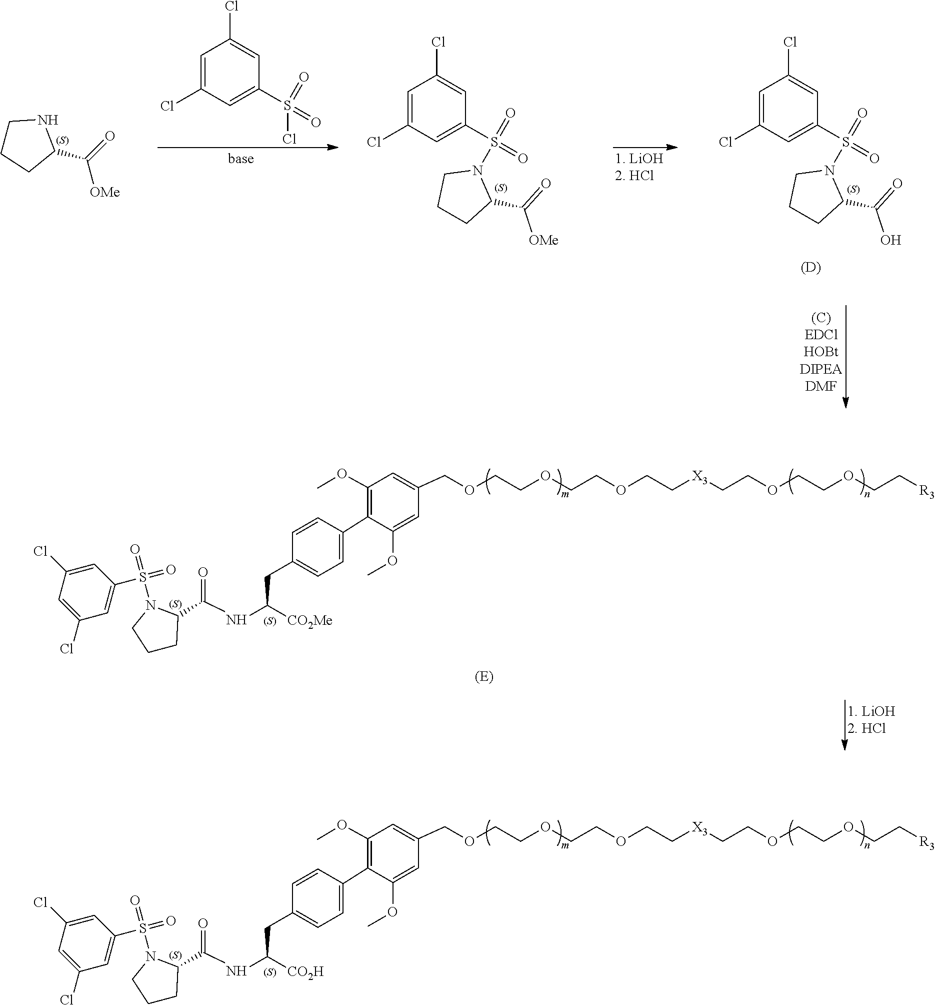

Scheme II depicts a general method for the synthesis of preferred embodiments contained within this disclosure. The methods and descriptions relevant to the synthesis of examples as depicted in Scheme II are the same as detailed in Scheme I and in the sub-Schemes I-A-E above, with R₁ and R₂ specified here as methoxy, R₄ and R₅ specified as chloro, R₆ specified as hydrogen, and with the (S) stereochemistry preferred, and as drawn accordingly in Scheme II above. X₃, m, n, and R₃ are as defined herein.

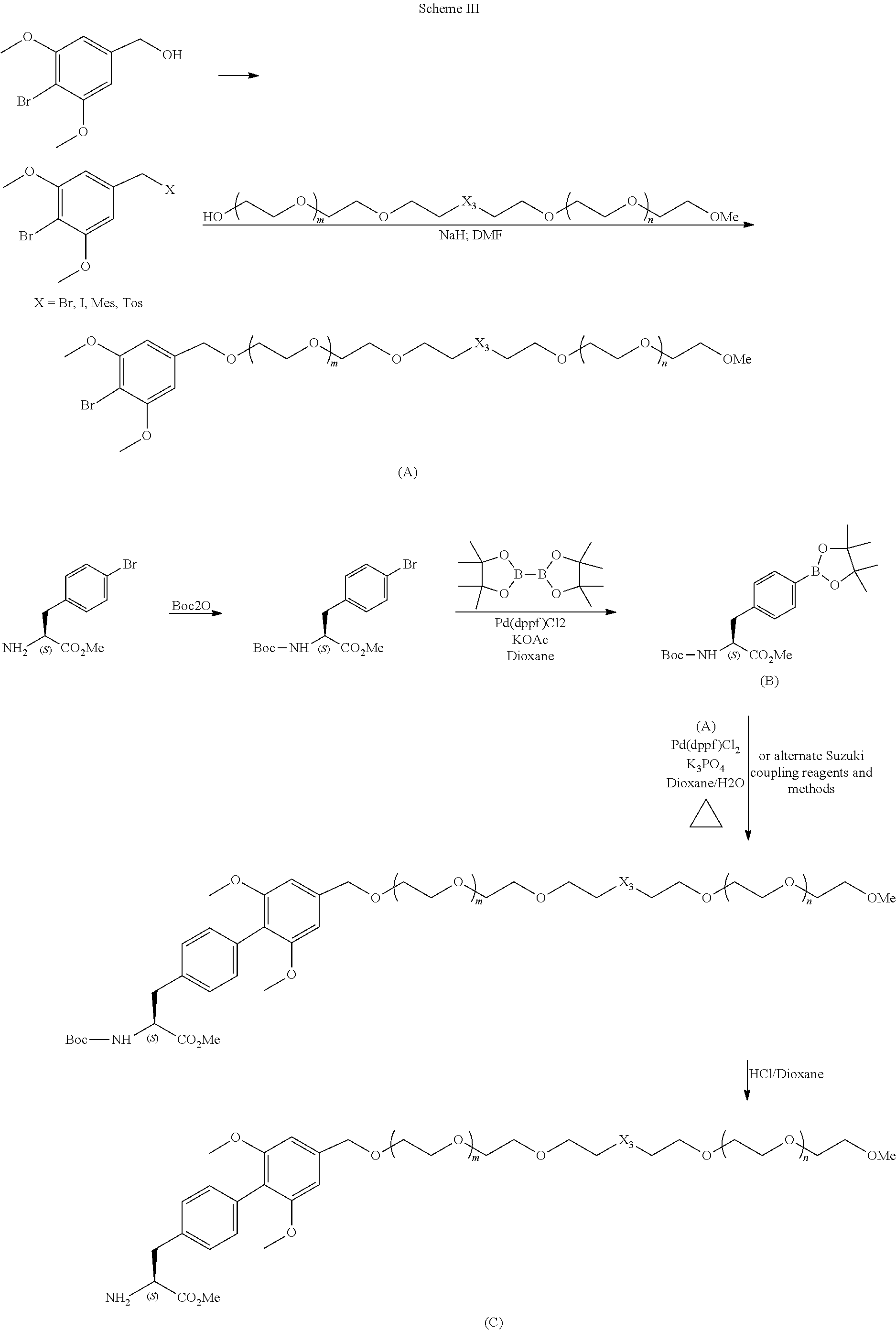

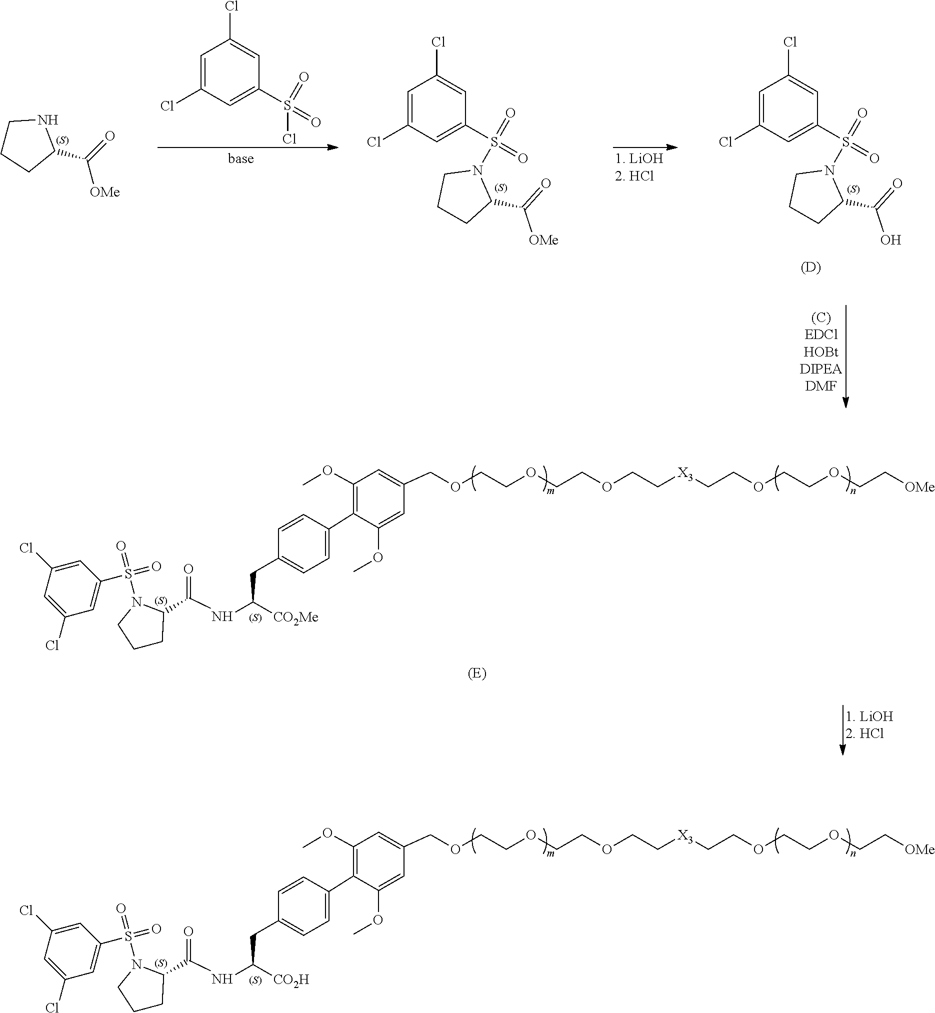

Scheme III depicts a general method for the synthesis of additional preferred embodiments contained within this disclosure. The methods and descriptions relevant to the synthesis of examples as depicted in Scheme III are the same as detailed in Scheme I and in the sub-Schemes I-A-E above, with R₁, R₂ and R₃ specified here as methoxy, R₄ and R₅ specified as chloro, R₆ specified as hydrogen, X₃ specified as oxygen or sulfur, and with the (S) stereochemistry preferred, and as drawn accordingly in Scheme III above. m and n are as defined herein.

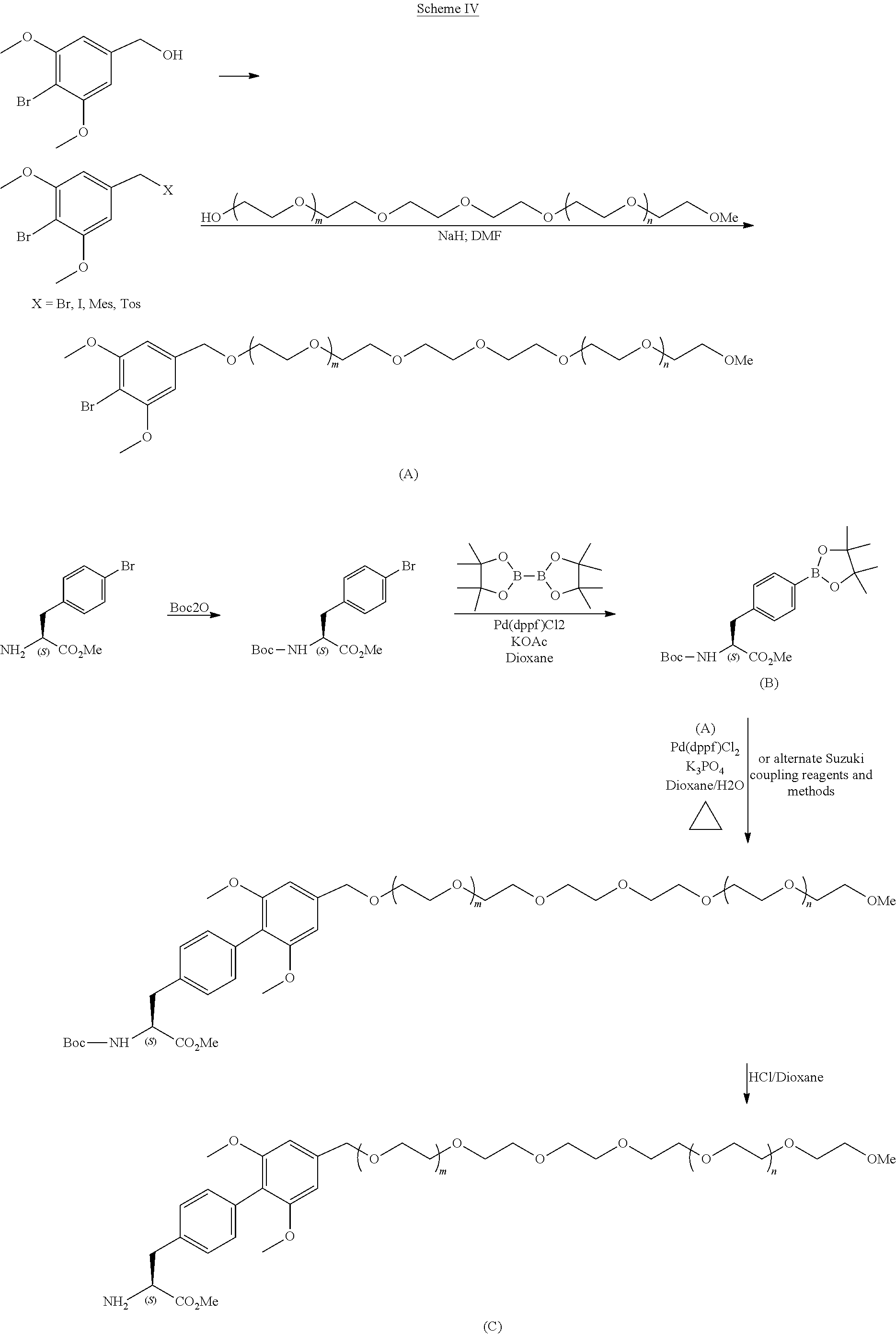

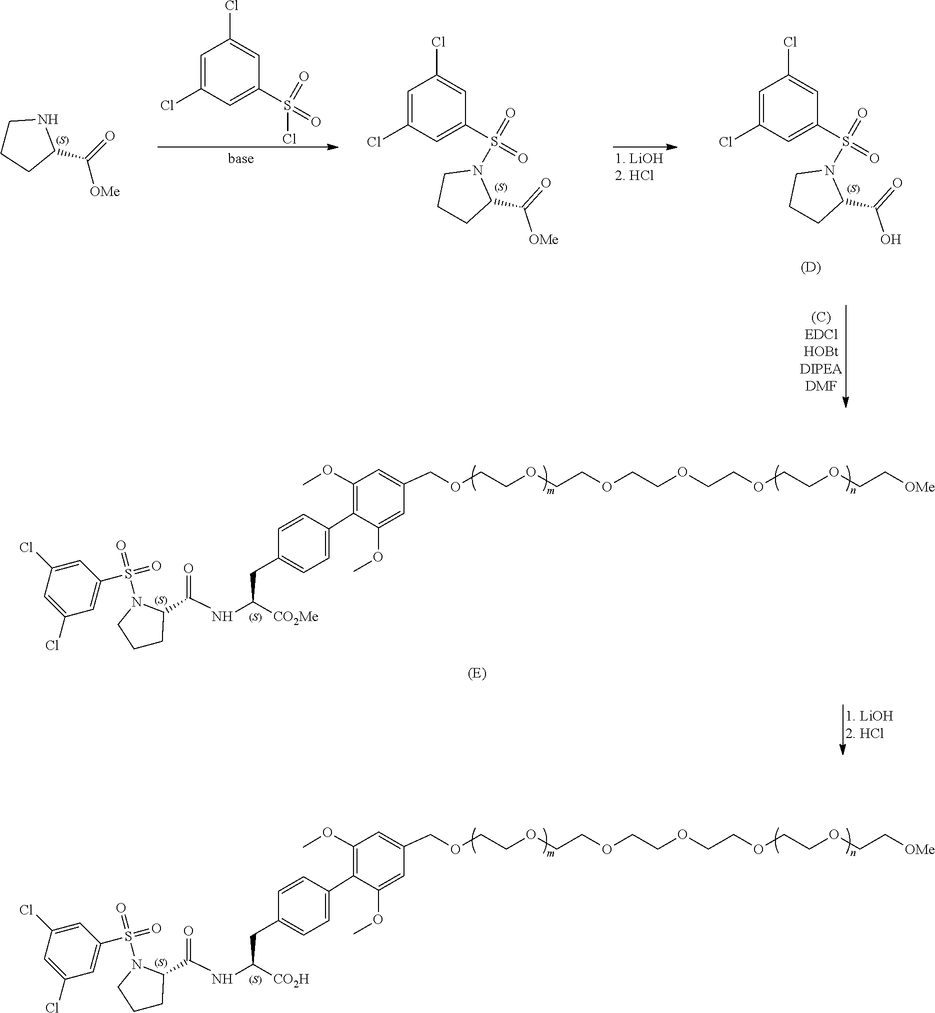

Scheme IV depicts a general method for the synthesis of additional preferred embodiments contained within this disclosure. The methods and descriptions relevant to the synthesis of examples as depicted in Scheme IV are the same as detailed in Scheme I and in the sub-Schemes I-A-E above, with R₁, R₂ and R₃ specified here as methoxy, R₄ and R₅ specified as chloro, R₆ specified as hydrogen, X₃ specified as oxygen, where m=1 and n=19-31, and with the (S) stereochemistry preferred, and as drawn accordingly in Scheme IV above.

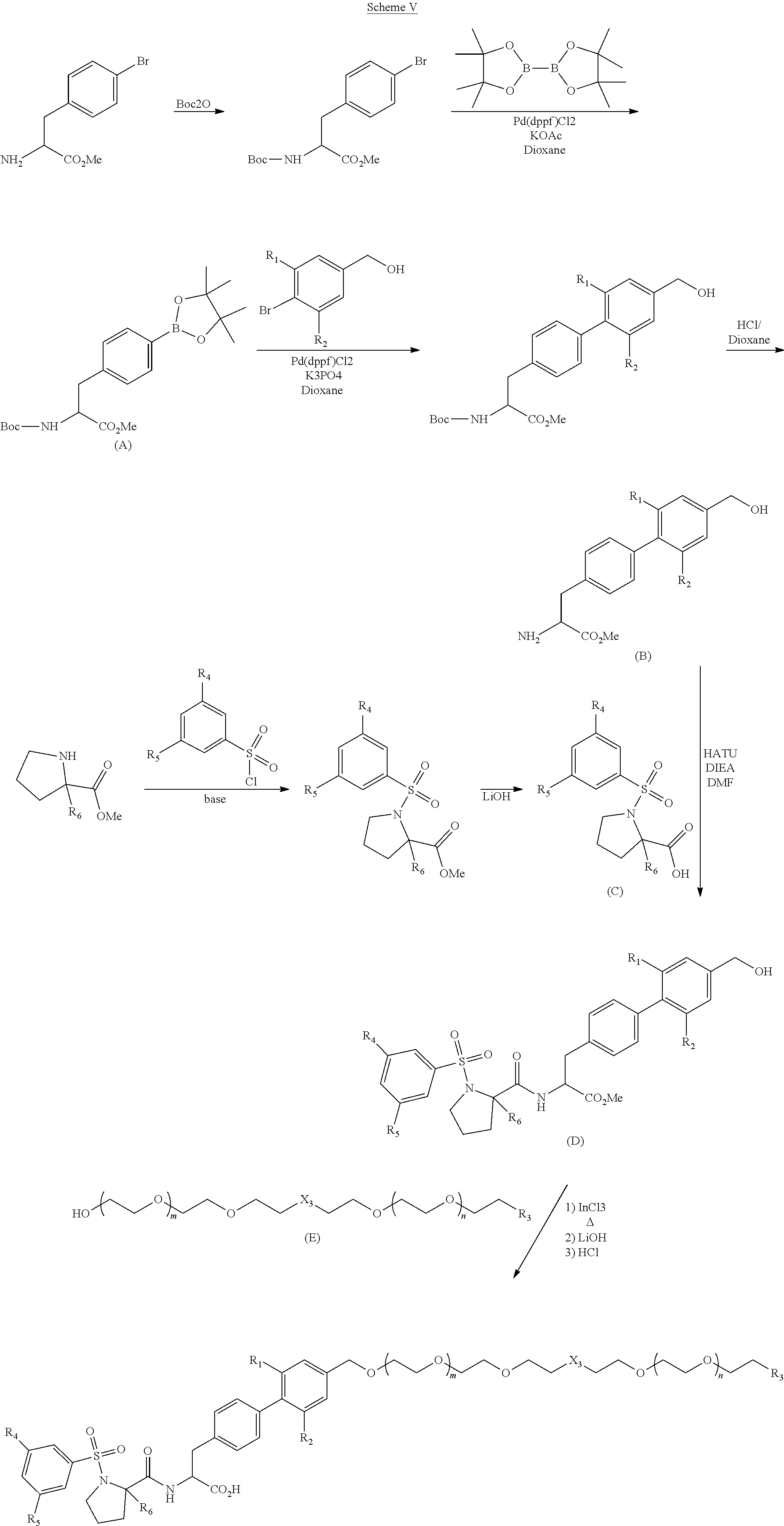

Scheme V depicts a general method for the synthesis of compounds of this disclosure as an alternative to the methods described in Schemes I-IV above. In this general method, Intermediate (D) of Scheme V can act as a common intermediate useful for the addition of multiple PEG “tail” groups, as defined in the general claims, without having to undergo multiple steps after the addition of each new PEG tail group, as depicted in Schemes I-IV. The method of Scheme V would consist of one, or minimal steps once the common intermediate (D) has been synthesized to arrive at the desired products of this disclosure. Such synthetic efficiency can be appreciated by those skilled in the art.

Briefly, the methods, reagents and reaction conditions utilized to synthesize Intermediates (A), (B), (C), and finally common Intermediate (D) are depicted in the descriptions and outlines of Schemes I-IV. Once Intermediate (D) is in hand, it can be reacted with the appropriate PEG alcohol depicted by Intermediate (E) and InCl₃ at elevated temperatures to yield the desired product or as the ester, which can be hydrolyzed to the free acid using reagents and conditions known to those skilled in the art. Often, the reaction of (D) and (E) with InCl₃ at elevated temperatures simultaneously converts to the free acid and no additional step is required.

This general reaction scheme is meant to provide an example of alternate steps and conditions to Schemes I-IV, but not to be limiting in nature. It can be appreciated that those skilled in the art can envision similar and related methods and conditions to arrive at products as detailed in the general claims of this disclosure, and with R₁₋₆, X₃, m, and n as defined within these claims.

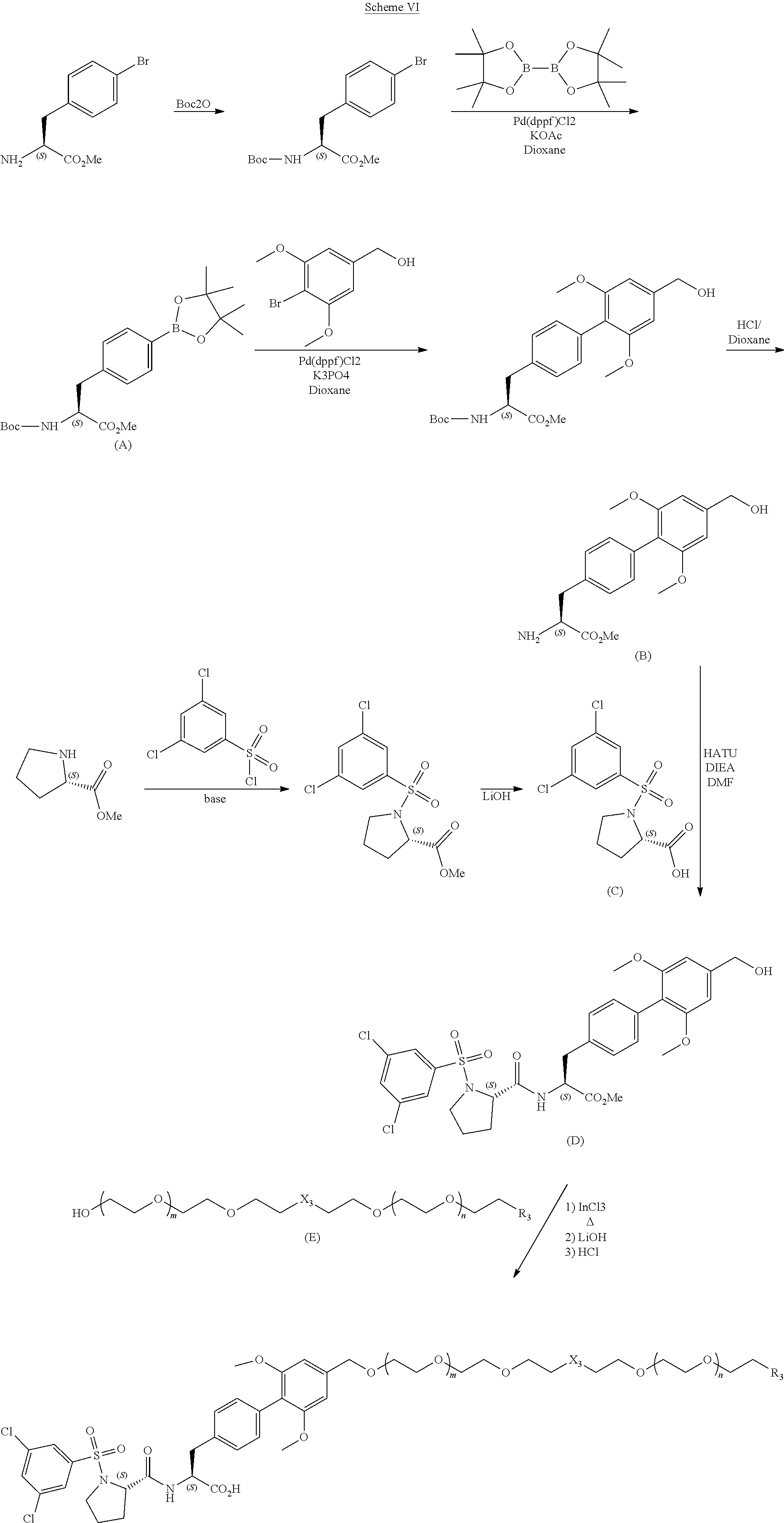

Scheme VI depicts a general method for the synthesis of preferred embodiments contained within this disclosure utilizing the alternate method as depicted in Scheme V above. The methods and descriptions relevant to the synthesis of examples as depicted in Scheme VI are the same as detailed in Scheme V, using Intermediate (D) as a common intermediate, and with R₁ and R₂ specified here as methoxy, R₄ and R₅ specified as chloro, R₆ specified as hydrogen, and with the (S) stereochemistry preferred, and as drawn accordingly in Scheme VI above. X₃, m, n, and R₃ are as defined herein.

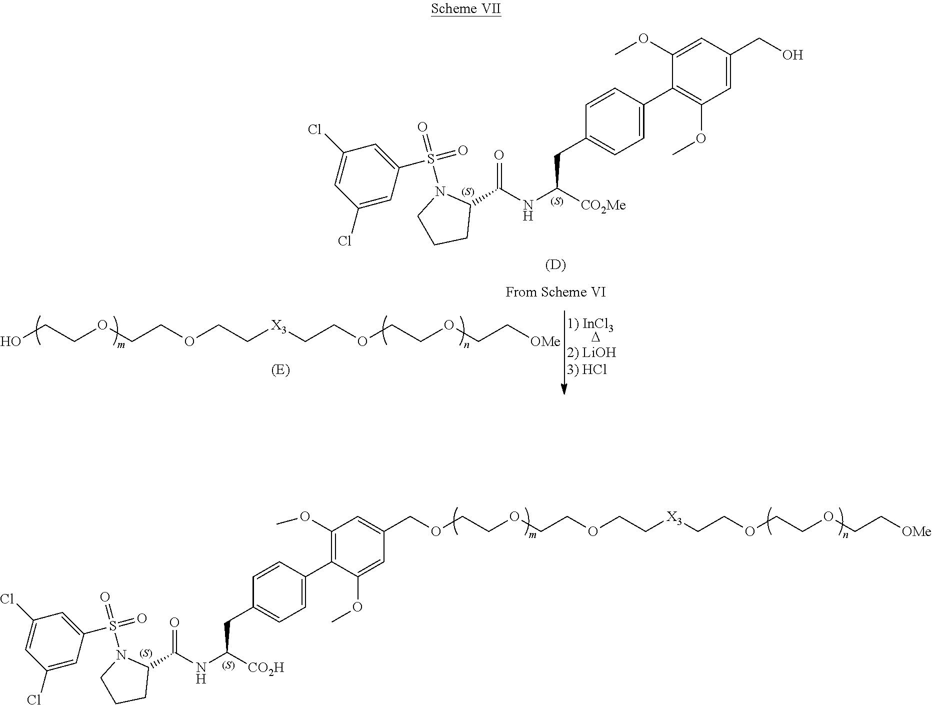

Scheme VII depicts a general method for the synthesis of additional preferred embodiments contained within this disclosure utilizing the alternate method as depicted in Scheme V above. The methods and descriptions relevant to the synthesis of examples as depicted in Scheme VII are the same as detailed in Scheme V, using Intermediate (D) as a common intermediate, and with R₁, R₂ and R₃ specified here as methoxy, R₄ and R₅ specified as chloro, R₆ specified as hydrogen, and with the (S) stereochemistry preferred, and as drawn accordingly in Scheme VII above. X₃, m, and n are as defined herein.

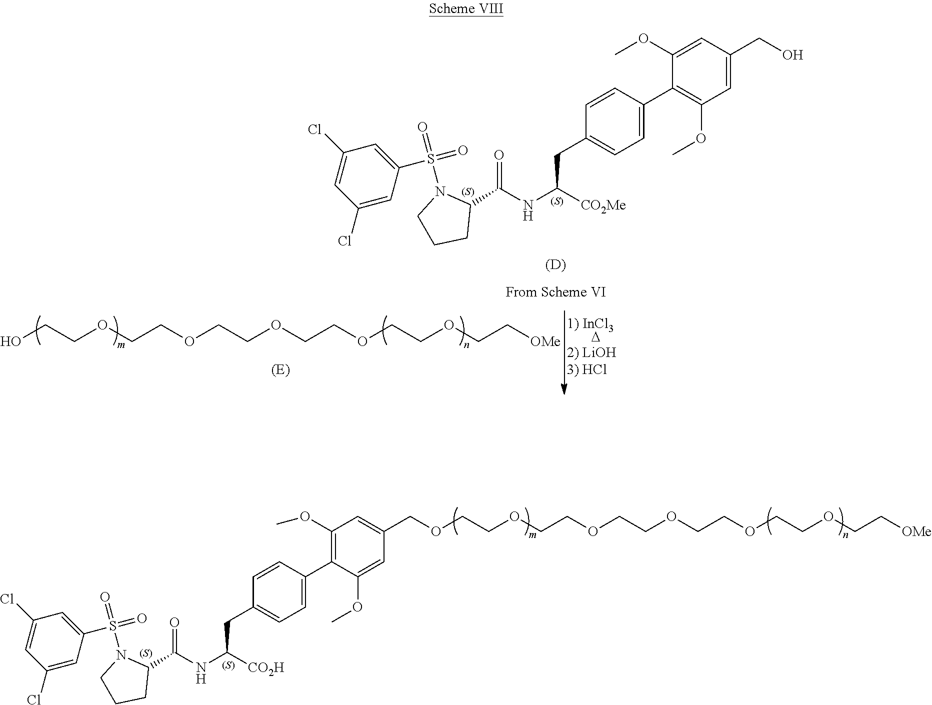

Scheme VIII depicts a general method for the synthesis of further preferred embodiments contained within this disclosure utilizing the alternate method as depicted in Scheme V above. The methods and descriptions relevant to the synthesis of examples as depicted in Scheme VII are the same as detailed in Scheme V, using Intermediate (D) as a common intermediate, and with R₁, R₂ and R₃ specified here as methoxy, R₄ and R₅ specified as chloro, R₆ specified as hydrogen, X₃ as oxygen, and with the (S) stereochemistry preferred, and as drawn accordingly in Scheme VIII above, and with m, and n as defined herein.

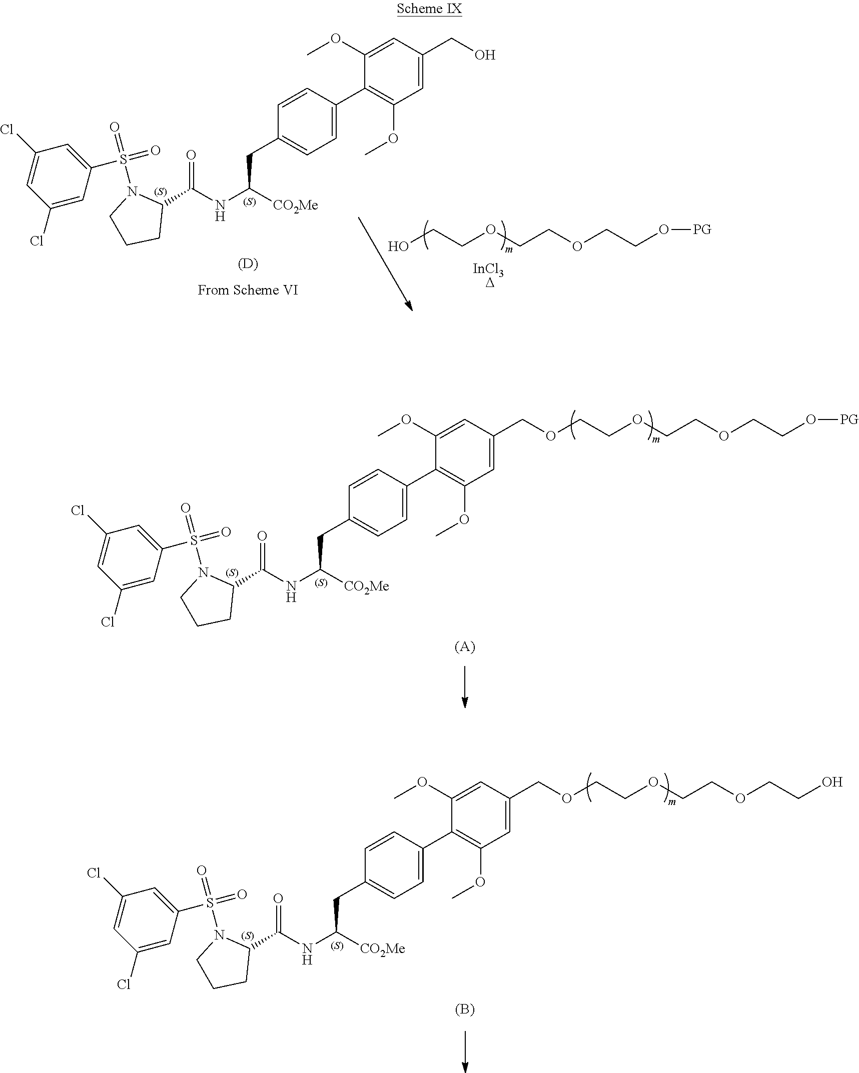

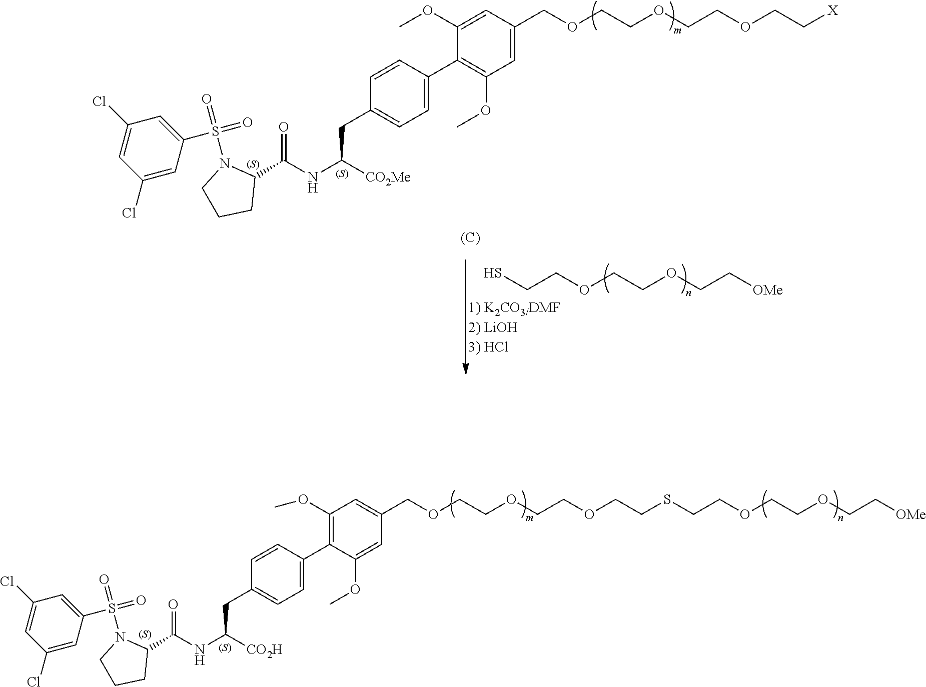

Scheme IX depicts a general method for the synthesis of additional preferred embodiments contained within this disclosure utilizing the alternate method as depicted in Scheme V above. In Scheme IX, X₃ is sulfur and Scheme IX represents a general example of how the insertion of sulfur into the “PEG tail” portion of the desired compound can be realized. Briefly, the appropriate PEG diol, with one of the hydroxyl groups protected with an appropriate protecting group, is reacted with Intermediate (D) and InCl₃ to yield Intermediate (A) in Scheme IX. Removal of the protecting group with an appropriate reagent specific to that protecting group leaves Intermediate (B), as the terminal alcohol. Intermediate (B) is then converted to Intermediate (C), where X is Br, I, mesylate or tosylate, using reagents and conditions known to those skilled in the art and appropriate to the nature of intermediate (B). Intermediate (C) is then reacted with the appropriate PEG mercaptan under appropriate basic conditions to yield, after hydrolysis of the ester to the free acid, the desired product, and with R₁, R₂ and R₃ specified here as methoxy, R₄ and R₅ specified as chloro, R₆ specified as hydrogen, X₃ as sulfur, and with the (S) stereochemistry preferred, and as drawn accordingly in Scheme IX above, and with m, and n as defined herein.

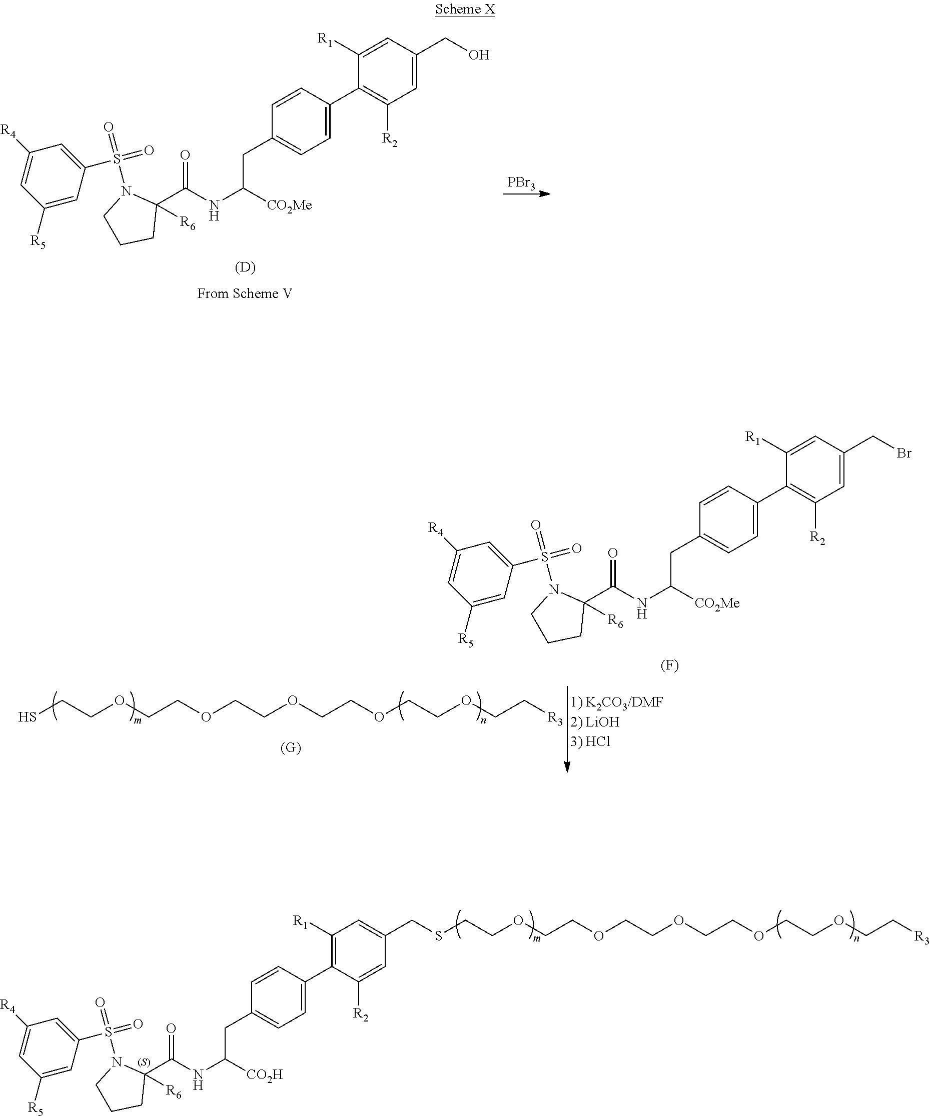

Scheme X depicts a general method for the synthesis of compounds of this disclosure where X₂ is sulfur. Briefly, generic intermediate (D), the method of which is depicted in Scheme V, is reacted with PBr₃, utilizing reagents and methods known to those skilled in the art, to form benzyl bromide (F). Alternatively, other leaving groups such as a mesylate, tosylate, or iodide can be substituted for bromide, utilizing reagents and methods known to those skilled in the art. Intermediate (F) is then reacted with the appropriate PEG mercaptan with a base such as K₂CO₃, or another suitable base compatible with the reactants and reaction conditions employed, to yield the desired thio PEG analogue as the methyl ester. Hydrolysis of the ester with LiOH or other suitable base followed by quenching with acid yields the desired product as the free acid. R₁₋₆, m, and n are as defined herein.

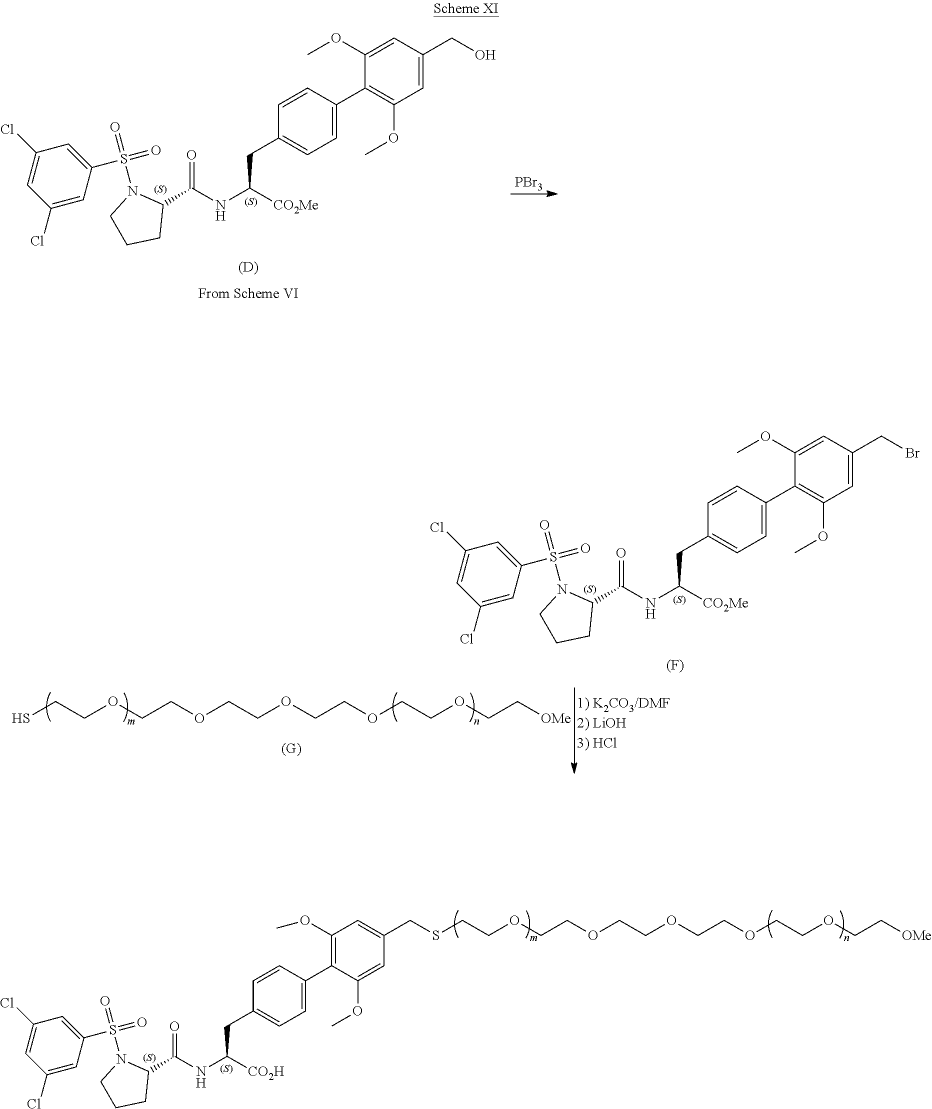

Scheme XI depicts a general method for the synthesis of preferred embodiments contained within this disclosure where X₂ is sulfur. The methods are the same as detailed in Scheme X, and with R₁, R₂ and R₃ specified here as methoxy, R₄ and R₅ specified as chloro, R₆ specified as hydrogen, X₃ is oxygen, and with the (S) stereochemistry preferred, and as drawn accordingly in Scheme XI above. m and n are as defined herein.

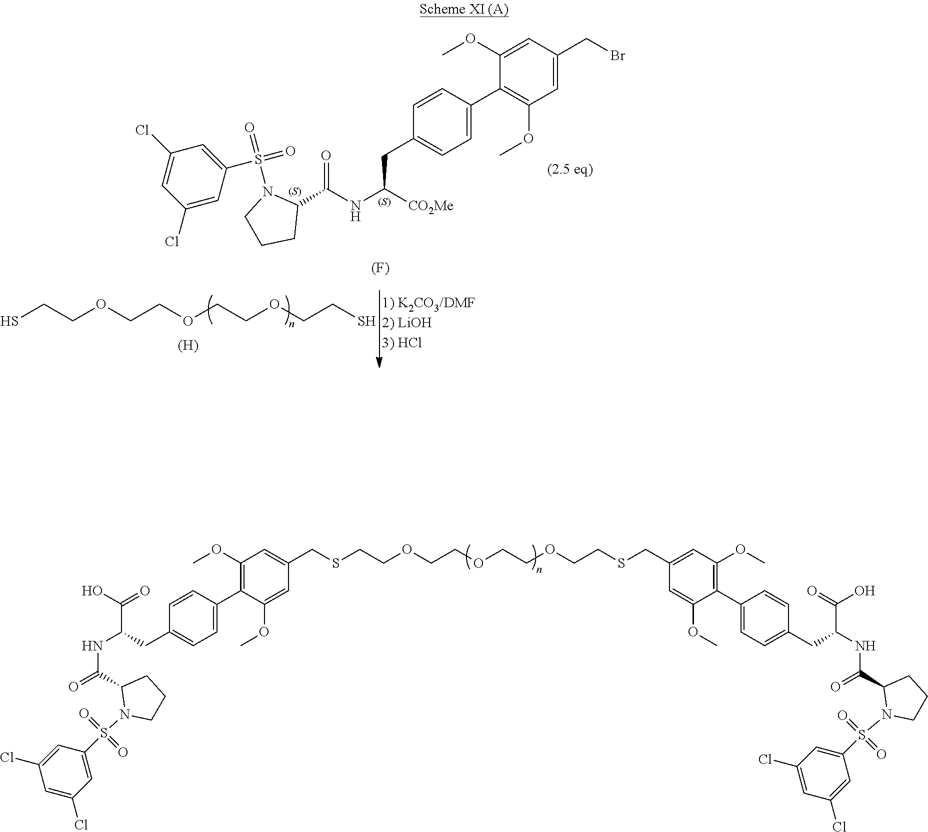

Scheme XI (A) depicts a general method for the synthesis of preferred embodiments contained within this disclosure where X₂ is sulfur and the final product is a bis-VLA4 inhibitor product linked via a PEG linker. The methods are the same as detailed in Scheme XI, with the exception that intermediate (H) is a bis-mercaptan PEG compound, as depicted, and starting with 2.5 equivalents of benzyl bromide (F).

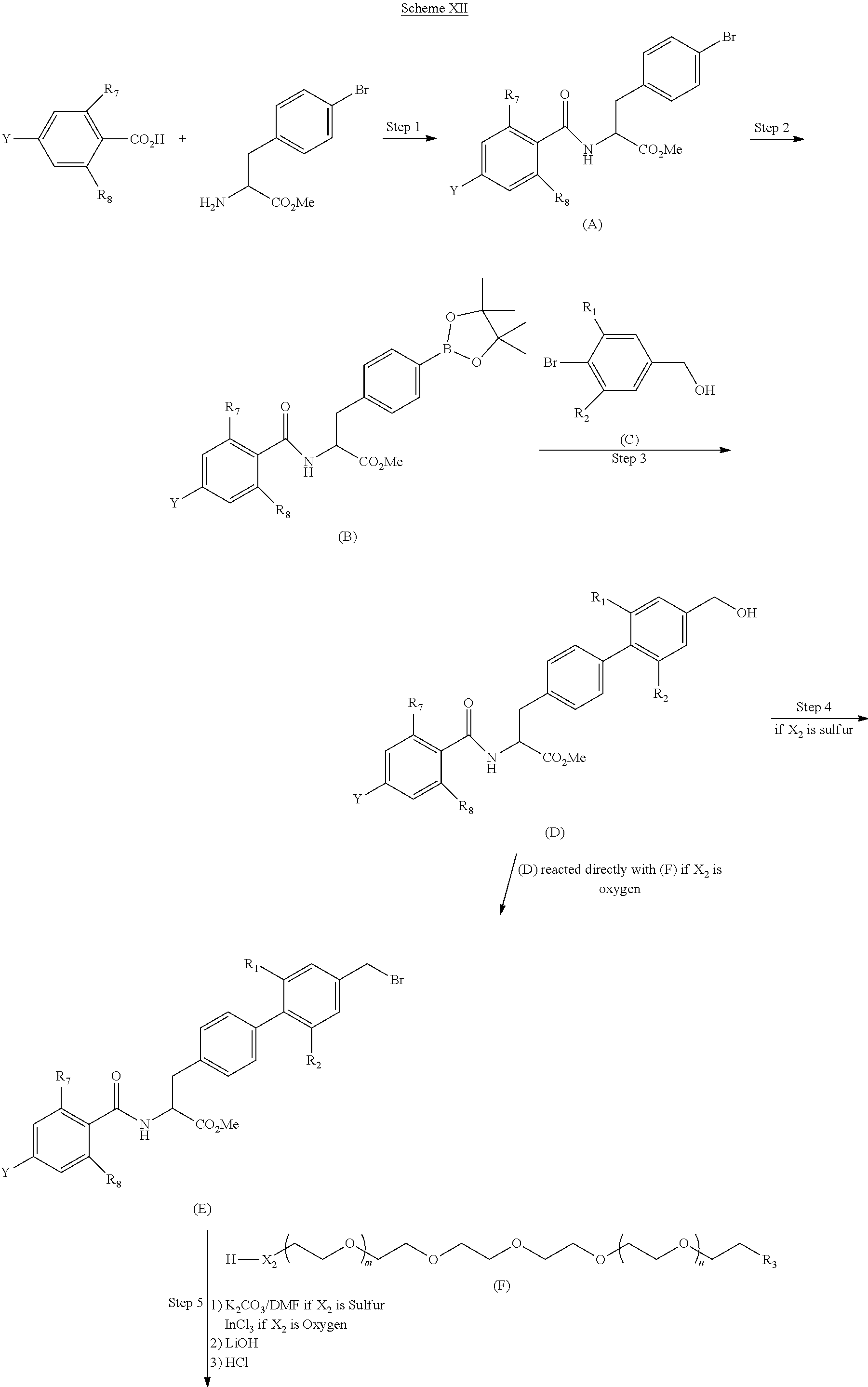

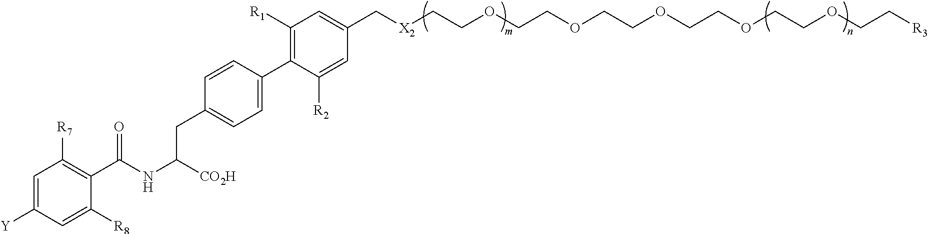

Scheme XII depicts a general method for the synthesis of compounds of this disclosure where

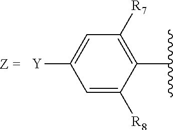

Briefly, the appropriately substituted benzoic acid is reacted with 4-bromo phenylalanine methyl ester utilizing coupling reagents and reaction conditions known to those skilled in the art to form the amide (A) depicted in Step 1 above. Y, R₇ and R₈ are as defined herein. In this Scheme XII, Y can be depicted as a precursor or is protected with an appropriate protecting group as needed to successfully complete all of the reactions in this scheme, and then de-protected or modified using reagents and methods known in the general art at the end, which then results in Y as defined herein. (A) is then reacted with Bis(pinacolato)diboron), [1,1′-Bis(diphenylphosphino)ferrocene]-palladium(II) dichloride, and potassium acetate in 1,4-dioxane to yield the boronic ester (B). (B) is then reacted with benzyl alcohol (C), using Suzuki palladium coupling methods and reagents known and commonly used to those skilled in the art to form Intermediate (D). If X₂ in the final product is oxygen, then (D) is reacted directly with the appropriate PEG alcohol as depicted as reagent (F), using the InCl₃ method as described in previous schemes above. If X₂ is sulfur, then Intermediate (D) is first converted to the benzyl bromide (E) by reaction of the benzyl alcohol with PBr₃ under conditions and methods known to those skilled in the art. Intermediate (E) is then reacted with the appropriate PEG mercaptan, depicted as reagent (F), and K₂CO₃ or other suitable base. The products from this step, with either X₂ as oxygen or sulfur, and as the methyl ester, is hydrolyzed to the desired product as the free acid with LiOH or other ester hydrolyzing base, followed by acidification with HCl or another suitable acid to yield the desired product, with R₁, R₂, R₃, R₇, R₈, X₂, Y, m, and n as defined herein.

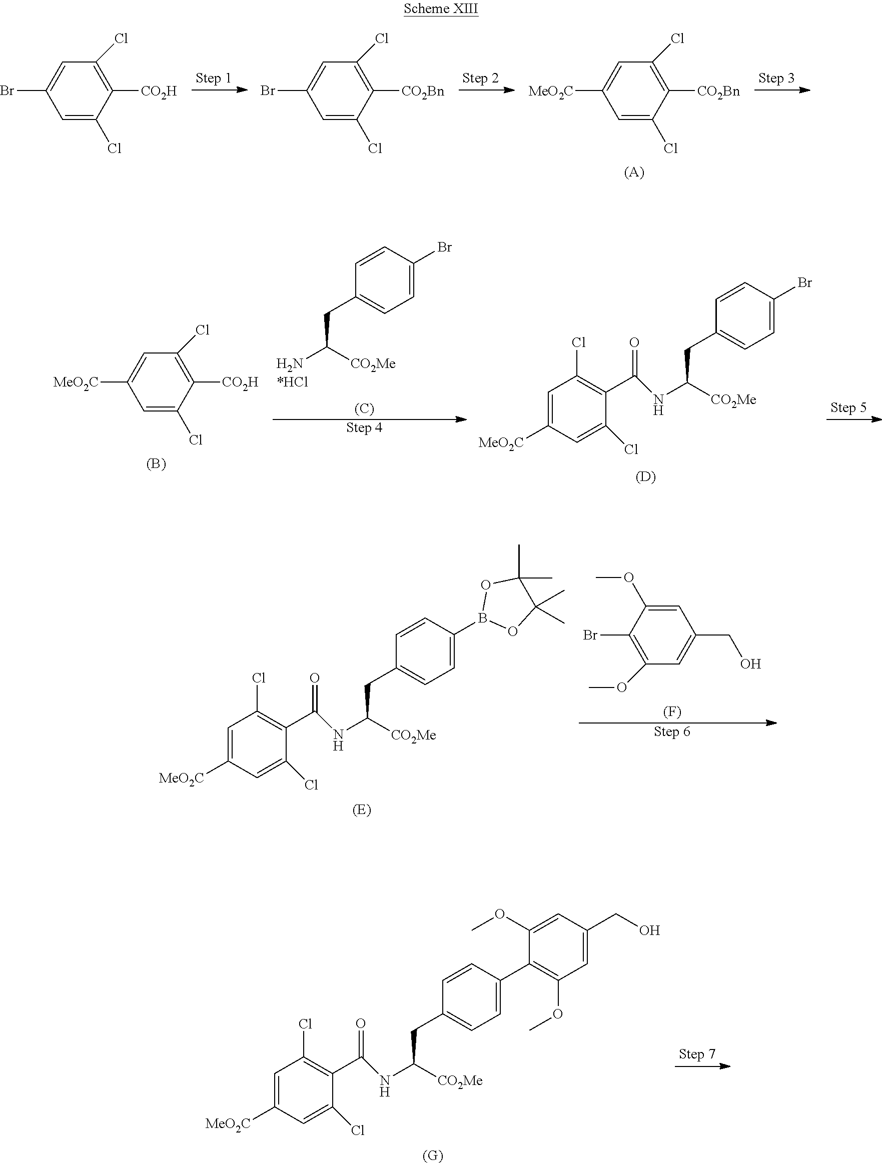

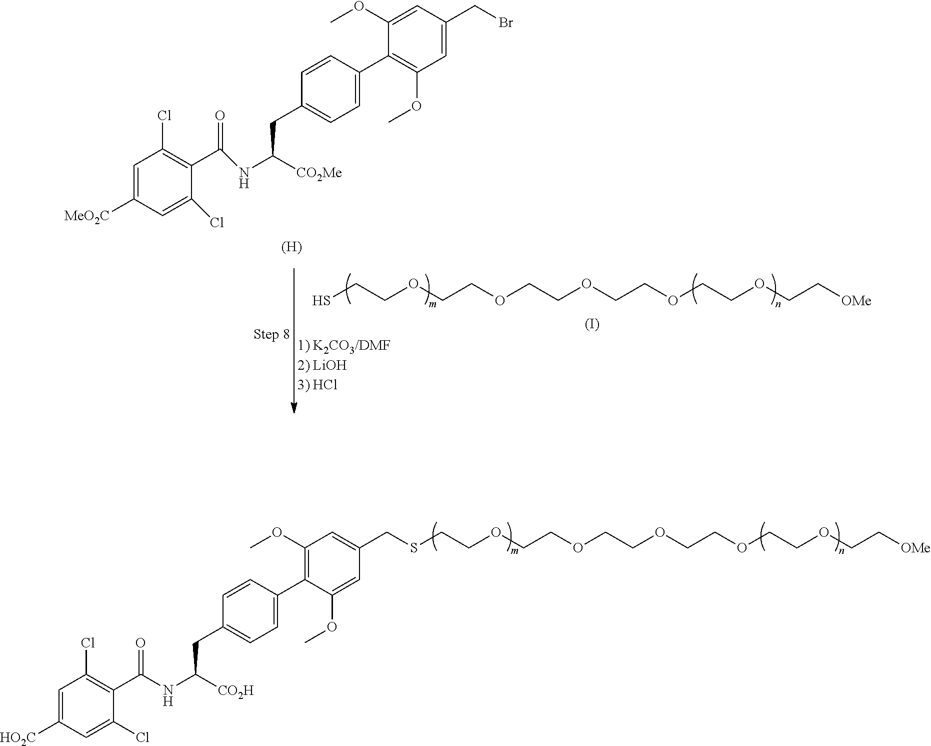

Scheme XIII depicts a general method for the synthesis of preferred embodiments contained within this disclosure where Z is:

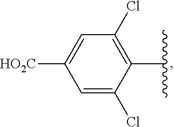 [SMILES: *c1c(Cl)cc(C(=O)O)cc1Cl]

X₂ is sulfur, R₁, R₂ and R₃ are methoxy, m and n are as defined herein and with the (S) stereochemistry preferred, and as drawn accordingly in Scheme XIII above.

Briefly, 4-Bromo-2,6-dichlorobenzoic acid (13.3 mmol) and cesium carbonate (23.2 mmol) are suspended in acetonitrile (50 mL) at 0° C., and then benzyl bromide (13.93 mmol) is added drop wise. The reaction is heated for 4 hours at 60° C. to yield the benzyl ester, as depicted in Step 1. This benzyl ester (4.17 mmol) is then added to a 20 mL microwave vial, along with palladium acetate (0.208 mmol), 4,5-Bis(diphenylphosphino)-9,9-dimethylxanthene (0.417 mmol), 4-dimethylaminopyridine (16.7 mmol), Octacarbonyldicobalt (3.33 mmol) and toluene/methanol (2:1, 15 mL). The vial is crimped shut and irradiated at 90° C. for 30 minutes using microwaves. The reaction is diluted with ethyl acetate, filtered through Celite®, and concentrated in vacuo. The residue is taken up in ethyl acetate and washed using a 10% citric acid solution, then brine. The ethyl acetate layer is dried over sodium sulfate, filtered, and concentrated in vacuo. The residue is purified by silica gel chromatography using ethyl acetate/hexanes as eluent to give Intermediate (A), as depicted in Step 2. To a solution of Intermediate (A) (6.78 mmol) in ethyl acetate (20 mL) is added 10% palladium on carbon (0.34 mmol) and the mixture is stirred at room temperature under a hydrogen atmosphere at ambient pressure for 1.5 hours. The reaction mixture is filtered through Celite® and concentrated in vacuo to give Intermediate (B), as depicted in Step 3. Intermediate (B) (8.38 mmol), benzotriazol-1-ol (1.59 mmol), 3-[Bis(dimethylamino)-methyliumyl]-3H-benzotriazol-1-oxide hexafluorophosphate (8.77 mmol) and DMSO (15 mL) are added to a round bottom flask. N,N-diisopropylethylamine (23.93 mmol) is then added and the reaction is stirred for 40 minutes at room temperature. Methyl (S)-2-amino-3-(4-bromophenyl)propanoate hydrochloride, Intermediate (C) (7.98 mmol), is then added and the reaction is stirred overnight at room temperature. The reaction is diluted with water (50 mL), stirred for 20 minutes, then extracted using ethyl acetate (100 mL). The ethyl acetate layer is washed using additional water, dried using sodium sulfate and concentrated in vacuo. The resulting oil is purified on silica gel using ethyl acetate and hexanes as eluent, to yield Intermediate (D), as depicted in Step 4. To a 20 mL microwave vial is added Intermediate (D) (2.04 mmole), Bis(pinacolato)diboron (2.66 mmol), [1,1′-Bis(diphenylphosphino)ferrocene]-palladium(II) dichloride (0.123 mmole), potassium acetate (6.13 mmol) and 1,4-dioxane (10 mL). The vial is crimped shut, sparged for 10 minutes with nitrogen gas, and then heated overnight at 80° C. The reaction is cooled to room temperature and filtered through Celite®, rinsing with ethyl acetate. The ethyl acetate layer is washed using additional water, dried using sodium sulfate, and concentrated in vacuo. The resulting oil is purified on silica gel using ethyl acetate and hexanes as eluent to yield Intermediate (E), as depicted in Step 5. Benzyl alcohol, Intermediate (F), is synthesized by adding Borane dimethylsulfide complex (7.6 mL of 2 M in tetrahydrofuran, 15.3 mmol) drop-wise at room temperature to an oven dried round bottom flask containing 4-bromo-3,5-dimethoxybenzoic acid (7.66 mmol) and anhydrous tetrahydrofuran (24 mL). The reaction is heated overnight at 40° C., then quenched using hydrochloric acid (1N) and partitioned between ethyl acetate and water. The organic layer was washed using brine, dried with sodium sulfate, filtered, and concentrated in vacuo to give Intermediate (F). To a microwave vial is added Intermediate (E) (0.186 mmol), Intermediate (F) (0.28 mmol, 1.5 equivalent), tetrakis(triphenylphosphane)palladium(0) (0.009 mmol), and cesium acetate (0.559 mmol), in 1,4-dioxane (1 mL) and water (0.25 mL). The vial is crimped shut and sparged with nitrogen for 10 minutes and then heated overnight at 115° C. The reaction is partitioned between ethyl acetate and water, layers are separated, and the ethyl acetate layer is dried using sodium sulfate, filtered, and concentrated in vacuo. The residue is chromatographed on silica gel using ethyl acetate and hexanes as eluent to yield Intermediate (G), as depicted in Step 6. Benzyl alcohol Intermediate (G) is converted to the benzyl bromide, Intermediate (H), using PBr₃ under conditions readily known to those skilled in the art, and as depicted in Step 7. In Step 8, Intermediate (H) is then reacted with the appropriate PEG mercaptan, as depicted by Intermediate (1), with potassium carbonate or another appropriate base in DMF or other suitable solvent, to yield the desired precursor product as the dimethyl ester, which is then hydrolyzed to the desired product as the free acid with LiOH, or another suitable base to hydrolyze the methyl ester precursors.

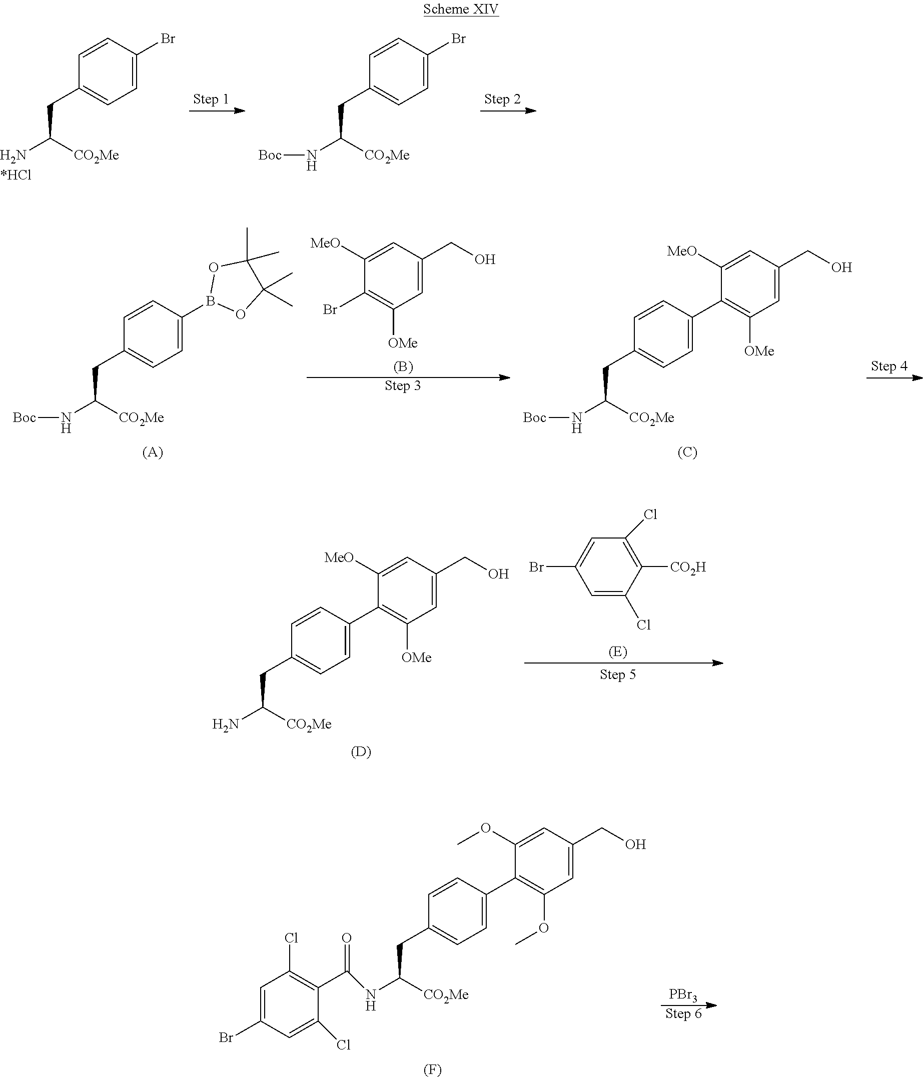

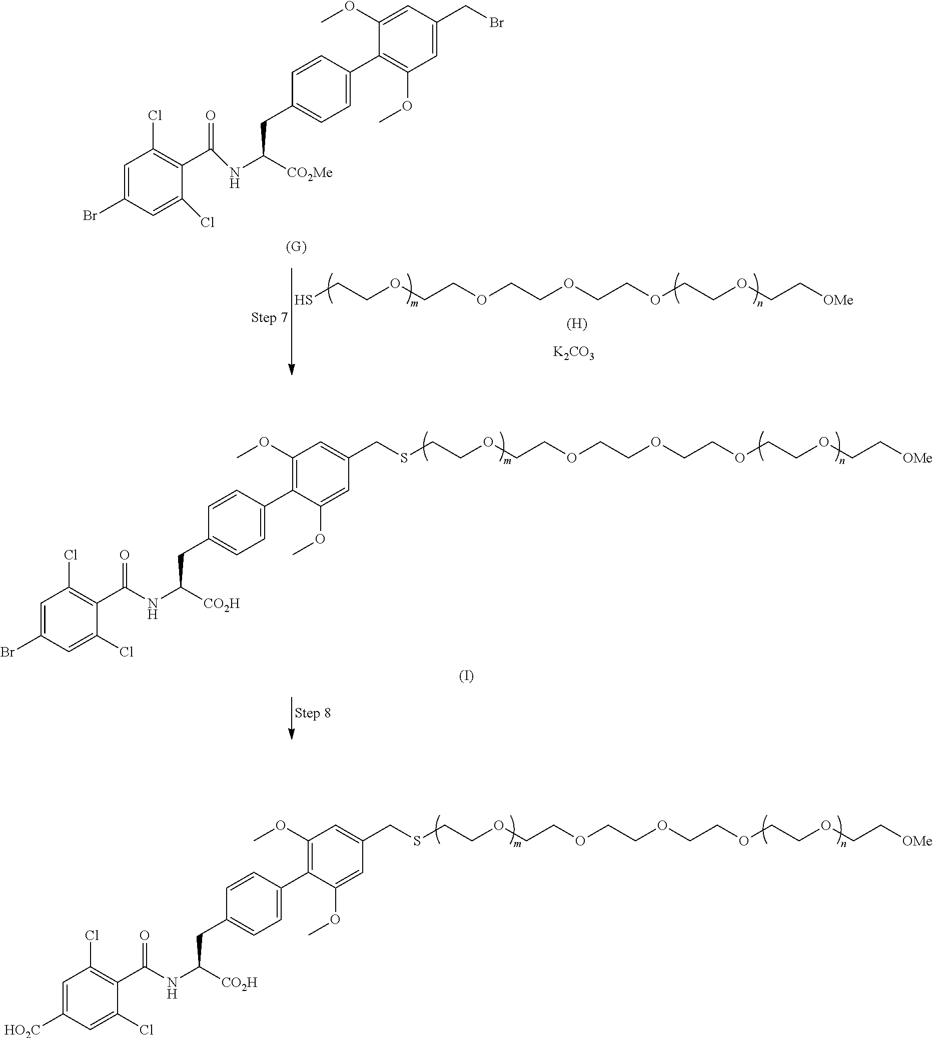

Scheme XIV depicts an alternate to Scheme XIII as a general method for the synthesis of preferred embodiments contained within this disclosure where Z is:

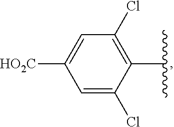 [SMILES: *c1c(Cl)cc(C(=O)O)cc1Cl]

X₂ is sulfur, R₁, R₂ and R₃ are methoxy, m and n are as defined herein and with the (S) stereochemistry preferred, and as drawn accordingly in Scheme XIV above.

Briefly, the methyl ester of 4-bromo phenylalanine HCl is converted to the Boc protected compound as depicted in Step 1, using reagents and methods known to those skilled in the art. This is converted to the boronic ester, Intermediate (A), as depicted in Step 2 and as follows. To a solution of the Boc intermediate (74.2 mmol), and 4,4,4′,4′,5,5,5′,5′-octamethyl-2,2′-bi(1,3,2-dioxaborolane) (111 mmol) in dioxane (250 mL) is added Pd(dppf)Cl2 (7.43 mmol) and KOAc (222 mmol). The mixture is stirred at 90° C. for 12 hrs under a N2 atmosphere. The reaction mixture is then filtered and concentrated under vacuum to give the desired Intermediate (A) as a yellow oil, which was purified by column chromatography (SiO2, Petroleum ether/Ethyl acetate). Intermediate (A) is then converted to Intermediate (C) by reaction with intermediate (B) as depicted in Step 3 and as follows. A solution of Intermediate (A) (23.7 mmol), Intermediate (B) (28.4 mmol), Pd(dppf)Cl2 (2.37 mmol) and K₃PO₄ (71.2 mmol) in dioxane (50.0 mL) and H₂O (10.0 mL) is stirred at 80° C. for 12 h. The reaction mixture is filtered. The filtrate is poured into water (50.0 mL) and extracted with ethyl acetate (50.0 mL*3). The combined organic layer is washed with brine (100 mL), dried over sodium sulfate, filtered, and concentrated to give Intermediate (C) as light yellow oil, which was purified by column chromatography and further purified by prep-HPLC. The Boc group from Intermediate (C) is removed with HCl/dioxane as depicted in Step 4 to give Intermediate (D), as the HCl salt. Intermediate (D) is then coupled with benzoic acid Intermediate (E) by taking a solution of Intermediate (E) (6.78 mmol, 1.00 eq) in DMF (30.0 mL) and adding DIEA (33.9 mmol, 5.00 eq), HATU (10.1 mmol, 1.50 eq) and HOBt (2.03 mmol, 0.300 eq). This mixture is stirred at 25° C. for 0.5 hr. Then Intermediate (D) (6.78 mmol, 1.00 eq) was added. The mixture is stirred at 25° C. for 12 hrs. The reaction mixture is poured into water (50.0 mL) and extracted with ethyl acetate (30.0 mL*3). The combined organic layer is washed with brine (80.0 mL), dried over Na2SO4, filtered and concentrated. The crude product was purified by prep-HPLC, as Intermediate (F), as depicted in Step 5. The benzyl alcohol of Intermediate (F) is converted to the benzyl bromide, Intermediate (G), using PBr₃ and reaction conditions readily known to those skilled in the art, and as depicted in Step 6. Intermediate (G) is then reacted with the appropriate PEG mercaptan, Intermediate (H), with potassium carbonate or other appropriate bases, in DMF, to give Intermediate (I), as depicted in Step 7.

A mixture of Intermediate (I) (51.5 μmol, 1.00 eq), TEA (206 μmol, 28.6 μL, 4.00 eq), Pd(dppf)Cl2 (5.15 μmol, 0.10 eq) in MeOH (20.0 mL) is heated to 80° C. under CO atmosphere (50 psi) for 16 hrs. The reaction mixture is filtered and the filtrate is concentrated under vacuum to give the penultimate product, which was hydrolyzed with LiOH in THE/water, as depicted in Step 8 to give the desired product as the di-acid, with m and n as defined herein.

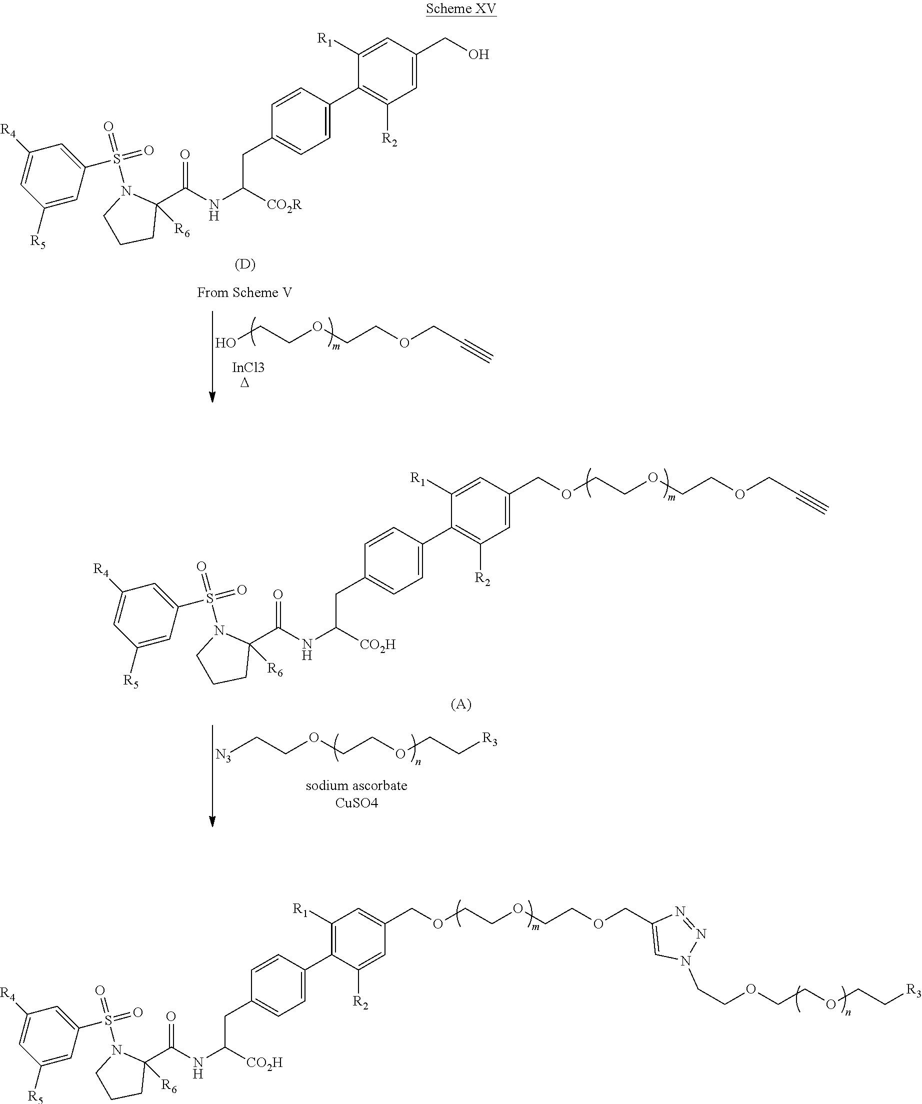

Scheme XV represents an additional synthetic efficiency to the enablement of the products of this disclosure. It takes advantage of “click chemistry” and the availability of a wide range of PEG azides that are commercially available. From a common acetylenic intermediate, depicted as Intermediate (A) in Scheme XV above, and with m as defined herein, many PEG analogues of various lengths and further functionalization can be synthesized, forming a triazole linker between different PEG chains. Briefly, the appropriate acetylenic PEG alcohol is reacted with Intermediate (D) and InCl₃ as shown in Scheme XV, to form Intermediate (A). Intermediate (A) is then reacted with the appropriate PEG azide under standard “click chemistry” conditions, as shown, to yield the desired products, with R₁₋₆, m, and n as defined herein, with m=1 as a preferred embodiment.

Further functionalization of products synthesized via Scheme XV at R₃ can be envisioned from the methods and examples illustrated in the previous Schemes, and as defined by R₃ herein. Scheme XV is meant to be an example illustrating the efficiency to be enabled by employing “click chemistry” as shown, and is not meant to be limiting in nature, as those skilled in the art can envision additional methods, reagents, and conditions to derive the triazole linked PEG products as defined in the general claims of this disclosure.

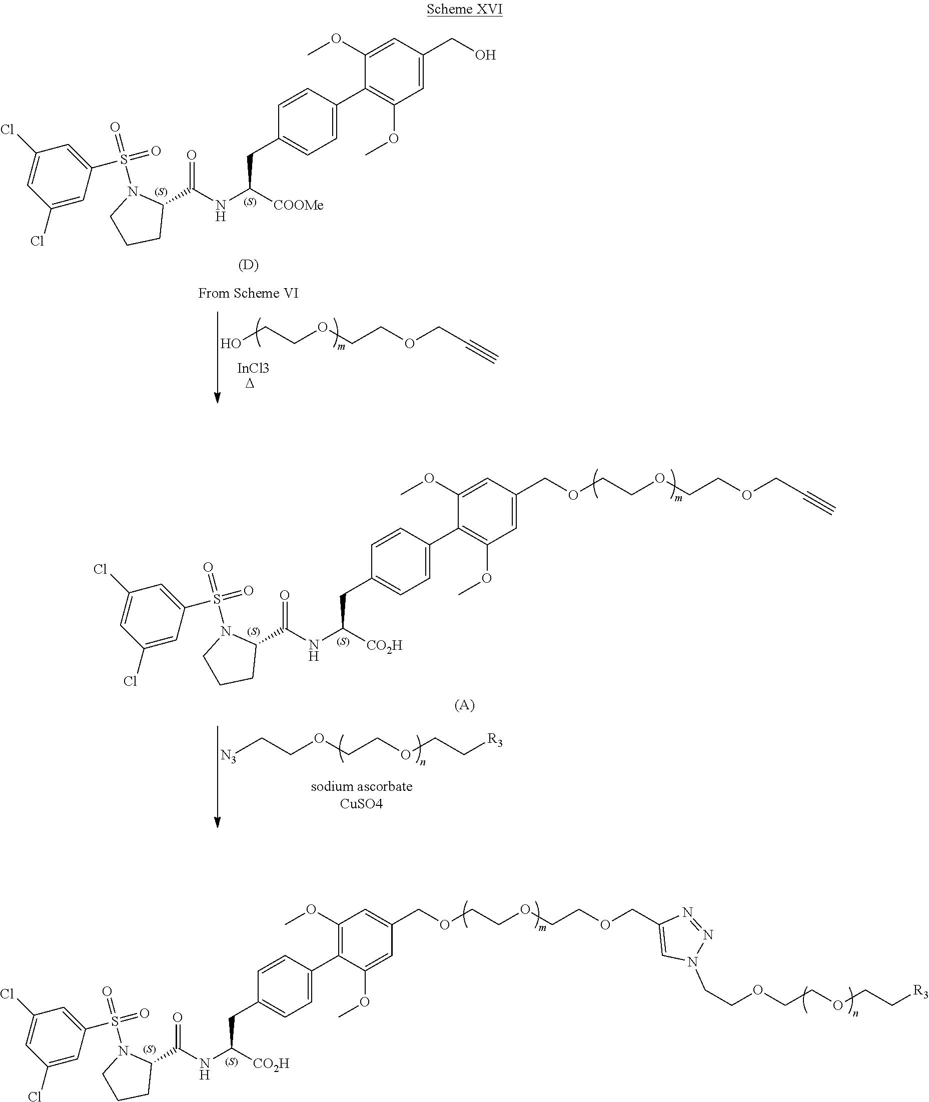

Scheme XVI depicts a general method for the synthesis of preferred embodiments as related to the “click chemistry” products of this disclosure. The methods and descriptions relevant to the synthesis of examples as depicted in Scheme XVI are the same as detailed in Scheme XV, with R₁ and R₂ specified here as methoxy, R₄ and R₅ specified as chloro, R₆ specified as hydrogen, and with the (S)stereochemistry preferred, and as drawn accordingly in Scheme XVI above. m, n, and R₃ are as defined herein, with m=1 as a further preferred embodiment.

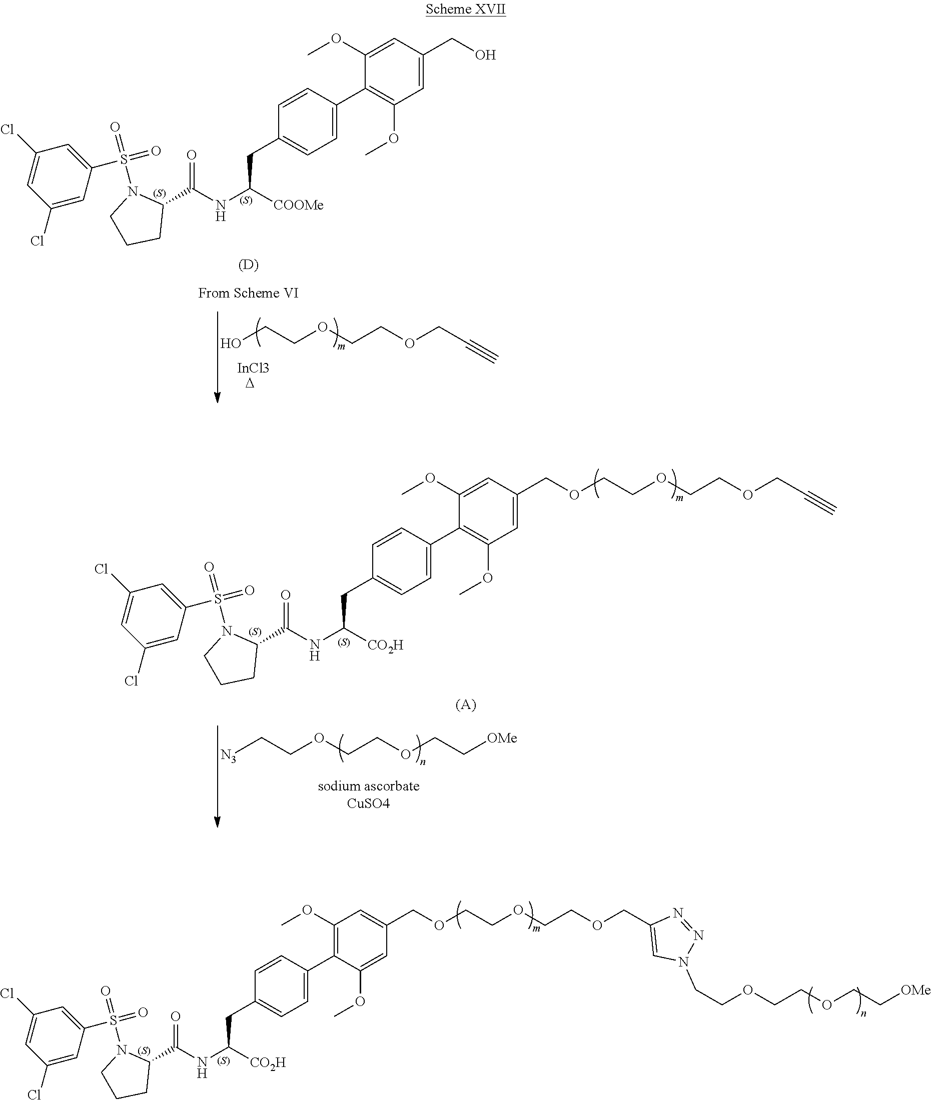

Scheme XVII depicts a general method for the synthesis of additional preferred embodiments as related to the “click chemistry” products of this disclosure. The methods and descriptions relevant to the synthesis of examples as depicted in Scheme XVII are the same as detailed in Scheme XVI, with R₁, R₂ and R₃ specified here as methoxy, R₄ and R₅ specified as chloro, R₆ specified as hydrogen, and with the (S) stereochemistry preferred, and as drawn accordingly in Scheme XVII above. m and n are as defined herein, with m=1 as a further preferred embodiment.

Similar “click chemistry” embodiments can be envisioned where Z=

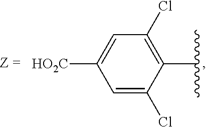 [SMILES: *c1c(Cl)cc(C(=O)O)cc1Cl]

by incorporating previous schemes and methods that would be recognized to those skilled in the art.

In some embodiments, the compounds of the present disclosure include the compounds described in the Exemplary Embodiments and claims listed below. Additionally, these compounds are listed in TABLE 1 below.

TABLE 1
Example Compounds of the Present Disclosure
Example Compounds

| Ex. | |
|---|---|
| No. | Compound Structure |
| 1 | 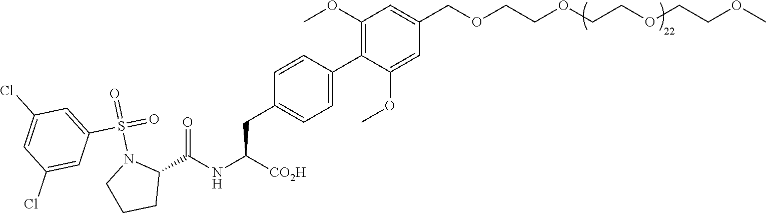 |
| 2 | 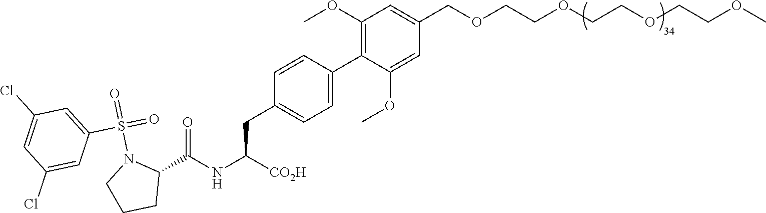 |
| 3 | 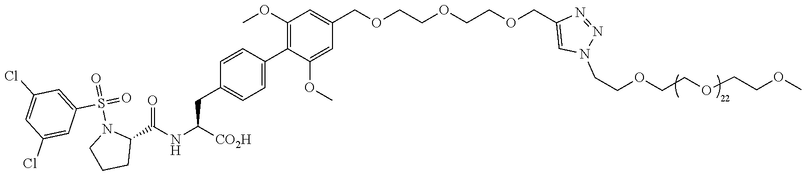 |
| 4 | 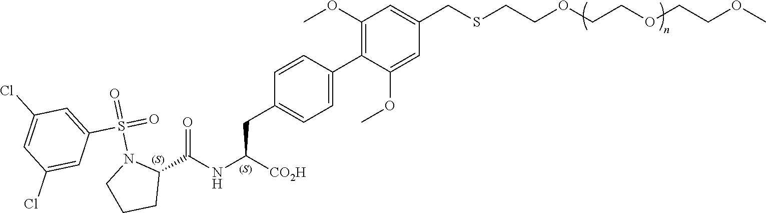 |
| | n = MW 5K |
| 5 | 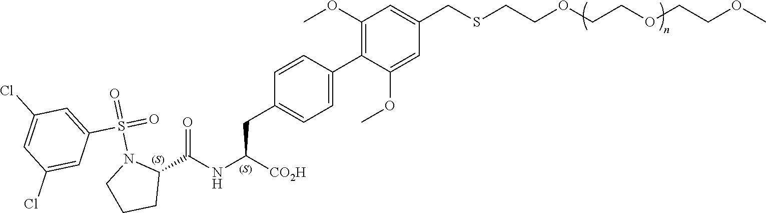 |
| | n = MW 20K |
| 6 | 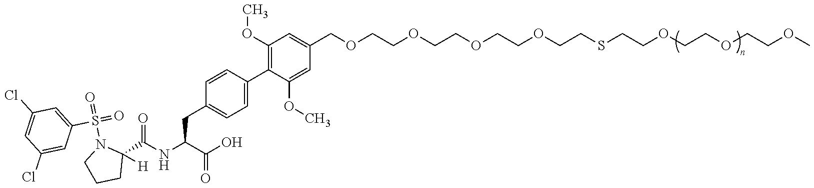 |
| | n = MW 5K |
| 7 | 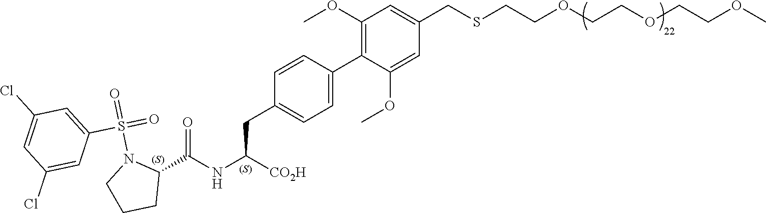 |
| 8 | 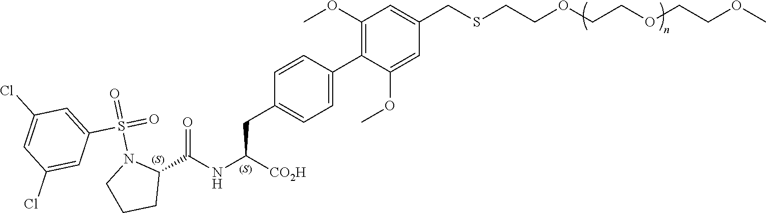 |
| | n = MW 40K |
| 9 | 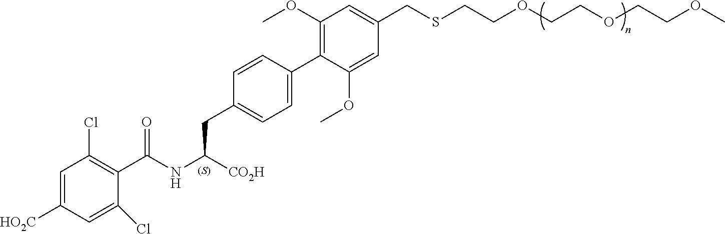 |
| | n = MW 20K |
| 10 | 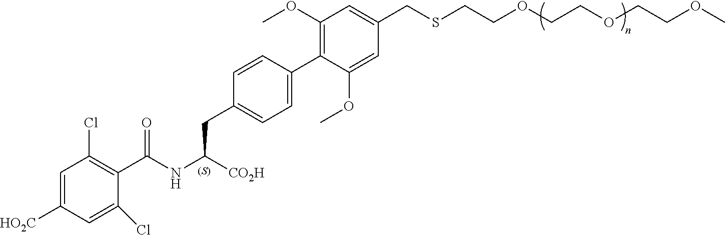 |
| | n = MW 40K |
| 11 | 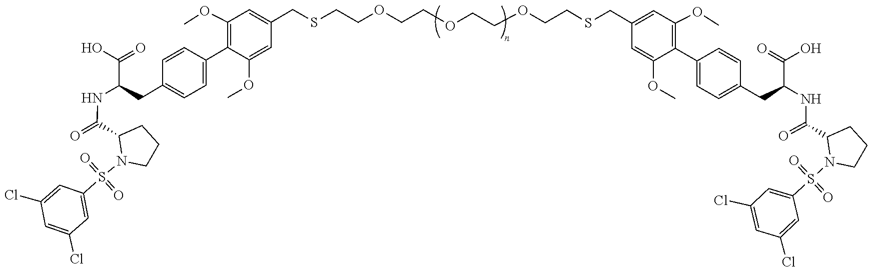 |
| | n = MW 10K |
| 12 | 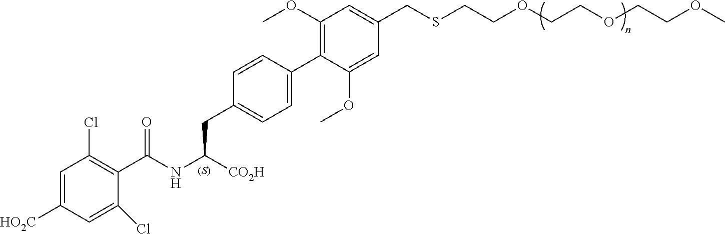 |
| | n = MW 10K |
| 13 | 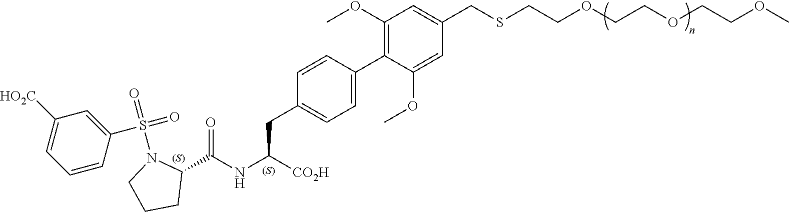 |
| | n = MW 20K |
| 14 | 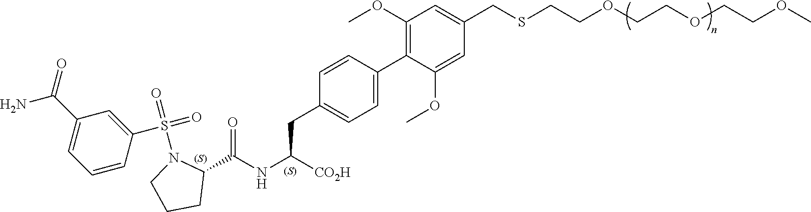 |
| | n = MW 20K |
| 15 | 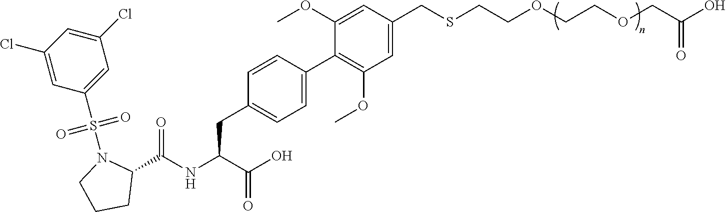 |
| | n = MW 20K |
| 16 | 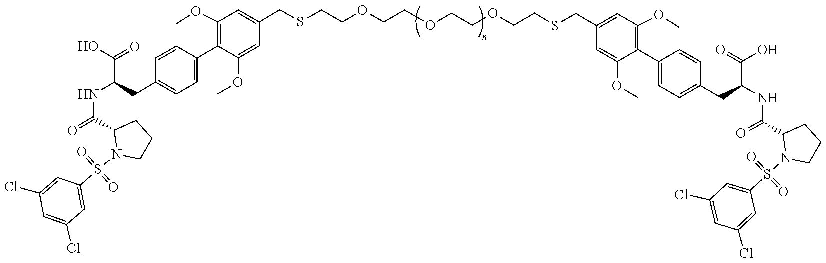 |
| | n = MW 2K |
| 17 | 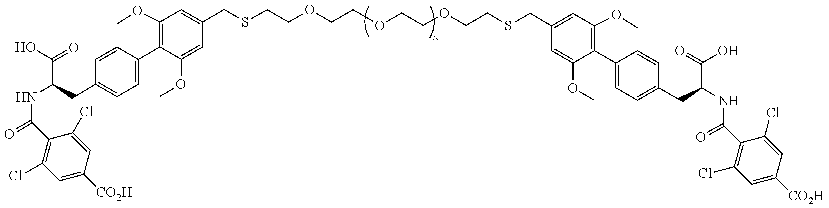 |
| | n = MW 10K |
| 18 | 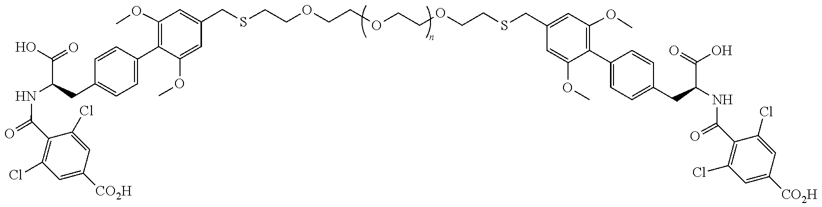 |
| | n = MW 2K |
| 19 | 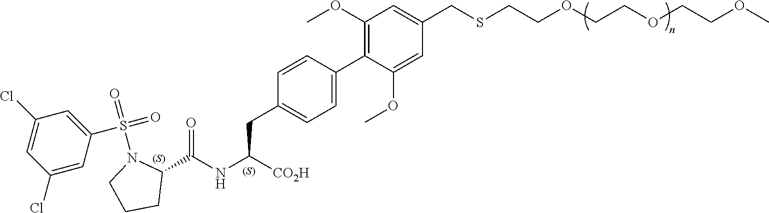 |
| | n = MW 10K |

Compounds of similar structural identity, some of which are described in the prior art, are described herein to demonstrate the novelty of the compounds of the present disclosure when compared to these compounds of structural similarity in regards to extended hematopoietic stem cell mobilization after a single dose. These will be referred to as Comparator Compounds and are listed in TABLE 2 below.

TABLE 2
Comparator Compounds

| Compara- | |
|---|---|
| tor | |
| No. | Compound Structure |
| C1 | 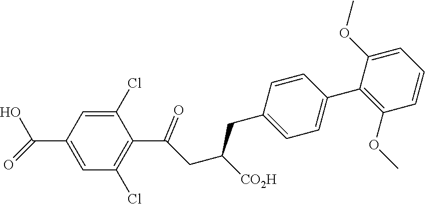 [SMILES: COc1cccc(OC)c1-c1ccc(C[C@H](CC(=O)c2c(Cl)cc(C(=O)O)cc2Cl)C(=O)O)cc1] |
| C2 | 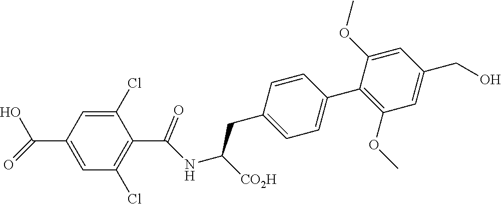 [SMILES: COc1cc(CO)cc(OC)c1-c1ccc(C[C@H](NC(=O)c2c(Cl)cc(C(=O)O)cc2Cl)C(=O)O)cc1] |
| C3 | 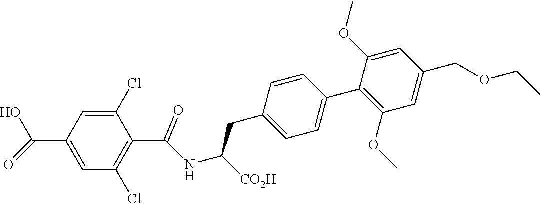 [SMILES: CCOCc1cc(OC)c(-c2ccc(C[C@H](NC(=O)c3c(Cl)cc(C(=O)O)cc3Cl)C(=O)O)cc2)c(OC)c1] |
| C4 | 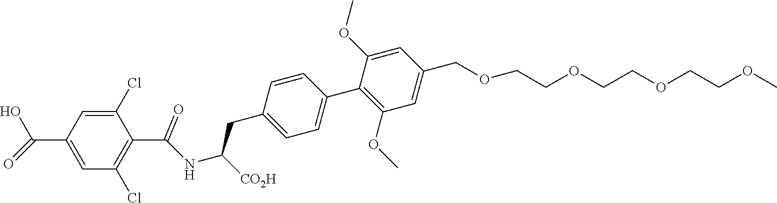 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1] |
| C5 | 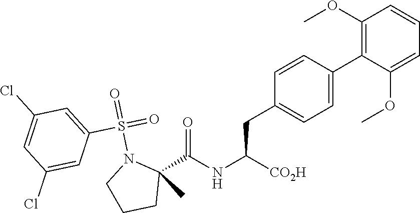 [SMILES: COc1cccc(OC)c1-c1ccc(C[C@H](NC(=O)[C@]2(C)CCCN2S(=O)(=O)c2cc(Cl)cc(Cl)c2)C(=O)O)cc1] |
| C6 | 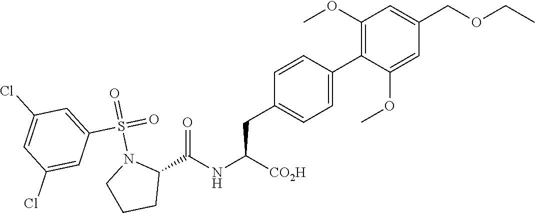 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] |
| C7 | 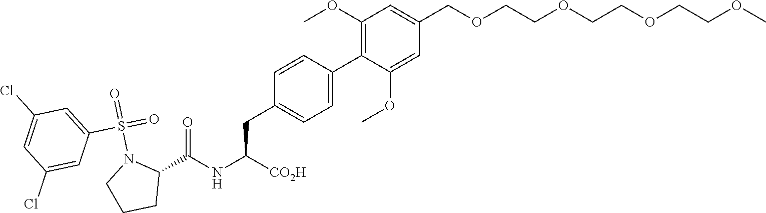 [SMILES: COCCOCCOCCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] |
| C8 | 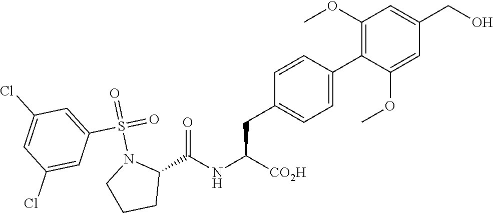 [SMILES: COc1cc(CO)cc(OC)c1-c1ccc(C[C@H](NC(=O)[C@@H]2CCCN2S(=O)(=O)c2cc(Cl)cc(Cl)c2)C(=O)O)cc1] |
| C9 | 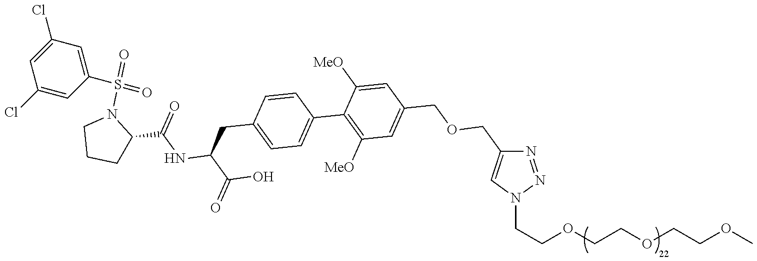 |
| C10 | 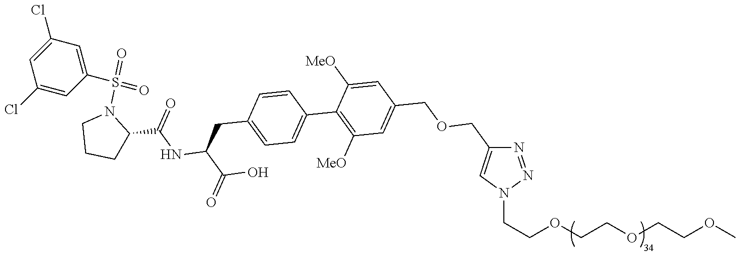 |
| C11 | 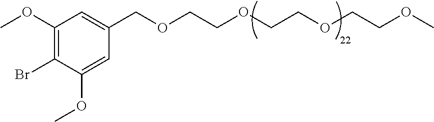 |
| C12 | 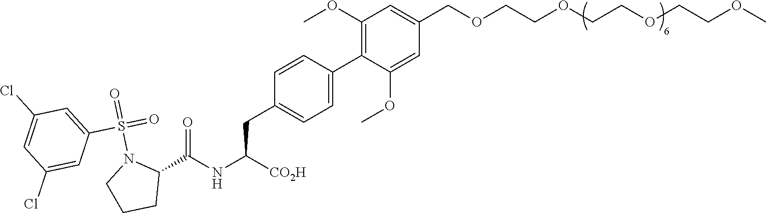 |
| C13 | 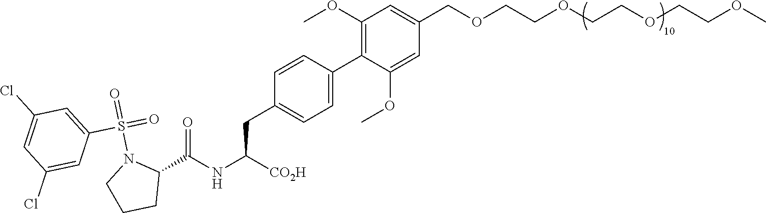 |
| C14 | 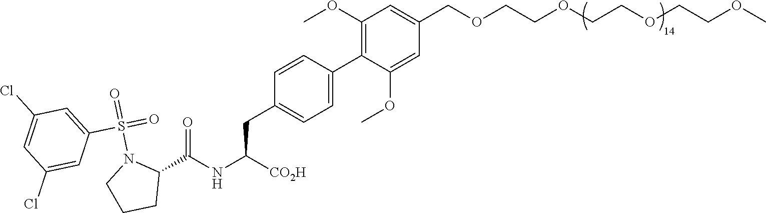 |
| C15 | 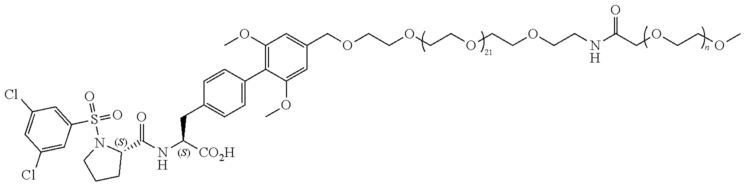 |
| | n = MW 20K |
| C16 | 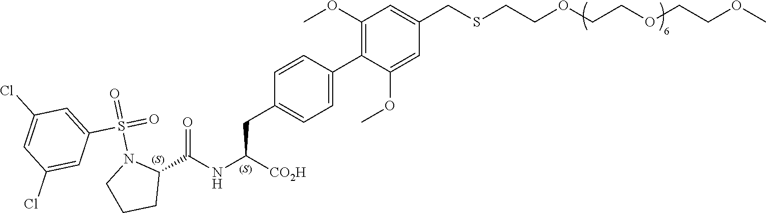 |
| C17 | 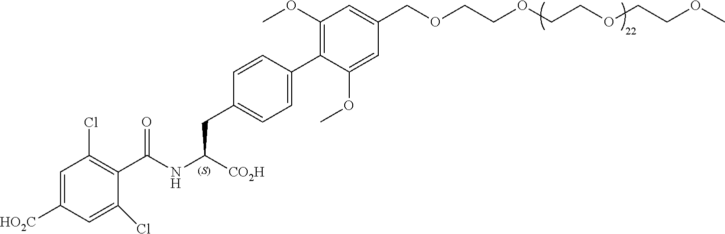 |
| C18 | 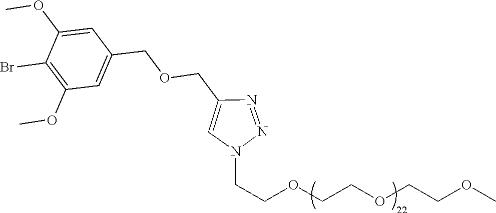 |
| C19 | 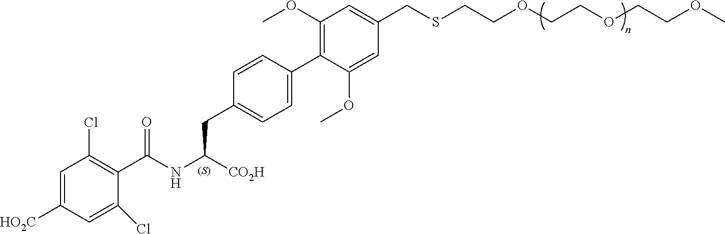 |
| | n = MW 2K |
| C20 | 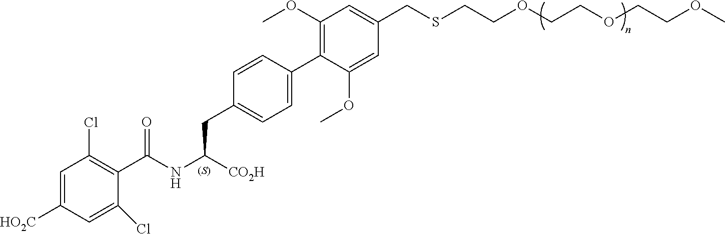 |
| | n = MW 5K |
| C21 | 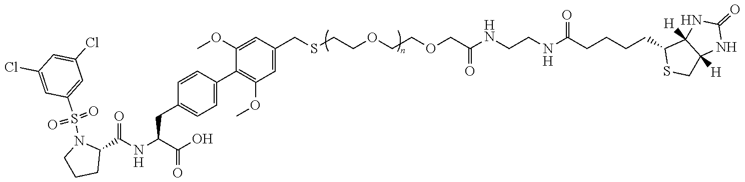 |
| | n = MW 20K |
| C22 | 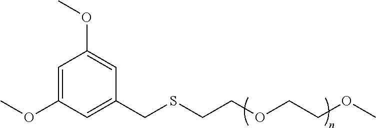 |
| | n = MW 20K |
| Firateg- rast | 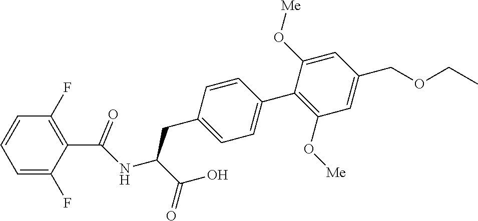 [SMILES: CCOCc1cc(OC)c(-c2ccc(C[C@H](NC(=O)c3c(F)cccc3F)C(=O)O)cc2)c(OC)c1] |
| BOP | 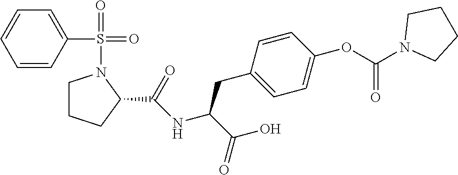 [SMILES: O=C(O)[C@H](Cc1ccc(OC(=O)N2CCCC2)cc1)NC(=O)[C@@H]1CCCN1S(=O)(=O)c1ccccc1] |
| Carote- grast (active metabo- lite of AJM300) | 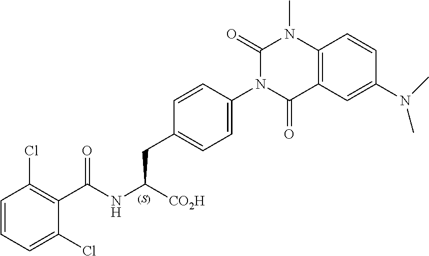 |
| TR- 14035 | 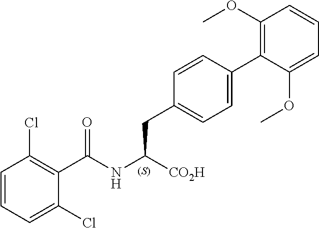 |
| RO0270608 (the active metabo- lite of Valate- grast | 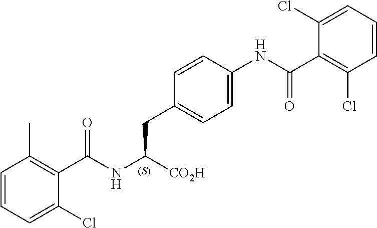 |

### Mobilizing Hematopoietic Stem Cells

Mobilizing hematopoietic stem cells from the bone marrow into the peripheral blood is a critical procedure for stem cell transplants in the treatment of blood cancers. The current mobilizing agent in clinical use is a protein called Granulocyte Colony Stimulating Factor (G-CSF). The patient or donor must come in for daily G-CSF injections for up to a week or more in order to mobilize a high enough number of blood stem cells that are sufficient for a safe stem cell transplant. Furthermore, G-CSF does not effectively mobilize a sufficient number or quality of stem cells in all donors and can produce undesirable side effects in some individuals. Additionally, when HSPC mobilization is utilized for gene editing of sickle cell disease patients, G-CSF is actually contraindicated in sickle cell anemia patients due to its ability to induce life-threatening acute chest syndrome and life-threatening vaso-occlusive episodes. Therefore, it is desirable to identify an alternative treatment that is safe, rapid, and cost-effective, thus making this procedure more inclusive across all patient populations.

The presently disclosed, novel VLA4 inhibitors would achieve the same degree of mobilization after a single injection, and particularly in combination with other agents, such as inhibitors of CXCR4, and collected over a 4-6 hour period, after which the patient or donor can go home. This would provide for a more efficient donor HSPC collection in contrast to having to come in daily for up to a week or more with the current protocol using G-CSF. This would not only be more convenient, but would dramatically reduce the cost of the mobilization procedure.

Clinical applications for these new compositions can be for hematopoietic stem cell transplantation and other applications. The disclosed compositions can mobilize donor stem cells to be harvested for a patient in need thereof. The disclosed compositions can mobilize a patient's cells (e.g., having leukemia, multiple myeloma) to make chemotherapy, radiation, and other cancer therapies more efficient. A subject having sickle cell anemia or other blood born genetic diseases, for example, can be administered the disclosed compositions for mobilization and harvesting of their own hematopoietic stem cells which then undergo gene editing to correct the mutated disease gene and then for reinfusion of the corrected hematopoietic stem cells as a means of curing the disease. Because of the mechanism of these new compositions as inhibitors of the integrins VLA4 (a4b1) and a4b7, other envisioned applications in addition to hematopoietic cell mobilization, are for treatment of graft vs. host disease (GvHD), inflammatory bowel disease (IBD), ulcerative colitis, Crohn's disease, multiple sclerosis, spinal cord injury, neurological diseases and other inflammatory pathologies associated with this mechanism.

### Hematopoietic Stem/Progenitor Cells (HSPCs) Cell Therapy

Hematopoietic stem/progenitor cells (HSPCs) (wild type or engineered) generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, infusing or transplanting hematopoietic stem/progenitor cells (HSPCs) can treat or prevent diseases, disorders, or conditions, or increase the effectiveness of cancer therapies.

Stem cell and cell transplantation have gained significant interest by researchers as a potential new therapeutic strategy for a wide range of diseases, in particular for proliferative, degenerative, and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation use cells from another species. For example, pig derived cells can be transplanted into humans. Xenogeneic cell therapies can involve human cell transplantation into experimental animal models for assessment of efficacy and safety or enable xenogeneic strategies to humans as well.

Hematopoietic stem cell transplantation (HSCT) is the major curative therapy available for many hematological diseases including hematological cancers. In this technique, HSCT is used to facilitate repopulation of healthy bone marrow and immune system cells after a high-dose chemotherapy treatment for cancers including but not limited to Hodgkin's and non-Hodgkin's lymphoma, multiple myeloma, or leukemia. In order to facilitate transplantation when the cells are needed, hematopoietic stem/progenitor cells (HSPCs) are collected from the patient's blood, harvested, frozen, and then stored while the patient receives high-dose chemotherapy and/or radiation therapy.

The amount of hematopoietic stem/progenitor cells (HSPCs) collected to be useful in a subject can be any amount that is therapeutically effective to the subject. For example, in order to achieve successful transplantation, an intravenous infusion of a minimum number of 2×10⁶ CD34+ stem cells/kg body weight is often needed; however, a dose of 5×10⁶ CD34+ cells/kg is considered preferable for early and long term multilineage engraftment in humans. As such, generally, at least 2 million CD34+ stem cells per kilogram human recipient body weight is needed to proceed to transplant and ensure multilineage engraftment in the recipient. If possible, it is preferable to transplant 5 million CD34+ cells per kilogram recipient body weight because it can result in more consistent neutrophil and platelet engraftment.

Currently, the stem cells for hematopoietic stem cell transplants are often harvested from peripheral blood. Due to the low amount of these cells in circulating peripheral blood, the stem cells often must be stimulated to increase the quantity in the peripheral blood, a process which can take almost a week. Even then, the collection is still done over several days to achieve sufficient concentrations of the stem cells for transplantation. This greatly increases the cost of the transplant and results in a significant burden on the patient. Currently, cytokines, such as granulocyte-colony forming unit (G-CSF), and immunostimulants, such as plerixafor, are used to increase the amount of hematopoietic stem cells in the peripheral blood but a single agent often results in insufficient mobilization of stem cells. Additional methods of harvesting hematopoietic stem cells have been developed which involve combining G-CSF with multiple other agents such as plerixafor or another cytokine. Unfortunately, even these combined therapies often fail to increase the concentrations to sufficient levels for transplantation in many patients even with multiple days of apheresis. Furthermore, several of these agents, like plerixafor, are extremely expensive adding over $25,000 per patient relative to using G-CSF alone. Therefore, a need remains for better methods to harvest hematopoietic stem cells.

### VLA-4 Inhibitors or Antagonists

The present disclosure provides compounds which are VLA-4 antagonists (i.e., inhibitors), pharmaceutical compositions, methods for their manufacture, and methods of use thereof.

Disclosed herein are new compounds and compositions which act as integrin antagonists (i.e., inhibitors) of, for example, α4β1 integrin (VLA-4) and a4b7, pharmaceutical compositions, methods for their manufacture, and methods for their use, including for the treatment and/or prevention of disease. In some embodiments, these compounds may be used in improving the harvesting of hematopoietic stem cells or progenitor cells or to enhance an anti-cancer therapy.

The VLA-4 inhibitors disclosed herein represent a novel composition of matter for the utility of significantly mobilizing hematopoietic stem/progenitor cells from the bone marrow to the peripheral blood over an extended period of time compared to previously disclosed molecules.

### PEG

It was discovered herein that increasing polyethylene glycol (PEG) lengths, covalently attached to specific chemical structures, actually improved inhibitors of the integrin VLA-4 for superior and extended mobilization of hematopoietic stem cells, and that the nature of the PEG itself, and the minimal PEG length required for such improvements, are dependent upon the nature of the specific chemical core structure the PEG is attached to. Multiple direct Comparator Compounds provided herein demonstrate the non-obvious nature of the disclosed compositions and support the novelty of the claimed matter presented.

Thus, it was shown that it was not the length of PEG per se, but the entire molecule itself that increased the mobilization time and cell mobilization. Triazole linker analogues, as described herein, provide convenient and facile synthetic utility by taking advantage of “click chemistry” reactions of an appropriate acetylenic precursor reacted with a PEG azide of desired composition and molecular weight. One skilled in the art can recognize the potential utility of such analogues in various biological processes and functional outcomes. However, as shown herein, the placement of the triazole linker in relation to the core inhibitor structure determines whether or not extended mobilization occurs, further supporting the non-obvious nature of the disclosed technology.

As shown herein, these new VLA-4 inhibitors are superior to previously produced molecules (see e.g., U.S. application Ser. No. 16/401,950, incorporated herein by reference in its entirety) in regards to providing more and extended mobilization of hematopoietic stem cells in mice.

The present disclosure provides for the covalent addition of defined lengths of polyethylene glycol (PEG) units to a specific attachment point on core VLA-4 inhibitor structures that unexpectedly retain high (sub nM) potency (inhibition) against VLA-4 whilst providing excellent solubility in saline and rapid, extended and significant mobilization after a single dose compared to analogues that lack the disclosed minimum PEG chain length. Furthermore, the minimum chain length required to achieve these properties varies, depending on the specific core structure, highlighting the fact that attaching a minimum PEG length to one core structure to achieve extended mobilization is not applicable to a different core structure, thereby rendering the disclosed compounds as non-obvious when compared to the prior art. To further highlight this point, numerous comparator compounds are provided herein to demonstrate that such extended HSPC mobilization is only achieved with a minimum PEG length as disclosed in the general claims, and this minimum PEG length is specific to the core structure as described in the definition of the claims. Multiple attached PEG length analogues that are below the minimum needed are highlighted within the Comparator Compounds described, along with the mobilization data showing a lack of extended mobilization with such Comparator Compounds. Also, traditionally attaching long PEG groups to drug molecules to improve pharmacokinetic properties, such as plasma half-life, reduces the drug's potency proportionate to the length of the PEG chain. Unexpectedly, the novel compositions disclosed herein demonstrate that attaching long PEG lengths to the particular attachment point on the core structures disclosed herein does not reduce potency in relation to VLA-4 inhibition, but retains high potency regardless of the PEG chain length, even up to 40 KD. Additionally, Comparator Compounds composed of truncated core structures with long PEG chains (Comparator Compounds C11 and C18) do not provide for mobilization to any degree, demonstrating that long PEG groups by themselves do not exhibit HSPC mobilization.

An additional property of the compositions disclosed herein is that they also inhibit the integrin a4b7 in addition to VLA-4 (a4b1). This dual inhibition affords the compounds of this technology the ability to act as a therapeutic for diseases and pathologies such as multiple sclerosis, inflammatory bowel diseases such as Crohn's disease and ulcerative colitis, graft vs host disease (GvHD), neurodegeneration, spinal cord injury, and other inflammatory diseases, in addition to HSPC mobilization, as will be further described herein. The fact that Examples disclosed within that contain 20 KD and 40 KD PEG attachments, yet still retain sub nM potency against VLA4 renders them as suitable candidates as therapies directed at diseases that will require a more chronic plasma exposure, such as multiple sclerosis, since extended plasma half-life of drug after a single s.c. injection can be achieved.

In some aspects the compositions can comprise a linker group. The linker group can be linked to a VLA-4 inhibitor or connect two VLA-4 inhibitors. In some embodiments, the linker can be a PEG, a triazole, a chemical linker, an enzymatic linker, a bond, or an electrostatic linker.

Polyethylene glycol (PEG) is a polyether compound derived from petroleum with many applications, from industrial manufacturing to medicine. PEG is also known as polyethylene oxide (PEO) or polyoxyethylene (POE), depending on its molecular weight. The structure of PEG is commonly expressed as H—(O-CH2-CH2)n-OH. PEG, PEO, and POE refer to an oligomer or polymer of ethylene oxide. The three names are chemically synonymous, but conventionally PEG is preferred in the biomedical field, whereas PEO is more prevalent in the field of polymer chemistry. PEGs can be prepared by polymerization of ethylene oxide and are commercially available over a wide range of molecular weights from 300 g/mol (300 Da) to 10,000,000 g/mol (10,000 kDa).

PEG and PEO can be liquids or low-melting solids, depending on their molecular weights. While PEG and PEO with different molecular weights find use in different applications, and have different physical properties (e.g., viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Very high-purity PEG has recently been shown to be crystalline, allowing determination of a crystal structure by x-ray crystallography. Since purification and separation of pure oligomers can be difficult, the price for this type of quality is often 10-1000 fold that of polydisperse PEG.

PEGs are also available with different geometries. Branched PEGs can have three to ten PEG chains emanating from a central core group. Star PEGs can have 10 to 100 PEG chains emanating from a central core group. Comb PEGs can have multiple PEG chains normally grafted onto a polymer backbone.

The numbers that are often included in the names of PEGs indicate their average molecular weights (e.g., a PEG with n=9 would have an average molecular weight of approximately 400 daltons, and would be labeled PEG 400). Some PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (DM). Mw and Mn can be measured by mass spectrometry.

PEGylation is the act of covalently coupling a PEG structure to another larger molecule, for example, a VLA4 inhibitor, which can be referred to as a PEGylated VLA4 inhibitor. PEG is soluble in water, methanol, ethanol, acetonitrile, benzene, and dichloromethane, and is insoluble in diethyl ether and hexane.

PEGs and methoxypolyethylene glycols are manufactured by Dow Chemical under the trade name Carbowax for industrial use, and Carbowax Sentry for food and pharmaceutical use. They vary in consistency from liquid to solid, depending on the molecular weight, as indicated by a number following the name. They are used commercially in numerous applications, including foods, in cosmetics, in pharmaceutics, in biomedicine, as dispersing agents, as solvents, in ointments, in suppository bases, as tablet excipients, and as laxatives. Some specific groups are lauromacrogols, nonoxynols, octoxynols, and poloxamers. Macrogol, MiraLax, GoLytely, Colace, is a form of polyethylene glycol. The name may be followed by a number which represents the average molecular weight (e.g., macrogol 3350, macrogol 4000, macrogol 6000).

The PEG as described herein can be between about 1 kDa and 100 kDa. The PEG lengths as described herein can be about 1 kDa; about 2 kDa; about 3 kDa; about 4 kDa; about 5 kDa; about 6 kDa; about 7 kDa; about 8 kDa; about 9 kDa; about 10 kDa; about 11 kDa; about 12 kDa; about 13 kDa; about 14 kDa; about 15 kDa; about 16 kDa; about 17 kDa; about 18 kDa; about 19 kDa; about 20 kDa; about 21 kDa; about 22 kDa; about 23 kDa; about 24 kDa; about 25 kDa; about 26 kDa; about 27 kDa; about 28 kDa; about 29 kDa; about 30 kDa; about 31 kDa; about 32 kDa; about 33 kDa; about 34 kDa; about 35 kDa; about 36 kDa; about 37 kDa; about 38 kDa; about 39 kDa; about 40 kDa; about 41 kDa; about 42 kDa; about 43 kDa; about 44 kDa; about 45 kDa; about 46 kDa; about 47 kDa; about 48 kDa; about 49 kDa; about 50 kDa; about 51 kDa; about 52 kDa; about 53 kDa; about 54 kDa; about 55 kDa; about 56 kDa; about 57 kDa; about 58 kDa; about 59 kDa; about 60 kDa; about 61 kDa; about 62 kDa; about 63 kDa; about 64 kDa; about 65 kDa; about 66 kDa; about 67 kDa; about 68 kDa; about 69 kDa; about 70 kDa; about 71 kDa; about 72 kDa; about 73 kDa; about 74 kDa; about 75 kDa; about 76 kDa; about 77 kDa; about 78 kDa; about 79 kDa; about 80 kDa; about 81 kDa; about 82 kDa; about 83 kDa; about 84 kDa; about 85 kDa; about 86 kDa; about 87 kDa; about 88 kDa; about 89 kDa; about 90 kDa; about 91 kDa; about 92 kDa; about 93 kDa; about 94 kDa; about 95 kDa; about 96 kDa; about 97 kDa; about 98 kDa; about 99 kDa; or about 100 kDa. Recitation of each of these discrete values is understood to include ranges between each value. Recitation of each range is understood to include discrete values within the range. Recitation of each of these discrete values is understood to include greater than, less than, and/or equal to the discrete value or range.

The PEG as described herein can have length (e.g., n, m, W) between 1 and 3, between 16 and 24, between 222 and 1000, between 19 and 1000, between 19 and 32, or between 100 and 900. For example, the PEG length can be greater than 16. As another example, the PEG length can be about 1; about 2; about 3; about 4; about 5; about 6; about 7; about 8; about 9; about 10; about 11; about 12; about 13; about 14; about 15; about 16; about 17; about 18; about 19; about 20; about 21; about 22; about 23; about 24; about 25; about 26; about 27; about 28; about 29; about 30; about 31; about 32; about 33; about 34; about 35; about 36; about 37; about 38; about 39; about 40; about 41; about 42; about 43; about 44; about 45; about 46; about 47; about 48; about 49; about 50; about 51; about 52; about 53; about 54; about 55; about 56; about 57; about 58; about 59; about 60; about 61; about 62; about 63; about 64; about 65; about 66; about 67; about 68; about 69; about 70; about 71; about 72; about 73; about 74; about 75; about 76; about 77; about 78; about 79; about 80; about 81; about 82; about 83; about 84; about 85; about 86; about 87; about 88; about 89; about 90; about 91; about 92; about 93; about 94; about 95; about 96; about 97; about 98; about 99; about 100; about 101; about 102; about 103; about 104; about 105; about 106; about 107; about 108; about 109; about 110; about 111; about 112; about 113; about 114; about 115; about 116; about 117; about 118; about 119; about 120; about 121; about 122; about 123; about 124; about 125; about 126; about 127; about 128; about 129; about 130; about 131; about 132; about 133; about 134; about 135; about 136; about 137; about 138; about 139; about 140; about 141; about 142; about 143; about 144; about 145; about 146; about 147; about 148; about 149; about 150; about 151; about 152; about 153; about 154; about 155; about 156; about 157; about 158; about 159; about 160; about 161; about 162; about 163; about 164; about 165; about 166; about 167; about 168; about 169; about 170; about 171; about 172; about 173; about 174; about 175; about 176; about 177; about 178; about 179; about 180; about 181; about 182; about 183; about 184; about 185; about 186; about 187; about 188; about 189; about 190; about 191; about 192; about 193; about 194; about 195; about 196; about 197; about 198; about 199; about 200; about 201; about 202; about 203; about 204; about 205; about 206; about 207; about 208; about 209; about 210; about 211; about 212; about 213; about 214; about 215; about 216; about 217; about 218; about 219; about 220; about 221; about 222; about 223; about 224; about 225; about 226; about 227; about 228; about 229; about 230; about 231; about 232; about 233; about 234; about 235; about 236; about 237; about 238; about 239; about 240; about 241; about 242; about 243; about 244; about 245; about 246; about 247; about 248; about 249; about 250; about 251; about 252; about 253; about 254; about 255; about 256; about 257; about 258; about 259; about 260; about 261; about 262; about 263; about 264; about 265; about 266; about 267; about 268; about 269; about 270; about 271; about 272; about 273; about 274; about 275; about 276; about 277; about 278; about 279; about 280; about 281; about 282; about 283; about 284; about 285; about 286; about 287; about 288; about 289; about 290; about 291; about 292; about 293; about 294; about 295; about 296; about 297; about 298; about 299; about 300; about 301; about 302; about 303; about 304; about 305; about 306; about 307; about 308; about 309; about 310; about 311; about 312; about 313; about 314; about 315; about 316; about 317; about 318; about 319; about 320; about 321; about 322; about 323; about 324; about 325; about 326; about 327; about 328; about 329; about 330; about 331; about 332; about 333; about 334; about 335; about 336; about 337; about 338; about 339; about 340; about 341; about 342; about 343; about 344; about 345; about 346; about 347; about 348; about 349; about 350; about 351; about 352; about 353; about 354; about 355; about 356; about 357; about 358; about 359; about 360; about 361; about 362; about 363; about 364; about 365; about 366; about 367; about 368; about 369; about 370; about 371; about 372; about 373; about 374; about 375; about 376; about 377; about 378; about 379; about 380; about 381; about 382; about 383; about 384; about 385; about 386; about 387; about 388; about 389; about 390; about 391; about 392; about 393; about 394; about 395; about 396; about 397; about 398; about 399; about 400; about 401; about 402; about 403; about 404; about 405; about 406; about 407; about 408; about 409; about 410; about 411; about 412; about 413; about 414; about 415; about 416; about 417; about 418; about 419; about 420; about 421; about 422; about 423; about 424; about 425; about 426; about 427; about 428; about 429; about 430; about 431; about 432; about 433; about 434; about 435; about 436; about 437; about 438; about 439; about 440; about 441; about 442; about 443; about 444; about 445; about 446; about 447; about 448; about 449; about 450; about 451; about 452; about 453; about 454; about 455; about 456; about 457; about 458; about 459; about 460; about 461; about 462; about 463; about 464; about 465; about 466; about 467; about 468; about 469; about 470; about 471; about 472; about 473; about 474; about 475; about 476; about 477; about 478; about 479; about 480; about 481; about 482; about 483; about 484; about 485; about 486; about 487; about 488; about 489; about 490; about 491; about 492; about 493; about 494; about 495; about 496; about 497; about 498; about 499; about 500; about 501; about 502; about 503; about 504; about 505; about 506; about 507; about 508; about 509; about 510; about 511; about 512; about 513; about 514; about 515; about 516; about 517; about 518; about 519; about 520; about 521; about 522; about 523; about 524; about 525; about 526; about 527; about 528; about 529; about 530; about 531; about 532; about 533; about 534; about 535; about 536; about 537; about 538; about 539; about 540; about 541; about 542; about 543; about 544; about 545; about 546; about 547; about 548; about 549; about 550; about 551; about 552; about 553; about 554; about 555; about 556; about 557; about 558; about 559; about 560; about 561; about 562; about 563; about 564; about 565; about 566; about 567; about 568; about 569; about 570; about 571; about 572; about 573; about 574; about 575; about 576; about 577; about 578; about 579; about 580; about 581; about 582; about 583; about 584; about 585; about 586; about 587; about 588; about 589; about 590; about 591; about 592; about 593; about 594; about 595; about 596; about 597; about 598; about 599; about 600; about 601; about 602; about 603; about 604; about 605; about 606; about 607; about 608; about 609; about 610; about 611; about 612; about 613; about 614; about 615; about 616; about 617; about 618; about 619; about 620; about 621; about 622; about 623; about 624; about 625; about 626; about 627; about 628; about 629; about 630; about 631; about 632; about 633; about 634; about 635; about 636; about 637; about 638; about 639; about 640; about 641; about 642; about 643; about 644; about 645; about 646; about 647; about 648; about 649; about 650; about 651; about 652; about 653; about 654; about 655; about 656; about 657; about 658; about 659; about 660; about 661; about 662; about 663; about 664; about 665; about 666; about 667; about 668; about 669; about 670; about 671; about 672; about 673; about 674; about 675; about 676; about 677; about 678; about 679; about 680; about 681; about 682; about 683; about 684; about 685; about 686; about 687; about 688; about 689; about 690; about 691; about 692; about 693; about 694; about 695; about 696; about 697; about 698; about 699; about 700; about 701; about 702; about 703; about 704; about 705; about 706; about 707; about 708; about 709; about 710; about 711; about 712; about 713; about 714; about 715; about 716; about 717; about 718; about 719; about 720; about 721; about 722; about 723; about 724; about 725; about 726; about 727; about 728; about 729; about 730; about 731; about 732; about 733; about 734; about 735; about 736; about 737; about 738; about 739; about 740; about 741; about 742; about 743; about 744; about 745; about 746; about 747; about 748; about 749; about 750; about 751; about 752; about 753; about 754; about 755; about 756; about 757; about 758; about 759; about 760; about 761; about 762; about 763; about 764; about 765; about 766; about 767; about 768; about 769; about 770; about 771; about 772; about 773; about 774; about 775; about 776; about 777; about 778; about 779; about 780; about 781; about 782; about 783; about 784; about 785; about 786; about 787; about 788; about 789; about 790; about 791; about 792; about 793; about 794; about 795; about 796; about 797; about 798; about 799; about 800; about 801; about 802; about 803; about 804; about 805; about 806; about 807; about 808; about 809; about 810; about 811; about 812; about 813; about 814; about 815; about 816; about 817; about 818; about 819; about 820; about 821; about 822; about 823; about 824; about 825; about 826; about 827; about 828; about 829; about 830; about 831; about 832; about 833; about 834; about 835; about 836; about 837; about 838; about 839; about 840; about 841; about 842; about 843; about 844; about 845; about 846; about 847; about 848; about 849; about 850; about 851; about 852; about 853; about 854; about 855; about 856; about 857; about 858; about 859; about 860; about 861; about 862; about 863; about 864; about 865; about 866; about 867; about 868; about 869; about 870; about 871; about 872; about 873; about 874; about 875; about 876; about 877; about 878; about 879; about 880; about 881; about 882; about 883; about 884; about 885; about 886; about 887; about 888; about 889; about 890; about 891; about 892; about 893; about 894; about 895; about 896; about 897; about 898; about 899; about 900; about 901; about 902; about 903; about 904; about 905; about 906; about 907; about 908; about 909; about 910; about 911; about 912; about 913; about 914; about 915; about 916; about 917; about 918; about 919; about 920; about 921; about 922; about 923; about 924; about 925; about 926; about 927; about 928; about 929; about 930; about 931; about 932; about 933; about 934; about 935; about 936; about 937; about 938; about 939; about 940; about 941; about 942; about 943; about 944; about 945; about 946; about 947; about 948; about 949; about 950; about 951; about 952; about 953; about 954; about 955; about 956; about 957; about 958; about 959; about 960; about 961; about 962; about 963; about 964; about 965; about 966; about 967; about 968; about 969; about 970; about 971; about 972; about 973; about 974; about 975; about 976; about 977; about 978; about 979; about 980; about 981; about 982; about 983; about 984; about 985; about 986; about 987; about 988; about 989; about 990; about 991; about 992; about 993; about 994; about 995; about 996; about 997; about 998; about 999; or about 1000. Recitation of each of these discrete values is understood to include ranges between each value. Recitation of each range is understood to include discrete values within the range. Recitation of each of these discrete values is understood to include greater than, less than, and/or equal to the discrete value or range.

### Formulas

In some aspects, the present disclosure provides compounds of the formula:

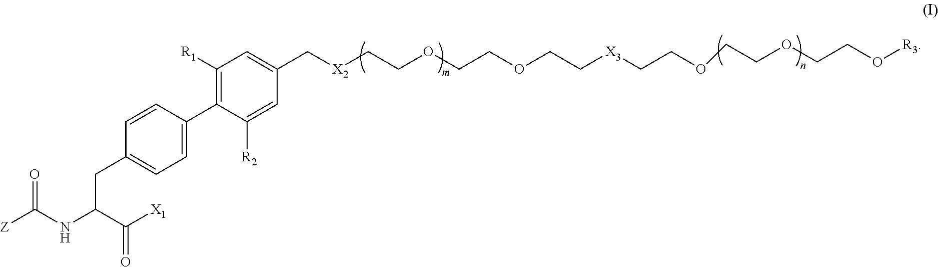

In some embodiments,

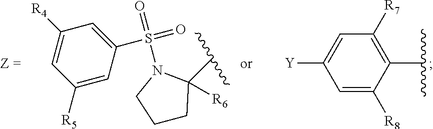

m=1-3; when

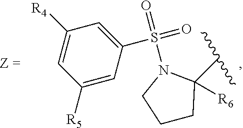

then n=19-1000; when

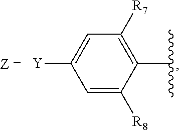

then n=222-1000;
R₁ and R₂ are each independently hydroxyl, alkoxy_{(C≤8)} or substituted alkoxy_{(C≤8)}; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), where R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and where R₉ is hydrogen, alkyl_{(C≤6)}, substituted alkyl_{(C≤6)}; or where R₃ may represents a biomarker tag for in vitro or in vivo utility, an antibody targeting a specific protein or receptor, or another entity, which when attached to the core molecule enhanced the biological effects of the combined entities, all attached to the core molecule at the R₃ site; if m=1, and n=19-32, and X₃ is oxygen, then R₃ can also be:

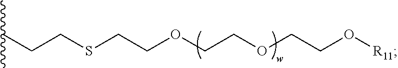

where w=100-900; and/or R₁₁ is defined as above for R₃ and/or, if X₃ is oxygen, m=1 and n=19-1000, then R₃ can also be

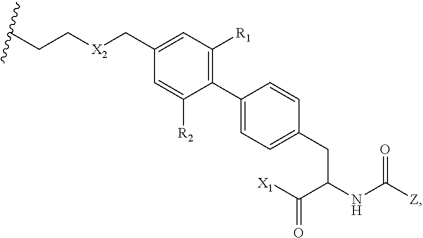

where R₁, R₂, X₁, and Z are defined above; R₄ and R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, or a substituent convertible in vivo to hydroxy; or a pharmaceutically acceptable salt thereof;
R₆ is hydrogen, alkyl_{(C≤6)}, or substituted alkyl_{(C≤6)}; R₇ and R₈ are each independently hydrogen, halo, haloalkyl_{(C≤8)}; Y is hydrogen, cyano, halo, haloalkyl, hydroxy, or —C(O)X₄, wherein: X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof; X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof; X₂ is oxygen or sulfur; X₃ is oxygen, sulfur, —NH(C═O)—, —(C═O)NH—, —N(R₁₂)—; or where R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

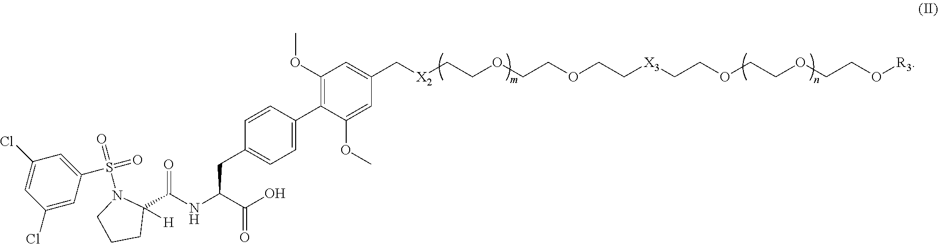

In some embodiments, m=1-3; n=19-1000; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), where R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and where R₉ is hydrogen, alkyl_{(C≤6)}, substituted alkyl_{(C≤6)}; or where R₃ may represent a biomarker tag for in vitro or in vivo utility, an antibody targeting a specific protein or receptor, or another entity, which when attached to the core molecule enhanced the biological effects of the combined entities, all attached to the core molecule at the R₃ site; or if m=1, and n=19-32, and X₃ is oxygen, then R₃ can also be:

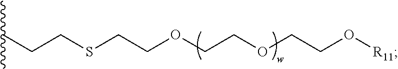

where w=100-900; and/or R₁₁ is defined as above for R₃; and/or, if X₃ is oxygen, m=1 and n=19-1000, then R₃ can also be:

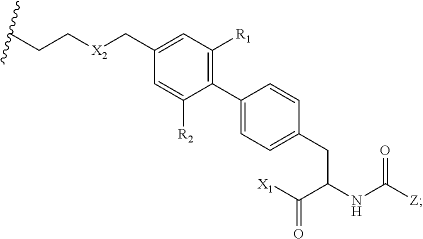

where R₁, R₂, X₁, and Z are defined above; X₂ is oxygen or sulfur; or X₃ is oxygen, sulfur, —NH(C═O)—, —(C═O)NH—, —N(R₁₂)—; where R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

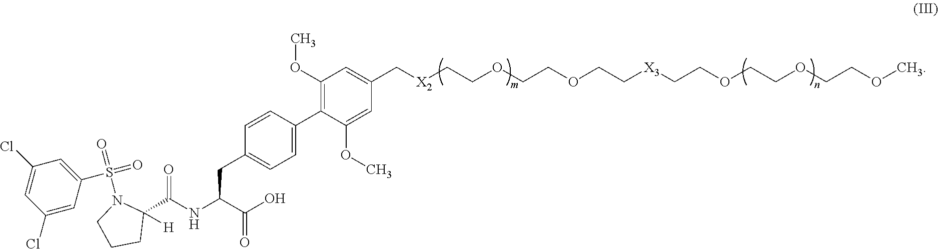

In some embodiments, m=1-3; n=19-1000; X₂ is oxygen or sulfur; or X₃ is oxygen, sulfur, —NH(C═O)—, —(C═O)NH—, —N(R₁₂)—; where R₁₂ is alkyl_{(C≤6)}, substituted alkyl_{(C≤6)}; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

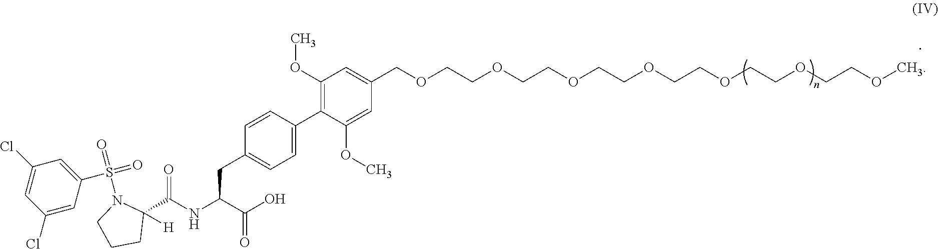

In some embodiments, n=19; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

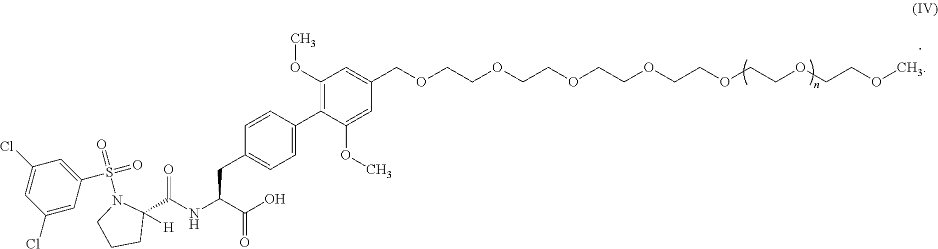

In some embodiments, n=31; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

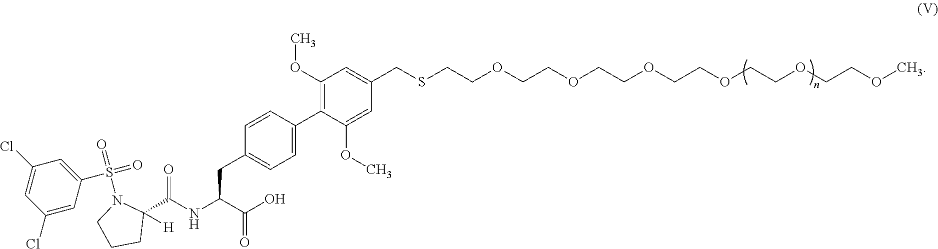

In some embodiments, n=19-1000; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

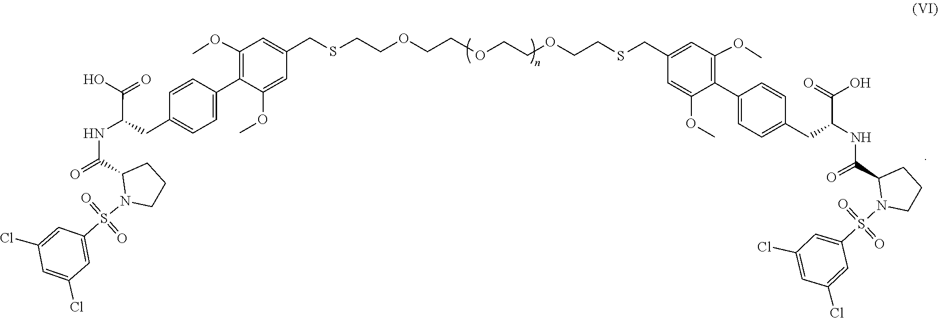

In some embodiments, n=19-1000; or a pharmaceutically acceptable salt, solvate, polymorph, tautomer, prodrug, analog, or stereoisomer thereof or optionally substituted analog thereof.

In some aspects, the present disclosure provides compounds of the formula:

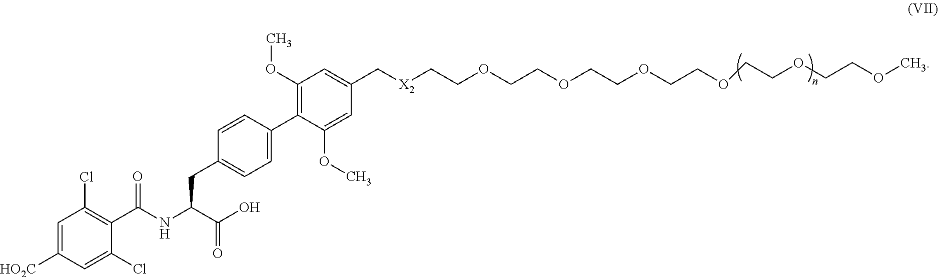

In some embodiments, n=222-1000; X₂ is oxygen or sulfur; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

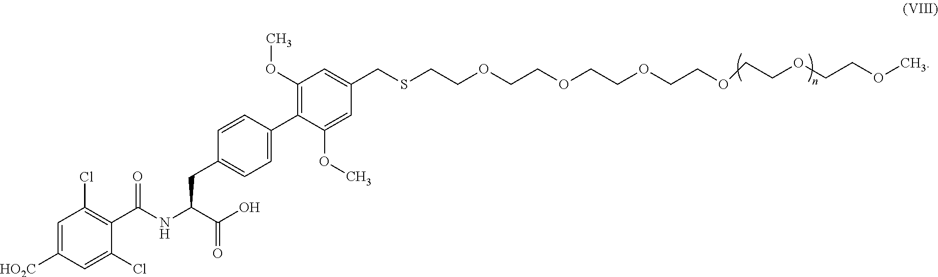

In some embodiments, n=222-1000; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

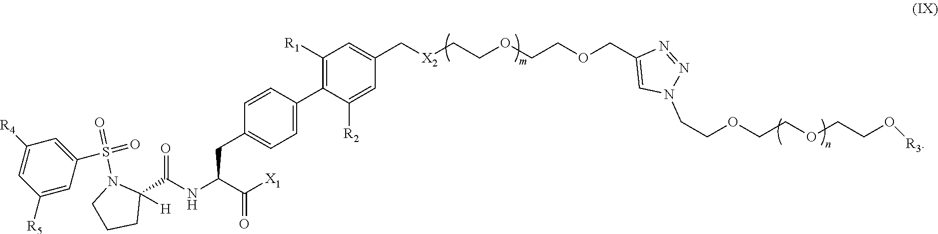

In some embodiments, m=1-3; n=19-1000; X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof; X₂ is oxygen or sulfur; R₁ and R₂ are each independently hydroxyl, alkoxy_{(C≤8)} or substituted alkoxy_{(C≤8)}; R₃ is hydrogen, alkyl_{(C≤6)}, substituted alkyl_{(C≤6)}, haloalkyl, aryl, substituted aryl, —CH₂—CH₂—SO₂-alkyl_{(C≤8)}, —CH₂—CH₂—N(R₉)(R₁₀), where R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and where R₉ is hydrogen, alkyl_{(C≤6)}, substituted alkyl_{(C≤6)}; or where R₃ may represent a biomarker tag for in vitro or in vivo utility, an antibody targeting a specific protein or receptor, or another entity, which when attached to the core molecule enhanced the biological effects of the combined entities, all attached to the core molecule at the R₃ site; or if m=1, and n=19-32, then R₃ can also be:

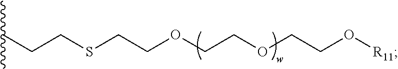

where w=100-900; and/or R₁₁ is defined as above for R₃; or R₄ and R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, or a substituent convertible in vivo to hydroxy; or a pharmaceutically acceptable salt thereof; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

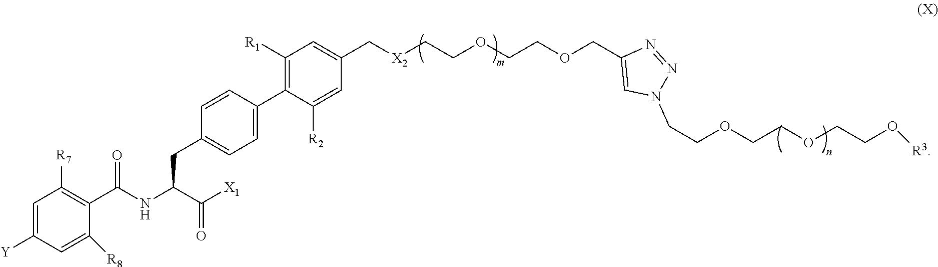

In some embodiments, m=1-3; n=222-1000; X₁, X₂, R₁, R₂, and R₃ are as defined above, R₇ and R₈ are each independently hydrogen, halo, haloalkyl_{(C≤8)}; Y is hydrogen, cyano, halo, haloalkyl, hydroxy, or —C(O)X₄, wherein: X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

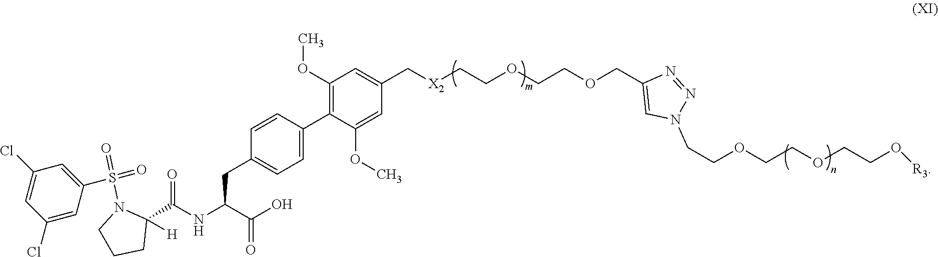

In some embodiments, m=1-3; n=19-1000; X₂ is oxygen or sulfur; R₃ is hydrogen, alkyl_{(C≤6)}, substituted alkyl_{(C≤6)}, haloalkyl, aryl, substituted aryl, —CH₂—CH₂—SO₂— alkyl_{(C≤8)}, —CH₂—CH₂—N(R₉)(R₁₀), where R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and where R₉ is hydrogen, alkyl_{(C≤6)}, substituted alkyl_{(C≤6)}; or where R₃ may represent a biomarker tag for in vitro or in vivo utility, an antibody targeting a specific protein or receptor, or another entity, which when attached to the core molecule enhanced the biological effects of the combined entities, all attached to the core molecule at the R₃ site; if m=1, and n=19-32, then R₃ can also be:

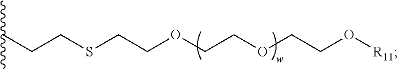

where w=100-900; and R₁₁ is defined as above for R₃; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

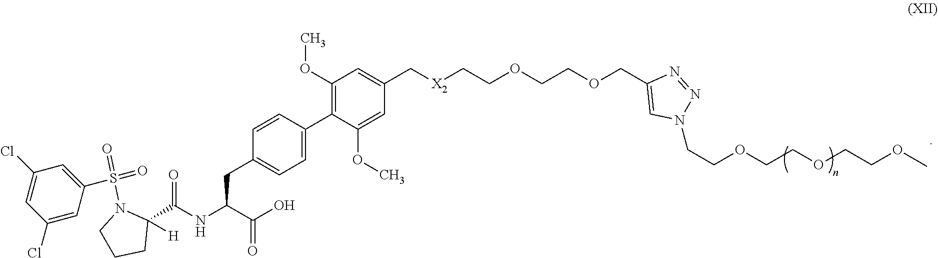

In some embodiments, n=19-1000; X₂ is oxygen or sulfur; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

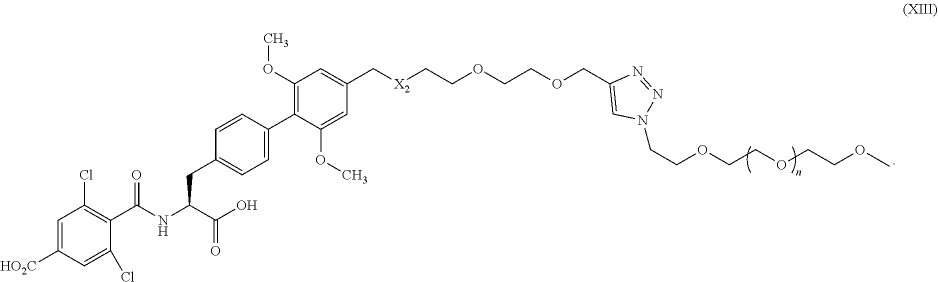

In some embodiments, n=222-1000; X₂ is oxygen or sulfur; or a pharmaceutically acceptable salt, solvate, polymorph, tautomer, prodrug, analog, or stereoisomer thereof or optionally substituted analog thereof or bis-derivative thereof.

In some aspects, the present disclosure provides compounds of the formula:

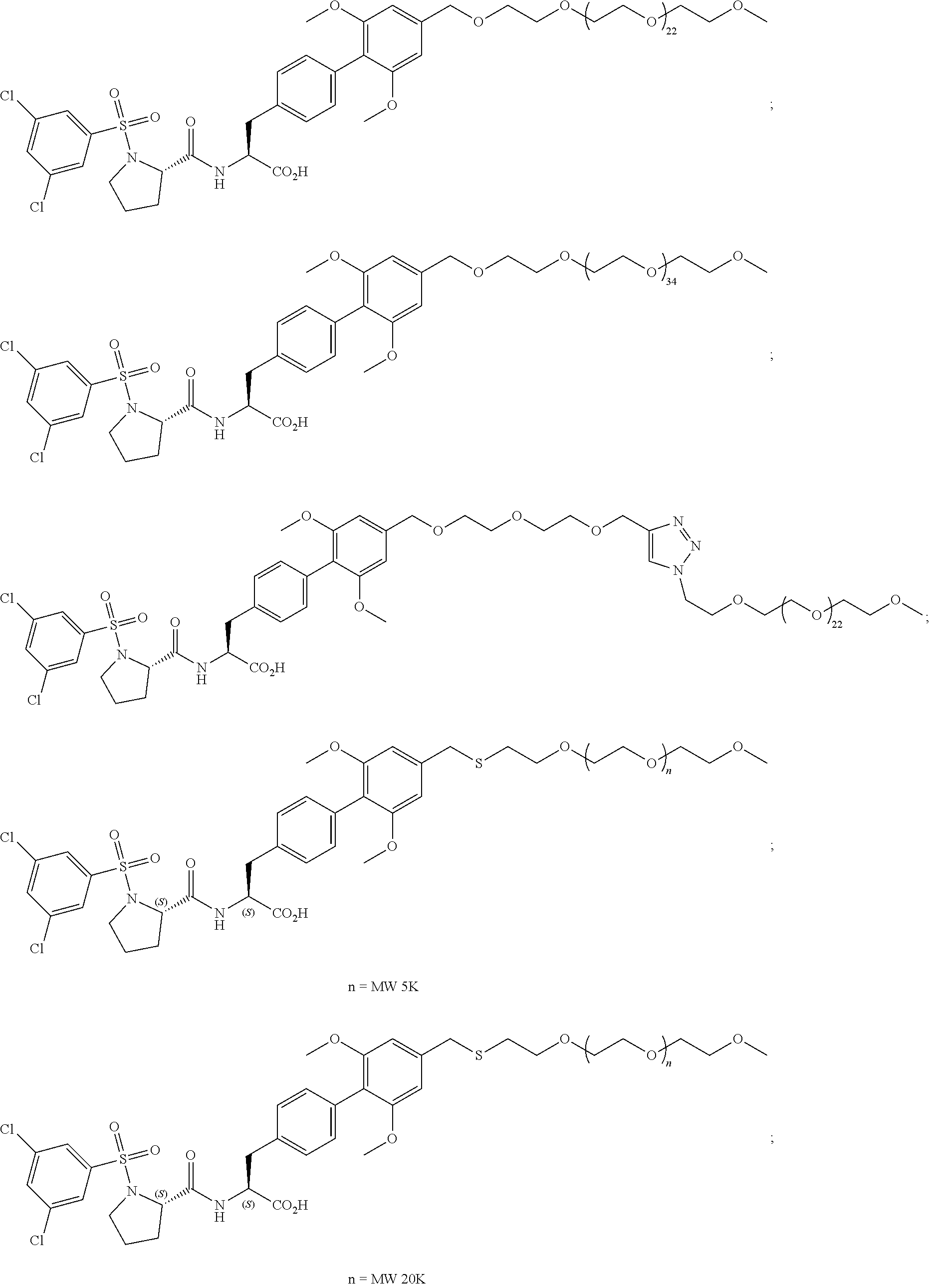

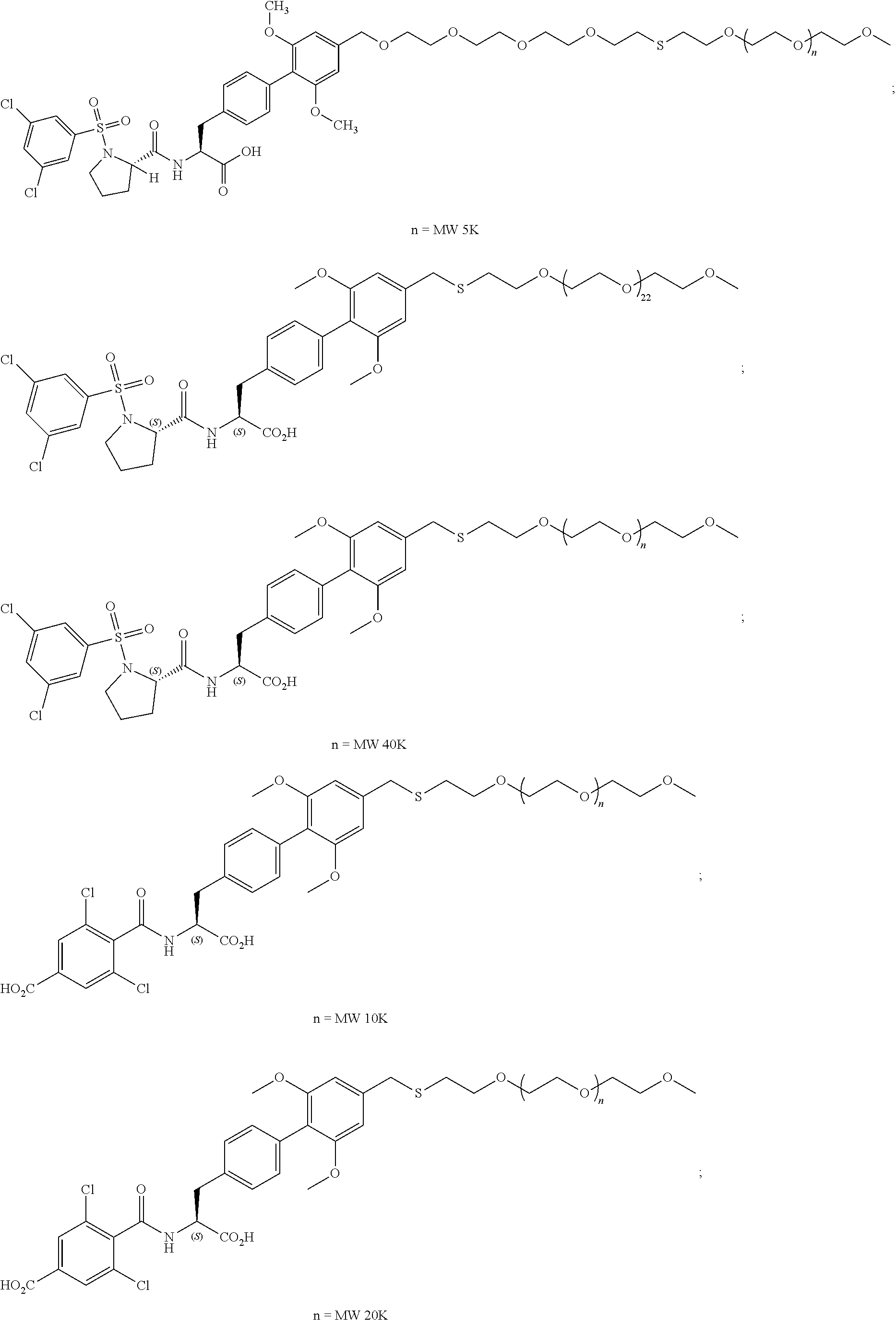

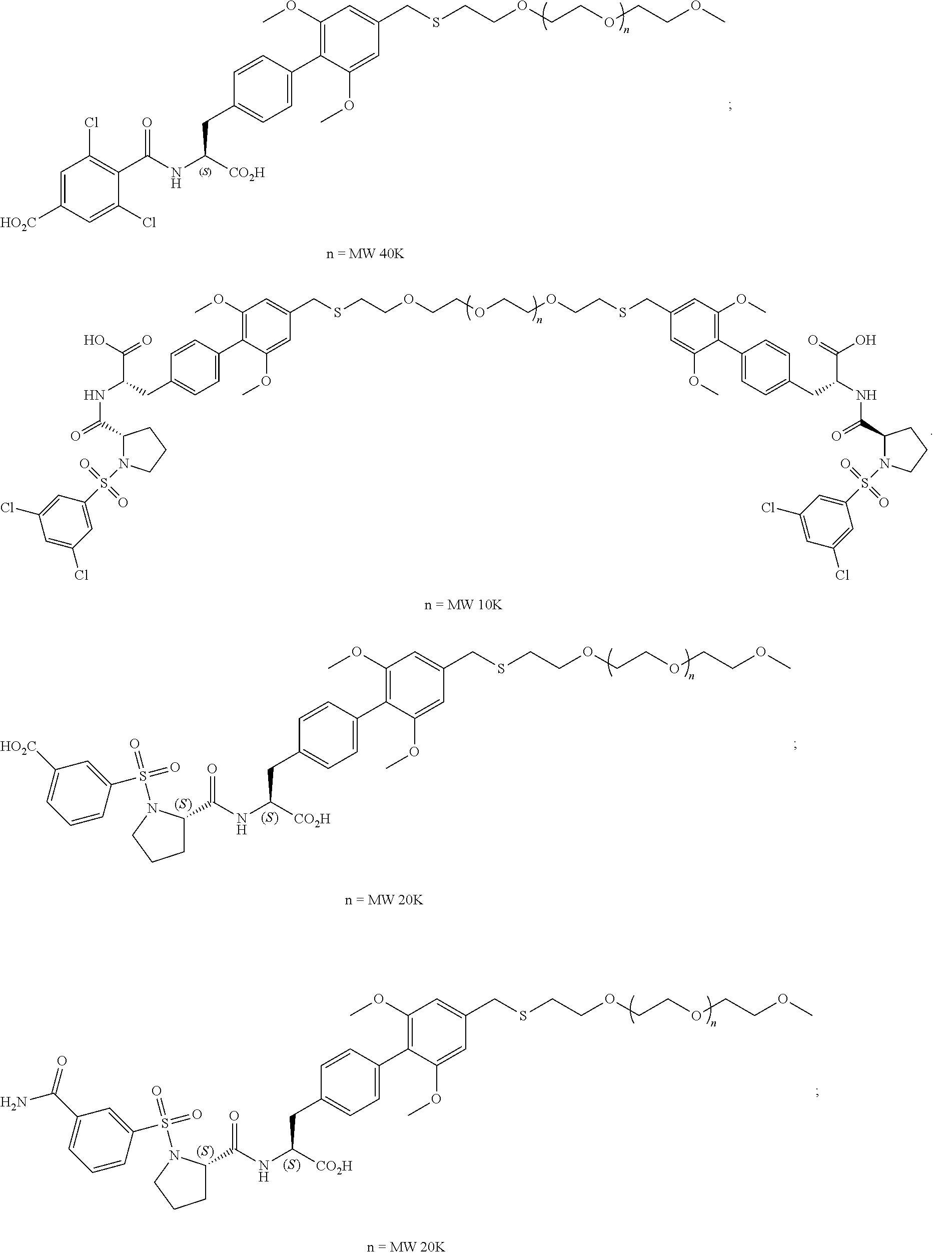

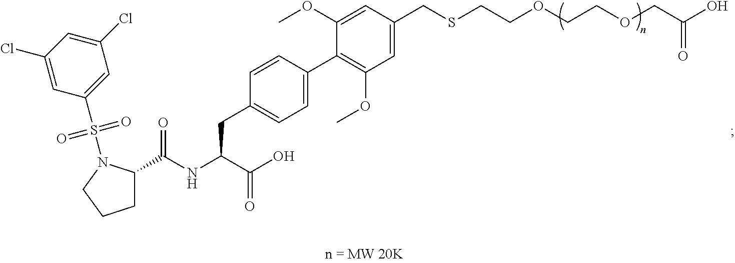

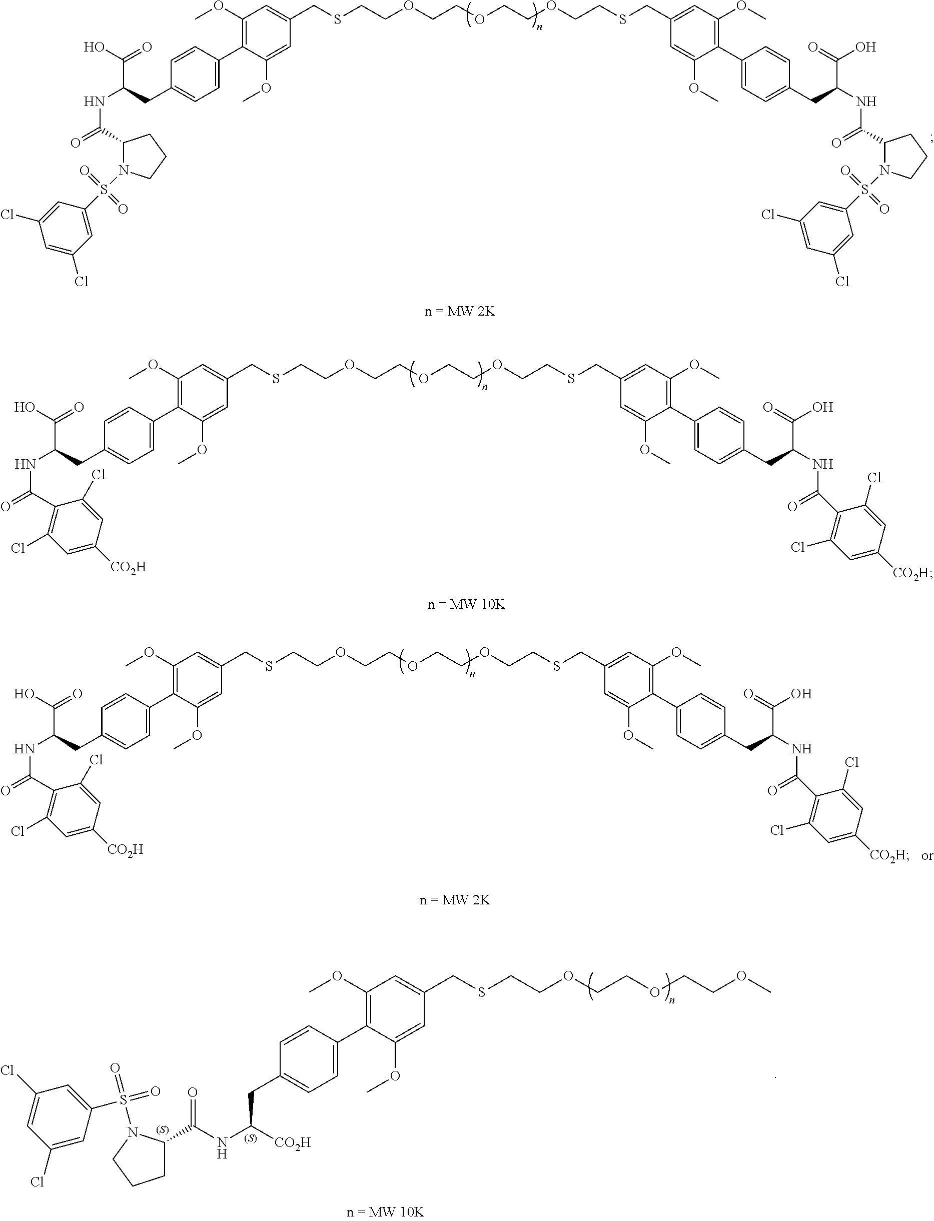

### Bis-Derivatives

As described herein, bis-derivatives or bis-analogs of VLA-4 inhibitors can be useful for the methods described herein. The bis-analog uses the “di-chloro sulfonamide” core (see e.g., Example 11). As another example, a bis analog or bis-derivative can have the “di-chloro benzoic acid” core.

### Chemical Definitions

Any of R groups R₁-R₁₂ can be optionally substituted or functionalized with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; a C₂₋₁₀ cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₁₀ cycloalkyl optionally containing unsaturation or one oxygen or nitrogen atom; straight chain or branched C₁₋₁₀ alkyl amine; heterocyclyl; heterocyclic amine; aryl comprising a phenyl; and heteroaryl containing from 1 to 4N, O, or S atoms; and the unsubstituted heterocyclyl or substituted heterocyclyl can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₁₀ cycloalkyl optionally containing unsaturation or one oxygen or nitrogen atom; heterocyclyl; straight chain or branched C₁₋₁₀ alkyl amine; heterocyclic amine; and aryl comprising a phenyl; and heteroaryl containing from 1 to 4N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “alkyl” can refer to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” can refer to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂—(methylene), —CH₂CH₂—, —CH₂C(CH₃)₂CH₂—, and —CH₂CH₂CH₂—are non-limiting examples of alkanediyl groups. The term “alkylidene” can refer to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e., —F, —Cl, —Br, or —I) such that no other atoms aside from carbon, hydrogen, and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen, and fluorine are present. The groups —CH₂F, —CF₃, and —CH₂CF₃ are non-limiting examples of fluoroalkyl groups.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of the alkenyl moiety. An alkenyl can be partially saturated or unsaturated. The “alkenyl” can be optionally substituted.

The term “alkenyl” can refer to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” can refer to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂—are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O— cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “cycloalkyl” can refer to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” can refer to a divalent saturated aliphatic group with two carbon atoms as points of attachment, no carbon-carbon double or triple bonds, and no atoms other than carbon and hydrogen. The group

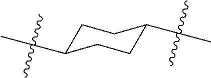 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The terms “alkylsulfonyl” and “alkylsulfinyl” can refer to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

When used in the context of a chemical group: “hydrogen” means —H; “hydroxy” means —OH; “oxo” means ═O; “carbonyl” means —C(═O)—; “carboxy” means —C(═O)OH (also written as -GOGH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “ [symbol] ” means triple bond. The symbol “ [symbol] ” represents an optional bond, which if present is either single or double. The symbol “ [symbol] ” represents a single bond or a double bond. Thus, the formula

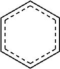

covers, for example,

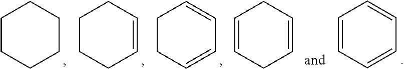

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

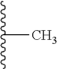

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

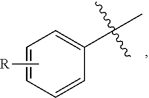

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

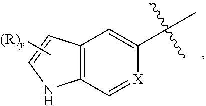

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in a moiety replacing a hydrogen atom is not counted. Thus methoxyhexyl, which has a total of seven carbon atoms, is an example of a substituted alkyl_{(C1-6)}.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” can signify that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic π system.

The term “aryl” can refer to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” can refer to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl, or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

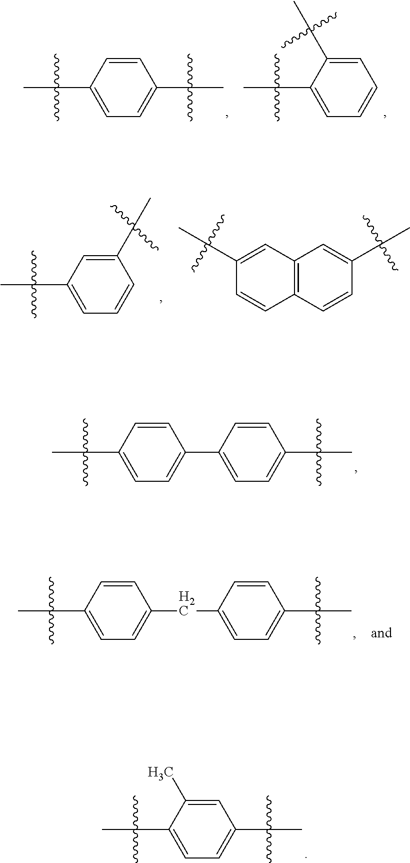

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” can refer to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “acyl” can refer to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” can refer to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “aryloxy”, “aralkoxy”, and “acyloxy”, can refer to groups, defined as —OR, in which R is cycloalkyl, alkenyl, aryl, aralkyl, and acyl, respectively. The term “alkylthio” and “acylthio” can refer to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” can refer to the group —NHR, in which R is an alkyl, as that term is defined above. Non-limiting examples include: —NHCH₃ and —NHCH₂CH₃. The term “dialkylamino” can refer to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “arylamino”, “aralkylamino”, “alkoxyamino”, and “alkylsulfonylamino” can refer to groups, defined as —NHR, in which R is cycloalkyl, alkenyl, aryl, aralkyl, alkoxy, and alkylsulfonyl, respectively. A non-limiting example of an arylamino group is —NHC₆H₅. The term “amido” (acylamino), can refer to the group —NHR, in which R is acyl, as that term is defined above. A non-limiting example of an amido group is —NHC(O)CH₃. The term “alkylimino” can refer to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The methods and compositions used herein may contain one or more VLA-4 inhibitors or VLA-4 antagonists. The terms VLA-4 inhibitor and VLA-4 antagonist are used interchangeably in the disclosure. Some non-limiting examples of VLA-4 inhibitors which may be used in the compositions and methods described herein include antibodies, such as humanized monoclonal antibody against a4, natalizumab (Antegren®), and small molecules such as those described in U.S. Pat. No. 5,510,332; WO 06/023396; WO 97/03094; WO 97/02289; WO 96/40781; WO 96/22966; WO 96/20216; WO 96/01644; WO 96/06108; WO 95/15973; WO 96/31206; WO 06/010054; WO 05/087760; WO 01/12186; WO 99/37605; WO 01/51487; WO 03/011288; WO 02/14272; WO 01/32610; and EP 0842943, the entire contents of which are hereby incorporated by reference. An example of a VLA-4 inhibitor that may be used herein is BIO5192 (also known as AMD15057) disclosed in PCT publication WO 01/12186, which is incorporated herein by reference. Alternatively, analogs of BIO5192, such as BIO1211, may be used. In other embodiments, the VLA-4 inhibitor is firategrast or a pharmaceutically acceptable salt thereof.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the technology in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the present disclosure. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the present disclosure, or a salt thereof, that further can include a stoichiometric or non-stoichiometric amount of water bound by non-covalent intermolecular forces.

In some embodiments, the compounds used in the compositions of the present disclosure include the compounds described in the Examples and claims listed below. All the synthesis methods described above can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures, and individual diastereomers. In some embodiments, a single diastereomer is obtained. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof. Tautomeric forms are also included as well as pharmaceutically acceptable salts of such isomers and tautomers.

Atoms making up the compounds of the present disclosure are intended to include all isotopic forms of such atoms. Compounds of the present disclosure include those with one or more atoms that have been isotopically modified or enriched, in particular those with pharmaceutically acceptable isotopes or those useful for pharmaceutical research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively. Additional details regarding prodrugs may be found in Smith and Williams, 1988, the entire contents of which are hereby incorporated by reference. Smith and Williams Introduction to the Principles of Drug Design, Smith, H. J.; Wright, 2nd ed., London (1988), the contents of which are hereby incorporated by reference.

It should be recognized that the particular anion or cation forming a part of any salt of this disclosure is not critical, so long as the salt, as a whole, is pharmacologically acceptable. Compounds useful in the disclosure which are amines, may be administered or prepared in the forms of their acid addition salts or metal complexes thereof. Suitable acid addition salts include salts of inorganic acids that are biocompatible, including HCl, HBr, sulfuric, phosphoric, and the like, as well as organic acids such as acetic, propionic, butyric, and the like, as well as acids containing more than one carboxyl group, such as oxalic, glutaric, adipic and the like. Compounds useful in the disclosure that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present disclosure include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃, Boc), benzyloxycarbonyl, p-methoxy-benzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃, Boc), and the like. Suitable peptide residues include peptide residues comprising two to five amino acid residues. The residues of these amino acids or peptides can be present in stereochemical configurations of the D-form, the L-form, or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr, and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl, and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl, and β-iodoethoxycarbonyl).

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Agents which Interact with Chemokine Receptors

As used herein, the “agent which interacts with a chemokine receptor” can include chemokines, cytokines, chemokine receptors, or an agent which modulates the activity of these molecules such as a fragment, an antibody, or a small organic molecule.

In some embodiments, the agent which interacts with a chemokine receptor is an agent which interacts with a CXC chemokine receptor. In one embodiment, the present disclosure relates to compositions that modulate the activity of a CXC chemokine receptor such as CXCR2 or CXCR4. In some embodiments, the present methods and compositions contain at least one CXCR2 agonist or CXCR4 antagonist. In some embodiments, the present methods and compositions contain at least one CXCR2 ligand or CXCR4 ligand. The ligand can be an agonist or antagonist.

As used herein “interacts with” means that the agent binds with a chemokine in a manner that modulates the activity of said chemokine, for example, by reducing, inhibiting, increasing, or activating the activity of the chemokine. In one embodiment, the present disclosure relates to compositions that modulate the activity of a CXC chemokine receptor such as CXCR2 or CXCR4. In some embodiments, the present methods and compositions comprise at least two agents which interact with a chemokine. In some embodiments, the present methods and compositions comprise a first agent comprising a CXCR2 agonist and a second agent comprising a CXCR4 inhibitor. In a preferred embodiment, the methods and compositions comprise a VLA-4 inhibitor or VLA-4 antagonist as disclosed herein, a CXCR4 inhibitor, and/or a CXCR2 agonist. In a particular embodiment, the methods and compositions comprise a VLA-4 inhibitor or VLA-4 antagonist as disclosed herein, AMD3100 (Plerixafor) or BL-8040 (Motixafortide), and/or Gro□.

In some embodiments, the agent is a CXCR2 agonist. In some embodiments, the agent is Groβ or a derivative of Groβ. In some embodiments, the derivative of Groβ is a truncated Groβ (tGroβ). In some embodiments, the truncated Groβ is SB-251353.

CXCR2 agonists include any molecule that activates the CXCR2 receptor. Such molecules include chemokines, cytokines, agonist antibodies or biologically active fragments thereof, or small organic molecules. Some non-limiting examples of chemokines acting via the CXCR2 receptor include, but are not limited to Groβ, Groα, Groγ, GCP-2 (granulocyte chemo-attractant protein 2), IL-8, NAP-2 (neutrophil activating peptide 2), ENA-78 (epithelial-cell derived neutrophil activating protein 78), and MGSA.

A CXCR2 receptor agonist that may be used in the compositions and methods described herein is SB-251353, a basic, heparin-binding protein with a molecular mass of approximately 7500 Da (King et al., 2000, Hepburn et al., 2001). In some embodiments, the CXCR2 agonists used in the methods and compositions described herein are Groβ and modified forms thereof. King et al., 2001 have demonstrated that a recombinant N-terminal 4-amino acid truncated form of the human chemokine Groβ (also known as SB-251353 or garnocestim or Groβt or tGroβ) can mobilize progenitor cells after administration of SB-251353 in combination with G-CSF. This combination resulted in the mobilization of neutrophils and platelets during these studies. Chemokines such as the SB-251353, Groα, Groβ, and Groγ are further discussed in WO 94/29341; WO 97/15594; WO 97/15595; WO 99/26645; WO 02/02132; U.S. Pat. Nos. 6,080,398; 6,399,053; and 6,447,766, which are incorporated herein by reference.

The “Groβ”, “Groβ protein”, or “Groβ chemokine” class includes Groβ itself as well as modified forms of Groβ. These modified forms include, but are not limited to, truncated, multimerized, amino-acid substituted, modified with amino-acid deletions and/or insertions, or combinations thereof. “Modified forms of Groβ” include truncated forms such as those described in U.S. Pat. Nos. 6,447,766; 6,399,053; 6,080,398; PCT publication 99/26645; PCT publication WO 97/15595; PCT publication WO 02/02132; PCT publication WO 97/15594; and PCT publication WO 94/29341, which are incorporated herein by reference. “Modified forms of Groβ” are multimeric forms of Groβ such as dimers, trimers, tetramers, or other versions containing multiple proteins or modified proteins. Some non-limiting examples of “modified forms” include modified forms of Groβ with truncation of between 2 to about 8 amino acids at the amino terminus of the mature protein, truncation of between about 2 to about 10 amino acids at the carboxy terminus of the mature protein, or multimeric forms of the modified and/or truncated proteins, e.g., dimers, trimers, tetramers, and other aggregated forms. Some non-limiting examples of truncated forms of Groβ may include SB-251353 which consists of amino acids 5-73 and forms thereof where amino acid 69 is deamidated.

Another specific CXCR2 receptor agonist that may be used in the compositions and methods described herein is SB-251353, a basic, heparin-binding protein with a molecular mass of approximately 7500 Da (King et al., J Immunol 2000; 164: 3774-3782, Hepburn et al., Journal of Pharmacology and Experimental Therapeutics 2001; 298: 886-893).

The compositions and methods described herein may comprise one or more CXCR4 inhibitors. Some non-limiting examples of CXCR4 inhibitors include AMD3100 (plerixafor), BL-8040 (Motixafortide), AMD3465, CTCE-0214, CTCE-9908, CP-1221 (linear peptides, cyclic peptides, natural amino-acids, unnatural amino acids, and peptidomimetic compounds), T140 and analogs, 4F-benzoyl-TN24003, KRH-1120, KRH-1636, KRH-2731, polyphemusin analogue, ALX40-4C, or CXCR4 inhibitors described in WO 01/85196, WO 99/50461, WO 01/94420, WO 03/090512, US 2005/0059702, US 2005027767, US 2003/9229341, U.S. Pat. Nos. 5,021,409, 6,001,826, and 5,583,131, each of which is incorporated by reference herein.

The compositions and methods described herein may comprise one or more CXCR4 inhibitors (e.g., antagonists). In some embodiments, the agent is a CXCR4 antagonist. In some embodiments, the agent is plerixafor or BL-8040 (Motixafortide), or a derivative thereof.

Some non-limiting examples of CXCR4 inhibitors include AMD3100 (plerixafor), BL-8040 (Motixafortide), AMD3465, CTCE-0214, CTCE-9908, CP-1221 (linear peptides, cyclic peptides, natural amino-acids, unnatural amino acids, and peptidomimetic compounds), T140 and analogs, 4F-benzoyl-TN24003, KRH-1120, KRH-1636, KRH-2731, polyphemusin analogue, ALX40-4C, or CXCR4 inhibitors described in WO 01/85196, WO 99/50461, WO 01/94420, WO 03/090512, US 2005/0059702, US 2005027767, US 2003/9229341, U.S. Pat. Nos. 5,021,409, 6,001,826, and 5,583,131, each of which is incorporated by reference herein.

The compositions or methods described herein can comprise G-CSF. It is contemplated that any suitable source of G-CSF may be employed. In some embodiments, the composition further comprises an inhibitor of integrin α9β1, G-CSF, a derivative of G-CSF, or a combination thereof. In some embodiments, the derivative of G-CSF is a pegylated G-CSF. In some embodiments, the inhibitor of integrin α9β1 is (N-benzenesulfonyl)-L-prolyl-L-O-(1-pyrrolidinylcarbonyl)tyrosine (BOP). In some embodiments, the G-CSF used in the compositions or methods may be either recombinant or purified using known techniques and includes, but is not limited to, Neupogen® filgrastim (Amgen), Neutrogin®/Granocyte® lenograstim (Chugai Pharmaceuticals), and Neulasta® pegylated filgrastim (Amgen). Additionally, biologically active fragments, variants, derivatives, or fusion proteins may also be employed provided these agents retain the ability to mobilize progenitor or stem cells.

In some aspects, the present disclosure provides a pharmaceutical composition comprising a composition disclosed herein and a pharmaceutically acceptable excipient. In some embodiments, the pharmaceutical composition is formulated for oral administration, intraarterial administration, intraperitoneal administration, intravenous administration, or subcutaneous administration. In some embodiments, the pharmaceutical composition is formulated for administration via intravenous infusion. In other embodiments, the pharmaceutical composition is formulated for administration via subcutaneous injection. In some embodiments, the composition consists substantially of the agent which interacts with one or more chemokine receptors, the VLA-4 inhibitor, and the pharmaceutically acceptable excipient. In some embodiments, the composition consists essentially of the agent which interacts with one or more chemokine receptors, the VLA-4 inhibitor, and the pharmaceutically acceptable excipient.

Disclosed herein are methods comprising an agent or agents which interact with a chemokine receptor, such as a CXCR2 agonist and a CXCR4 antagonist, and a compound that act as an integrin antagonist or inhibitor, such as an α4β1 integrin (VLA-4) antagonist as well as pharmaceutical compositions thereof. These pharmaceutical compositions may result in the mobilization of progenitor and/or stem cells from bone marrow to peripheral circulation. Additionally, provided herein are methods for the treatment and/or prevention of disease using these two therapeutic agents in combination. These compositions described herein may be used to stimulate progenitor and/or stem cells (e.g., hematopoietic stem cells such as CD34+ hematopoietic stem cells) and result in such stimulation in a shorter amount of time relative to either agent alone or other known agents or combinations. These compositions may also have the added advantage that they result in mobilization in higher numbers, begin mobilization in a shorter period of time or over a more prolonged period of time, or mobilize increased numbers of early progenitor and/or stem cells, LSK-SLAM cells, CFU—C cells, or other progenitor and/or stem cells which are competent to achieve successful engraftment into the patient. In some embodiments, these compositions may be used in improving the harvest of hematopoietic stem cells or progenitor cells. These methods, compositions, or uses are described in more detail below.

VLA-4 Inhibitors in Combination with Agents which Interact with Chemokine Receptors

The present disclosure provides methods using a compound that is a VLA-4 antagonist in combination with agents which interact with chemokine receptors. The chemokine receptors can be a CXCR2 (e.g., tGroβ) and/or a CXCR4 (e.g., AMD3100 (plerixafor), BL-8040 (Motixafortide)) including methods of use and methods of treatment therewith. Also, provided herein are compositions comprising these drugs. For example, the VLA-4 inhibitor compositions described herein in combination with chemokine interacting agents, such as plerixafor (AMD3100) or BL-8040 (Motixafortide) and/or tGroβ were shown to increase the amount or number of cells mobilized.

In some aspects, the present disclosure provides a method of treating a disease or disorder in a patient comprising administering to the patient a therapeutically effective amount of agents which interact with chemokine receptors and a VLA-4 inhibitor. In some embodiments, the disease or disorder is associated with integrin α₄β₁. In some embodiments, the disease or disorder is associated with hematopoietic stem cells. In some embodiments, the hematopoietic stem cells are LSK-SLAM cells. In some embodiments, the disease or disorder is cancer or a reduced blood cell count such as a reduced blood cell count resulting from a cancer therapy. In some embodiments, the disease or disorder is a reduced blood cell count resulting from a cancer therapy such as chemotherapy or radiation therapy. In some embodiments, the disease or disorder is cancer. In some embodiments, the patient is also administered a chemotherapy or a radiotherapy. In some embodiments, the effective combined amount of agents which interact with chemokine receptors and a VLA-4 inhibitor results in improved efficacy of the chemotherapy or radiotherapy. In some embodiments, the therapeutically effective amount is a therapeutically effective combined amount.

In some aspects, the present disclosure provides a method of inducing the mobilization of hematopoietic stem cells or progenitor cells comprising contacting the hematopoietic stem cells with an effective combined amount of agents which interact with chemokine receptors and a VLA-4 inhibitor. In some embodiments, the method is ex vivo. In other embodiments, the method is in vitro. In yet other embodiments, the method is in vivo.

In some aspects, the present disclosure provides methods of collecting hematopoietic stem cells or progenitor cells from a patient comprising administering to the patient agents which interact with chemokine receptors and a VLA-4 inhibitor disclosed herein in an amount sufficient to mobilize hematopoietic stem cells or progenitor cells to the peripheral blood of the patient and subsequently drawing peripheral blood from the patient to collect the hematopoietic stem cells or progenitor cells.

In some aspects, the present disclosure provides methods of collecting hematopoietic stem cells or progenitor cells from a patient who has been administered agents which interact with chemokine receptors and a VLA-4 inhibitor in an amount sufficient to mobilize hematopoietic stem cells or progenitor cells to the peripheral blood of the patient comprising subsequently drawing peripheral blood from the patient to collect the hematopoietic stem cells or progenitor cells.

In some aspects, the present disclosure provides a method of improving the harvest of hematopoietic stem cells or progenitor cells comprising administering to a patient a therapeutically effective combined amount of agents which interact with chemokine receptors and a VLA-4 inhibitor. In some aspects, the present disclosure provides a method of transplanting hematopoietic stem cells or progenitor cells comprising administering to a first patient a therapeutically effective combined amount of agents which interact with chemokine receptors and a VLA-4 inhibitor, collecting hematopoietic stem cells or progenitor cells from the first patient, and transplanting the hematopoietic stem cells or progenitor cells to a second patient. In some embodiments, the hematopoietic stem cells are collected from the patient before an event which results in a reduction of the amount of the first patient's hematopoietic stem cells or progenitor cells. In some embodiments, the first patient is a compatible hematopoietic stem cell donor.

In some aspects, the present disclosure provides a method of transplanting hematopoietic stem cells or progenitor cells comprising transplanting the hematopoietic stem cells or progenitor cells collected from a first patient who has been administered a therapeutically effective combined amount of agents which interact with a chemokine receptor and a VLA-4 inhibitor to a second patient. In some embodiments, the hematopoietic stem cells are collected from the patient before an event which results in a reduction of the amount of the first patient's hematopoietic stem cells or progenitor cells. In some embodiments, the first patient is a compatible hematopoietic stem cell donor.

In some aspects, the present disclosure provides a method of transplanting to a patient hematopoietic stem cells or progenitor cells comprising administering to the patient a therapeutically effective combined amount of agents which interact with a chemokine receptor and a VLA-4 inhibitor, collecting hematopoietic stem cells or progenitor cells from the patient, and transplanting the hematopoietic stem cells or progenitor cells in the patient. In some embodiments, the hematopoietic stem cells or progenitor cells are transplanted after an event which results in a reduction of the amount of the patient's hematopoietic stem cells or progenitor cells.

In some aspects, the present disclosure provides a method of transplanting to a patient hematopoietic stem cells or progenitor cells comprising transplanting the hematopoietic stem cells or progenitor cells collected from the patient who has been administered a therapeutically effective combined amount of agents which interact with a chemokine receptor and a VLA-4 inhibitor. In some embodiments, the hematopoietic stem cells or progenitor cells are transplanted after an event which results in a reduction of the amount of the patient's hematopoietic stem cells or progenitor cells.

In some aspects, the present disclosure provides a method of improving the effectiveness of a treatment of cancer in a patient administered a chemotherapy or a radiotherapy comprising administering to the patient a therapeutically effective combined amount of agents which interact with chemokine receptors and a VLA-4 inhibitor, and administering a chemotherapy or a radiotherapy to the patient. In some embodiments, the chemotherapy or radiotherapy is administered simultaneously with the agent which interacts with a chemokine receptor and the VLA-4 inhibitor. In some embodiments, the chemotherapy or radiotherapy is administered before the agent which interacts with a chemokine receptor and the VLA-4 inhibitor. In other embodiments, the chemotherapy or radiotherapy is administered after the agent which interacts with a chemokine receptor and the VLA-4 inhibitor.

In some aspects, the present disclosure provides a method of improving the effectiveness of a treatment of cancer in a patient who has been or will be administered a chemotherapy or radiotherapy and a therapeutically effective combined amount of agents which interact with chemokine receptors and a VLA-4 inhibitor. In some embodiments, the chemotherapy or radiotherapy is administered simultaneously with the agent which interacts with a chemokine receptor and the VLA-4 inhibitor. In some embodiments, the chemotherapy or radiotherapy is administered before the agent which interacts with a chemokine receptor and the VLA-4 inhibitor. In other embodiments, the chemotherapy or radiotherapy is administered after the agent which interacts with a chemokine receptor and the VLA-4 inhibitor.

In some embodiments, the method comprises administering the agent which interacts with a chemokine receptor once. In other embodiments, the method comprises administering the agent which interacts with a chemokine receptor two or more times.

In some embodiments, the method comprises administering the VLA-4 inhibitor once. In other embodiments, the method comprises administering the VLA-4 inhibitor two or more times.

In some embodiments, the VLA-4 inhibitor and the agent which interacts with a chemokine receptor are administered simultaneously. In further embodiments, the method comprises administering a composition comprising the agent which interacts with a chemokine receptor and VLA-4 inhibitor. In other embodiments, the method comprises administering the agent which interacts with a chemokine receptor before administering the VLA-4 inhibitor. In some embodiments, the agent which interacts with a chemokine receptor is administered from 15 minutes to 0 minutes before the VLA-4 inhibitor. In other embodiments, the method comprises administering the agent which interacts with a chemokine receptor after administering the VLA-4 inhibitor.

In some embodiments, the agent which interacts with a chemokine receptor is administered subcutaneously and the VLA-4 inhibitor is administered intravenously. In other embodiments, both the agent which interacts with a chemokine receptor and the VLA-4 inhibitor are administered subcutaneously.

In some embodiments, the method produces effects equivalent to the sum of the effects of each of the agents that interacts with a chemokine receptor or VLA-4 inhibitor when administered independently. In other embodiments, the method produces a synergistic effect relative to the effects of each of the agents that interacts with a chemokine receptor or VLA-4 inhibitor when administered independently.

In some embodiments, the hematopoietic stem cells or progenitor cells are LSK-SLAM cells.

In some embodiments, the agent which interacts with a chemokine receptor is selected from plerixafor, Groβ, or a derivative of Groβ. In some embodiments, the derivative of Groβ is a truncated version of Groβ. In some embodiments, the truncated version of Groβ is SB-251353.

In some embodiments, the method further comprises administering an inhibitor of integrin α9β1, G-CSF, a derivative of G-CSF, or a combination thereof. In some embodiments, the inhibitor of integrin α9β1 is (N-benzenesulfonyl)-L-prolyl-L-O-(1-pyrrolidinylcarbonyl)tyrosine (BOP).

Disclosed herein are compositions and methods comprising a first agent which interacts with a chemokine, such as a CXCR2 agonist, a second agent which interacts with a chemokine, such as a CXCR4 inhibitor and a compound that act as integrin antagonists or inhibitors, such as α4β1 integrin (VLA-4) antagonists as well as compositions thereof. These compositions may result in the mobilization of progenitor and/or stem cells from bone marrow to peripheral circulation. Additionally, provided herein are methods for the treatment and/or prevention of disease using these two therapeutic agents in combination. These compositions described herein may be used to stimulate progenitor and/or stem cells and result in such stimulation in a shorter amount of time relative to either agent alone or other known agents or combinations. These compositions may also have the added advantage that they result in the mobilization in higher numbers, begin mobilization in a shorter period of time, over a more prolonged period of time, or mobilize increased numbers of early progenitor and/or stem cells, LSK-SLAM cells, CFU—C cells, or other progenitor and/or stem cells which are competent to achieve a successful engraftment into the patient. In some embodiments, these compositions may be used in improving the harvest of hematopoietic stem cells or progenitor cells. These methods, compositions, or uses are described in more detail below.

### Pharmaceutical Formulations and Routes of Administration

In some aspects, the present disclosure relates to compositions containing one or more VLA-4 inhibitors and one or more agents that interact with a chemokine such as a CXCR2 agonist, a CXCR4 inhibitor, or G-CSF. In some aspects, the present disclosure relates to compositions containing one or more VLA-4 inhibitors and at least two agents which interact with a chemokine. In some aspects, the present disclosure relates to compositions containing one or more VLA-4 inhibitors, at least one CXCR4 inhibitor, and at least one CXCR2 agonist. These compositions may further comprise an excipient such as solvent or diluent which renders the composition suitable for administration via injection. In some embodiments, the components of these compositions may be formulated independently and then administered simultaneously to a patient. In other embodiments, these compositions are formulated with additional therapeutic agents or excipients. In other embodiments, these compositions consists substantially of, consists essentially of, or consists of one or more VLA-4 inhibitors, one or more agents which interact with a chemokine, and one or more excipients. Each of the compositions described herein contain a pharmaceutically effective amount of each of these agents combined. In particular, the compositions may contain a pharmaceutically effective combined amount of a VLA-4 inhibitor, a CXCR4 inhibitor, and a CXCR2 agonist. The pharmaceutically effective combined amount results when each agent is present in an amount such that the effect of the combination results in increased activity relative to a similar amount of a single agent. In some embodiments, the effect of the combination results in an additive increase in activity. In some embodiments, the effect of the combination results in synergistic activity.

For administration to a subject in need of such treatment, the compounds in a therapeutically effective amount are ordinarily combined with one or more excipients appropriate to the indicated route of administration. The compounds of the present disclosure are contemplated to be formulated in a manner amenable to treatment of a veterinary patient as well as a human patient. In some embodiments, the veterinary patient may be an avian such as chicken, turkey, or duck, a companion animal such as a cat or dog, livestock animals such as a cow, horse, pig, or goat, zoo animals, and wild animals. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of animal being treated. Description of potential administration routes which may be used to formulate the compositions described herein can include those taught in Remington's Pharmaceutical Sciences, which is incorporated herein by reference.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g., subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intramuscularly, intraarterially, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water-soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as glycerol, propylene glycol, liquid polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

For intravenous or parenteral administration, the compounds are formulated in suitable liquid form with excipients as required. The compositions may contain liposomes or other suitable carriers. For injection intravenously, the solution is made isotonic using standard preparations such as Hank's solution or other isotonic solutions.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation. Alternatively, the therapeutic agents may be administered transdermally.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see, e.g., Reagan-Shaw et al., FASEB J., 22(3):659-661, 2008, which is incorporated herein by reference):

HED(mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In one aspect, the present methods or compositions may be administered such that the VLA-4 inhibitor is administered intravenously and the agent (e.g., the first and/or second agent) which interacts with a chemokine receptor is administered subcutaneously. Alternatively, the VLA-4 inhibitor and the agent which interacts with a chemokine receptor may be both administered subcutaneously. In still another embodiment, both the VLA-4 inhibitor and the agent which interacts with a chemokine receptor are administered subcutaneously in a single formulation. In some embodiments, the VLA-4 inhibitor and the agent which interacts with a chemokine receptor are administered as a single formulation subcutaneously or intravenously. The protocols for administration to a particular patient may be further optimized by a skilled practitioner.

An effective amount typically can vary from about 0.0001 mg/kg to about 1000 mg/kg, from about 0.001 mg/kg to about 100 mg/kg, from about 0.01 mg/kg to about 750 mg/kg, from about 0.001 mg/kg to about 50 mg/kg, from about 100 mg/kg to about 500 mg/kg, from about 1.0 mg/kg to about 250 mg/kg, from about 10.0 mg/kg to about 150 mg/kg, or from about 1.0 mg/kg to about 15 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

In some embodiments, the VLA-4 antagonist (i.e., inhibitors) may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a specific VLA-4 inhibitor such as a compound of formula I may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

In some embodiments, the agent which interacts with a chemokine receptor may be administered in an amount from about 1 mg/kg to about 10 mg/kg, or about 1 mg/kg to about 5 mg/kg, or about 2.5 mg/kg. The agent which interacts with a chemokine receptor may be Groβ or a derivative thereof. Similarly, the VLA-4 inhibitor may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a specific VLA-4 inhibitor such as a compound of formula I or a specific compound described in the examples such as firategrast or compounds of the VLA-4 inhibitors described herein, such as compounds listed in Table 1, may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount of the inhibitors or agents may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis, or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

The compositions and methods described herein may include one or more additional agents that are therapeutically or nutritionally useful such as antibiotics, vitamins, herbal extracts, anti-inflammatories, glucose, antipyretics, analgesics, cyclophosphamide, recombinant stem cell factor (Stemgen®), granulocyte-macrophage colony stimulating factor (GM-CSF) (such as Leukine®, and Leucomax®), ETRX-101, TLK 199/TILENTRA™, Interleukin-1 (IL-1), Interleukin-3 (IL-3), Interleukin-8 (IL-8), PIXY-321 (GM-CSF/IL-3 fusion protein), macrophage inflammatory protein, thrombopoietin, or a similar agent. Additionally, the compositions may contain one or more agents that prevent microbial growth to increase the storage of the composition. Such agents may be an anti-parasitic, an antifungal, an antibiotic, or anti-viral. Additionally, the compositions may further comprise one or more chemotherapeutic agents.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Diseases, Disorders, or Conditions Treatable with Wild Type or Genetically Engineered Hematopoietic Stem/Progenitor Cells

The compositions described herein can be used to mobilize hematopoietic stem/progenitor cells to treat or prevent numerous diseases, disorders, or conditions. The disease, disorder, or condition can be associated with impaired production of hematopoietic progenitor and/or stem cells resulting from a high dose of chemotherapy, radiotherapy, another therapeutic agent, such as for treating blood cancers or a genetic abnormality. The disease, disorder, or condition can be reduced blood cell count resulting from a cancer therapy such as chemotherapy or radiation therapy. The disease, disorder, or condition can be associated with cell adhesion-mediated inflammatory pathways including, but not limited to, asthma, multiple sclerosis, rheumatoid arthritis, atherosclerosis, inflammatory bowel disease, Crohn's disease, ulcerative colitis, graft vs. host disease, neuroinflammation, neurodegeneration, spinal cord injury, neurological diseases, or other inflammatory pathologies associated with this mechanism.

The disease, disorder, or condition can be a cancer, blood cancer, or a genetic abnormality. The disease, disorder, or condition can be a blood borne disease (e.g., sickle cell disease). The disease, disorder, or condition can be a hematological disease such as a hematological cancer. Hematological cancers can include Hodgkin's and non-Hodgkin's lymphoma, multiple myeloma, or leukemia. The disease, disorder, or condition can be a hematopoietic malignancy (e.g., leukemia, lymphoma, or myeloma, such as multiple myeloma or acute myeloid leukemia).

### Therapeutic Methods

These compositions may be used in a variety of indications such as the mobilization of hematopoietic stem cells or progenitor cells. These indications include elevating the number of progenitor and/or stem cells which are circulating in the patient especially elevating the number of these cells in the peripheral blood of a patient. Alternatively, these compositions may be used to treat a patient with cancer including sensitizing the patient to a chemotherapy and/or radiotherapy, for the treatment of hematopoietic cancer such as leukemias, myelomas, or lymphoma, or the harvesting of hematopoietic progenitor and/or stem cells which may be transplanted into a patient who has impaired production of hematopoietic progenitor and/or stem cells. A patient may have impaired production of hematopoietic progenitor and/or stem cells resulting from a high dose of chemotherapy, radiotherapy, another therapeutic agent, or a genetic abnormality. Alternatively, the compositions described herein may be used to mobilize pre-cancerous or cancerous cells from the bone marrow into the peripheral blood. In some embodiments, the mobilization of pre-cancerous or cancerous cells from the bone marrow is used to potentiate or increase the effects of a standard cancer therapy such as a chemotherapeutic and/or radiotherapy. Furthermore, each of these compositions may be used in the manufacture of medicament for these indications.

The present disclosure relates to the fields of pharmaceuticals, medicine, and cell biology. In another aspect, this disclosure provides methods of inhibiting or antagonizing VLA-4 and a4b7 using one or more of the compounds disclosed herein, as well as pharmaceutical compositions thereof. In several aspects of the present disclosure, the compounds provided herein may be used in a variety of biological, prophylactic, or therapeutic areas, including those in wherein VLA-4 and/or a4b7 plays a role.

In one aspect, this disclosure provides methods of inhibiting or antagonizing VLA-4 and a4b7 using one or more of the compounds disclosed herein, as well as pharmaceutical compositions thereof containing one or more VLA-4 and a4b7 antagonists in the presence of one or more agents that interact with a chemokine, such as Groβ, G-CSF, or a derivative thereof. The therapeutic methods described herein may be used to enhance or elevate the circulation of hematopoietic progenitor and/or stem cells. These therapeutic methods may be used to improve stem cell transplantation, tissue repair, improve the efficacy of cancer therapy, or other situations in which in vivo stimulation of hematopoiesis is desirable. The compositions or methods described herein wherein the VLA-4 inhibitor and an agent which interacts with a chemokine receptor combine to act synergistically to induce rapid mobilization of progenitor and stem cells. For example, peak mobilization, when these combined therapeutic agents are used, may occur at about 10 minutes, about 15 minutes, about 20 minutes, about 25 minutes, about 30 minutes, about 45 minutes, about 1 hour, about 2 hours, about 3 hours, about 4 hours, about 5 hours, or about 6 hours after administration of the combination. In one embodiment, these compositions or methods result in a composition that acts synergistically to induce rapid mobilization of progenitor and stem cells with peak mobilization at about 15 minutes after administration of the combination. In contrast, this mobilization is significantly shorter than the 4-5 days needed to achieve maximum mobilization using G-CSF.

In one aspect, the compounds and compositions described herein may be used to increase the harvest of HSPCs for a variety of different applications. These compounds and compositions may be used to treat a patient who requires a transplantation. Alternatively, the compounds and compositions may be used to treat a patient who does not require a transplantation. The patient who needs a transplant of HSPCs requires either an allogenic, autologous, or tandem transplant of HSPCs. In some embodiments, the HSPCs may be used in either allogenic or autologous transplants. In another aspect, the present compounds and compositions described herein may be used to improve the circulation of cells to tissues that need repair. The increased circulation of HSPCs may be used to improve the repair of the target tissue in the patient.

If the HSPCs are harvested, these cells may be returned to the donor patient (autologous transplant) or may be donated to another patient that is sufficiently compatible to prevent rejection (allogeneic transplant). One non-limiting application of autologous transplantation is in combination with radiation or chemotherapy in patients bearing tumors since the radiotherapeutic or chemotherapeutic methods deplete the patient's normal cells. In this application, the patient's cells may be harvested prior to or during the therapeutic treatments, fractionated if necessary, cultured and optionally expanded, and then returned to the patient to restore the damaged immune system depleted by the therapy. Allogeneic recipients may receive the cells for the same purpose, or may have a condition that may be benefited by enhancing their hematopoietic systems. In a typical protocol for these types of transplants, the mobilized cells are collected from the donor by, for example, apheresis and then stored/cultured/expanded/fractionated as desired. In some embodiments, the compounds and compositions described herein may result in the need for apheresis being eliminated.

In some aspects, the present compounds, compositions, and methods described herein may be used to increase the circulation of pre-cancerous or cancerous cells out of the bone marrow into the peripheral blood. Without wishing to be bound by any theory, it is believed that increasing the circulation of pre-cancerous or cancerous cells out of the bone marrow may increase the effectiveness of an anti-cancer therapy. In particular, these compounds and compositions may be used to treat patients who have or are at risk of a hematopoietic malignancy such as lymphoma, myeloma, or leukemia. The compounds and compositions described herein may be administered or employed prior to, during, or subsequent to the anti-cancer therapy. Two non-limiting examples of anti-cancer therapies that may be used in the methods described herein or conjunction with the compounds and compositions described herein include chemotherapeutic agents or radiotherapy.

In another aspect, the compounds, compositions, and methods described herein may be used to decrease inflammation which may result in increasing tissue repair. Thus, the compounds and compositions described herein may be used to treat graft versus host disease. Additionally, these compounds and compositions may be used to treat diseases or disorders associated with cell adhesion-mediated inflammatory pathways. Some non-limiting examples of cell adhesion-mediated inflammatory pathologies include asthma, multiple sclerosis, rheumatoid arthritis, atherosclerosis, inflammatory bowel disease, neuroinflammation, neurodegeneration, and spinal cord injury.

In some aspects, the present disclosure provides methods of treating a disease or disorder in a patient comprising administering to the patient a therapeutically effective amount of a compound or composition disclosed herein. In some embodiments, the disease or disorder is associated with integrin α4β1. In other embodiments, the disease or disorder is associated with inflammation. In yet other embodiments, the disease or disorder is an autoimmune disorder. In still other embodiments, the disease or disorder is associated with hematopoietic stem cells such as LSK-SLAM cells. In yet other embodiments, the disease or disorder is cancer or a reduced blood cell count such as reduced blood cell count resulting from a therapy for cancer. In some embodiments, the disease or disorder is a reduced blood cell count resulting from a therapy for cancer such as chemotherapy or radiation therapy. In other embodiments, the disease or disorder is cancer. In some embodiments, the compound or composition results in improved efficacy of the chemotherapy or radiotherapy.

Such pharmaceutical compositions can further comprise one or more non-toxic, pharmaceutically acceptable carriers and/or diluents and/or adjuvants (collectively referred to herein as “carrier” materials) and if desired other active ingredients. These methods may be used to treat a blood disease or disorder such as sickle cell anemia or as a part of hematopoietic stem cell therapy to promote the development of stem cells. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants, and vehicles. The term parenteral as used herein includes, for example, subcutaneous, intravenous, intramuscular, intrasternal, infusion techniques, or intraperitoneally. In some embodiments, a composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

In some aspects, the present disclosure provides methods of inducing the mobilization of hematopoietic stem cells or progenitor cells comprising contacting the hematopoietic stem cells or progenitor cells with an effective amount of a compound or composition disclosed herein. In some embodiments, the method is ex vivo or in vitro. In some embodiments, method is in vivo.

In some aspects, the present disclosure provides methods of collecting hematopoietic stem cells or progenitor cells from a patient comprising:
(A) administering to the patient a compound or composition disclosed herein in an amount sufficient to mobilize hematopoietic stem cells or progenitor cells to the peripheral blood of the patient; and
(B) subsequently drawing peripheral blood from the patient to collect the hematopoietic stem cells or progenitor cells.

In some aspects, the present disclosure provides methods of collecting hematopoietic stem cells or progenitor cells from a patient who has been administered a compound or composition disclosed herein in an amount sufficient to mobilize hematopoietic stem cells or progenitor cells to the peripheral blood of the patient comprising subsequently drawing peripheral blood from the patient to collect the hematopoietic stem cells or progenitor cells.

In other aspects, the present disclosure provides methods of improving the harvest of hematopoietic stem cells or progenitor cells comprising administering to a patient a therapeutically effective amount of a compound or composition disclosed herein.

In yet other aspects, the present disclosure provides methods of transplanting to a patient hematopoietic stem cells or progenitor cells comprising:
(A) administering to the patient a compound or composition disclosed herein;
(B) collecting hematopoietic stem cells or progenitor cells from the patient;
(C) transplanting the hematopoietic stem cells or progenitor cells in the patient.

In some aspects, the present disclosure provides methods of transplanting to a patient hematopoietic stem cells or progenitor cells comprising transplanting the hematopoietic stem cells or progenitor cells collected from the patient who has been administered a therapeutically effective amount of a compound or composition disclosed herein.

In some aspects, the present disclosure provides methods of transplanting hematopoietic stem cells or progenitor cells comprising:
(A) administering to a first patient a compound or composition described herein;
(B) collecting hematopoietic stem cells or progenitor cells from the first patient;
(C) transplanting the hematopoietic stem cells or progenitor cells in the second patient.

In some aspects, the present disclosure provides methods of transplanting hematopoietic stem cells or progenitor cells comprising transplanting the hematopoietic stem cells or progenitor cells collected from a first patient who has been administered a therapeutically effective amount of a compound or composition disclosed herein to a second patient.

In some embodiments, the hematopoietic stem cells are collected from the patient before an event which results in a reduction of the amount of the patient's hematopoietic stem cells or progenitor cells. In some embodiments, the hematopoietic stem cells or progenitor cells are transplanted after an event which results in a reduction of the amount of the patient's hematopoietic stem cells or progenitor cells. In some embodiments, the first patient is a compatible hematopoietic stem cell donor. In some embodiments, the hematopoietic stem cells or progenitor cells are LSK-SLAM cells.

In some aspects, the present disclosure provides methods of improving the effectiveness of a treatment of cancer in a patient administered a chemotherapy or a radiotherapy comprising:
(A) administering to the patient a therapeutically effective amount of a compound or composition disclosed herein;
(B) administering a chemotherapy or a radiotherapy to the patient.

In some aspects, the present disclosure provides methods of improving the effectiveness of a treatment of cancer in a patient who has been administered a chemotherapy or radiotherapy and a compound or composition disclosed herein.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

### Combinational Therapies

The present disclosure may relate to one or more agents used in combination with a VLA-4 antagonist. The present disclosure describes combinations of VLA-4 antagonists with other therapeutic modalities as combination therapies to increase the mobilization of hematopoietic stem cells.

To increase the mobilization of hematopoietic stem cells using the methods and compositions of the present disclosure, one would generally administer to the subject with a VLA-4 antagonist and at least one other therapy. These therapies would be provided in a combined amount effective to achieve an increased activity. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the VLA-4 antagonist and the other includes the other agent.

Alternatively, the individual compounds in the compositions described herein may precede or follow the other compound treatment by time intervals ranging from seconds to days. One would generally ensure that a significant period of time did not expire between the time of each delivery, such that the therapies would still be able to exert an advantageously combined effect on the cell/subject. In such instances, it is contemplated that one would administer the two or more modalities within about 12-24 hours of each other, within about 6-12 hours of each other, with a delay time of only about 1-2 hours, or less than 1 hour. Additionally, the agents which interact with a chemokine may be administered about 10-15 minutes, about 5-10 minutes, or about 0-5 minutes prior to administration of the VLA-4 inhibitor. For example, the agents which interact with a chemokine may be administered from about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, to about 1 minute, or any range derivable therein before the VLA-4 inhibitor. Alternatively, the components may be administered at the same time.

The compositions and combination of agents used in the methods described herein may be administered as a single bolus dose, a dose over time such as an infusion, as in intravenous, subcutaneous, or transdermal administration, or in multiple dosages. If infusion is used, the combination may be infused for about 15 minutes to about 6 hours. In one embodiment, the infusion may occur for the duration of length of the apheresis. Additionally, the compositions or combination may be administered once daily for multiple days including from 1 to 4 days.

Furthermore, the compositions or combinations may be administered to the patient for one day or less than one day and then HSPCs isolated from the patient. The compositions or combinations described herein may be administered and then HSPCs may be isolated for about 1 hour, about 2 hours, about 3 hours, about 4 hours, about 5 hours, about 6 hours, about 7 hours, or about 8 hours following administration.

It also is conceivable that more than one administration of either the compound or the other therapy will be desired. Various combinations may be employed, where a compound of the present disclosure is “A,” and the other compound or therapy is “B,” as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. In some aspects of the present disclosure, the agent may be a CXC chemokine, a CXC chemokine receptor, or a derivative thereof. Some non-limiting examples of the agent include Groβ, truncated Groβ (Groβt), plerixafor (AMD3100), a granulocyte-colony stimulating factor (G-CSF) such as filgrastim, PEG-filgrastim, or lenograstim, or an inhibitor of integrin α9β1 such as BOP (N-benzenesulfonyl)-L-prolyl-L-O-(1-pyrrolidinylcarbonyl)tyrosine.

In other embodiments, the compositions or methods used herein may be administered with an anti-cancer therapy such as those described below. The methods or compositions described herein may be used in conjunction with standard methods or variations as practiced by a person of ordinary skill in the art. These anti-cancer agents may be administered prior to and/or concomitant with the compositions or methods described herein. Some non-limiting examples of anti-cancer therapies which may be used herein include carmustine, etoposide, cytarabine, melphalan, cyclophosphamide, busulfan, thiotepa, bleomycin, platinum (cisplatin), cytarabine, cyclophosphamide, buside, daunorubicin, doxorubicin, agent ara-C, cyclosporin; Rituxan®; thalidomide; clofarabine; Velcade®; Antegren®; Ontak®; Revlimid® (thalidomide analog); Prochymal®; Genasense® (oblimersen sodium); Gleevec®; Glivec® (imatinib); tamibarotene; nelarabine; gallium nitrate; PT-100; Bexxar®; Zevalin®; pixantrone; Onco-TCS; and agents that are topoisomerase inhibitors, or another specific anti-cancer therapy.

### Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁ and calicheamicin ω₁; dynemicin, including dynemicin A; uncialamycin and derivatives thereof; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-I-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, and methotrexate and pharmaceutically acceptable salts, acids or derivatives of any of the above.

### Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter can repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of DNA damage, on the precursors of DNA, on the replication and repair of DNA, and the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets that are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area from being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including cytokines such as IL-2, IL-4, IL-12, GM-CSF, γ-IFN, chemokines such as MIP-1, MCP-1, IL-8, and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al. 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

### Biological Activity

It is another object of the disclosure to provide pharmaceutical compositions comprising compounds described above. These compounds and pharmaceutical compositions may be used to improve the harvest of hematopoietic stem cells or progenitor cells. Additionally, the compounds or compositions may be used to elevate the circulation of hematopoietic progenitor and/or stem cells, improve the collection of hematopoietic stem cells or progenitor cells for a transfusion, increase the sensitization of an anti-cancer therapy such as a chemotherapeutic or radiotherapy, or mobilize pre-cancerous or cancerous cells into the peripheral blood which may increase their sensitivity to an anti-cancer therapy.

Hematopoietic stem cell transplant (HSCT) is used to facilitate repopulation of healthy bone marrow and immune system cells after high-dose chemotherapy treatment for cancers such as Hodgkin's and non-Hodgkin's lymphoma, multiple myeloma, and leukemia. In HSCT, hematopoietic stem/progenitor cells (HSPCs) are collected from the patient's blood, harvested, frozen, and then stored while the patient receives high-dose chemotherapy and/or radiation therapy. Successful HSCT requires the intravenous infusion of a minimum number of 2×10⁶ CD34+ stem cells/kg body weight; however, a dose of 5×10⁶ CD34+ cells/kg is considered preferable for early and long term multilineage engraftment.

Stem cells harvested from peripheral blood are the most commonly used graft source in HSCT. While granulocyte colony-stimulating factor (G-CSF) is the most frequently used agent for stem cell mobilization, the use of G-CSF alone results in suboptimal stem cell yields in a significant proportion of patients. Plerixafor (AMD3100), a small molecule CXCR4 antagonist, in combination with G-CSF increases total CD34+ HSPCs compared to G-CSF alone and is FDA approved for stem cell mobilization in Non-Hodgkin's lymphoma and multiple myeloma. However, a significant disadvantage of plerixafor is cost, adding $25,567 per patient compared to G-CSF alone. Furthermore, up to 24% of patients receiving plerixafor and G-CSF still fail to collect ≥2×10⁶ CD34+ cells/kg in 4 days of apheresis. Recent economic analysis has determined that reducing apheresis by 1 day has the potential to decrease medical costs by $6,600. Thus improved/alternative mobilizing agents and strategies are needed.

The compositions or methods disclosed herein may also have the added advantage that the compositions or methods result in the mobilization in higher numbers, begin mobilization in a shorter period of time, over a more prolonged period of time, or mobilize increased numbers of early progenitor and/or stem cells, LSK-SLAM cells, CFU—C cells, or other progenitor and/or stem cells which are competent to achieve successful engraftment into the patient. For example, the number of progenitor and/or stem cells mobilized when using the combination or methods described herein may be at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, or at least about 15-fold greater than when using a single agent alone. Specifically, the number of early progenitor and/or stem cells (e.g., LSK-SLAM cells) mobilized when using the combination of at least one VLA-4 inhibitor and at least one CXCR2 agonist is at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, or at least about 25-fold greater then when using a single agent alone.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In exemplary embodiments, the term “about” is used to indicate that a value includes plus or minus 10% of the value. In some embodiments, the term “about” is used to indicate that a value includes plus or minus one standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXEMPLARY EMBODIMENTS

The following non-limiting exemplary embodiments are provided to further illustrate the present disclosure. It should be appreciated by those of skill in the art that the techniques disclosed in the exemplary embodiments that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Exemplary Embodiment 1: VLA-4 Compounds, Comparator Compounds, and Synthetic Methods

The following exemplary embodiment describes the instrumentation and general methods; preparation of compounds; and comparative compounds.

### Instrumentation and General Methods.

Commercially available reagents and solvents were used without further purification unless stated otherwise. LC-MS analyses were performed on an Agilent 1100 or 1200HPLC/MSD electrospray mass spectrometer in positive ion mode with scan range was 100-1000d. Preparative normal phase chromatography was performed on a CombiFlash Rf+ (Teledyne Isco) with pre-packed RediSep Rf silica gel cartridges. Preparative reverse phase HPLC was performed on a CombiFlash Rf+ (Teledyne Isco) equipped with RediSep Rf Gold pre-packed C18 cartridges and an acetonitrile/water/0.05% TFA gradient. The purity of tested compounds was ≥95% as determined by HPLC analysis conducted on an Agilent 1100 or 1260 system using a reverse phase C18 column with diode array detector unless stated otherwise. NMR spectra were recorded on a Bruker 400 MHz spectrometer. The signal of the deuterated solvent was used as internal reference. Chemical shifts (δ) are given in ppm and are referenced to residual not fully deuterated solvent signal. Coupling constants (J) are given in Hz.

### Preparation of Compounds

### Example 1

### Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoic Acid

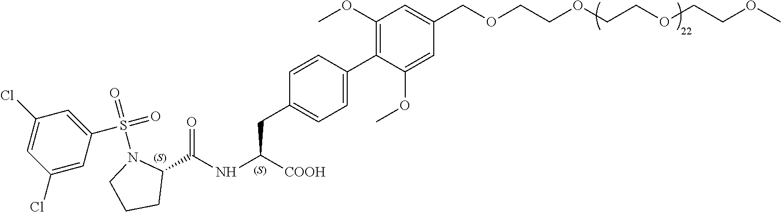

### The Synthesis of Example 1 is Depicted in Scheme A:

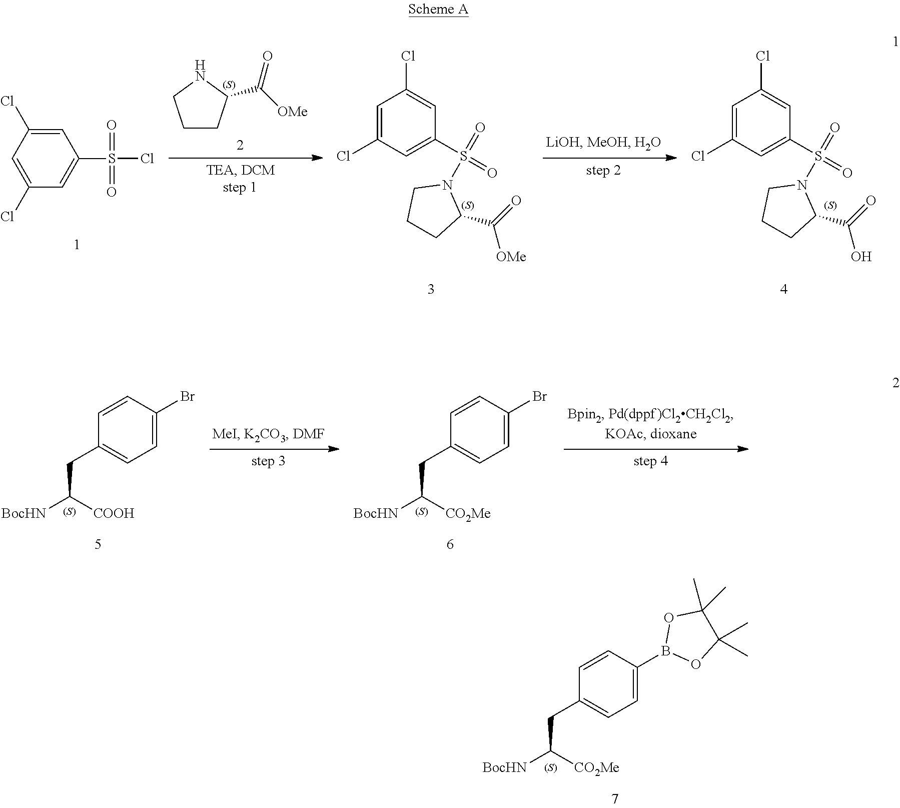

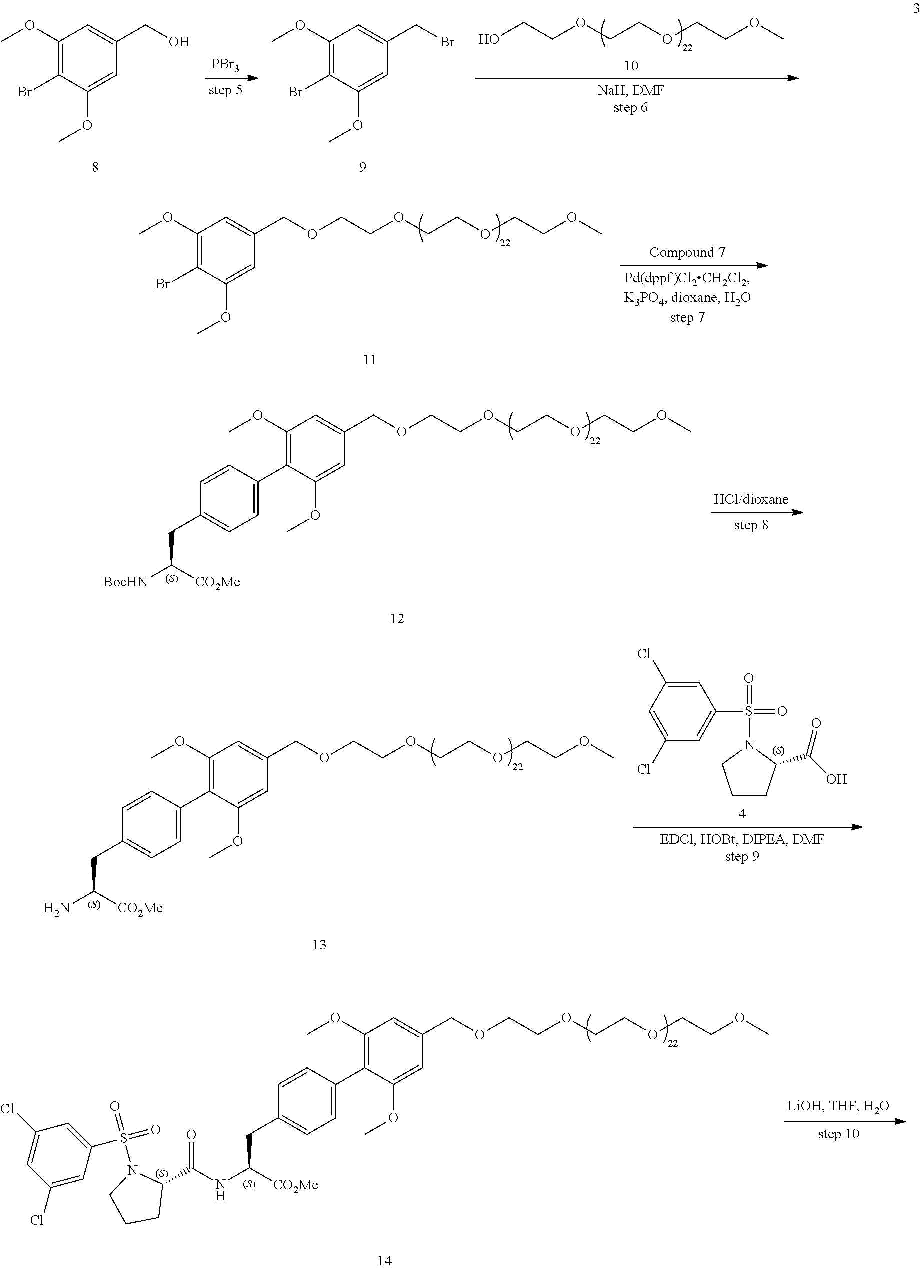

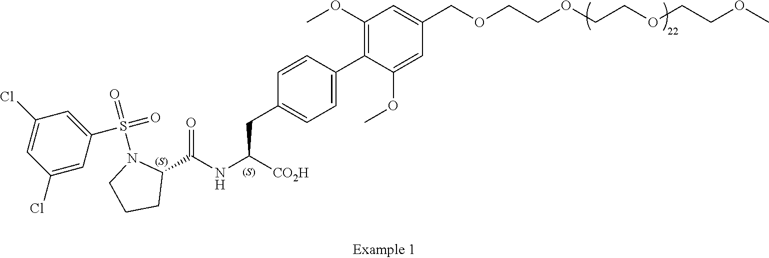

### Step 1. Preparation of (S)-methyl 1-((3,5-dichlorophenyl)sulfonyl)pyrrolidine-2-carboxylate

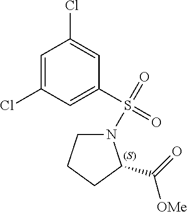

3,5-Dichlorobenzenesulfonyl chloride (25 g, 101.83 mmol, 1 eq) (in 50 mL of DCM) was added portion-wise over 30 min to a solution of methyl (2S)-pyrrolidine-2-carboxylate (18.55 g, 112.01 mmol, 1.1 eq, HCl) and DIPEA (28.95 g, 224.02 mmol, 39.02 mL, 2.2 eq) in DCM (250 mL) at 0° C. After addition, the reaction mixture was stirred 20 h at 20° C. under N₂. The solution was concentrated in vacuo. The residue was dissolved in EtOAc (300 ml) and washed with water (3×50 mL) and brine (3×50 mL). The organic phase was dried (Na2SO₄) and concentrated in vacuo. The residue was purified by flash silica gel chromatography (ISCO®; 330 g SepaFlash® Silica Flash Column, Eluent of 0˜30% Ethyl acetate/Petroleum ether gradient @85 mL/min) to give methyl (2S)-1-(3,5-dichlorophenyl)sulfonylpyrrolidine-2-carboxylate (30 g, 88.70 mmol, 87.11% yield) as a white solid. ¹H NMR (400 MHz, CDCl₃): δ 7.76 (d, J=2.0 Hz, 2H), 7.57 (t, J=2.0 Hz, 1H), 4.41 (dd, J=8.4, 3.6 Hz, 1H), 3.67-3.79 (m, 3H), 3.35-3.52 (m, 2H), 1.84-2.26 (m, 4H).

### Step 2. Preparation of (S)-1-((3,5-dichlorophenyl)sulfonyl)pyrrolidine-2-carboxylic Acid

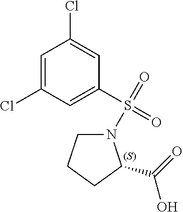

To a solution of methyl (2S)-1-(3,5-dichlorophenyl)sulfonylpyrrolidine-2-carboxylate (10 g, 29.57 mmol, 1 eq) in MeOH (200 mL) was added LiOH (1 M, 44.35 mL, 1.5 eq) in one portion. The reaction mixture was stirred 4 h at 20° C. The solution was concentrated in vacuo. The residue was dissolved in water (25 mL), acidified with 2N HCl to pH=5 and extracted with EtOAc (100 mL*3). The combined organic layer was washed with brine (50 mL*2), dried with Na2SO₄ and concentrated in vacuo to give the crude product (2S)-1-(3,5-dichlorophenyl)sulfonylpyrrolidine-2-carboxylic acid (7.5 g, 23.14 mmol, 78.25% yield) as a white solid, which was used into the next step without further purification. ¹H NMR (400 MHz, DMSO-d₆): δ 12.90 (br s, 1H), 8.01 (t, J=2.0 Hz, 1H), 7.85 (d, J=2.0 Hz, 2H), 4.31 (dd, J=8.8, 4.0 Hz, 1H), 3.22-3.31 (m, 1H), 1.98-2.14 (m, 1H), 1.65-1.95 (m, 4H).

### Step 3. Preparation of (S)-methyl 3-(4-bromophenyl)-2-((tert-butoxycarbonyl)amino)propanoate

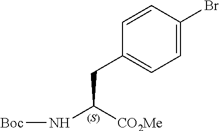

A mixture of (2S)-3-(4-bromophenyl)-2-(tert-butoxycarbonylamino)propanoic acid (25 g, 72.63 mmol, 1 eq), Mel (14.25 g, 100.40 mmol, 6.25 mL, 1.38 eq), K₂CO₃ (20.08 g, 145.26 mmol, 2 eq) in DMF (250 mL) was stirred at 20° C. for 4 hr. The mixture was filtered, the cake was washed with EtOAc (100 mL). The filtrate was diluted with EtOAc (500 mL), washed with water (150 mL*3) and brine (150 mL*3), dried over Na₂SO₄, filtered and concentrated in vacuo to give the crude product methyl (2S)-3-(4-bromophenyl)-2-(tert-butoxycarbonylamino)propanoate (27 g, crude) as a yellow gum, which was used into the next step without further purification. ¹H NMR (400 MHz, CDCl₃): δ 7.43 (d, J=8.4 Hz, 2H), 7.02 (d, J=8.0 Hz, 2H), 5.00 (br d, J=8.0 Hz, 1H), 4.64-4.54 (m, 1H), 3.74 (s, 3H), 3.16-3.07 (m, 1H), 3.05-2.98 (m, 1H), 1.44 (s, 9H).

### Step 4. Preparation of (S)-methyl 2-((tert-butoxycarbonyl)amino)-3-(4-(4,4,5,5-tetramethyl-1,3,2-dioxaborolan-2-yl)phenyl)propanoate

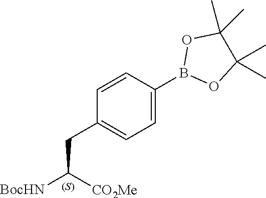

A mixture of methyl (2S)-3-(4-bromophenyl)-2-(tert-butoxycarbonylamino)propanoate (26 g, 72.58 mmol, 1 eq), 4,4,5,5-tetramethyl-2-(4,4,5,5-tetramethyl-1,3,2-dioxaborolan-2-yl)-1,3,2-dioxaborolane (27.65 g, 108.87 mmol, 1.5 eq), Pd(dppf)Cl₂·CH₂Cl₂ (2.96 g, 3.63 mmol, 0.05 eq), KOAc (15.00 g, 152.84 mmol, 2.11 eq) in dry dioxane (250 mL) was degassed and purged with N₂ 3 times, and then the mixture was stirred at 100° C. for 20 hr under N₂ atmosphere. The mixture was filtered through celite, the cake was washed with EtOAc (100 mL). The filtrate was concentrated in vacuo to give the crude product, which was purified by flash silica gel chromatography (ISCO®; 330 g SepaFlash® Silica Flash Column, Eluent of 0˜35% Ethyl acetate/Petroleum ether gradient @85 mL/min) to give the compound methyl (2S)-2-(tert-butoxycarbonylamino)-3-[4-(4,4,5,5-tetramethyl-1,3,2-dioxaborolan-2-yl)phenyl]propanoate (27.5 g, 67.85 mmol, 93.49% yield) as a light yellow gum. ¹H NMR (400 MHz, CDCl₃): δ 7.66 (d, J=8.0 Hz, 2H), 7.05 (d, J=7.6 Hz, 2H), 4.89 (br d, J=8.0 Hz, 1H), 4.57-4.42 (m, 1H), 3.63 (s, 3H), 3.11-2.95 (m, 2H), 1.35 (s, 9H), 1.26 (s, 12H).

### Step 5. Preparation of 2-bromo-5-(bromomethyl)-1,3-dimethoxybenzene

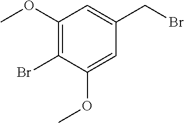

A solution of (4-bromo-3,5-dimethoxy-phenyl)methanol (5 g, 20.24 mmol, 1 eq) in toluene (100 mL) was added PBr₃ (6.03 g, 22.26 mmol, 1.1 eq) in portions at 0° C. under N₂. After addition, the solution was stirred at 20° C. for 2 hr under N₂. The reaction was quenched with water (50 mL), extracted with EtOAc (100 mL*3). The combined organic layer washed with water (50 mL*3), brine (50 mL*3), dried over Na2SO₄, filtered and concentrated in vacuo to give the crude product, which was purified by flash silica gel chromatography (ISCO®; 40 g SepaFlash® Silica Flash Column, Eluent of 0-20% Ethyl acetate/Petroleum ether gradient @40 mL/min) to give the compound 2-bromo-5-(bromomethyl)-1,3-dimethoxy-benzene (3.5 g, 11.29 mmol, 55.80% yield) as a white solid. ¹H NMR (400 MHz, CDCl₃): δ 6.62 (s, 2H), 4.47 (s, 2H), 3.93 (s, 6H).

### Step 6. Preparation of 1-(4-bromo-3,5-dimethoxyphenyl)-2,5,8,11,14,17,20,23,26,29,32,35,38,41,44,47,50,53,56,59,62,65,68,71,74-pentacosaoxapentaheptacontane

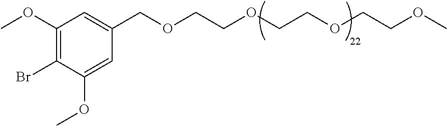

To a suspension of NaH (120.00 mg, 3.00 mmol, 60% purity, 2.18 eq) in dry DMF (10 mL) was added 2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethanol (BroadPharm) (1500 mg, 1.38 mmol, 1 eq) (in 2.5 mL of dry DMF) slowly at 0° C. under argon. After addition, the mixture was stirred for 1 h at 20° C. 2-bromo-5-(bromomethyl)-1,3-dimethoxy-benzene (800.0 mg, 2.58 mmol, 1.87 eq) (in 2.5 mL of dry DMF) was added slowly at 0° C. After addition, the mixture was stirred at 20° C. for 19 hr under argon atmosphere. The reaction was quenched with 2N HCl (1.6 mL). The solution was concentrated in vacuo. The residue was purified by flash silica gel chromatography (ISCO®; 40 g SepaFlash® Silica Flash Column, Eluent of 0˜10% MeOH/DCM gradient @40 mL/min) to give the compound 2-bromo-1,3-dimethoxy-5-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]benzene (1.55 g, 1.18 mmol, 85.38% yield) as an off-white solid. ¹H NMR (400 MHz, CDCl₃): δ 6.60 (s, 2H), 4.55 (s, 2H), 3.92 (s, 6H), 3.70-3.64 (m, 94H), 3.58-3.55 (m, 2H), 3.40 (s, 3H).

### Step 7. Preparation of (S)-methyl 2-((tert-butoxycarbonyl)amino)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate

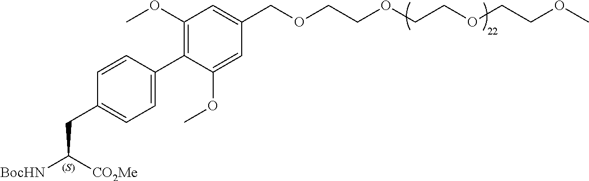

A mixture of methyl (2S)-2-(tert-butoxycarbonylamino)-3-[4-(4,4,5,5-tetramethyl-1,3,2-dioxaborolan-2-yl)phenyl]propanoate (953.00 mg, 2.35 mmol, 2 eq), 2-bromo-1,3-dimethoxy-5-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxymethyl]benzene (1.55 g, 1.18 mmol, 1 eq), Pd(dppf)Cl₂·CH₂Cl₂ (96.01 mg, 117.57 umol, 0.1 eq) and K₃PO₄ (499.12 mg, 2.35 mmol, 2 eq) in dioxane (20 mL) and H₂O (2 mL) was degassed and purged with N₂ 3 times, and then the mixture was stirred at 100° C. for 20 hr under N₂ atmosphere. The mixture was filtered through celite, the cake was washed with DCM (25 mL). The filtrate was concentrated in vacuo to give the crude product, which was purified by flash silica gel chromatography (ISCO®; 40 g SepaFlash® Silica Flash Column, Eluent of 0˜10% MeOH/DCM gradient @40 mL/min) to give the compound methyl (2S)-2-(tert-butoxycarbonylamino)-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.4 g, 923.00 umol, 78.51% yield) as a brown gum.

### Step 8. Preparation of (S)-methyl 2-amino-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate

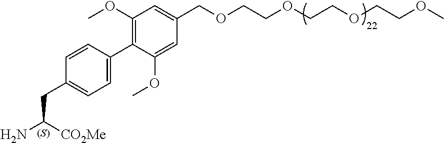

A solution of methyl (2S)-2-(tert-butoxycarbonylamino)-3-[4-[2,6-dimethoxy-4-[2-[2-[2-[2-[2-[2-[2-[2-[2-[2-[2-[2-[2-[2-[2-[2-[2-[2-[2-[2-[2- [2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.4 g, 923.00 umol, 1 eq) in MeOH (5 mL) was added HCl/dioxane (4 M, 10 mL, 43.34 eq), the solution was stirred at 20° C. for 1 hr. The solution was concentrated in vacuo to give the crude product methyl (2S)-2-amino-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.34 g, crude, HCl) as brown gum, which was used into the next step without further purification.

### Step 9. Preparation of (S)-methyl 2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate

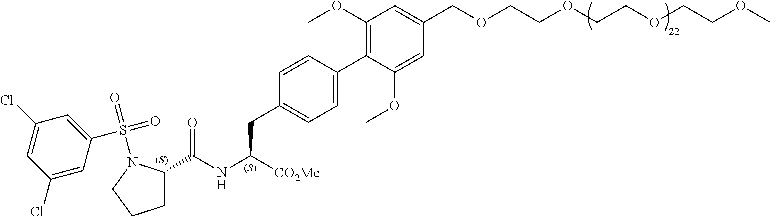

A mixture of (2S)-1-(3,5-dichlorophenyl)sulfonylpyrrolidine-2-carboxylic acid (358.73 mg, 1.11 mmol, 1.2 eq), methyl (2S)-2-amino-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2- methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.34 g, 922.14 umol, 1 eq, HCl), EDCl (353.55 mg, 1.84 mmol, 2 eq) and HOBt (186.90 mg, 1.38 mmol, 1.5 eq) in DMF (15 mL) was added DIPEA (371.00 mg, 2.87 mmol, 0.5 mL, 3.11 eq) in one portion. The mixture was stirred at 20° C. for 20 hr under N₂ atmosphere. The solution was concentrated in vacuo to give the crude product, which was purified by flash silica gel chromatography (ISCO®; 20 g SepaFlash® Silica Flash Column, Eluent of 0˜10% MeOH/DCM gradient @35 mL/min) to give the product methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.4 g, 812.61 umol, 88.12% yield) as a brown gum.

### Step 10. Preparation of (S)-2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoic Acid

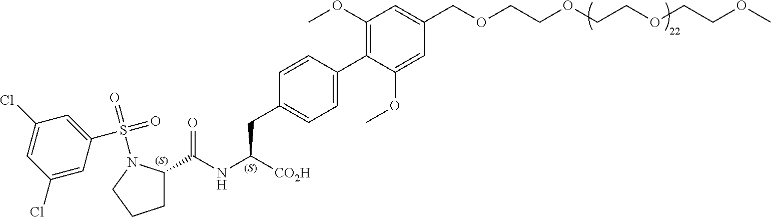

A solution of methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (1.4 g, 812.61 umol, 1 eq) in THF (10 mL) was added LiOH (1 M, 1.5 mL, 1.85 eq) in one portion at 20° C., and then the mixture was stirred at 20° C. for 1 hr. The mixture was diluted with 40 mL of water and extracted with diisopropyl ether (10 mL*3). The aqueous layer was acidified with 2N HCl to pH=6 and lyophilized to give the crude product, which was purified by prep-HPLC (HCOOH condition; column: Boston Uni C18 40*150*5 um; mobile phase: [water (0.225% FA)-ACN]; B %: 42%-72%, 7.7 min) to give the compound (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoic acid, Example 1 (1 g, 585.20 umol, 72.01% yield) as a light yellow gum.

¹H NMR (400 MHz, CD₃OD): δ 7.84 (d, J=2.0 Hz, 2H), 7.82-7.79 (m, 1H), 7.30-7.25 (m, 2H), 7.23-7.18 (m, 2H), 6.74 (s, 2H), 4.78 (dd, J=5.2, 8.4 Hz, 1H), 4.61 (s, 2H), 4.24 (t, J=6.4 Hz, 1H), 3.72-3.61 (m, 101H), 3.57-3.53 (m, 2H), 3.48-3.44 (m, 1H), 3.37 (s, 3H), 3.30-3.24 (m, 1H), 3.16-3.11 (m, 1H), 1.93-1.82 (m, 2H), 1.79-1.60 (m, 2H). HRMS (ESI), mass calcd. for C₇₈H₁₂₈Cl₂N₂O₃₂S 1706.7548, m/z found 1707.7508 [M+H]⁺.

The desired product was confirmed by QTOF-MS with a mass of 1706.7565.

### Example 2

### Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoic Acid

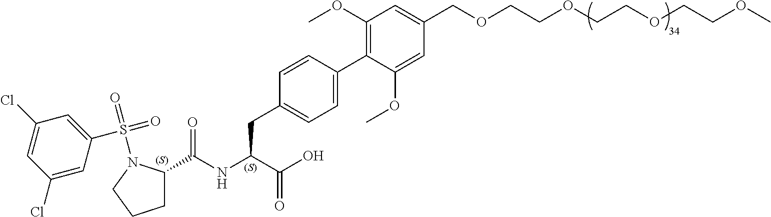

### The Synthesis of Example 2 is Depicted in Scheme B:

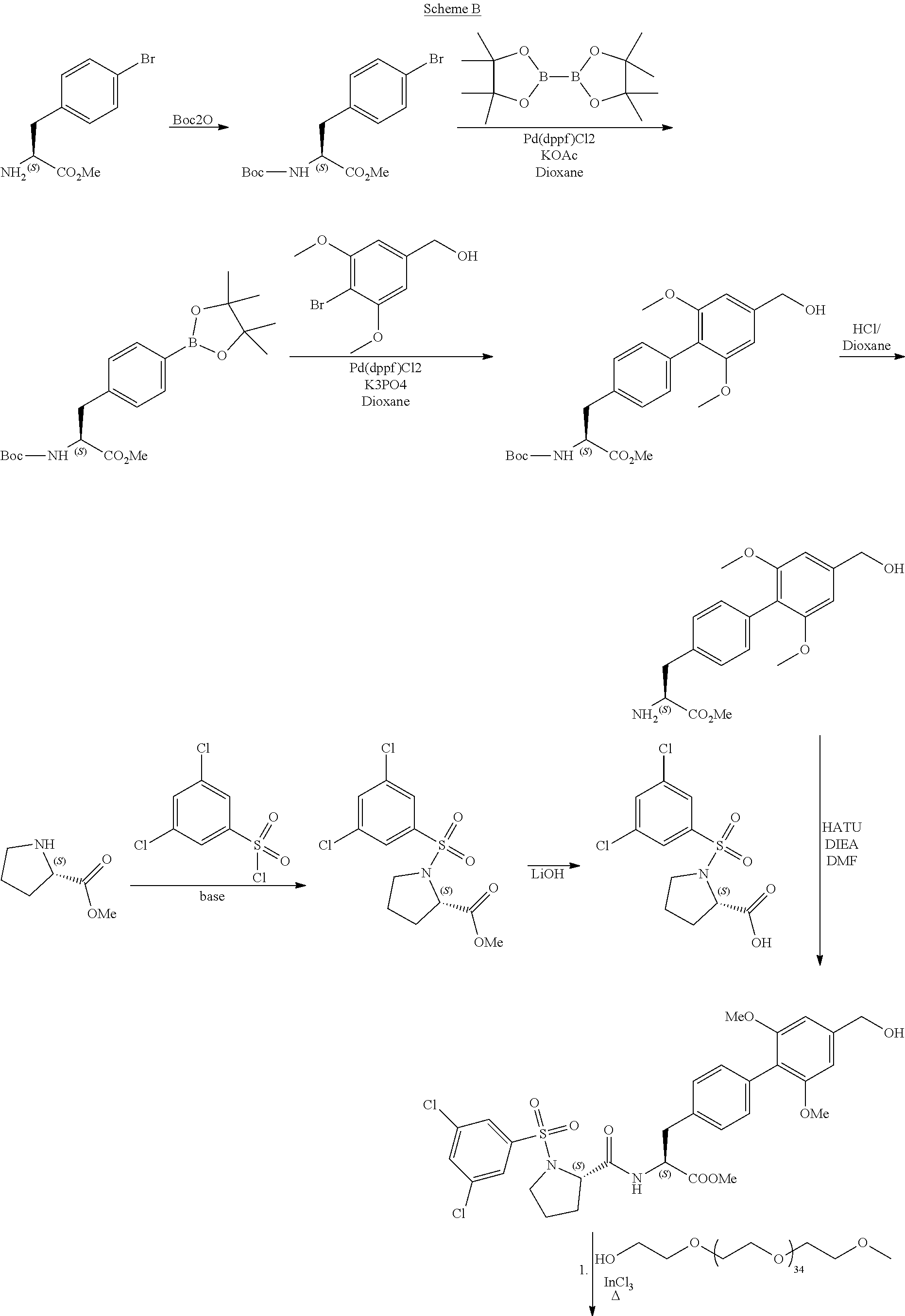

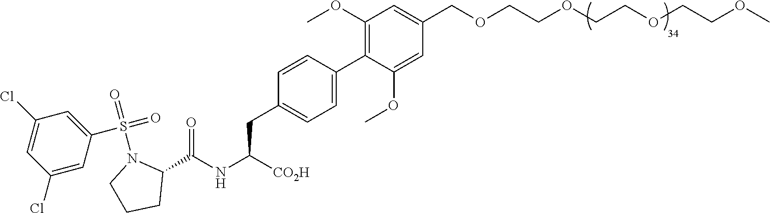

### Step 1. Preparation of (S)-methyl 3-(4-bromophenyl)-2-((tert-butoxycarbonyl)amino)propanoate

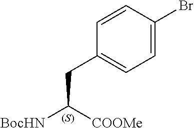

To the solution of (S)-methyl 2-amino-3-(4-bromophenyl)propanoate (45.0 g, 152 mmol, HCl) in DCM (250 mL) was added Boc₂O (50.0 g, 229 mmol, 52.6 mL) and TEA (54.1 g, 534 mmol, 74.4 mL). The mixture was stirred at 25° C. for 3 h. TLC showed the reaction was complete. The reaction mixture was washed with water (500 mL*3). The organic layer was dried by sodium sulfate, filtered and concentrated in vacuum to give the desired product (26.6 g, yield 48.6%) as white solid, which was recrystallized by Petroleum ether/Ethyl acetate=100/1 (300 mL) ¹H NMR (400 MHz, CDCl₃): δ 7.42 (d, J=8.4 Hz, 2H), 7.01 (d, J=8.2 Hz, 2H), 4.97-4.98 (m, 1H), 4.56-4.58 (m, 1H), 3.72-3.73 (m, 3H), 2.97-3.12 (m, 2H), 1.42-1.57 (m, 9H).

### Step 2. Preparation of (S)-methyl 2-((tert-butoxycarbonyl)amino)-3-(4-(4,4,5,5-tetramethyl-1,3,2-dioxaborolan-2-yl)phenyl)propanoate

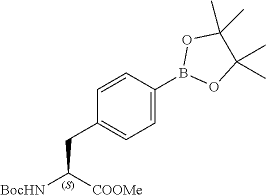

To the solution of (S)-methyl 3-(4-bromophenyl)-2-((tert-butoxycarbonyl)amino)propanoate (26.6 g, 74.2 mmol) and 4,4,4′,4′,5,5,5′,5′-octamethyl-2,2′-bi(1,3,2-dioxaborolane) (28.2 g, 111 mmol) in dioxane (250 mL) was added Pd(dppf)Cl₂ (5.43 g, 7.43 mmol) and KOAc (21.8 g, 222 mmol). The mixture was stirred at 90° C. for 12 h under N₂ atmosphere. TLC and LCMS showed the reaction was complete. The reaction mixture was filtered and concentrated in vacuum to give the desired product (20.0 g, yield 63.9%) as yellow oil, which was purified by column chromatography (SiO₂, Petroleum ether/Ethyl acetate=100/1 to 10/1, Rt=0.50). ¹H NMR (400 MHz, CDCl₃): δ 7.74 (d, J=8.0 Hz, 2H), 7.13 (d, J=7.8 Hz, 2H), 4.96 (d, J=7.8 Hz, 1H), 4.56-4.61 (m, 1H), 3.70 (s, 3H), 3.05-3.15 (m, 2H), 2.05 (s, 1H), 1.42 (s, 9H), 1.34 (s, 12H), 1.24-1.27 (m, 3H).

### Step 3. Preparation of (S)-methyl 2-((tert-butoxycarbonyl)amino)-3-(4′-(hydroxymethyl)-2′,6′-dimethoxy-[1,1′-biphenyl]-4-yl)propanoate

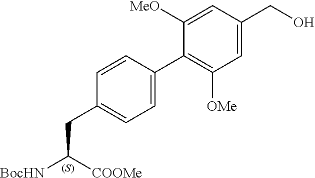

To a solution of (S)-methyl 2-((tert-butoxycarbonyl)amino)-3-(4-(4,4,5,5-tetramethyl-1,3,2-dioxaborolan-2-yl)phenyl)propanoate (10.0 g, 23.7 mmol), (4-bromo-3,5-dimethoxyphenyl) methanol (7.04 g, 28.4 mmol), Pd(dppf)Cl₂ (1.74 g, 2.37 mmol) and K₃PO₄ (15.1 g, 71.2 mmol) in dioxane (50.0 mL) and H₂O (10.0 mL) was stirred at 80° C. for 12 h. TLC and LCMS showed the reaction was complete. The reaction mixture was filtered. The filtrate was poured into water (50.0 mL) and extracted with ethyl acetate (50.0 mL*3). The combined organic layer was washed with brine (100 mL), dried over sodium sulfate, filtered and concentrated to give the desired product (6.27 g, yield 59.1%) as light yellow oil, which was purified by column chromatography (SiO₂, Petroleum ether/Ethyl acetate=20/1 to 1/1, Rt=0.20) and further purified by prep-HPLC (column: Phenomenex luna C18 250*50 mm*10 um; mobile phase: [water (0.1% TFA)-ACN]; B %: 30%-55%, 24 min). ¹H NMR (400 MHz, CDCl₃): δ 7.27-7.29 (m, 2H), 7.17 (d, J=8.0 Hz, 2H), 6.68 (s, 2H), 5.09 (d, J=7.8 Hz, 1H), 4.73 (s, 2H), 4.62-4.65 (m, 1H), 3.74 (s, 9H), 3.06-3.17 (m, 2H), 1.44 (s, 9H).

### Step 4. Preparation of (S)-methyl 2-amino-3-(4′-(hydroxymethyl)-2′,6′-dimethoxy-[1,1′-biphenyl]-4-yl)propanoate

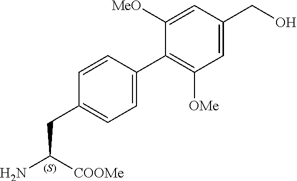

A solution of (S)-methyl 2-((tert-butoxycarbonyl)amino)-3-(4′-(hydroxymethyl)-2′,6′-dimethoxy -[1,1′-biphenyl]-4-yl)propanoate (10.2 g, 23.1 mmol) in HCl/dioxane (4.00 M, 100 mL) was stirred at 25° C. for 0.5 h. TLC and LCMS showed the reaction was complete. The reaction mixture was concentrated under vacuum to give the desired product (8.17 g, crude, HCl) as white solid, which was directly used for next step without further purification.

### Step 5. Preparation of (S)-methyl 1-((3,5-dichlorophenyl)sulfonyl)pyrrolidine-2-carboxylate

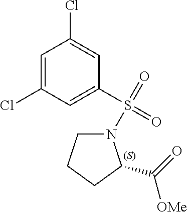

To a solution of (S)-methyl pyrrolidine-2-carboxylate (20.0 g, 120 mmol) in DCM (100 mL) and TEA (36.6 g, 362 mmol, 50.4 mL) was drop-wise added 3,5-dichlorobenzene-1-sulfonyl chloride (26.6 g, 108 mmol) at 0° C. The mixture was stirred at 25° C. for 16 h. TLC showed the reaction was complete. The reaction mixture was washed with 1N HCl (100 mL*3) and brine (100 mL). The organic layer was dried by sodium sulfate, filtered and concentrated in vacuum to give the desired product (30.5 g, yield 68.8%) as light yellow oil, which was directly used for next step without further purification.

### Step 6. Preparation of (S)-1-((3,5-dichlorophenyl)sulfonyl)pyrrolidine-2-carboxylic Acid

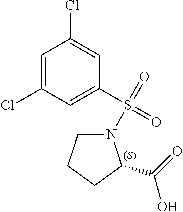

To a solution of (S)-methyl 1-((3,5-dichlorophenyl)sulfonyl)pyrrolidine-2-carboxylate (30.5 g, 83.1 mmol) in THF (100 mL) and MeOH (25.0 mL) was drop-wise added LiOH·H₂O (13.9 g, 332 mmol) in H₂O (25.0 mL). The mixture was stirred at 25° C. for 1 h. TLC showed the reaction was complete. The reaction mixture was acidified by 1N HCl to pH=3, and then extracted with ethyl acetate (200 mL*3) and washed with brine (200 mL). The organic layer was dried over anhydrous sodium sulfated, filtered and concentrated under vacuum to give the desired product (27.6 g, crude) as white solid, which was used directly for next step without further purification. ¹H NMR (400 MHz, CDCl₃): δ 7.77 (d, J=1.8 Hz, 2H), 7.58 (t, J=1.8 Hz, 1H), 4.41 (dd, J=4.6, 7.8 Hz, 1H), 3.55-3.49 (m, 1H), 3.34-3.40 (m, 1H), 2.26-2.12 (m, 2H), 2.11-1.99 (m, 1H), 1.98-1.88 (m, 1H).

### Step 7. Preparation of (S)-methyl 2-((S)-1-((3,5-dichlorophenyl)sulfonyl)pyrrolidine-2-carboxamido)-3-(4′-(hydroxymethyl)-2′,6′-dimethoxy-[1,1′-biphenyl]-4-yl)propanoate

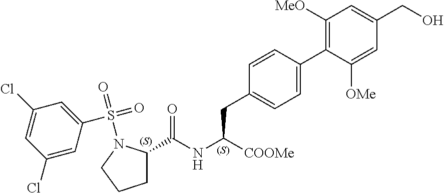

To a solution of (S)-1-((3,5-dichlorophenyl)sulfonyl)pyrrolidine-2-carboxylic acid, from Step 6 above (6.39 g, 19.4 mmol), HATU (8.87 g, 23.3 mmol) and DIEA (12.5 g, 97.2 mmol, 16.9 mL) in DMF (150 mL) was added (S)-methyl 2-amino-3-(4′-(hydroxymethyl)-2′,6′-dimethoxy-[1,1′-biphenyl]-4-yl)propanoate, from Step 4 above (8.17 g, 21.4 mmol, HCl). Then the reaction mixture was stirred at 25° C. for 12 h. TLC and LCMS showed the reaction was complete. The mixture was poured into water (150 mL) and extracted with ethyl acetate (200 mL*3). The combined organic layer was washed with brine (200 mL), dried over sodium sulfate, filtered and concentrated to give the desired product (13.0 g, yield 95.0%) as yellow oil, which was purified by column chromatography (SiO₂, Petroleum ether/Ethyl acetate=10/1 to 1/2, Rt=0.60). ¹H NMR (400 MHz, CDCl₃): δ 7.72 (d, J=1.8 Hz, 2H), 7.60 (t, J=1.8 Hz, 1H), 7.27-7.23 (m, 2H), 7.15 (d, J=8.2 Hz, 2H), 7.08 (d, J=8.0 Hz, 1H), 6.66 (s, 2H), 4.88 (dt, J=5.6, 8.0 Hz, 1H), 4.71 (d, J=5.6 Hz, 2H), 4.11-4.09 (m, 1H), 3.80 (s, 3H), 3.70 (s, 6H), 3.40-3.30 (m, 2H), 3.14-3.01 (m, 2H), 2.30 (t, J=5.8 Hz, 1H), 1.63-1.43 (m, 3H)

### Step 8. Preparation of methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2- [2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxymethyl]phenyl]phenyl]propanoate

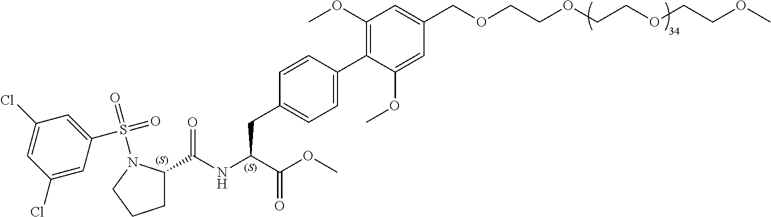

To a mixture of methyl (2S)-2-[[(2S)-1-(3,5-dichlorophenyl)sulfonylpyrrolidine-2-carbonyl]amino]-3-[4-[4-(hydroxymethyl)-2,6-dimethoxy-phenyl]phenyl]propanoate (84.8 mg, 123 μmol) and m-PEG36-alcohol (BroadPharm) (200 mg, 123 μmol) was added InCl₃ (21.8 mg, 98.8 μmol, 6.32 μL). The reaction mixture was stirred at 120° C. for 12 hrs. LCMS showed the reaction was complete. The reaction was concentrated under vacuum to give the desired product (100 mg, 7.39% yield) as yellow oil, which was purified by prep-HPLC (column: Phenomenex luna C18 150*40 mm*15 um; mobile phase: [water (0.1% TFA)-ACN]; B %: 52%-82%, 11 min).

### Step 9. Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2- [2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoic Acid

### Example 2

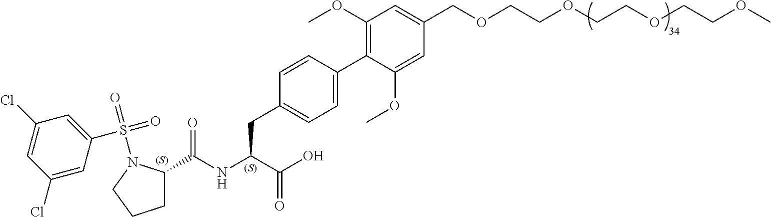

To a solution of methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2- [2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoate (100 mg, 36.5 μmol) in THF (5.00 mL) was drop-wise added LiOH·H₂O (6.14 mg, 146 μmol) in H₂O (1.00 mL). The mixture was stirred at 25° C. for 0.5 hr. LCMS showed the reaction was complete. The reaction mixture was diluted with water (20.0 mL) and adjusted pH to 3 with 2 M HCl. The mixture was extracted with ethyl acetate (20.0 mL*3). The aqueous phase was freeze-drying to give the desired product, Example 2 (42.03 mg, 51.3% yield) as white solid, which was purified by prep-HPLC (column: Waters Xbridge 150*25 mm*5 um; mobile phase: [water (10 mM NH₄HCO₃)-ACN]; B %: 23%-53%, 9 min) and further purified by prep-HPLC (column: Waters Xbridge 150*25 mm*5 um; mobile phase: [water (10 mM NH₄HCO₃)-ACN]; B %: 23%-53%, 9 min). ¹H NMR (400 MHz, CDCl₃): δ 7.73 (d, J=1.6 Hz, 2H), 7.59 (t, J=1.6 Hz, 1H), 7.45 (d, J=7.2 Hz, 1H), 7.24-7.18 (m, 4H), 6.63 (s, 2H), 4.76-4.65 (m, 1H), 4.58 (s, 2H), 4.11 (d, J=6.0 Hz, 1H), 3.87-3.77 (m, 1H), 3.72-3.61 (m, 142H), 3.57-3.54 (m, 3H), 3.49-3.45 (m, 1H), 3.43 (d, J=6.4 Hz, 1H), 3.39 (s, 3H), 3.26-3.20 (m, 1H), 3.11-3.05 (m, 1H), 2.07-2.01 (m, 1H), 1.62-1.42 (m, 3H).

The desired product was confirmed by QTOF-MS with a mass of 2233.0753.

Example 2 may also be synthesized according to the method described for the synthesis of Example 1 above, by substituting an equivalent amount of m-PEG36-alcohol (BroadPharm) for m-PEG24-alcohol (BroadPharm) in Step 6.

### Example 3

### Preparation of (2S)-2-[[(2S)-1-(3,5-dichlorophenyl)sulfonylpyrrolidine-2-carbonyl]amino]-3-[4-[2,6-dimethoxy-4-[2-[2-[[1-[2-[2-[2-[2-[2-[2-[2-[2-[2-[2-[2- [2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethyl]triazol-4-yl]methoxy]ethoxy]ethoxymethyl]phenyl]phenyl]propanoic Acid

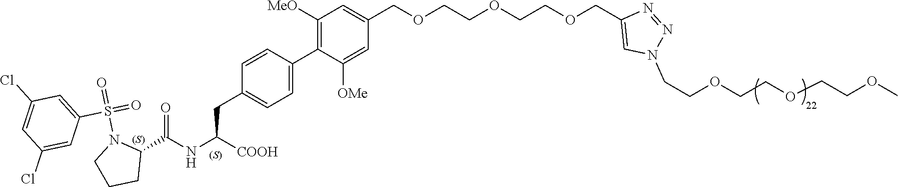

### The Synthesis of Example 3 is Depicted in Scheme C:

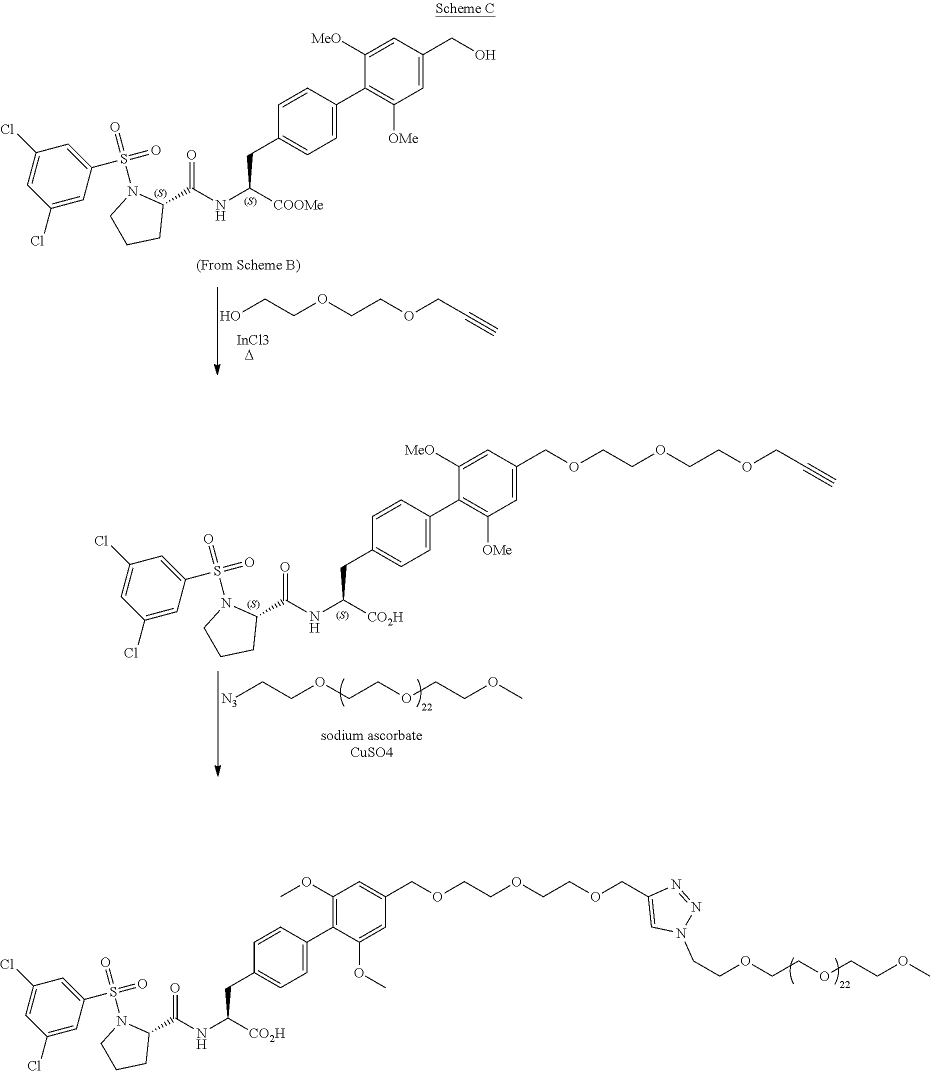

### Step 1. Preparation of (S)-2-((S)-1-((3,5-dichlorophenyl)sulfonyl)pyrrolidine-2-carboxamido)-3-(2′,6′-dimethoxy-4′-((2-(2-(prop-2-yn-1-yloxy)ethoxy)ethoxy)methyl)-[1,1′-biphenyl]-4-yl)propanoic acid

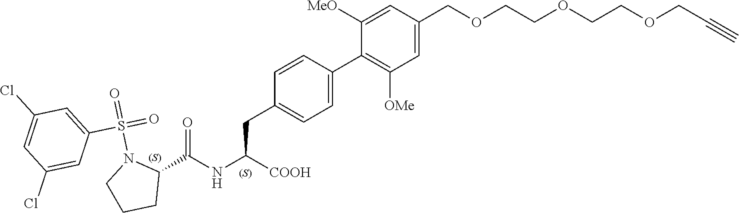

To a solution of (S)-methyl 2-((S)-1-((3,5-dichlorophenyl)sulfonyl)pyrrolidine-2-carboxamido)-3-(4′-(hydroxymethyl)-2′,6′-dimethoxy-[1,1′-biphenyl]-4-yl)propanoate, from Step 7 in Example 2 (200 mg, 291 μmol) and InCl₃ (51.5 mg, 233 μmol, 14.9 μL) in 2-(2-(prop-2-yn-1-yloxy)ethoxy)ethanol (419 mg, 2.91 mmol) was stirred at 80° C. for 12 h. LCMS showed the reaction was complete. The reaction mixture was diluted with acetonitrile (8.00 mL) and filtered to give the desired product (160 mg, yield 17.3%) as brown oil, which was purified with prep-HPLC (column: Phenomenex luna C18 150*40 mm*15 um; mobile phase: [water (0.1% TFA)-ACN]; B %: 50%-70%, 10 min).

### Step 2. Preparation of (2S)-2-[[(2S)-1-(3,5-dichlorophenyl)sulfonylpyrrolidine-2-carbonyl]amino]-3-[4-[2,6-dimethoxy-4-[2-[2-[[1-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethyl]triazol-4-yl]methoxy]ethoxy]ethoxymethyl]phenyl]phenyl]propanoic acid

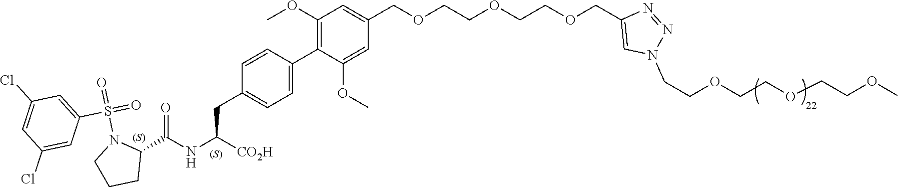

To a solution of (S)-2-((S)-1-((3,5-dichlorophenyl)sulfonyl)pyrrolidine-2-carboxamido)-3-(2′,6′-dimethoxy-4′-((2-(2-(prop-2-yn-1-yloxy)ethoxy)ethoxy)methyl)-[1,1′-biphenyl]-4-yl)propanoic acid, from Step 1 above (71.0 mg, 89.7 μmol, 131 mL), m-PEG24-azide (BroadPharm) (100 mg, 89.7 μmol), CuSO₄ (14.3 mg, 89.7 μmol, 13.7 μL) and sodium ascorbate (17.7 mg, 89.7 μmol) in H₂O (400 μL) and THF (2.00 mL) was stirred at 100° C. for 12 h. LCMS showed the reaction was complete. The reaction mixture was diluted with acetonitrile (4.00 mL) and filtered to give the desired product (51.0 mg, yield 30.1%) as yellow gum, which was purified with prep-HPLC (column: Phenomenex Synergi C18 150*25*10 um; mobile phase: [water (0.225% FA)-ACN]; B %: 36%-66%, 10 min). ¹H NMR (400 MHz, CDCl₃): δ 7.73 (d, J=2.0 Hz, 2H), 7.70 (s, 1H), 7.61 (t, J=1.8 Hz, 1H), 7.17-7.27 (m, 5H), 6.65 (d, J=16.0 Hz, 2H), 4.92 (d, J=6.0 Hz, 1H), 4.69 (d, J=30.0 Hz, 4H), 4.51 (t, J=5.2 Hz, 2H), 4.34-4.36 (m, 2H), 4.11 (d, J=5.6 Hz, 1H), 3.86 (t, J=5.2 Hz, 2H), 3.65 (s, 105H), 3.38 (s, 5H), 3.08-3.13 (m, 2H), 2.03-2.08 (m, 1H), 1.59-1.61 (m, 2H).

The desired product was confirmed by QTOF-MS with a mass of 1875.8408.

### Example 4

### Preparation of (2S)-2-[[(2S)-1-(3,5-dichlorophenyl)sulfonylpyrrolidine-2-carbonyl]amino]-3-[4-[2,6-dimethoxy-4-[5K MW PEG-thiomethyl]phenyl]phenyl]propanoic Acid

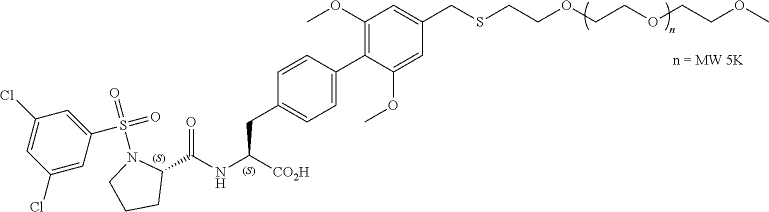

### Step 1. Preparation of (S)-methyl 2-((S)-1-((3,5-15 dichlorophenyl)sulfonyl)pyrrolidine-2-carboxamido)-3-(4′-(bromomethyl)-2′,6′-dimethoxy-[1,1′-biphenyl]-4-yl)propanoate

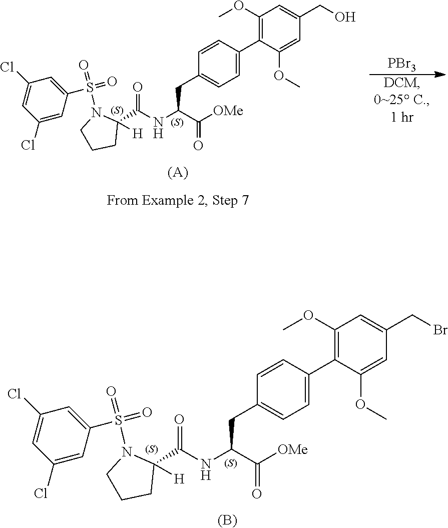

To a solution of PBr₃ (99.7 mg, 368 μmol, 1.20 eq) in DCM (2.00 mL) was added compound (A) (from Example 2, Step 7 above) (200 mg, 306 μmol, 1.00 eq) in DCM (0.50 mL) at 0° C. The mixture was stirred at 25° C. for 1 hr. LCMS showed that compound (A) was consumed and desired mass was detected. The reaction mixture was quenched with water (20.0 mL) and extracted with DCM (20.0 mL*3). The combined organic phase was washed with brine (20.0 mL), dried over Na2SO₄, filtered and concentrated under vacuum. The desired compound (B) (200 mg, crude) was obtained as white solid. The crude product was directly used for next step without purification.

### Step 2. Preparation of (S)-methyl 2-((S)-1-((3,5-dichlorophenyl)sulfonyl)pyrrolidine-2-carboxamido)-3-(4′-(5K MW PEG thiomethyl)-2′,6′-dimethoxy-[1,1′-biphenyl]-4-yl)propanoate

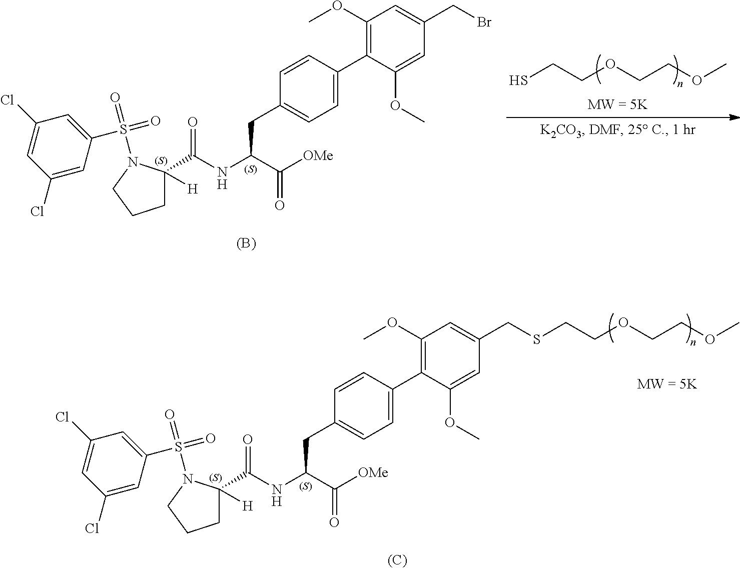

To a solution of compound (B) (28.4 mg, 36.4 μmol, 91.8% purity, 1.00 eq) and m-PEG-thiol, MW 5K (BroadPharm, Catalogue #BP-23721) (200 mg, 1.47 mmol, 40.2 eq) in DMF (5.00 mL) was added K₂CO₃ (10.0 mg, 72.9 μmol, 2.00 eq). The mixture was stirred at 25° C. for 1 hr. LCMS showed that compound (B) was consumed. The mixture was concentrated under vacuum. The compound (C) (238 mg, crude) was obtained as yellow oil. The crude product was directly used for next step without purification.

### Step 3. Preparation of (2S)-2-[[(2S)-1-(3,5-dichlorophenyl)sulfonylpyrrolidine-2-carbonyl]amino]-3-[4-[2,6-dimethoxy-4-[5K MW PEG thiomethyl]phenyl]phenyl]propanoic Acid

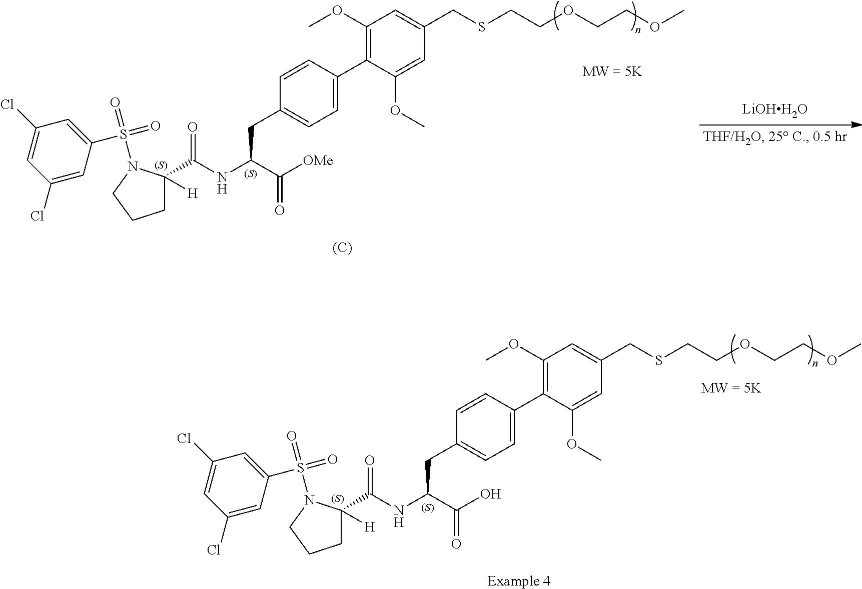

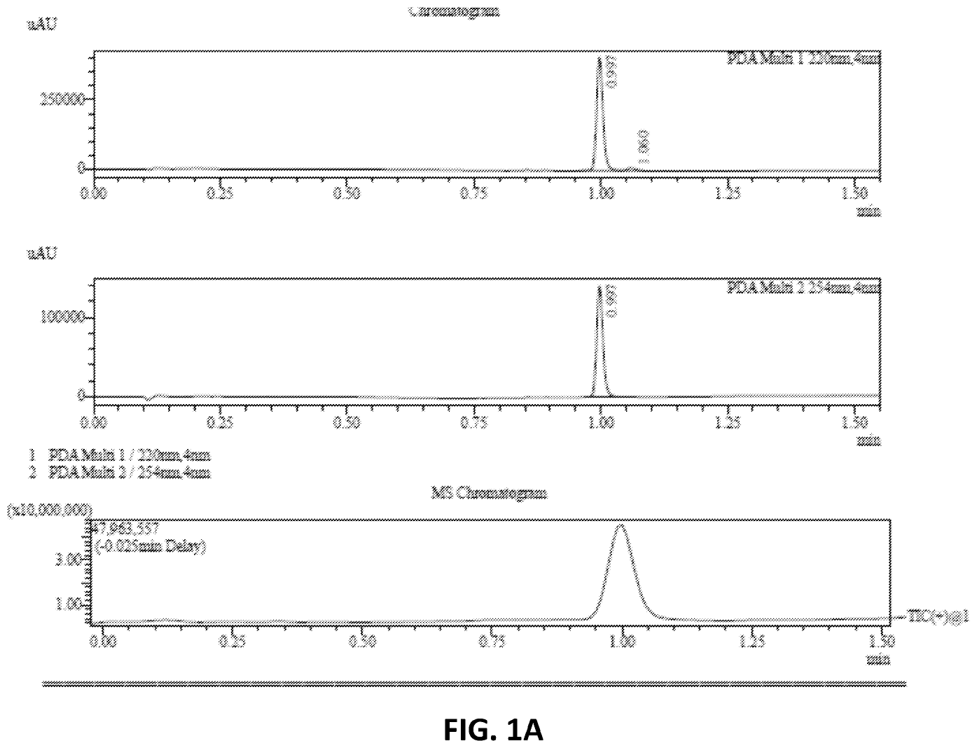

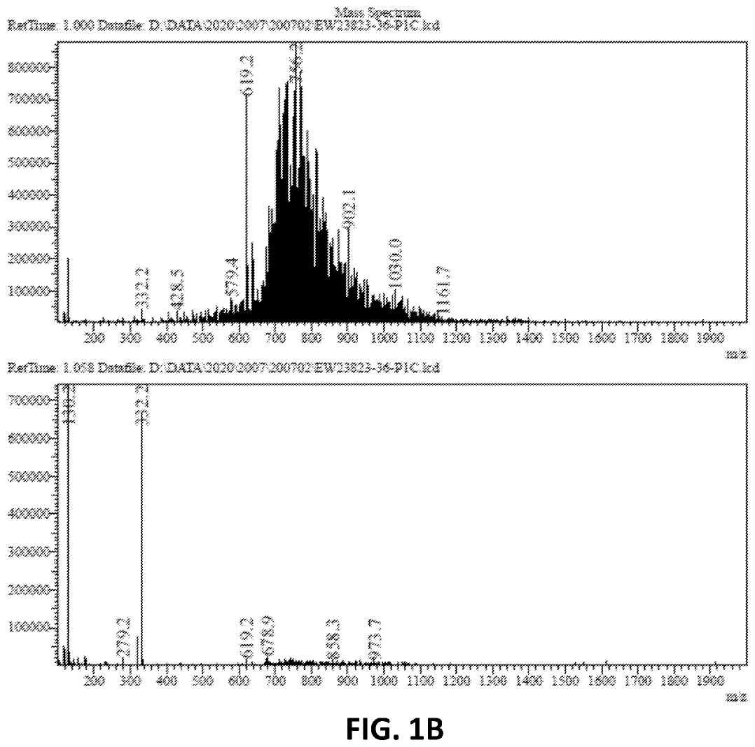

To a solution of compound (C) (238 mg, 309 μmol, 1.00 eq) in DMF (2.00 mL) was dropwise added LiOH·H₂O (7.08 mg, 168 μmol) in H₂O (0.50 mL). The mixture was stirred at 25° C. for 0.5 hr. LCMS showed that compound (C) was consumed. The mixture was poured into water (20.0 mL) and extracted with ethyl acetate (20.0 mL*2). The aqueous phase was adjusted pH=3 with 1N HCl and dried by lyophilization. The residue was purified by prep-HPLC (column: 3_Phenomenex Luna C18 75*30 mm*3 um; mobile phase: [water (0.1% TFA)-ACN]; B %: 38%-68%, 7 min). From ¹H NMR, LCMS and HPLC (one peak) the Example 4 (91.35 mg, 116 μmol, 37.7% yield, 96.5% purity) was obtained as off-white solid (see e.g., FIG. 1A-FIG. 1B).

¹H NMR: δ 7.74-7.72 (m, 2H), 7.63-7.60 (m, 1H), 7.25 (s, 2H), 7.22-7.17 (m, 2H), 7.11 (d, J=7.2 Hz, 1H), 6.63 (s, 2H), 4.88-4.80 (m, 1H), 4.13 (dd, J=2.4, 8.4 Hz, 1H), 3.85-3.77 (m, 6H), 3.71 (s, 6H), 3.69-3.61 (m, 571H), 3.59-3.54 (m, 4H), 3.47 (dd, J=4.0, 5.6 Hz, 3H), 3.13 (td, J=7.2, 14.4 Hz, 3H), 2.71 (t, J=6.8 Hz, 2H), 2.09-2.04 (m, 1H), 1.67-1.55 (m, 2H), 1.53-1.41 (m, 1H)

### Example 5

### Preparation of (2S)-2-[[(2S)-1-(3,5-dichlorophenyl)sulfonylpyrrolidine-2-carbonyl]amino]-3-[4-[2,6-dimethoxy-4-[20K MW PEG-thiomethyl]phenyl]phenyl]propanoic Acid

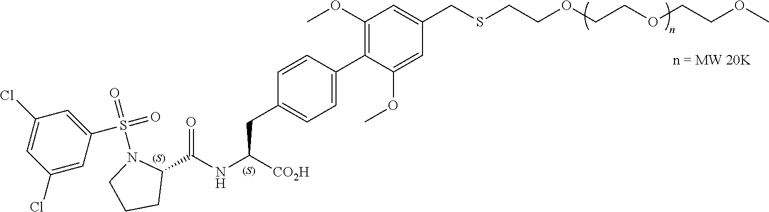

### Step 1. Preparation of (S)-methyl 2-((S)-1-((3,5-dichlorophenyl)sulfonyl)pyrrolidine-2-carboxamido)-3-(4′-(20K MW PEG thiomethyl)-2′,6′-dimethoxy-[1,1′-biphenyl]-4-yl)propanoate

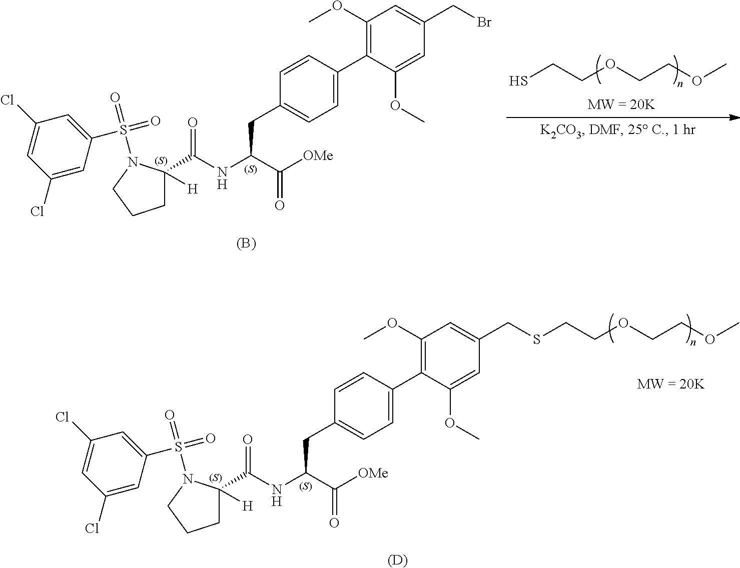

To a solution of compound (B) (from Example 4, Step 1 above) (7.10 mg, 9.12 μmol, 91.8% purity, 1.00 eq) and m-PEG-thiol, MW 20K (BroadPharm, Catalogue #BP-23723) (200 mg, 1.47 mmol, 160 eq) in DMF (5.00 mL) was added K₂CO₃ (2.52 mg, 18.2 μmol, 2.00 eq). The mixture was stirred at 25° C. for 1 hr. LCMS) showed that compound (B) was consumed. The mixture was concentrated under vacuum. The compound (D) (210 mg, crude) was obtained as colorless oil. The crude product was directly used for next step without purification.

### Step 2. Preparation of (2S)-2-[[(2S)-1-(3,5-dichlorophenyl)sulfonylpyrrolidine-2-carbonyl]amino]-3-[4-[2,6-dimethoxy-4-[20K MW PEG thiomethyl]phenyl]phenyl]propanoic Acid

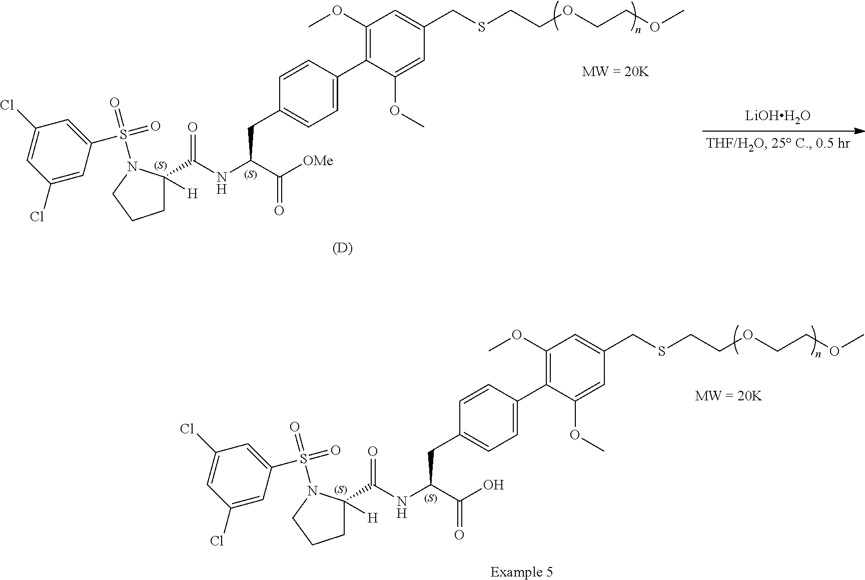

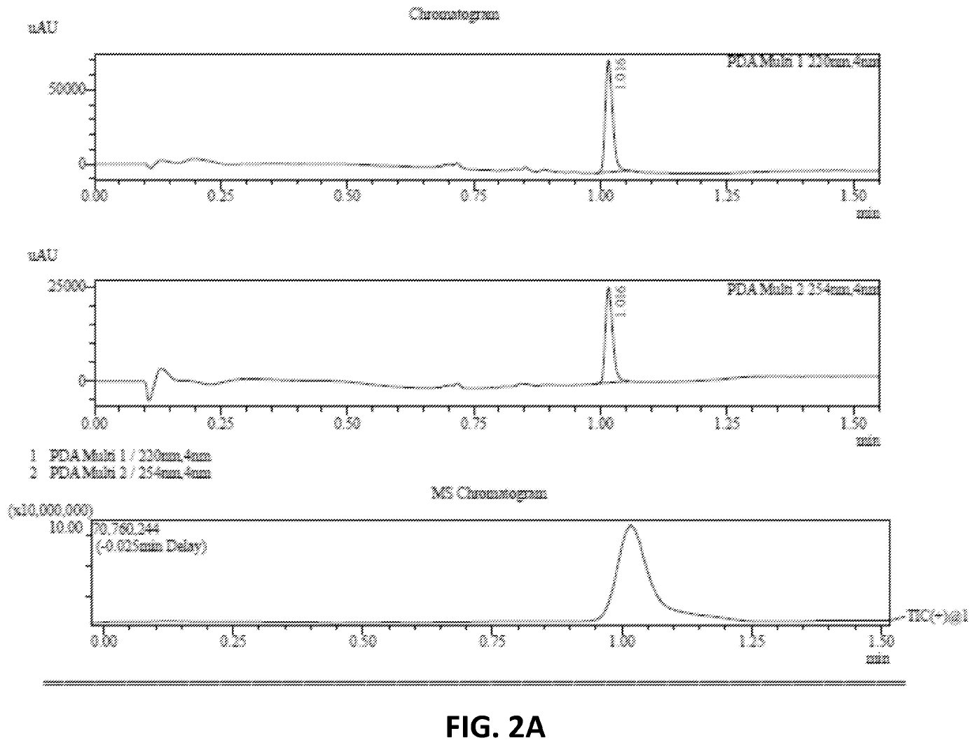

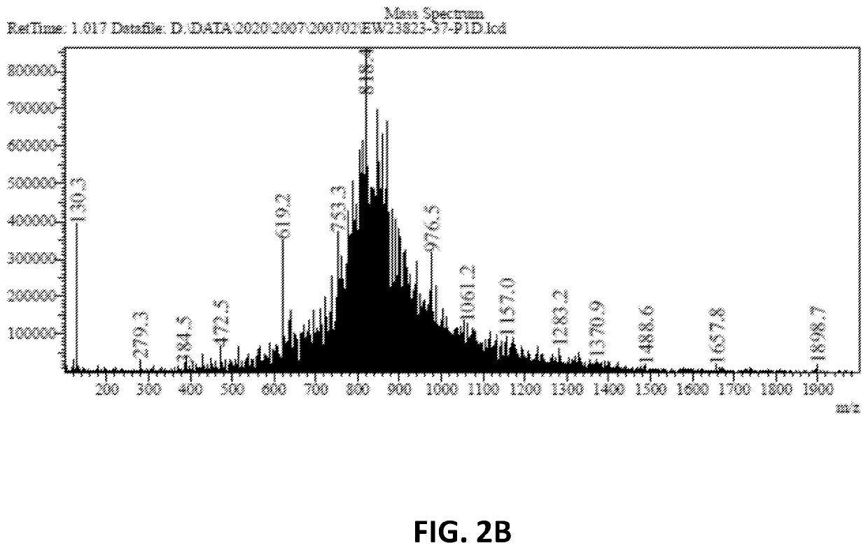

To a solution of compound (D) (210 mg, 272 μmol, 1.00 eq) in DMF (2.00 mL) was dropwise added LiOH·H₂O (1.77 mg, 42.1 μmol) in H₂O (0.50 mL). The mixture was stirred at 25° C. for 0.5 hr. LCMS showed that compound (D) was consumed. The mixture was poured into water (20.0 mL) and extracted with ethyl acetate (20.0 mL*2). The aqueous phase was adjusted pH=3 with 1N HCl and dried by lyophilization. The residue was purified by prep-HPLC (column: 3_Phenomenex Luna C18 75*30 mm*3 um; mobile phase: [water (0.1% TFA)-ACN]; B %: 34%-64%, 7 min). From ¹H NMR, LCMS and HPLC, Example 5 (74.45 mg, 92.4 μmol, 33.8% yield, 93.8% purity) was obtained as a white solid (see e.g., FIG. 2A-FIG. 2B).

¹H NMR: δ 7.72 (d, J=2.0 Hz, 2H), 7.63-7.60 (m, 1H), 7.25 (s, 2H), 7.22-7.17 (m, 2H), 6.63 (s, 2H), 4.89-4.77 (m, 1H), 4.18-4.06 (m, 1H), 3.86-3.78 (m, 16H), 3.71 (s, 9H), 3.47 (dd, J=4.4, 5.6 Hz, 2334H), 3.46-3.48 (m, 14H), 2.70 (t, J=6.8 Hz, 3H)

### Example 6

### Preparation of (S)-2-((S)-1-((3,5-dichlorophenyl)sulfonyl)pyrrolidine-2-carboxamido)-3-(2′,6′-dimethoxy-4′-(2,5,8,11,17,20,23-heptaoxa-14-thia-[5K MW PEG]-tetracosyl)-[1,1′-biphenyl]-4-yl)propanoic Acid

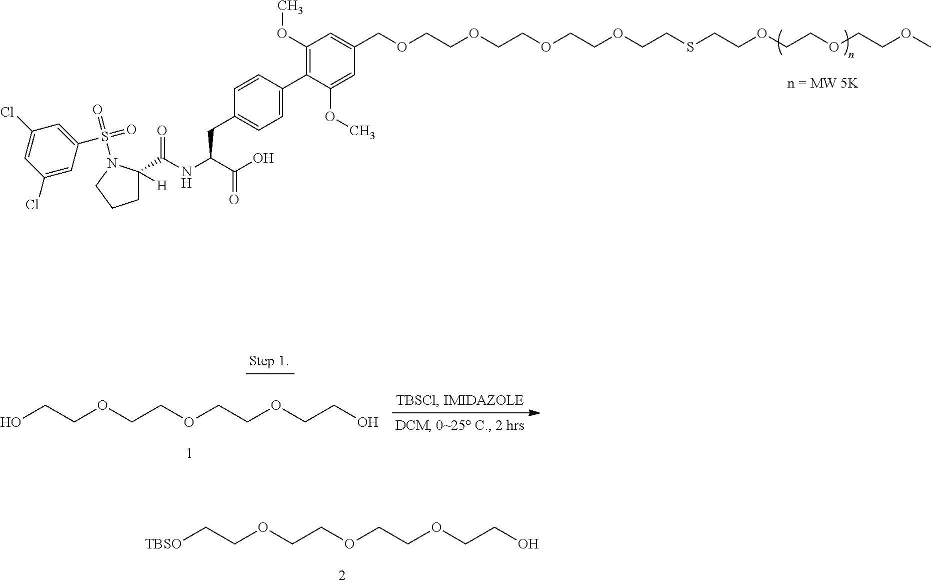

To a solution of compound 1 (38.6 g, 199 mmol, 34.2 mL, 3.00 eq) and IMIDAZOLE (13.5 g, 199 mmol, 3.00 eq) in DCM (100 mL) was dropwise added TBSCl (10.0 g, 66.3 mmol, 8.13 mL, 1.00 eq) at 0° C. The mixture was stirred at 25° C. for 2 hrs. TLC (Dichloromethane/Methanol=10/1) showed TBSCl (Rt=0.70) was consumed completely and a main new spot (Rt=0.50) was formed. The mixture was washed with water (200 mL*2) and brine (200 mL*2). The organic phase was dried by Na₂SO₄, filtered and concentrated under vacuum. The compound 2 (19.0 g, 61.5 mmol, 92.8% yield) was obtained as colorless oil. This crude product was directly used for next step without purification.

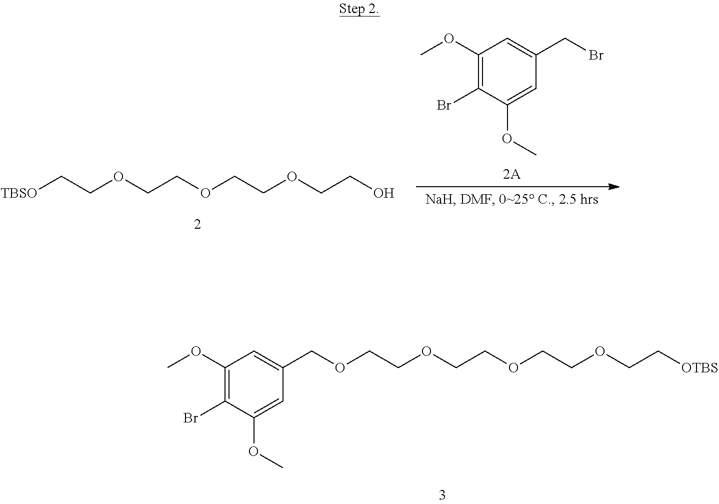 [SMILES: COc1cc(CBr)cc(OC)c1Br ; CC(C)(C)[Si](C)(C)OCCOCCOCCOCCO ; COc1cc(COCCOCCOCCOCCO[Si](C)(C)C(C)(C)C)cc(OC)c1Br]

To a solution of compound 2 (5.00 g, 16.2 mmol, 1.00 eq) in DMF (50.0 mL) was added NaH (648 mg, 16.2 mmol, 60.0% purity, 1.00 eq) at 0° C. The mixture was stirred at 0° C. for 0.5 hr. Then compound 2A (from Example 1, Step 5 above) (5.02 g, 16.2 mmol, 1.00 eq) in DMF (10.0 mL) was added dropwise. The mixture was stirred at 25° C. for 2 hrs. TLC (Petroleum ether/Ethyl acetate=1/1) showed compound 2A (Rt=0.80) was consumed completely and desired product (Rt=0.40) was formed. The mixture was poured into saturated NH₄Cl solution (200 mL) and extracted with ethyl acetate (200 mL*3). The combined organic phase was washed with brine (200 mL), dried over Na2SO₄, filtered and concentrated. The crude product was purified by MPLC (SiO₂, Petroleum ether/Ethyl acetate=1/1, Rt=0.40. The compound 3 (2.80 g, 5.21 mmol, 32.1% yield) was obtained as light yellow oil.

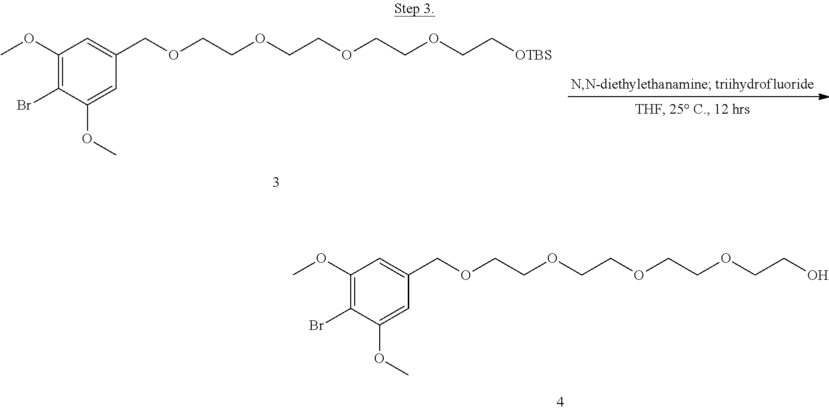 [SMILES: COc1cc(COCCOCCOCCOCCO[Si](C)(C)C(C)(C)C)cc(OC)c1Br ; COc1cc(COCCOCCOCCOCCO)cc(OC)c1Br]

To a solution of compound 3 (2.60 g, 4.84 mmol, 1.00 eq) in THF (26.0 mL) was added N,N-diethylethanamine; trihydrofluoride (1.17 g, 7.26 mmol, 1.18 mL, 1.50 eq). The mixture was stirred at 25° C. for 12 hrs. LCMS showed that compound 3 was consumed and the desired mass was detected. The mixture was poured into saturated NaHCO₃ aqueous (100 mL) and extracted with ethyl acetate (50.0 mL*3). The combined organic phase was washed with brine (100 mL), dried over Na₂SO₄, filtered and concentrated under vacuum. The compound 4 (1.90 g, 4.43 mmol, 91.6% yield, 98.7% purity) was obtained as yellow oil. This crude product was directly used for next step without purification.

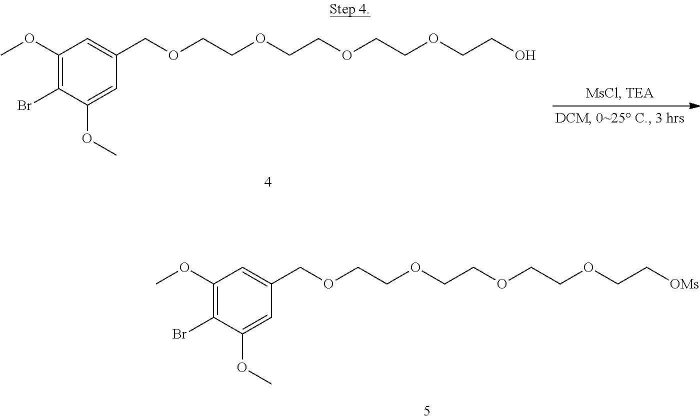 [SMILES: COc1cc(COCCOCCOCCOCCO)cc(OC)c1Br ; COc1cc(COCCOCCOCCOCCOS(C)(=O)=O)cc(OC)c1Br]

To a solution of compound 4 (1.70 g, 3.96 mmol, 98.7% purity, 1.00 eq) and TEA (802 mg, 7.93 mmol, 1.10 mL, 2.00 eq) in DCM (20.0 mL) was added MsCl (710 mg, 6.20 mmol, 479 μL, 1.56 eq) at 0° C. The mixture was stirred at 25° C. for 2 hrs. LCMS showed that 23.8% of compound 4 (RT=0.799 min) was retained and the desired mass was detected (RT=0.845 min). Then MsCl (400 mg, 3.49 mmol, 270 μL, 8.81^{e-1} eq) was added at 0° C. and the mixture was stirred at 25° C. for 1 hr. LCMS showed that compound 4 was consumed and the desired mass was detected (RT=0.853 min). The mixture was quenched with ice-water (20.0 mL), extracted with DCM (20.0 mL*3). The combined organic layers were washed with brine (50.0 mL), dried over Na₂SO₄ and concentrated. The compound 5 (1.80 g, crude) was obtained as yellow oil. The crude product was directly used for next step without purification

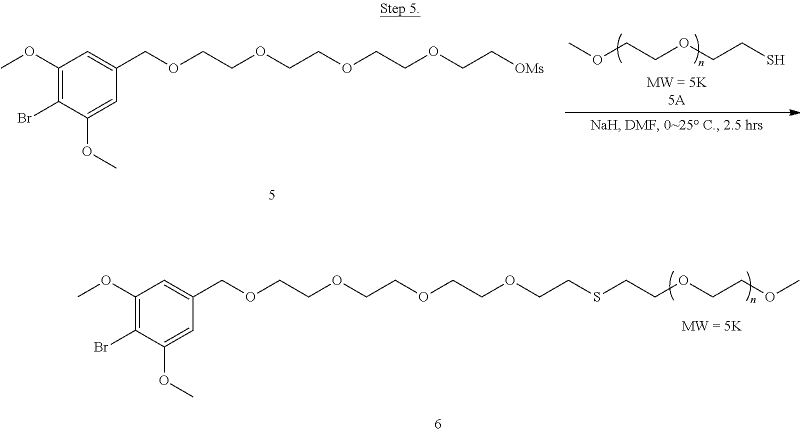

To a solution of compound 5A, m-PEG-thiol, MW 5K (BroadPharm, Catalogue #BP-23721), (2.80 g, 20.5 mmol, 36.8 eq) in DMF (40.0 mL) was added NaH (26.8 mg, 670 μmol, 60% purity, 1.20 eq) at 0° C. The mixture was stirred at 0° C. for 0.5 hr. Then compound 5 (299 mg, 558 μmol, 93.6% purity, 1.00 eq) was added dropwise. The mixture was stirred at 25° C. for 2 hrs. LCMS showed that compound 5 was consumed and possible mass (RT=0.974 min) was detected. The mixture was poured into saturated NH₄Cl solution (40.0 mL) and extracted with ethyl acetate (40.0 mL*3). The aqueous phase was dried by lyophilization. The residue was purified by prep-HPLC (column: Phenomenex luna C18 (250*70 mm, 10 um); mobile phase: [water (0.1% TFA)-ACN]; B %: 42%-65%, 24 min). The compound 6 (1.00 g, 1.85 mmol, 100% purity) was obtained as white solid.

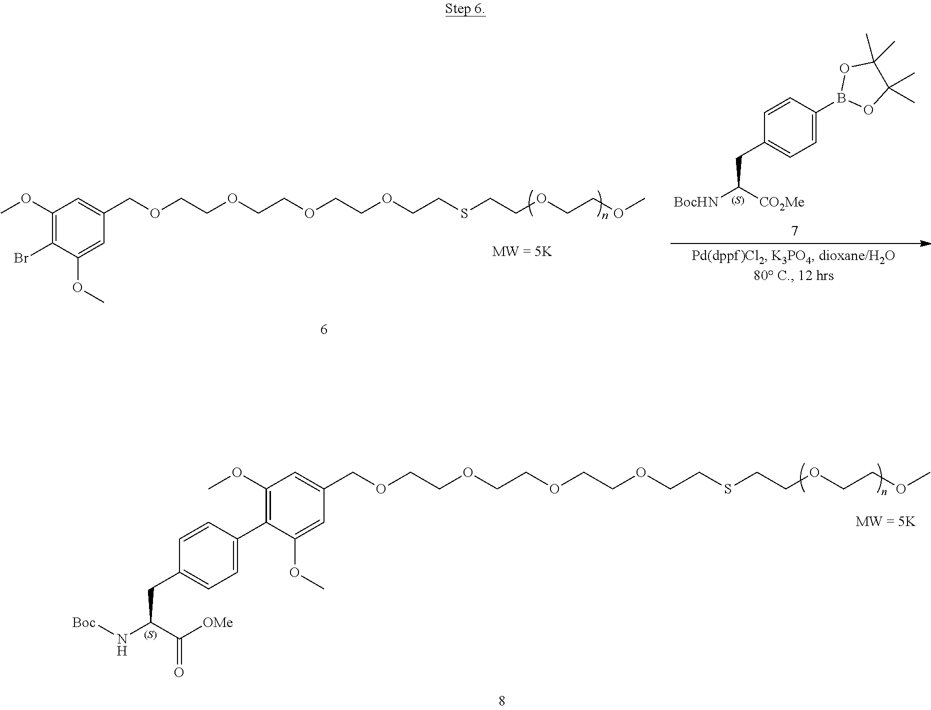

A solution of compound 6 (1.00 g, 1.85 mmol, 100% purity, 9.08 eq), compound 7 (from Example 1, Step 3 above) (88.2 mg, 203 μmol, 93.4% purity, 1.00 eq), Pd(dppf)Cl₂ (14.8 mg, 20.3 μmol, 0.10 eq), K₃PO₄ (129 mg, 610 μmol, 3.00 eq) in dioxane (10.0 mL) and H₂O (2.00 mL) was stirred at 80° C. for 12 hrs. LCMS showed that possible mass (RT=0.990 min) was detected. The mixture was filtered by celatum and the filtrate was poured into water (20.0 mL). The mixture was extracted with ethyl acetate (20.0 mL*3). The combined organic phase was washed with brine (20.0 mL), filtered and concentrated. The residue was purified by prep-HPLC (column: 3_Phenomenex Luna C18 75*30 mm*3 um; mobile phase: [water (0.1% TFA)-ACN]; B %: 40%-70%, 7 min). The compound 8 (470 mg, 635 μmol, 100% purity) was obtained as light yellow gum.

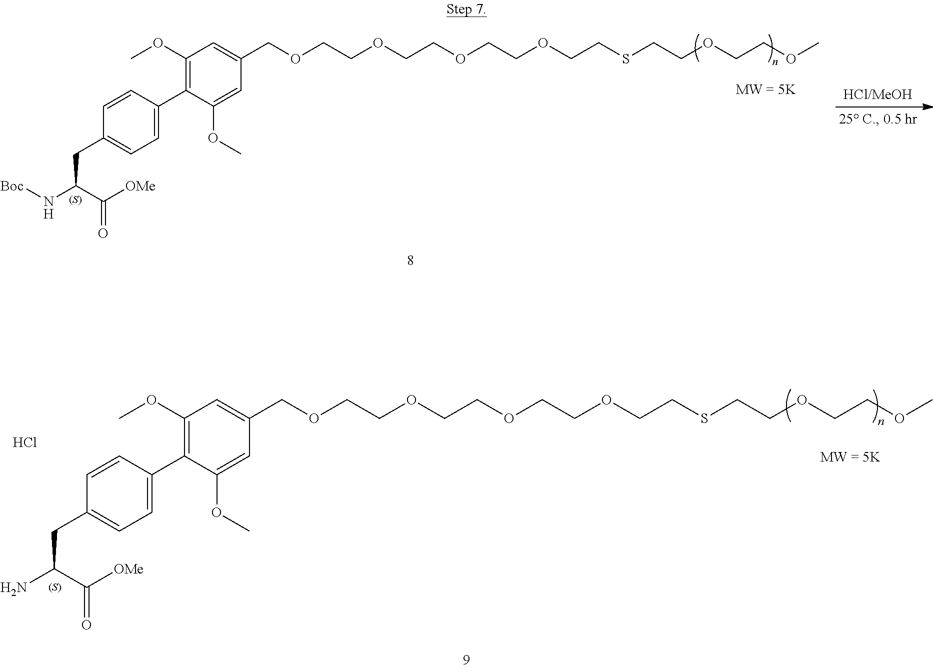

A solution of compound 8 (470 mg, 635 μmol, 100% purity, 1.00 eq) in HCl/MeOH (4 M, 6.67 mL, 41.9 eq) was stirred at 25° C. for 0.5 hr. LCMS showed that possible mass (RT=0.898 min) was detected. The mixture was concentrated under vacuum. The compound 9 (429 mg, crude, HCl) was obtained as light yellow oil. This crude product was directly used for next step.

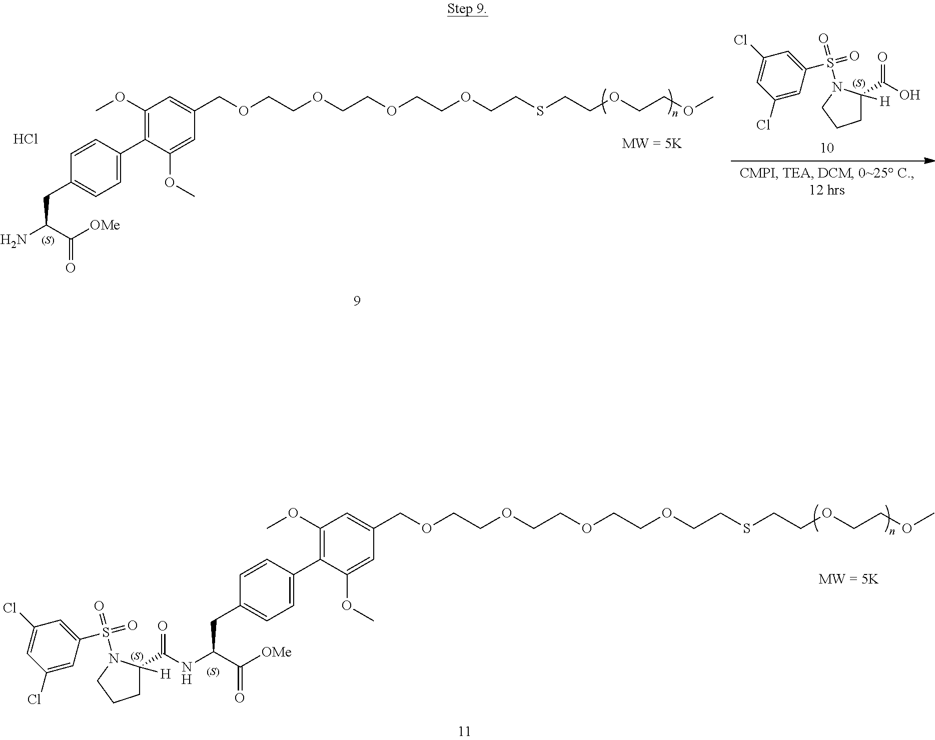

To a solution of compound 10 (from Example 1, Step 2 above) (20.0 mg, 61.4 μmol, 99.6% purity, 1.00 eq), CMPI (23.5 mg, 92.1 μmol, 1.50 eq) and TEA (18.6 mg, 184 μmol, 25.6 μL, 3.00 eq) in DCM (8.00 mL) was dropwise added compound 9 (330 mg, 487 μmol, 7.94 eq, HCl) in DCM (2.00 mL) at 0° C. The mixture was stirred at 25° C. for 12 hrs. LCMS showed that possible mass (RT=1.026 mins) was detected. The mixture was poured into water (20.0 mL) and extracted with DCM (20.0 mL*3). The combined organic phase was washed with brine (20.0 mL), dried over Na₂SO₄, filtered and concentrated. The crude product was purified by prep-HPLC (column: 3_Phenomenex Luna C18 75*30 mm*3 um; mobile phase: [water (0.1% TFA)-ACN]; B %: 38%-78%, 10 min) and further purified by prep-HPLC (column: 3_Phenomenex Luna C18 75*30 mm*3 um; mobile phase: [water (0.1% TFA)-ACN]; B %: 38%-68%, 7 min). The compound 11 (28.0 mg, 29.6 μmol) was obtained as light yellow gum.

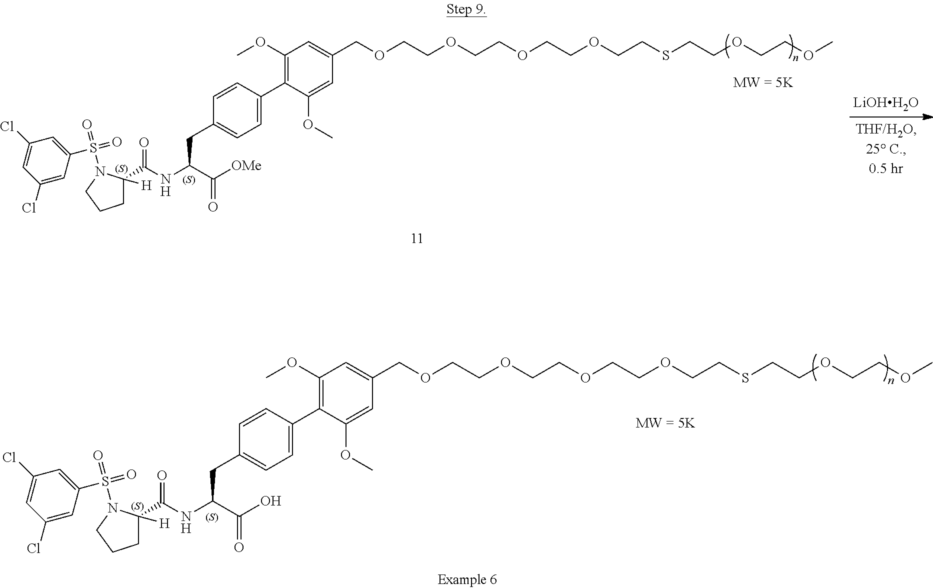

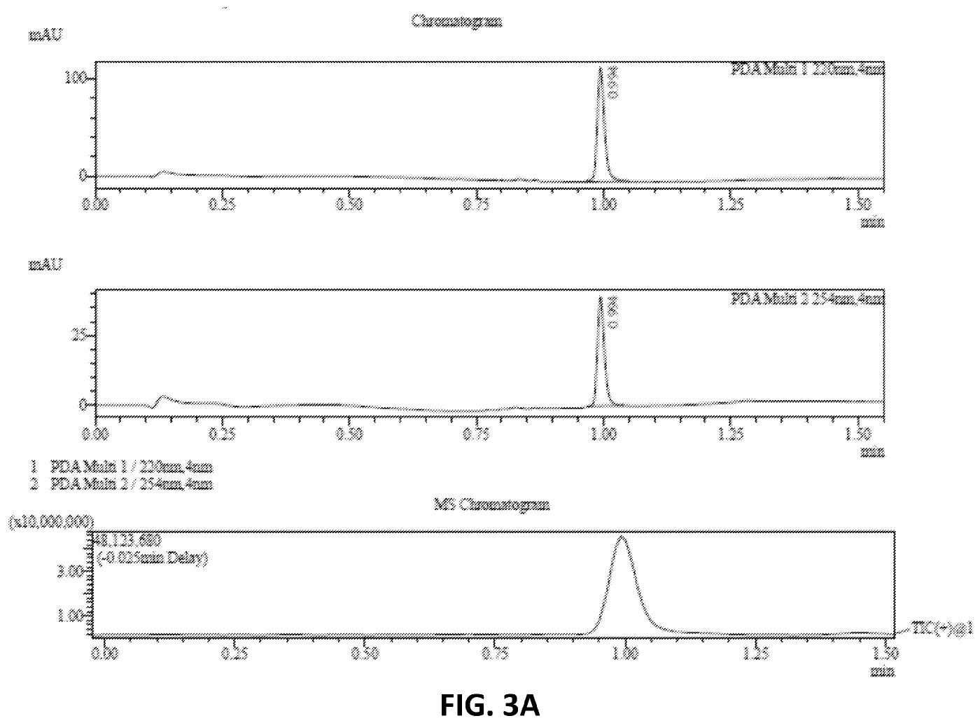

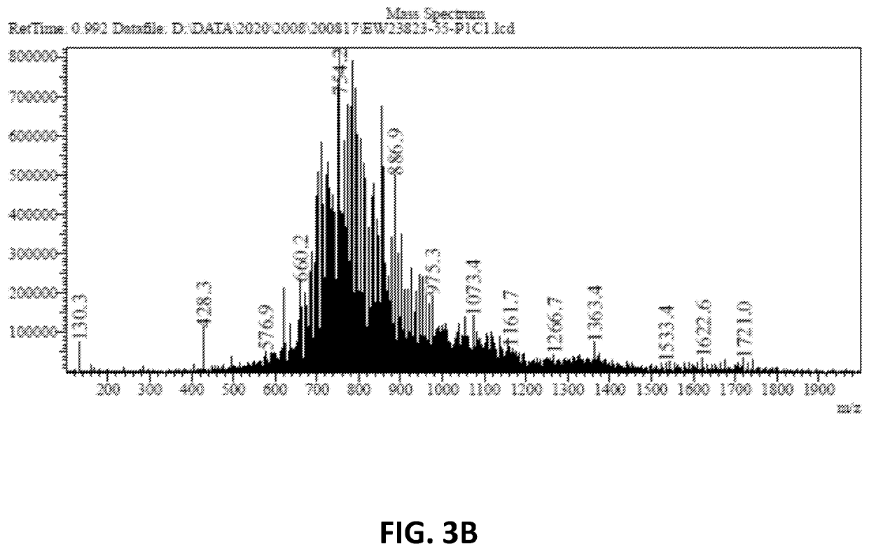

To a solution of compound 11 (28.0 mg, 29.6 μmol, 1.00 eq) in THF (1.00 mL) was dropwise added LiOH·H₂O (1.50 mg, 35.6 μmol, 1.21 eq) in H₂O (0.20 mL). The mixture was stirred at 25° C. for 0.5 hr. Comparing HPLC RTs, the main new peak (RT=2.330 mins) was not compound 11 (RT=2.417 mins). The reaction mixture was concentrated under vacuum. The crude product was purified by prep-HPLC (column: 3_Phenomenex Luna C18 75*30 mm*3 um; mobile phase: [water (0.1% TFA)-ACN]; B %: 38%-68%, 7 min). The desired product, Example 6 (29.68 mg, 27.5 μmol, 97.2% purity) was obtained as white solid (see e.g., FIG. 3A-FIG. 3B).

¹H NMR:

δ 7.72 (d, J=2.0 Hz, 2H), 7.62-7.60 (m, 1H), 7.25 (s, 2H), 7.22-7.17 (m, 2H), 7.13 (d, J=7.2 Hz, 1H), 6.64 (s, 2H), 4.86-4.82 (m, 1H), 4.59 (s, 2H), 4.15-4.10 (m, 1H), 3.86-3.80 (m, 5H), 3.77-3.53 (m, 810H), 3.50-3.45 (m, 6H), 3.39 (s, 6H), 3.17-3.02 (m, 4H), 2.73 (t, J=6.8 Hz, 4H), 1.74-1.40 (m, 5H), 1.30-1.21 (m, 1H)

### Example 7

### Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-[2-[2-[2-[2-[2-[2-[2-[2-[2-[2-[2-[2-[2-[2-[2-[2-(2-thiomethylethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]phenyl]phenyl]propanoic acid

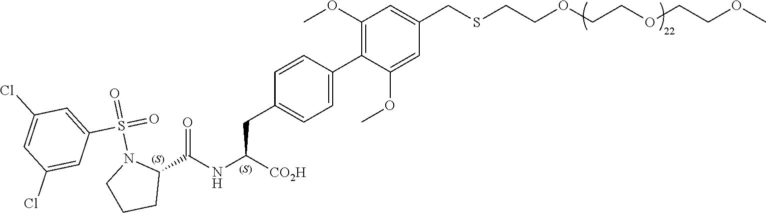

Example 7 was prepared according to the method of Example 4, substituting m-PEG24-thiol for m-PEG-thiol, MW 5K in Step 2 yielding a white solid.

¹H NMR (400 MHz, CDCl₃): δ 1.37-1.50 (m, 1H), 1.51-1.63 (m, 2H), 1.64-1.75 (m, 2H), 2.05 (d, J=11.29 Hz, 1H), 2.65-2.76 (appt/m, 2H), 3.39 (s, 3H), 3.53-3.58 (m, 2H), 3.59-3.85 (m, 100H), 4.06-4.21 (m, 2H), 4.79-4.91 (m, 1H), 6.62 (s, 2H), 7.16-7.26 (m/dd, 4H), 7.31 (s, 2H), 7.61 (s, 1H), 7.70-7.76 (m, 2H). LC-MS analysis of the solid showed the desired product's mass: m/z 1724 (^{35Cl} M+H), m/z 1726 (^{37Cl} M+H); Calcd Mass for C₇₈H₁₂₈C₁₂N₂O₃₁S₂:1724.88.

### Example 8

### Preparation of (2S)-2-[[(2S)-1-(3,5-dichlorophenyl)sulfonylpyrrolidine-2-carbonyl]amino]-3-[4-[2,6-dimethoxy-4-[40K MW PEG-thiomethyl]phenyl]phenyl]propanoic Acid

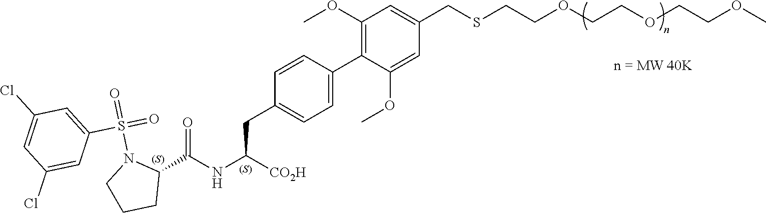

Example 8 was prepared according to the method of Example 4, substituting m-PEG-thiol, MW 40K (Creative PEG Works; Catalogue #PJK-6010) for m-PEG-thiol, MW 5K in Step 2, yielding the desired product as a white solid.

¹H NMR (400 MHz, CDCl₃): δ 1.64 (s, 1H), 2.00-2.50 (very broad s, PEG —CH₂CH₂O—), 3.12 (s, 1H), 3.40 (d, J=7.79 Hz, 2H), 3.50-3.75 (very broad s, PEG —CH₂CH₂O—), 3.78-3.86 (m, 2H), 3.98-4.03 (m, 2H), 4.10 (d, J=5.84 Hz, 1H), 4.75 (s, 1H), 6.88 (s, 1H), 7.00 (s, 2H), 7.11-7.16 (m, 1H), 7.33 (d, J=8.56 Hz, 2H), 7.53 (s, 2H), 7.62 (s, 1H), 7.71 (s, 2H), 7.70-7.72 (m, 2H). LC-MS analysis of the solid showed the desired product's mass: m/z 619 (^{35Cl} M+H—S-PEG-m), m/z 621 (^{37Cl} M+H—S-PEG-m); Calcd Mass for the Product: 40619.51 (Scaffold+m-PEG-S).

### Example 9

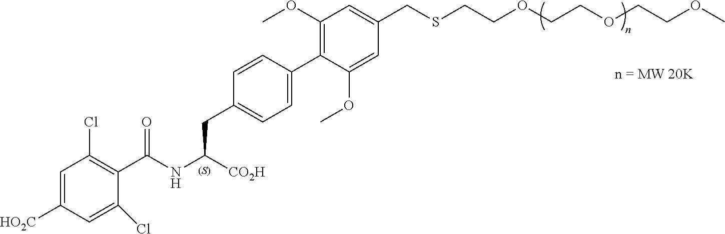

### Preparation of (S)-4-((1-carboxy-2-(2′,6′-dimethoxy-4′-20K MW PEG-thiomethyl)-[1,1′-biphenyl]-4-yl)ethyl)carbamoyl)-3,5-dichlorobenzoic Acid

### Step 1. Preparation of Benzyl 4-bromo-2,6-dichlorobenzoate

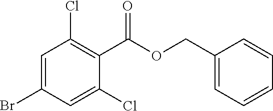 [SMILES: O=C(OCc1ccccc1)c1c(Cl)cc(Br)cc1Cl]

4-Bromo-2,6-dichlorobenzoic acid (3.58 g, 13.3 mmol) and cesium carbonate (7.56 g, 23.2 mmol) were suspended in acetonitrile (50 mL) at 0° C. and benzyl bromide (2.38 g, 13.93 mmol) was added drop wise. The reaction was heated for 4 hours at 60° C. and then the reaction was cooled to room temperature and solids were filtered off and rinsed using additional acetonitrile. The filtrate was concentrated in vacuo and purified by chromatography on silica gel, eluting with ethyl acetate/hexanes. Product, benzyl 4-bromo-2,6-dichlorobenzoate, 4.8 g, 99% yield, was isolated as a clear oil. LC-MS: t_{R}=3.03 min; m+23=381, 382, 383, 385 (bromine/bis-chlorine isotope pattern). ¹H NMR (400 MHz, DMSO-d₆) δ ppm 7.95 (s, 2H) 7.35-7.51 (m, 5H) 5.42 (s, 2H).

### Step 2. Preparation of 1-Benzyl 4-methyl 2,6-dichlorobenzene-1,4-dicarboxylate

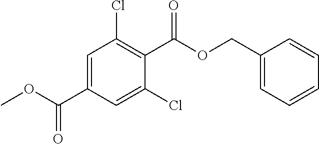 [SMILES: COC(=O)c1cc(Cl)c(C(=O)OCc2ccccc2)c(Cl)c1]

To a 20 mL microwave vial was added benzyl 4-bromo-2,6-dichlorobenzoate from Step 1 above (1.5 g, 4.17 mmol), palladium acetate (0.047 g, 0.208 mmol), 4,5-Bis(diphenylphosphino)-9,9-dimethylxanthene (0.241 g, 0.417 mmol), 4-dimethylaminopyridine (2 g, 16.7 mmol), Octacarbonyldicobalt (0.60 g, 3.33 mmol) and toluene/methanol (2:1, 15 mL). The vial was crimped shut and irradiated at 90° C. for 30 minutes using microwaves. The reaction was diluted with ethyl acetate, filtered through Celite® and concentrated in vacuo. The residue was taken up in ethyl acetate and washed using a 10% citric acid solution, then brine. The ethyl acetate layer was dried over sodium sulfate, filtered and concentrated in vacuo. The residue was purified by silica gel chromatography using ethyl acetate/hexanes as eluent to give product, 1-benzyl 4-methyl 2,6-dichlorobenzene-1,4-dicarboxylate, 0.7 g, 81% yield, as a clear oil. LC-MS: t_{R}=2.88 min; m+23=361, 363 (chlorine isotope).

### Step 3. Preparation of 2,6-Dichloro-4-(methoxycarbonyl)benzoic Acid

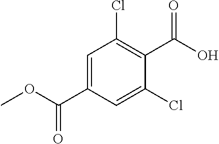

To a solution of 1-benzyl 4-methyl 2,6-dichlorobenzene-1,4-dicarboxylate from Step 2 above (2.3 g, 6.78 mmol) in ethyl acetate (20 mL) was added 10% palladium on carbon (0.35 g, 0.34 mmol). The mixture was stirred at room temperature under a hydrogen atmosphere at ambient pressure for 1.5 hours. The reaction was filtered through Celite® and concentrated in vacuo to give product, 2,6-dichloro-4-(methoxycarbonyl)benzoic acid, (1.69 g, quantitative yield) as a crystalline solid. LC-MS: t_{R}=1.65 min; m+H=249, 251 (chlorine isotope).

### Step 4. Preparation of methyl (S)-4-((3-(4-bromophenyl)-1-methoxy-1-oxopropan-2-yl)carbamoyl)-3,5-dichlorobenzoate

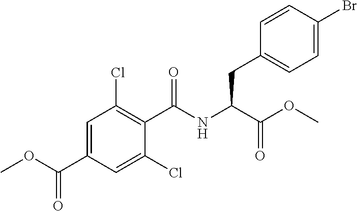 [SMILES: COC(=O)c1cc(Cl)c(C(=O)N[C@@H](Cc2ccc(Br)cc2)C(=O)OC)c(Cl)c1]

To a round bottom flask was added 2,6-dichloro-4-(methoxycarbonyl)benzoic acid from Step 3 above (2.08 g, 8.38 mmol), benzotriazol-1-ol (0.25 g, 1.59 mmol), 3-[Bis(dimethylamino)-methyliumyl]-3H-benzotriazol-1-oxide hexafluorophosphate (3.33 g, 8.77 mmol) and DMSO (15 mL). N,N-diisopropylethylamine (3.09 g, 23.93 mmol) was added and the reaction was stirred for 40 minutes at room temperature. At this time, methyl (S)-2-amino-3-(4-bromophenyl)propanoate hydrochloride (purchased from Ark Pharm) (2.35 g, 7.98 mmol) was added and the reaction was stirred overnight at room temperature. The reaction was diluted with water (50 mL), stirred for 20 minutes, then extracted using ethyl acetate (100 mL). The ethyl acetate layer was washed using additional water, dried using sodium sulfate and concentrated in vacuo. The resulting oil was purified on silica gel using ethyl acetate and hexanes as eluent. Product, methyl 4-[[(1 S)-1-[(4-bromophenyl)methyl]-2-methoxy-2-oxo-ethyl]carbamoyl]-3,5-dichloro-benzoate, 3.42 g, 87% yield, was isolated as a white foam. LC-MS: t_{R}=²0.61 min; m+H=488, 489, 490, 491, 492 (bromine/bis-chlorine isotope pattern). ¹H NMR (400 MHz, DMSO-d₆) δ ppm 9.33 (d, J=8.31 Hz, 1H) 7.91 (s, 2H) 7.45-7.54 (d, 2H) 7.26 (d, J=8.31 Hz, 2H) 4.80 (ddd, J=10.09, 8.25, 4.89 Hz, 1H) 3.89 (s, 3H) 3.68 (s, 3H) 3.16 (dd, J=14.06, 5.01 Hz, 1H) 2.93 (dd, J=14.06, 10.15 Hz, 1H).

### Step 5. Preparation of methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate

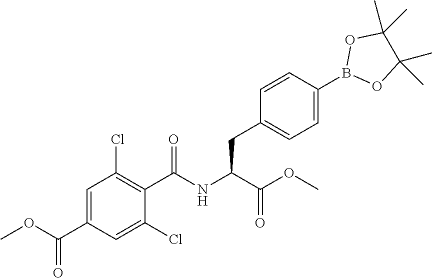

To a 20 mL microwave vial was added methyl 4-[[(1 S)-1-[(4-bromophenyl)methyl]-2-methoxy-2-oxo-ethyl]carbamoyl]-3,5-dichloro-benzoate from Step 4 above (1 g, 2.04 mmole), Bis(pinacolato)diboron (0.675 g, 2.66 mmol), [1,1′-Bis(diphenylphosphino)ferrocene]-palladium(II) dichloride (0.100 g, 0.123 mmole, potassium acetate (0.6 g, 6.13 mmol) and 1,4-dioxane (10 mL). The vial was crimped shut, sparged for 10 minutes with nitrogen gas and then heated overnight at 80° C. The reaction was cooled to room temperature and filtered through Celite®, rinsing with ethyl acetate. The ethyl acetate layer was washed using additional water, dried using sodium sulfate and concentrated in vacuo. The resulting oil was purified on silica gel using ethyl acetate and hexanes as eluent. Product, methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate, 0.9 g, 82% yield, was isolated as a white foam. LC-MS: t_{R}=2.76 min; m+H=536.1, 538.0 (bis chlorine isotope pattern). ¹H NMR (400 MHz, DMSO-d₆) δ ppm 9.36 (d, J=8.07 Hz, 1H) 7.91 (d, J=0.49 Hz, 2H) 7.60 (d, J=7.58 Hz, 2H) 7.30 (d, J=7.58 Hz, 2H) 4.73-4.84 (m, 1H) 3.89 (d, J=0.49 Hz, 3H) 3.67 (s, 3H) 3.16 (dd, J=5.40 Hz, 1H) 3.01 (dd, J=9.80 Hz, 1H) 1.30 (s, 12H).

### Step 6. Preparation of (4-bromo-3,5-dimethoxyphenyl)methanol

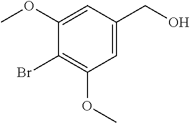

To an oven dried round bottom flask was added 4-bromo-3,5-dimethoxybenzoic acid (2 g, 7.66 mmol) and anhydrous tetrahydrofuran (24 mL). Borane dimethylsulfide complex (7.6 mL of 2 M in tetrahydrofuran, 15.3 mmol) was added drop-wise at room temperature. The reaction was heated overnight at 40° C. The reaction was quenched using hydrochloric acid (1N) and partitioned between ethyl acetate and water. The organic layer was washed using brine, dried with sodium sulfate, filtered and concentrated in vacuo. Product, (4-bromo-3,5-dimethoxyphenyl)methanol, (1.89 g, quantitative yield) was isolated as a white solid. LC-MS: t_{R}=1.748 min; m/z=229.0, 231.0 (dehydration). ¹H NMR (400 MHz, DMSO-d₆) δ ppm 6.71 (s, 2H) 4.49 (s, 2H) 3.82 (s, 6H).

### Step 7. Preparation of 4-{[(1S)-1-methoxy-1-oxo-2-{4-[4-(hydroxymethyl)-2,6-dimethoxyphenyl]phenyl}ethyl]carbamoyl}-3,5-dichloro-methylbenzoate

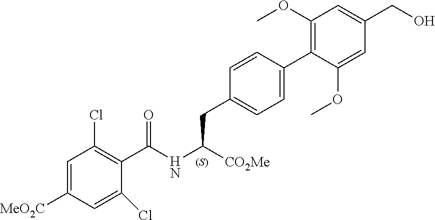

To a microwave vial was added methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate from Step 5 above (100 mg, 0.186 mmol), 4-bromo-3,5-dimethoxyphenyl)methanol from Step 6 above (69 mg, 0.28 mmol, 1.5 equivalent), tetrakis(triphenylphosphane)palladium(0) (11 mg, 0.009 mmol), cesium acetate (108 mg, 0.559 mmol), 1,4-dioxane (1 mL) and water (0.25 mL). The vial was crimped shut and sparged with nitrogen for 10 minutes and then heated overnight at 115° C. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. The isolated product of Step 7 was carried forward to the next step without further purification.

### Step 8. Preparation of 4-{[(1S)-1-methoxy-1-oxo-2-{4-[4-(bromomethyl)-2,6-dimethoxyphenyl]phenyl}ethyl]carbamoyl}-3,5-dichloro-methylbenzoate

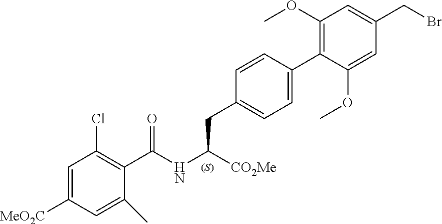

The product from Step 7 above, 4-{[(1 S)-1-methoxy-1-oxo-2-{4-[4-(hydroxymethyl)-2,6-dimethoxyphenyl]phenyl}ethyl]carbamoyl}-3,5-dichloro-methylbenzoate, is reacted with PBr₃ according to the method described in Example 1, Step 5 to yield a white solid that is used as is in the next step.

### Step 9. Preparation of (S)-4-((1-methoxy-1-oxo-2-(2′,6′-dimethoxy-4′-20K MW PEG-thiomethyl)-[1,1′-biphenyl]-4-yl)ethyl)carbamoyl)-3,5-dichloro-methylbenzoate

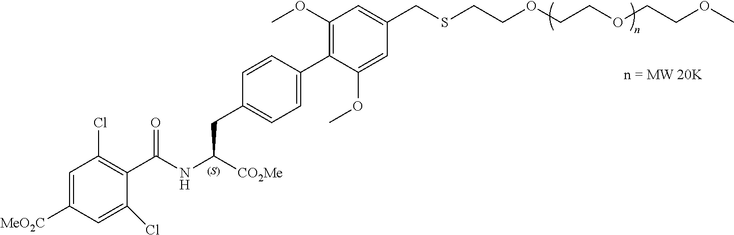

The benzyl bromide product from Step 8 above was reacted with m-PEG-thiol, MW 20K (BroadPharm, Catalogue #BP-23723) according to the method described in Example 5, Step 1. The crude product was directly used in the next step.

### Step 10. Preparation of (S)-4-((1-carboxy-2-(2′,6′-dimethoxy-4′-20K MW PEG-thiomethyl)-[1,1′-biphenyl]-4-yl)ethyl)carbamoyl)-3,5-dichlorobenzoic Acid

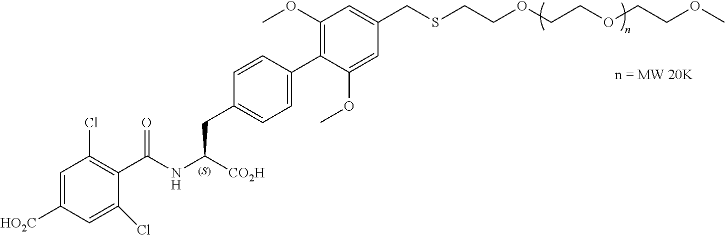

To a flask was added the product from Step 9 above, acetonitrile (1 mL), water (1 mL) and lithium hydroxide monohydrate (5 equivalents). The solution was stirred at room temperature for 2 hours. Acetonitrile was removed in vacuo and the reaction was acidified using hydrochloric acid (1N). Water was then removed in vacuo and the residue was purified on C18 using acetonitrile and water (both with 0.1% formic acid as modifier). Pure fractions were pooled and concentrated in vacuo and the resulting material was lyophilized from acetonitrile and water (1:4) overnight to yield the desired product as a white solid.

¹H NMR (400 MHz, CDCl₃): δ 2.25-3.05 (very broad s, PEG —CH₂CH₂O—). 3.38 (s, 2H), 3.44-3.49 (m, 2H), 3.50-3.80 (very broad s, PEG —CH₂CH₂O—), 3.79-3.85 (m, 2H), 4.75 (s, 1H), 5.19 (dd, 1H), 6.62 (d, J=5.06 Hz, 1H), 6.88 (s, 1H), 7.01 (s, 1H), 7.20-7.33 (dd/m, 4H), 7.53 (s, 1H), 7.94 (s, 1H). LC-MS analysis of the solid showed the desired product's mass: m/z 530 (^{35Cl} M+H—S-PEG-m), m/z 532 (^{37Cl} M+H—S-PEG-m); Calcd Mass for the Product: 20528.07 (Scaffold+m-PEG-S).

### Example 10

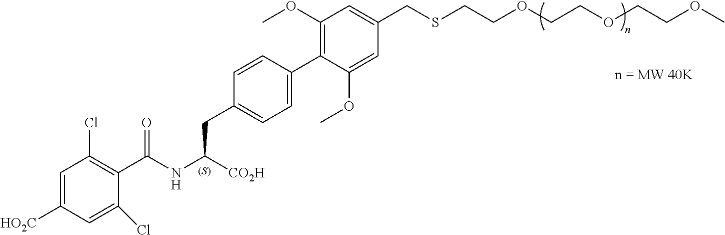

### Preparation of (S)-4-((1-carboxy-2-(2′,6′-dimethoxy-4′-40K MW PEG-thiomethyl)-[1,1′-biphenyl]-4-yl)ethyl)carbamoyl)-3,5-dichlorobenzoic Acid

The method for the synthesis of Example 10 is as described for Example 9 above, substituting m-PEG-thiol, MW 40K (Creative PEG Works Catalogue #PJK-6010) for m-PEG-thiol, MW 20K in Step 9, yielding a white solid.

¹H NMR (400 MHz, CDCl₃): δ 3.38 (s, 3H), 3.45-3.49 (m, 2H), 3.50-3.78 (very broad s, PEG-CH₂CH₂O—), 3.80-3.85 (m, 2H), 6.62 (s, 2H), 7.00 (s, 1H), 7.18-7.38 (dd/m, 4H), 7.53 (s, 1H), 7.95 (s, 1H). LC-MS analysis of the solid showed the desired product's mass: m/z 530 (^{35Cl} M+H-SPEG-m), m/z 532 (^{37Cl} M+H-SPEG-m); Calcd Mass for the Product: 40528.07(Scaffold+m-PEG-S).

### Example 11

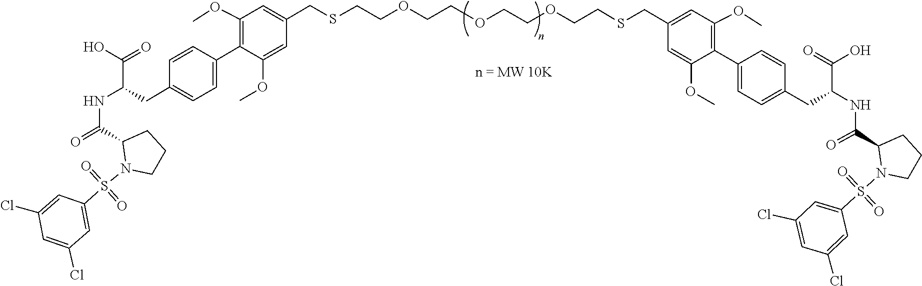

### Preparation of (S)-3-(4′-(15-(4′-((R)-2-carboxy-2-((R)-1-((3,5-dichlorophenyl)sulfonyl)pyrrolidine-2-carboxamido)ethyl)-2,6-dimethoxy-[1,1′-biphenyl]-4-yl)-(2,14-dithia-PEG-10000)-2′,6′-dimethoxy-[1,1′-biphenyl]-4-yl)-2-((S)-1-((3,5-dichlorophenyl)sulfonyl)pyrrolidine-2-carboxamido)propanoic Acid

### Step 1. Preparation of Methyl (R)-2-((R)-1-((3,5-dichlorophenyl)sulfonyl)pyrrolidine-2-carboxamido)-3-(4′-(15-(4′-((S)-2-((S)-1-((3,5-dichlorophenyl)sulfonyl)pyrrolidine-2-carboxamido)-3-methoxy-3-oxopropyl)-2,6-dimethoxy-[1,1′-biphenyl]-4-yl)-5,8,11-trioxa-2,14-dithiapentadecyl(PEG-bis-Thiol-10000)-2′,6′-dimethoxy-[1,1′-biphenyl]-4-yl)propanoate

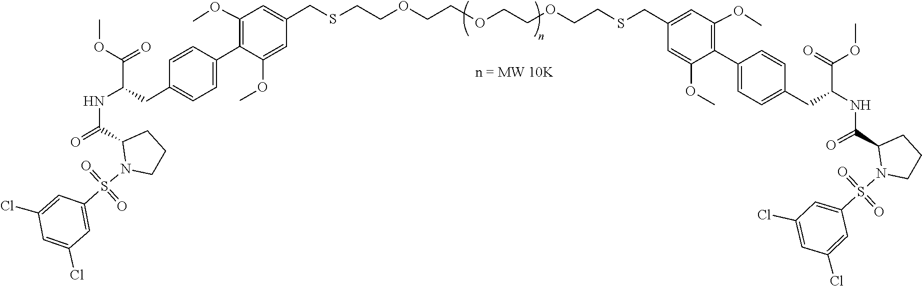

To a solution of a mixture of 10K PEG-Bis-Thiol (BioPharm) (0.200 g; 0.020 mmol) and methyl (S)-3-(4′-(bromomethyl)-2′,6′-dimethoxy-[1,1′-biphenyl]-4-yl)-2-((S)-1-((3,5-dichlorophenyl) sulfonyl)pyrrolidine-2-carboxamido)propanoate, from Example 4, Step 1 (0.0285 g; 0.040 mmol) in anhydrous DMF (3.0 mL) was added anhydrous K₂CO₃ (0.276 g, 0.200 mmol) containing a small crystal of KI and the reaction mixture was heated at 35° C. under nitrogen atmosphere overnight. The reaction mixture was evaporated in-vacuo. The residue was dissolved in acetonitrile, filtered through a 0.45 uM syringe filter and evaporated in-vacuo to afford a colorless solid (0.241 g). LC-MS analysis of the solid showed the desired product's mass: m/z 633 (^{35Cl} M+H—S-PEG-S—) and m/z 635 (^{37Cl} M+H—S-PEG-S—). The crude product was used as such for the saponification with LiOH·H₂O.

### Step 2. Preparation of (S)-3-(4′-(15-(4′-((R)-2-carboxy-2-((R)-1-((3,5-dichlorophenyl)sulfonyl)pyrrolidine-2-carboxamido)ethyl)-2,6-dimethoxy-[1,1′-biphenyl]-4-yl)-(2,14-dithia-PEG-10000)-2′,6′-dimethoxy-[1,1′-biphenyl]-4-yl)-2-((S)-1-((3,5-dichlorophenyl)sulfonyl)pyrrolidine-2-carboxamido)propanoic Acid

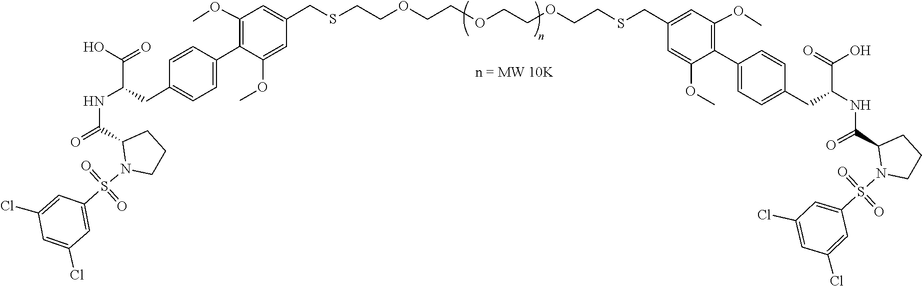

To a solution of the above crude product in a mixture of acetonitrile/water (6.0 mL) was added LiOH·H₂O (0.010 g, 0.238 mmol) and the reaction mixture was stirred at room temperature overnight. The crude product was purified by reverse-phase preparative HPLC (acetonitrile/water, 0.05% TFA) to afford the desired product, Example 11, as a colorless solid after lyophilization (0.121 g). LC-MS analysis of the solid showed the desired product's mass: m/z 619 (^{35Cl} M+H—S-PEG-S—) and m/z 621 (^{37Cl} M+H—S-PEG-S—). Calcd Mass for the Product: 11239.08 (Scaffold+PEG-Bis-Thiol). ¹H NMR (400 MHz, CDCl₃): δ 2.71 (s, 2H), 2.86-2.94 (m, 2H), 3.12 (dt, J=14.50, 7.35 Hz, 4H), 3.30-3.42 (m, 2H), 3.50-3.78 (very broad s, PEG-CH₂CH₂O—), 4.11 (d, J=8.17 Hz, 1H), 4.84 (d, J=6.23 Hz, 1H), 6.58-6.66 (m, 2H), 7.01 (s, 1H), 7.08 (d, J=7.79 Hz, 2H), 7.53 (s, 1H), 7.61 (s, 1H), 7.72 (brs, 2H).

### Example 12

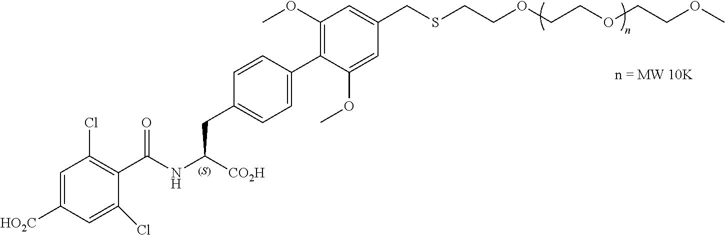

### Preparation of (S)-4-((1-carboxy-2-(2′,6′-dimethoxy-4′-10K MW PEG-thiomethyl)-[1,1′-biphenyl]-4-yl)ethyl)carbamoyl)-3,5-dichlorobenzoic Acid

The method for the synthesis of Example 12 is as described for Example 9 above, substituting m-PEG-thiol, MW 10K (Biopharma PEG, Catalogue #MF001003-10K) for m-PEG-thiol, MW 20K in Step 9, yielding a white solid.

¹H NMR (400 MHz, CDCl₃): δ 2.65-2.70 (m/appt, 2H), 3.25 (dd, J=14.20 and 6.80 Hz, 2H), 3.38 (s, 3H), 3.44-3.50 (m, 2H), 3.55-3.58 (m, 2H), 3.65 (very broad s, PEG-CH₂CH₂O—), 3.72 (s, 6H), 3.78-3.87 (m, 2H), 5.19 (dd, J=13.10 and 6.80 Hz, 1H), 6.61 (s, 2H), 7.24 (d, J=8.20 Hz, 2H), 7.30 (d, J=8.17 Hz, 2H), 7.93 (s, 2H). LC-MS analysis of the solid showed the desired product's mass: m/z 619 (35CIM+H—S-PEG-m), m/z 621 (37CIM+H—S-PEG-m); Calcd Mass for the Product: 10619.54 (Scaffold-S-PEG-m).

### Example 13

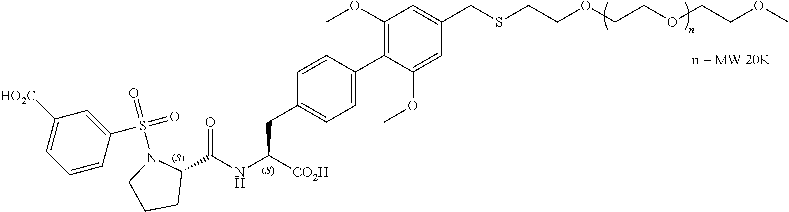

### Preparation of 3-((S)-2-((S)-1-carboxy-2-(2′,6′-dimethoxy-4′-((mPEG20000)thio)methyl)-[1,1′-biphenyl]-4-yl)ethyl)carbamoyl)pyrrolidin-1-yl)sulfonyl)benzoic Acid

The method for the synthesis of Example 13 is as described in the methods exemplified in the examples above for Example 5, but substituting 3-benzoic acid methyl ester sulfonyl chloride for 3,5-di-Chlorophenyl sulfonyl chloride, yielding a colorless Solid; LC-MS analysis of the solid showed the desired product's mass: m/z 595 (M+H—S-PEG-m), m/z 549 (M+H—S-PEG-m-COOH); Calcd Mass for the Product: 20594.64 (Scaffold-S-PEG-m).

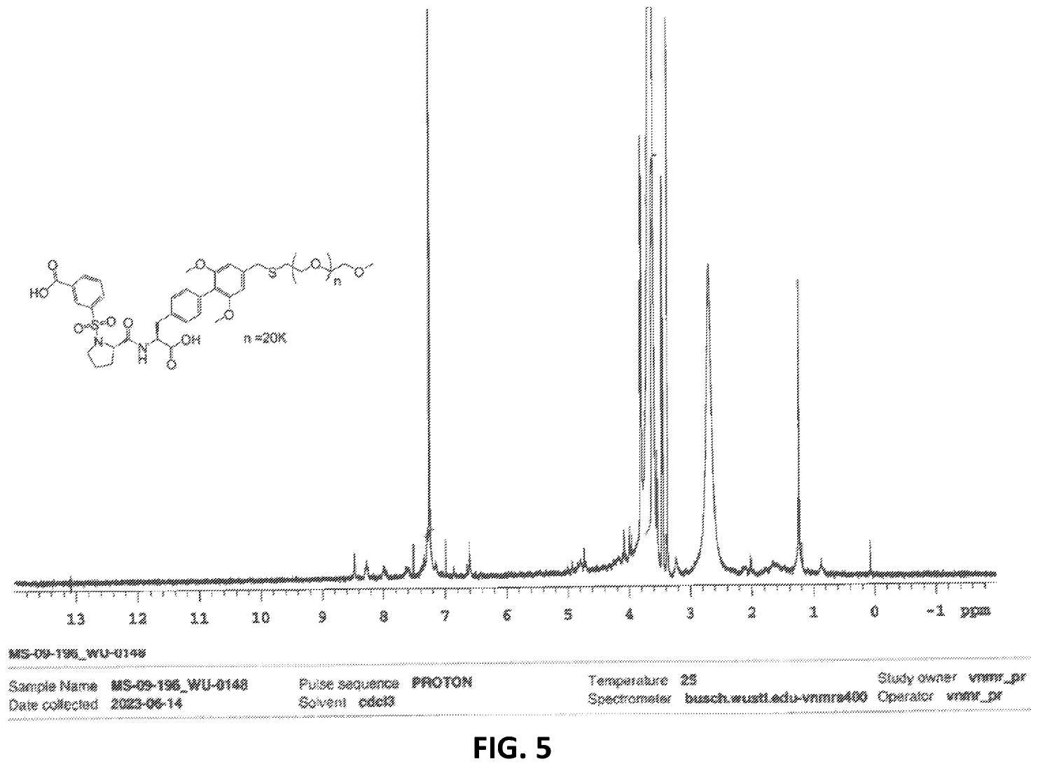

The 1HNMR spectrum for Example 13 is shown in FIG. 5.

### Example 14

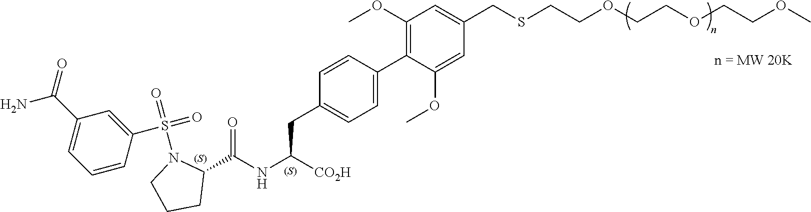

### Preparation of (S)-2-((S)-1-((3-carbamoylphenyl)sulfonyl)pyrrolidine-2-carboxamido)-3-(2′,6′-dimethoxy-4′-((2-(mPEG20000)thio)methy)-[1,1′-biphenyl]-4-yl)propanoic Acid

The method for the synthesis of Example 14 is as described in the methods exemplified in the examples above for Example 5, but substituting 3-benzamide sulfonyl chloride for 3,5-di-Chlorophenyl sulfonyl chloride, yielding a colorless Solid; LC-MS analysis of the solid showed the desired product's mass: m/z 594 (M+H—S-PEG-m), m/z 612 (M+H—S-PEG+H₂O), m/z 577 (M+H—S-PEG-H₂O), m/z 548 (M+H—S-PEG-COOH); Calcd Mass for the Product: 20593.68 (Scaffold-S-PEG-m).

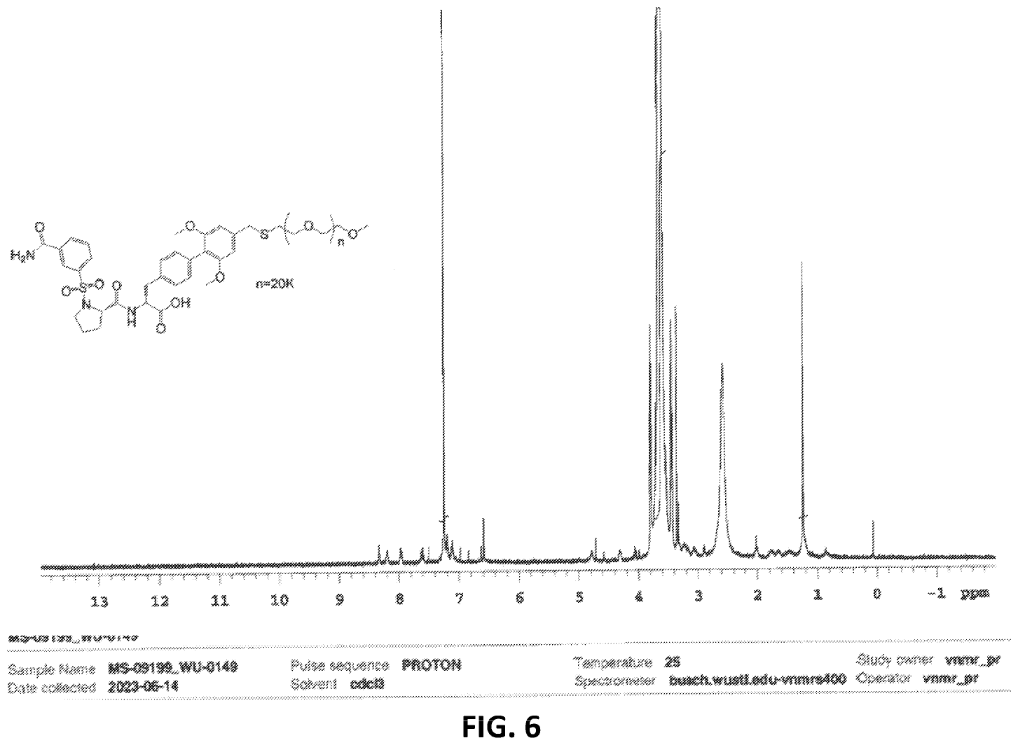

The 1HNMR spectrum for Example 14 is shown in FIG. 6.

### Example 15

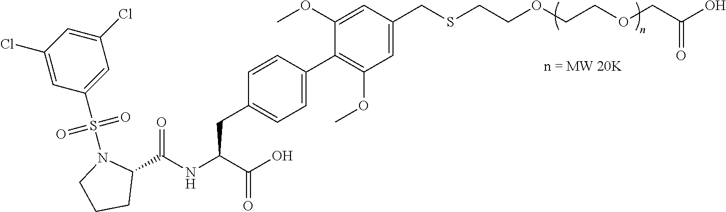

### Preparation of (S)-3-(4′-(((2-(2-(carboxymethoxy)thio)methyl(PEG20000))-2′,6′-dimethoxy-[1,1′-biphenyl]-4-yl)-2-((S)-1-((3,5-dichlorophenyl)sulfonyl)pyrrolidine-2-carboxamido) propanoic Acid

The method for the synthesis of Example 15 is as described in the methods exemplified for the synthesis of example 5 above, but substituting m-PEG thiol carboxylic acid, MW 20K for m-PEG thiol, MW 20K, yielding a Colorless Solid; LC-MS analysis of the solid showed the desired product's mass: m/z 619 (35CIM+H—S-PEG-GOGH), m/z 621 (37CIM+H—S-PEG-COOH); Calcd Mass for the Product: 20619.51 (Scaffold-S-PEG-COOH).

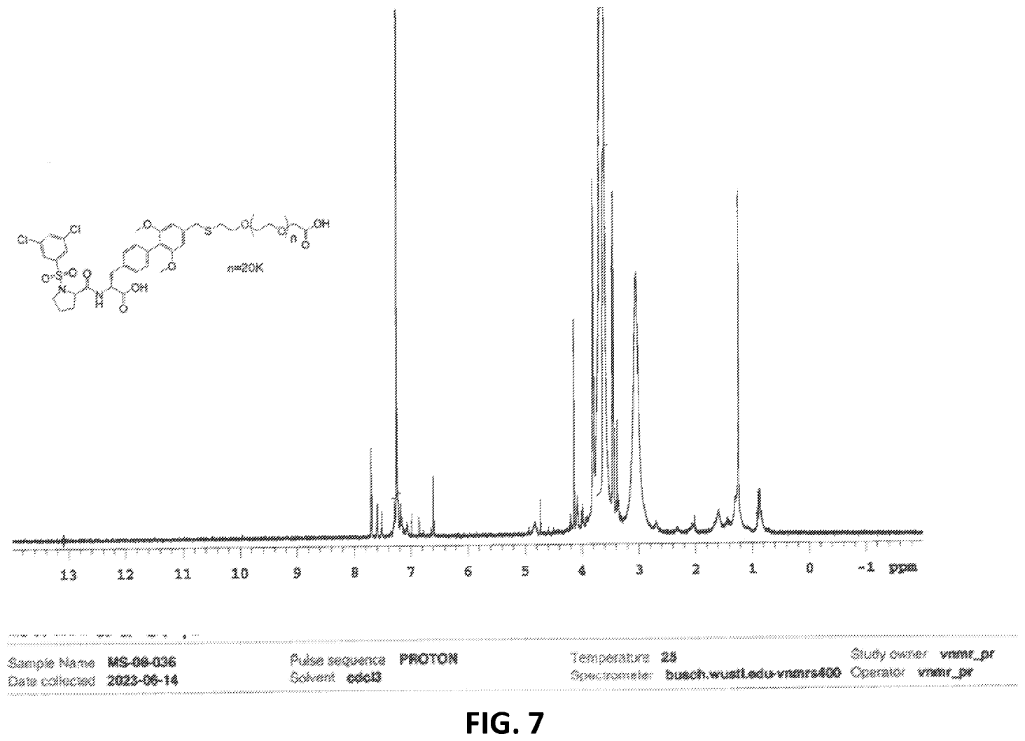

The 1HNMR spectrum for Example 15 is shown in FIG. 7.

### Example 16

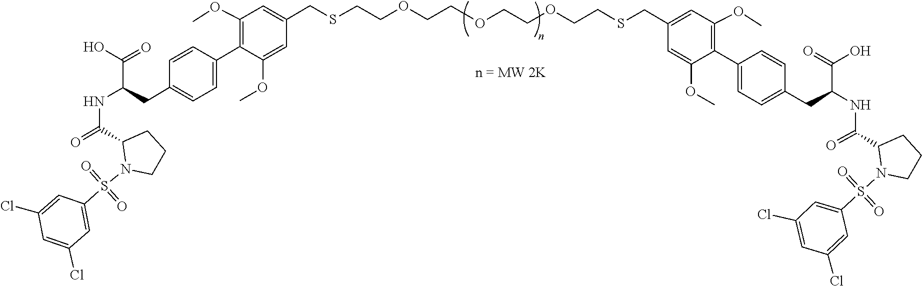

### Preparation of (S)-3-(4′-(15-(4′-((R)-2-carboxy-2-((R)-1-((3,5-dichlorophenyl)sulfonyl)pyrrolidine-2-carboxamido)ethyl)-2,6-dimethoxy-[1,1′-biphenyl]-4-yl)-(2,14-dithia-PEG-2000)-2′,6′-dimethoxy-[1,1′-biphenyl]-4-yl)-2-((S)-1-((3,5-dichlorophenyl)sulfonyl)pyrrolidine-2-carboxamido)propanoic Acid

The method for the synthesis of Example 16 is as described in the methods exemplified for the synthesis of example 11 above, but substituting 2K PEG-Bis-Thiol for 10K PEG-Bis-Thiol, yielding a colorless Solid; LC-MS analysis of the solid showed the desired product's mass: m/z 619 (35CIM+H—S-PEG-S-Scaffold), m/z 621 (37CIM+H—S-PEG-S-Scaffold); Calcd Mass for the Product: 3239.08 (Scaffold-S-PEG-S-Scaffold).

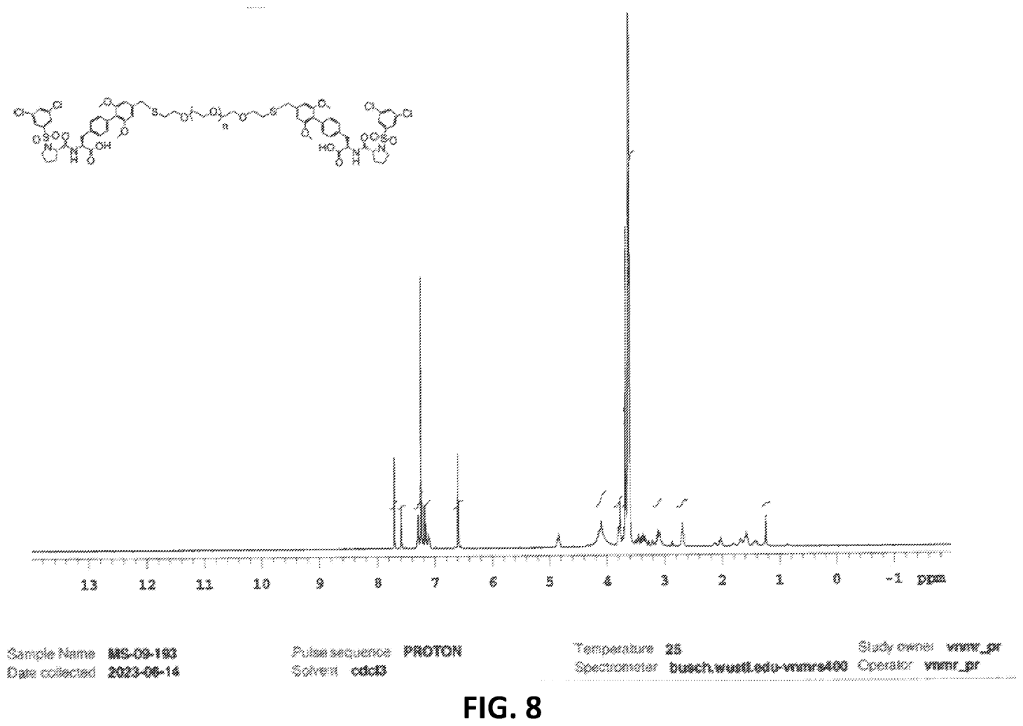

The 1HNMR spectrum for Example 16 is shown in FIG. 8.

### Example 17

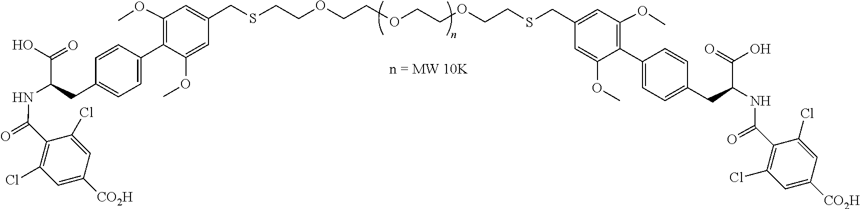

### Preparation of 4-(((S)-1-carboxy-2-(4′-(15-(4′-((R)-2-carboxy-2-(4-carboxy-2,6-dichlorobenzamido)ethyl)-2,6-dimethoxy-[1,1′-biphenyl]-4-yl)-5,8,11-trioxa-2,14-dithiapentadecyl)(PEG-10000-2′,6′-dimethoxy-[1,1′-biphenyl]-4-yl)ethyl)carbamoyl)-3,5-dichlorobenzoic Acid

The method for the synthesis of Example 17 is as described in the methods exemplified for the synthesis of example 9 above, but substituting 10K PEG-Bis-Thiol for 20K m-PEG-Thiol and reacting 2 equivalents of the product of Step 8 instead of 1 equivalent, yielding a colorless Solid; LC-MS analysis of the solid showed the desired product's mass: m/z 530 (35CIM+H—S-PEG-S-Scaffold), m/z 532 (37CIM+H—S-PEG-S-Scaffold); Calcd Mass for the Product: 11060.82 (Scaffold-S-PEG-S-Scaffold).

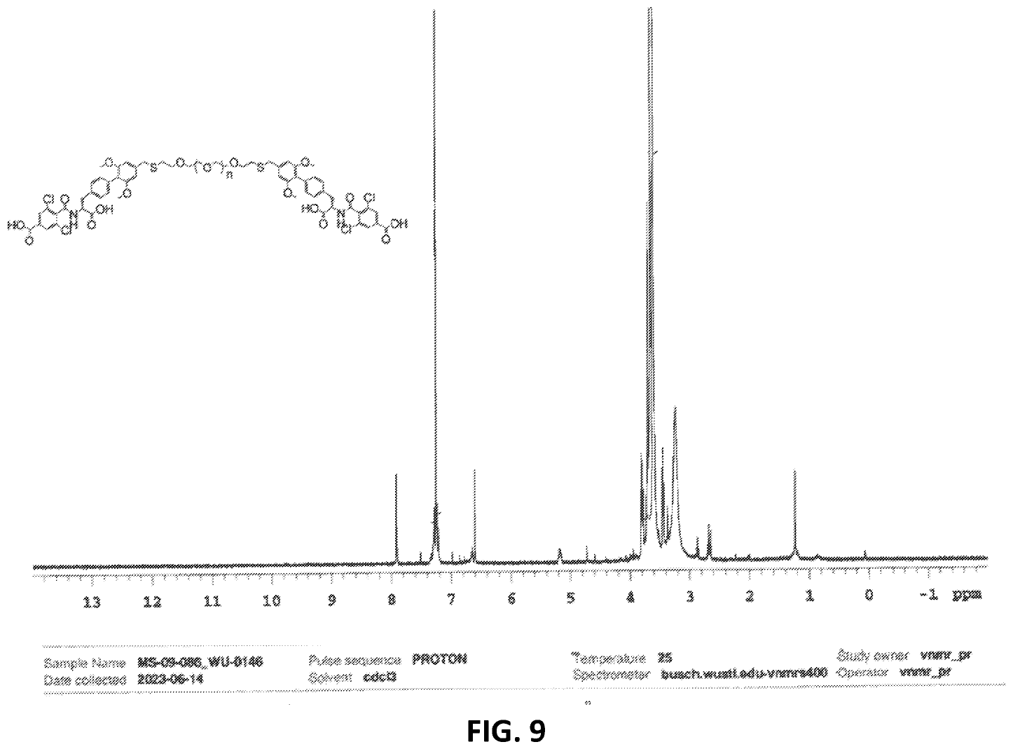

The 1HNMR spectrum for Example 17 is shown in FIG. 9.

### Example 18

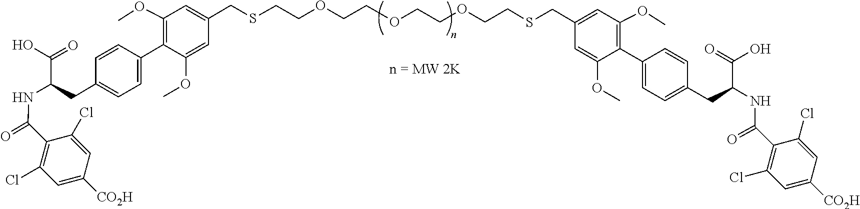

### Preparation of 4-(((S)-1-carboxy-2-(4′-(15-(4′-((R)-2-carboxy-2-(4-carboxy-2,6-dichlorobenzamido)ethyl)-2,6-dimethoxy-[1,1′-biphenyl]-4-yl)-(dithia-PEG2000)-2′,6′-dimethoxy-[1,1′-biphenyl]-4-yl)ethyl)carbamoyl)-3,5-dichlorobenzoic Acid

The method for the synthesis of Example 18 is as described in the methods exemplified for the synthesis of example 9 above, but substituting 2K PEG-Bis-Thiol for 20K m-PEG-Thiol and reacting 2 equivalents of the product of Step 8 instead of 1 equivalent, yielding a colorless Gummy Solid; LC-MS analysis of the solid showed the desired product's mass: m/z 530 (35CIM+H—S-PEG-S-Scaffold), m/z 532 (37CIM+H—S-PEG-S-Scaffold); Calcd Mass for the Product: 3060.71 (Scaffold-S-PEG-S-Scaffold).

### Example 19

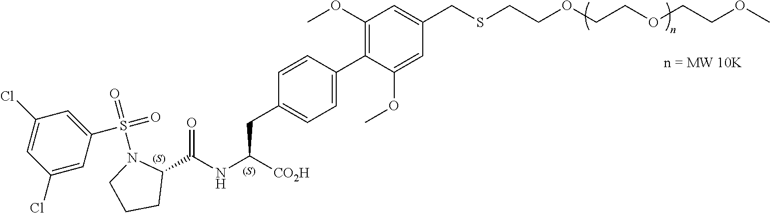

### Preparation of (2S)-2-[[(2S)-1-(3,5-dichlorophenyl)sulfonylpyrrolidine-2-carbonyl]amino]-3-[4-[2,6-dimethoxy-4-[1 OK MW PEG-thiomethyl]phenyl]phenyl]propanoic Acid

The method for the synthesis of Example 19 is as described in the methods exemplified for the synthesis of example 5 above, but substituting m-PEG thiol, MW 10K for m-PEG thiol, MW 20K, yielding a colorless Solid; LC-MS analysis of the solid showed the desired product's mass: m/z 619 (35CIM+H—S-PEG-m), m/z 621 (37CIM+H—S-PEG-m); Calcd Mass for the Product: 10619.54 (Scaffold-S-PEG-m).

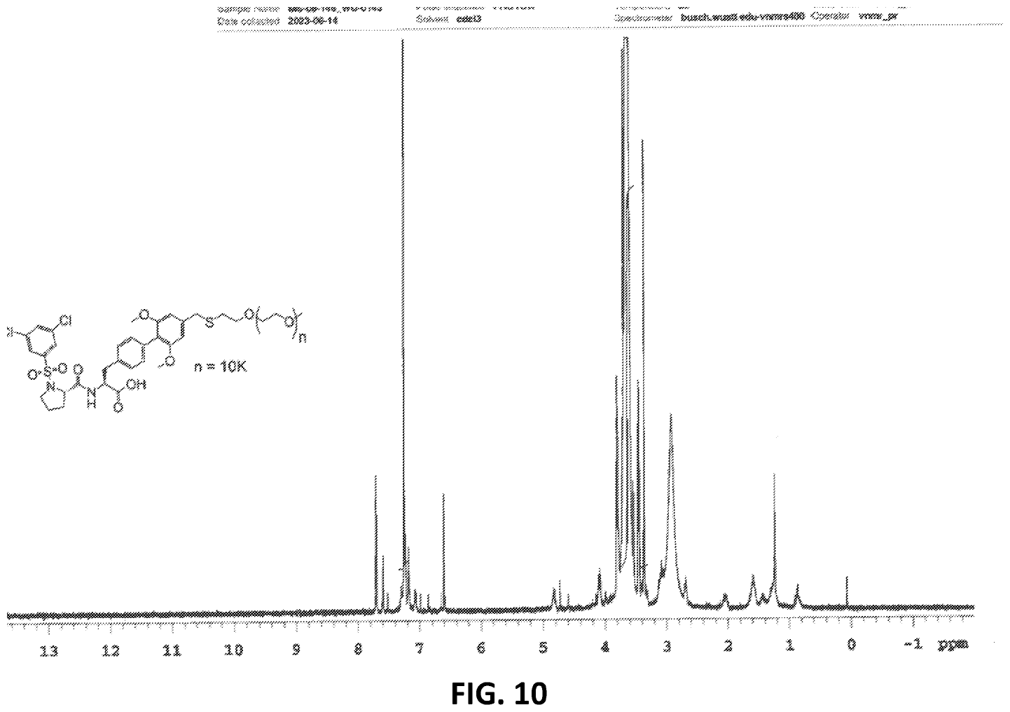

The 1HMNR spectrum for Example 19 is shown in FIG. 10.

### Comparator Compound 9

### Preparation of (S)-3-(4′-(((1-(2,5,8,11,14,17,20,23,26,29,32,35,38,41,44,47,50,53,56,59,62,65,68,71-tetracosaoxatriheptacontan-73-yl)-1H-1,2,3-triazol-4-yl)methoxy)methyl)-2′,6′-dimethoxy-[1,1′-biphenyl]-4-yl)-2-((S)-1-((3,5-dichlorophenyl)sulfonyl)pyrrolidine-2-carboxamido)propanoic Acid

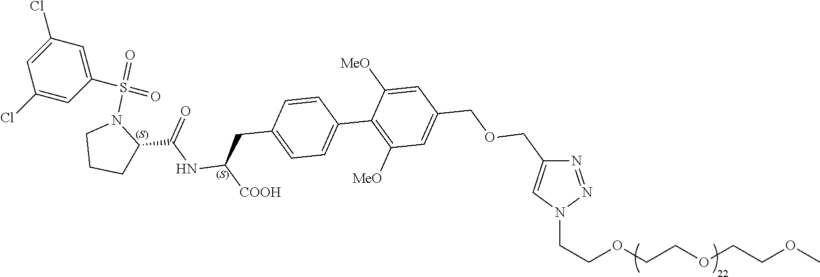

### The Synthesis of Comparator Example 9 is Depicted in Scheme D:

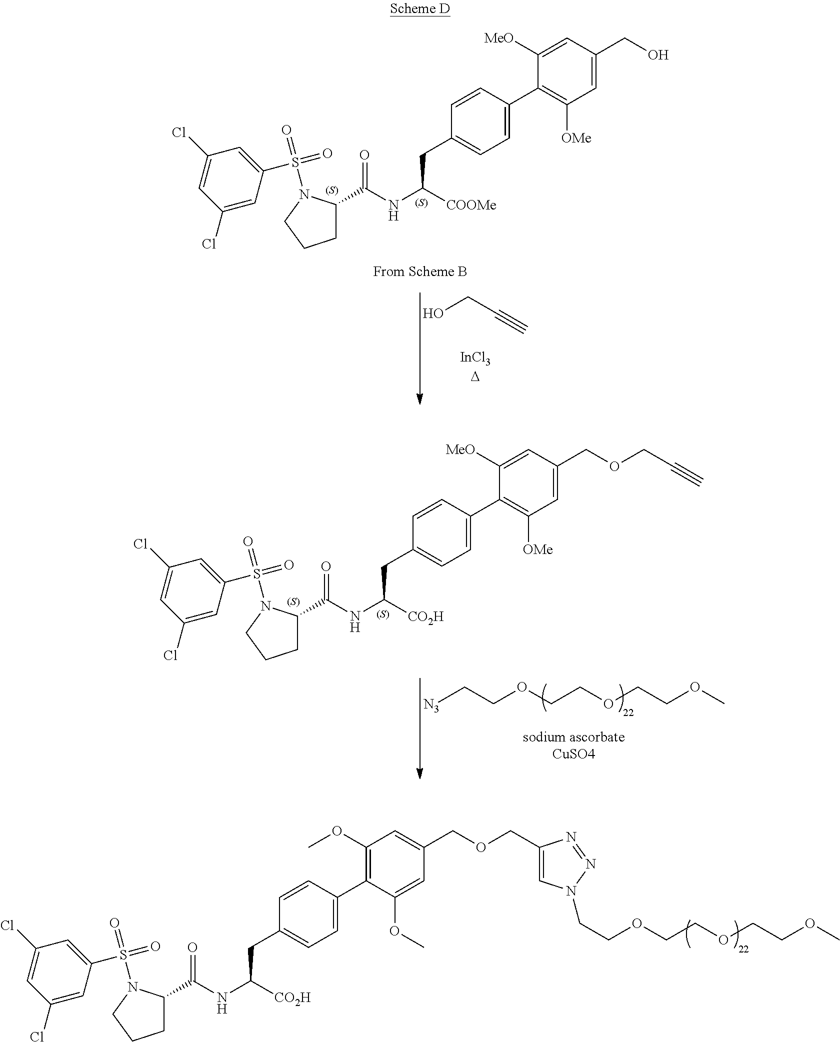

### Step 1. Preparation of (S)-2-((S)-1-((3,5-dichlorophenyl)sulfonyl)pyrrolidine-2-carboxamido)-3-(2′,6′-dimethoxy-4′-((prop-2-yn-1-yloxy)methyl)-[1,1′-biphenyl]-4-yl)propanoic Acid

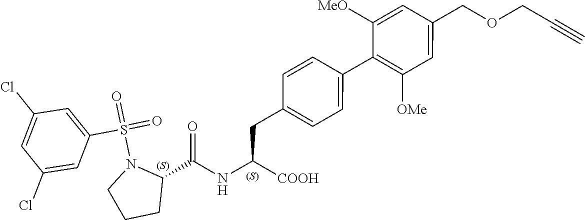

To a solution of prop-2-yn-1-ol (1.21 g, 21.5 mmol, 1.28 mL) in (S)-methyl 2-((S)-1-((3,5-dichlorophenyl)sulfonyl)pyrrolidine-2-carboxamido)-3-(4′-(hydroxymethyl)-2′,6′-dimethoxy-[1,1′-biphenyl]-4-yl)propanoate, from Step 7 in Example 2 (200 mg, 291 μmol) was added InCl₃ (51.5 mg, 233 μmol, 14.9 μL). The mixture was stirred at 120° C. for 12 h. LCMS showed the reaction was complete. The reaction was concentrated under vacuum to give the desired product (134 mg, yield 10.9%) as brown oil, which was purified by prep-HPLC (column: Phenomenex luna C18 150*40 mm*15 um; mobile phase: [water (0.1% TFA)-ACN]; B %: 55%-85%, 10 min) and further purified by prep-HPLC (column: Phenomenex luna C18 150*25 mm*10 um; mobile phase: [water (0.1% TFA)-ACN]; B %: 49%-79%, 10 min). ¹H NMR (400 MHz, CDCl₃): δ 7.73 (d, J=1.8 Hz, 2H), 7.61 (t, J=1.8 Hz, 1H), 7.26 (br s, 2H), 7.22-7.15 (m, 3H), 6.67 (s, 2H), 4.95-4.89 (m, 1H), 4.74 (s, 2H), 3.83 (s, 1H), 3.72 (s, 6H), 3.42-3.35 (m, 2H), 3.13-3.09 (m, 3H), 2.55 (br t, J=2.4 Hz, 1H), 1.68-1.56 (m, 3H), 1.51-1.40 (m, 2H).

### Step 2. Preparation of (S)-3-(4′-(((1-(2,5,8,11,14,17,20,23,26,29,32,35,38,41,44,47,50,53,56,59,62,65,68,71-tetracosaoxatriheptacontan-73-yl)-1H-1,2,3-triazol-4-yl)methoxy)methyl)-2′,6′-dimethoxy-[1,1′-biphenyl]-4-yl)-2-((S)-1-((3,5-dichlorophenyl)sulfonyl)pyrrolidine-2-carboxamido)propanoic Acid

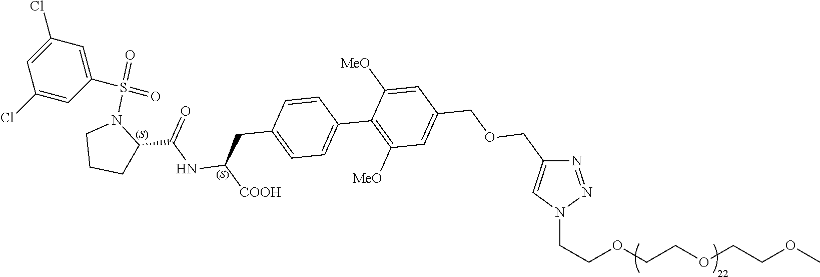

To a solution of m-PEG24-azide (BroadPharm) (100 mg, 87.0 μmol) in THF (5.00 mL) and H₂O (1.00 mL) was added (S)-2-((S)-1-((3,5-dichlorophenyl)sulfonyl)pyrrolidine-2-carboxamido)-3-(2′,6′-dimethoxy-4′-((prop-2-yn-1-yloxy)methyl)-[1,1′-biphenyl]-4-yl)propanoic acid, from Step 1 above (75.6 mg, 87.0 μmol), CuSO₄ (13.8 mg, 87.0 μmol, 13.3 μL) and sodium ascorbate (17.2 mg, 87.0 μmol). The mixture was stirred at 100° C. for 12 h. LCMS showed the reaction was complete. The reaction mixture was concentrated under vacuum to give the desired product, Comparator Example 9 (40.7 mg, yield 25.3%) as yellow gum, which was purified by prep-HPLC (column: Waters Xbridge 150*25 mm*5 um; mobile phase: [water (10 mM NH₄HCO₃)-ACN]; B %: 31%-61%, 10 min). ¹H NMR (400 MHz, CDCl₃): δ. 7.81 (s, 1H), 7.73 (d, J=1.8 Hz, 2H), 7.62 (t, J=1.8 Hz, 1H), 7.22 (d, J=8.0 Hz, 2H), 7.13-7.08 (m, 3H), 6.67 (s, 2H), 5.36 (s, 2H), 4.98-4.83 (m, 1H), 4.73 (s, 2H), 4.53 (t, J=5.2 Hz, 2H), 4.10 (dd, J=2.4, 8.6 Hz, 1H), 3.87 (t, J=5.2 Hz, 2H), 3.72 (s, 6H), 3.67-3.63 (m, 96H), 3.39 (s, 3H), 3.34-3.29 (m, 1H), 3.12-3.04 (m, 2H), 2.07-2.01 (m, 1H).

The desired product was confirmed by QTOF-MS with a mass of 1787.7846

### Comparator Compound 10

### Preparation of (S)-3-(4′-(((1-(2,5,8,11,14,17,20,23,26,29,32,35,38,41,44,47,50,53,56,59,62,65,68,71,74,77,80,83, 86,89,92,95,98,101,104,107-hexatriacontaoxanonahectan-109-yl)-1H-1,2,3-triazol-4-yl)methoxy)methyl)-2′,6′-dimethoxy-[1,1′-biphenyl]-4-yl)-2-((S)-1-((3,5-dichlorophenyl)sulfonyl)pyrrolidine-2-carboxamido)propanoic Acid

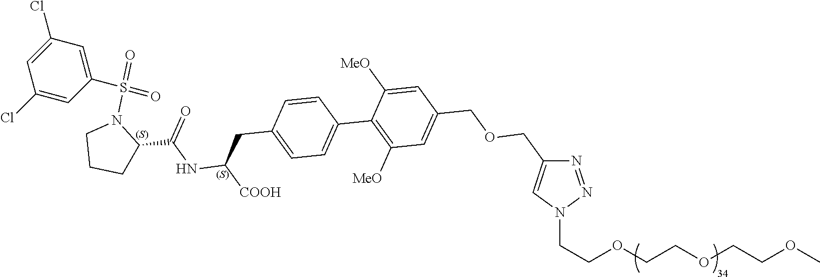

The Scheme D shown for the synthesis of Comparator Example 9 is the same Scheme outlining the synthesis of Comparator Example 10, substituting m-PEG36-azide for m-PEG24-azide in the last step.

### Step 1. Preparation of (S)-3-(4′-(((1-(2,5,8,11,14,17,20,23,26,29,32,35,38,41,44,47,50,53,56,59,62, 65,68,71,74,77,80,83,86,89,92,95,98,101,104,107-hexatriacontaoxanonahectan-109-yl)-1H-1,2,3-triazol-4-yl)methoxy)methyl)-2′,6′-dimethoxy-[1,1′-biphenyl]-4-yl)-2-((S)-1-((3,5-dichlorophenyl)sulfonyl)pyrrolidine-2-carboxamido)propanoic Acid

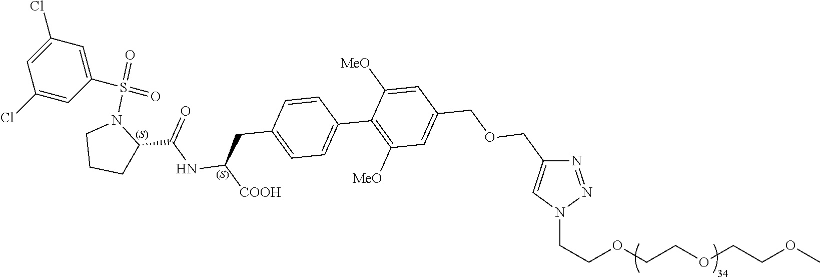

To a solution of m-PEG36-azide (BroadPharm) (100 mg, 59.0 μmol) in THF (5.00 mL) and H₂O (1.00 mL) was added (S)-2-((S)-1-((3,5-dichlorophenyl)sulfonyl)pyrrolidine-2-carboxamido)-3-(2′,6′-dimethoxy-4′-((prop-2-yn-1-yloxy)methyl)-[1,1′-biphenyl]-4-yl)propanoic acid, from Step 1 in Comparator Example 9 (39.9 mg, 59.0 μmol), CuSO₄ (9.42 mg, 59.0 μmol, 9.06 μL) and sodium ascorbate (11.7 mg, 59.0 μmol). The mixture was stirred at 100° C. for 12 h. LCMS showed the reaction was complete. The reaction mixture was concentrated under vacuum to give the desired product, Comparator Example 10 (34.4 mg, yield 25.0%) as colorless oil, which was purified by prep-HPLC (column: Waters Xbridge 150*25 mm*5 um; mobile phase: [water (10 mM NH₄HCO₃)-ACN]; B %: 31%-61%, 10 min) and further purified by prep-HPLC (column: Phenomenex Synergi C18 150*25*10 um; mobile phase: [water (0.225% FA)-ACN]; B %: 40%-67%, 10 min). ¹H NMR (400 MHz, CDCl₃): δ. 7.81 (s, 1H), 7.72 (d, J=1.8 Hz, 2H), 7.62 (t, J=1.8 Hz, 1H), 7.22 (d, J=8.0 Hz, 2H), 7.10 (br d, J=8.2 Hz, 3H), 6.67 (s, 2H), 5.36 (s, 2H), 4.95-4.87 (m, 1H), 4.73 (s, 2H), 4.53 (t, J=5.2 Hz, 2H), 4.10 (br dd, J=2.2, 8.4 Hz, 1H), 3.87 (br t, J=5.2 Hz, 3H), 3.72 (s, 6H), 3.68-3.58 (m, 144H), 3.39 (s, 3H), 3.32 (br dd, J=5.8, 14.2 Hz, 1H), 3.13-3.02 (m, 2H).

The desired product was confirmed by LC/MS with an exact mass of 2316.10.

### Comparator Compound 11

### Preparation of 2-bromo-1,3-dimethoxy-5-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]benzene

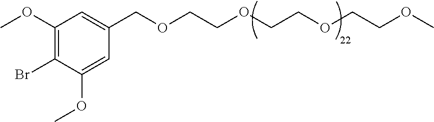

### Step 1. Preparation of 2-bromo-5-(bromomethyl)-1,3-dimethoxybenzene

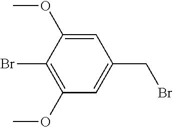

To a solution of (4-bromo-3,5-dimethoxyphenyl)methanol (18.0 g, 72.8 mmol) in toluene (126 mL) was added PBr₃ (20.7 g, 76.4 mmol) at 0˜10° C. The mixture was stirred at 20° C. for 4 h. TLC showed the reaction was complete. The mixture was cooled to 15° C., and diluted with ethyl acetate (200 mL). The organic phase was quenched with ice water (200 mL), and then extracted with ethyl acetate (200 mL*2). The combined organic layer was washed with brine (500 mL), dried over sodium sulfate, filtered and concentrated under vacuum to give the desired product (13.5 g, yield 59.1%) as a white solid, which was purified by flash silica gel chromatography (ISCO®; 30 g Sepa Flash® Silica Flash Column, Eluent of 0˜30% Ethyl acetate/Petroleum ether gradient @100 mL/min). ¹H NMR (400 MHz, DMSO-d₆): δ 6.86 (s, 2H), 4.68 (s, 2H), 3.88-3.82 (m, 6H)

### Step 2. Preparation of 2-bromo-1,3-dimethoxy-5-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]benzene

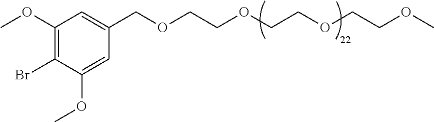

To a solution of m-PEG24-alcohol (BroadPharm) (100 mg, 91.8 μmol) in DMF (5.00 mL) was added NaH (4.41 mg, 110 μmol, 60.0% purity) at 0° C. The mixture was stirred at 25° C. for 0.5 h. Then 2-bromo-5-(bromomethyl)-1,3-dimethoxybenzene (34.5 mg, 110 μmol) in DMF (1.00 mL) was drop-wise added and the mixture was stirred at 25° C. for 12 h. LCMS showed the reaction was completed. The mixture was poured into saturated NH₄Cl solution (50.0 mL) and washed with ethyl acetate (50.0 mL*3). The aqueous phase was concentrated to give the desired product, Comparator Example 11 (29.45 mg, yield 24.2%) as an off-white solid, which was purified by prep-HPLC (column: Boston Green ODS 150*30 mm*5 um; mobile phase: [water (0.225% FA)-ACN]; B %: 45%-75%, 10 min). ¹H NMR (400 MHz, CDCl₃): δ 6.59 (s, 2H), 4.54 (s, 2H), 3.91 (s, 6H), 3.71-3.62 (m, 96H), 3.39 (s, 3H)

The desired product was confirmed by LC/MS with an exact mass of 1316.63

### Comparator Compound 12

### Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-(2-methoxyethoxy)ethoxy]ethoxy]ethoxy]ethoxy]ethoxy]ethoxy]ethoxymethyl]phenyl]phenyl]propanoic Acid

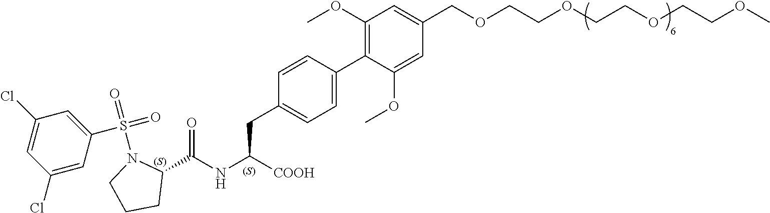

The Scheme B shown for the synthesis of Example 2 is the same Scheme outlining the synthesis of Comparator Example 12, substituting m-PEG8-alcohol for m-PEG24-alcohol in the second to last step.

### Step 1. Preparation of methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-(2-methoxyethoxy)ethoxy]ethoxy]ethoxy]ethoxy]ethoxy]ethoxy]ethoxymethyl]phenyl]phenyl]propanoate

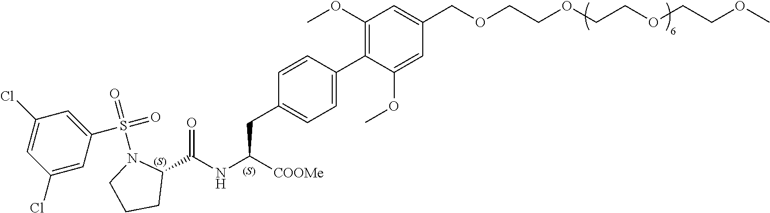

To a mixture of methyl (2S)-2-[[(2S)-1-(3,5-dichlorophenyl)sulfonylpyrrolidine-2-carbonyl]amino]-3-[4-[4-(hydroxymethyl)-2,6-dimethoxy-phenyl]phenyl]propanoate, from Step 7 in Example 2, (357 mg, 520 μmol) and m-PEG8-alcohol (BroadPharm) (200 mg, 520 μmol) was added InCl₃ (92.0 mg, 416 μmol, 26.6 μL). The reaction mixture was stirred at 120° C. for 12 hrs. LCMS showed the reaction was complete. The reaction was concentrated under vacuum to give the crude product, which was purified by prep-HPLC (column: Phenomenex luna C18 150*40 mm*15 um; mobile phase: [water (0.1% TFA)-ACN]; B %: 55%-85%, 11 min) and further purified by prep-HPLC (column: Phenomenex Gemini NX—C18(75*30 mm*3 um); mobile phase: [water (10 mM NH₄HCO₃)-ACN]; B %: 58%-88%, 8 min) to get the desired product (250 mg, 9.71% yield) as light yellow gum.

### Step 2. Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-(2-methoxyethoxy)ethoxy]ethoxy]ethoxy]ethoxy]ethoxy]ethoxy]ethoxymethyl]phenyl]phenyl]propanoic Acid

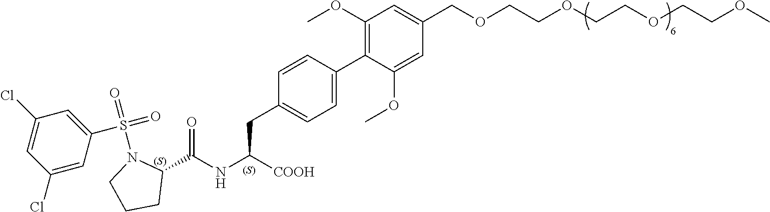

To a solution of methyl (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-(2-methoxyethoxy)ethoxy]ethoxy]ethoxy]ethoxy]ethoxy]ethoxy]ethoxymethyl]phenyl]phenyl]propanoate (250 mg, 202 μmol) in THF (10.0 mL) was drop-wise added LiOH·H₂O (33.9 mg, 808 μmol) in H₂O (2.00 mL). The mixture was stirred at 25° C. for 0.5 hr. LCMS showed the reaction was complete. The reaction mixture was diluted with water (20.0 mL) and adjusted pH to 3 with 2 M HCl. The mixture was extracted with ethyl acetate (20.0 mL*3). The aqueous phase was freeze-dried to give a residue. The residue was purified by prep-HPLC (column: Waters Xbridge C18 150*50 mm*10 um; mobile phase: [water (10 mM NH₄HCO₃)-ACN]; B %: 24%-54%, 11.5 min) and further purified by prep-HPLC (column: Waters Xbridge 150*25 mm*5 um; mobile phase: [water (10 mM NH₄HCO₃)-ACN]; B %: 20%-50%, 9 min). Because of a small quantity of TFA was detected by FNMR, the residue was finally purified by prep-HPLC (column: Waters Xbridge 150*25 mm*5 um; mobile phase: [water (10 mM NH₄HCO₃)-ACN]; B %: 20%-50%, 9 min) to give the desired product, Comparator Example 12 (56.58 mg, 27.7% yield) as white solid. ¹H NMR (400 MHz, CDCl₃): δ 7.80 (s, 2H), 7.55 (t, J=2.0 Hz, 1H), 7.35 (d, J=6.0 Hz, 1H), 7.20 (s, 4H), 6.62 (s, 2H), 4.67-4.58 (m, 1H), 4.56 (s, 2H), 4.35-4.21 (m, 1H), 3.71 (s, 6H), 3.68-3.62 (m, 30H), 3.58-3.53 (m, 2H), 3.45-3.40 (m, 1H), 3.38 (s, 3H), 3.36-3.31 (m, 1H), 3.15-3.10 (m, 1H), 3.02-2.97 (m, 1H), 2.12-2.07 (m, 1H), 1.61-1.38 (m, 3H).

The desired product was confirmed by LC/MS with an exact mass of 1002.34.

### Comparator Compound 13

### Preparation of (S)-2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoic Acid

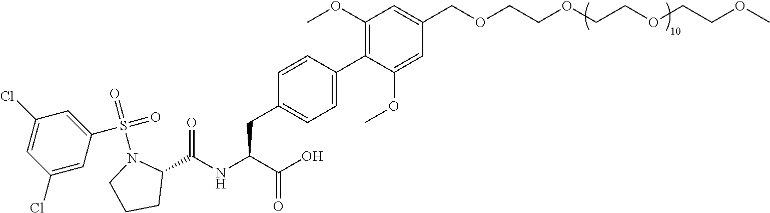

The Scheme B shown for the synthesis of Example 2 is the same Scheme outlining the synthesis of Comparator Example 13, substituting m-PEG12-alcohol for m-PEG24-alcohol in the second to last step.

### Step 1. Preparation of methyl (S)-methyl2-((S)-1-((3,5-dichlorophenyl)sulfonyl) pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoate

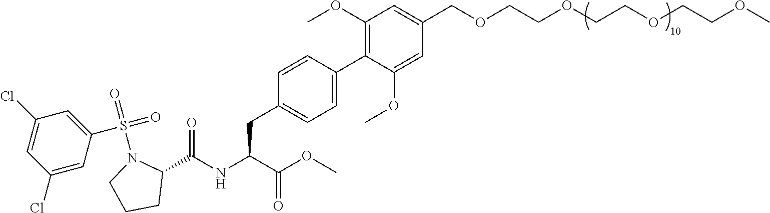

Two reactions were carried out in parallel.

To a mixture of (S)-methyl 2-((S)-1-((3,5-dichlorophenyl)sulfonyl)pyrrolidine-2-carboxamido)-3-(4′-(hydroxymethyl)-2′,6′-dimethoxy-[1,1′-biphenyl]-4-yl)propanoate, from Step 7 in Example 1, (244 mg, 356 μmol, 1.00 eq) and m-PEG12-alcohol (BroadPharm) (200 mg, 356 μmol, 1.00 eq) was added InCl₃ (63.1 mg, 285 μmol, 18.2 μL, 0.80 eq). The reaction mixture was stirred at 120° C. for 12 hrs. The reaction was concentrated under vacuum to give the crude product. The crude product was purified by prep-HPLC (column: Phenomenex luna C18 150*40 mm*15 um; mobile phase: [water (0.1% TFA)-ACN]; B %: 47%-57%, 11 mins) to give methyl (S)-methyl2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoate (21.0 mg, 17.1 μmol, 2.41% yield, 97.7% purity) as light yellow oil.

### Step 2. Preparation of (S)-2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoic Acid

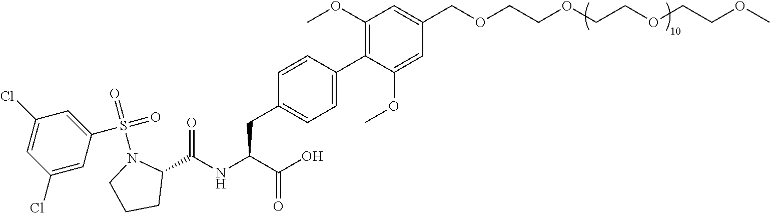

To a solution of methyl (S)-methyl2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoate (21.0 mg, 17.1 μmol, 1.00 eq) in THF (5.00 mL) was dropwise added LiOH·H₂O (2.88 mg, 68.7 μmol, 4.00 eq) in H₂O (1.00 mL). The mixture was stirred at 15° C. for 0.5 hr. The reaction mixture was diluted with water (20.0 mL) and adjusted pH to 3 with 2 M HCl. The mixture was extracted with ethyl acetate (20.0 mL*3). The combined organic layers were washed with brine (20.0 mL), dried over Na₂SO₄, filtered and concentrated. The crude product was purified by prep-HPLC (column: Waters Xbridge 150*25 mm*5 um; mobile phase: [water (10 mM NH₄HCO₃)-ACN]; B %: 21%-51%, 9 mins) to give (S)-2-((S)-1-((3,5-dichlorophenyl)sulfonyl)pyrrolidine-2-carboxamido)-3-(2′,6′-dimethoxy-4′-(2,5,8,11,14,17,20,23,26,29,32,35,38-tridecaoxanonatriacontyl)-[1,1′-biphenyl]-4-yl)propanoic acid, Comparator Example 13 (37.09 mg, 30.5 μmol, 44.4% yield, 97.1% purity) as white gum. ¹H NMR (400 MHz, CDCl₃): δ 7.80 (br s, 2H), 7.56 (s, 1H), 7.25-7.14 (m, 4H), 6.62 (s, 2H), 4.64-4.51 (m, 3H), 4.43-4.12 (m, 1H), 3.71 (s, 6H), 3.68-3.62 (m, 42H), 3.56 (dd, J=3.2, 5.6 Hz, 2H), 3.43-3.34 (m, 5H), 3.27-3.06 (m, 2H), 3.02-2.95 (m, 1H), 2.16-2.01 (m, 3H), 1.62-1.41 (m, 3H), 1.26 (s, 1H)

The desired product was confirmed by QTOF-MS with a mass of 1178.4402

### Comparator Compound 14

### Preparation of (S)-3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoic Acid

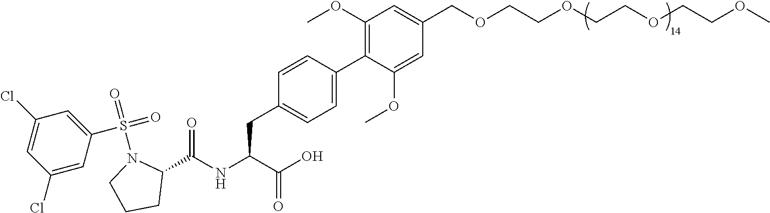

The Scheme B shown for the synthesis of Example 2 is the same Scheme outlining the synthesis of Comparator Example 14, substituting m-PEG16-alcohol for m-PEG24-alcohol in the second to last step.

### Step 1. Preparation of (S)-methyl 3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoate

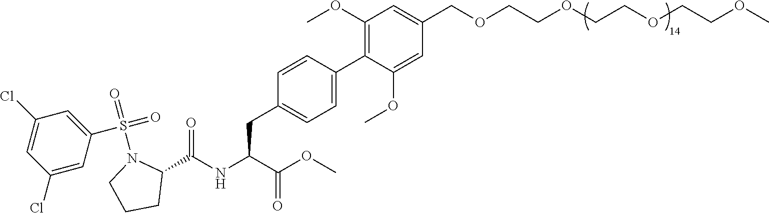

Four reactions were carried out in parallel.

To a mixture of compound (S)-methyl 2-((S)-1-((3,5-dichlorophenyl)sulfonyl)pyrrolidine-2-carboxamido)-3-(4′-(hydroxymethyl)-2′,6′-dimethoxy-[1,1′-biphenyl]-4-yl)propanoate, from Step 7 in Example 1, (186 mg, 271 μmol, 1.00 eq) and m-PEG16-alcohol (BroadPharm) (200 mg, 271 μmol, 1.00 eq) was added InCl₃ (48.0 mg, 217 μmol, 13.8 μL, 0.80 eq). The reaction mixture was stirred at 120° C. for 12 hrs. The reaction was concentrated under vacuum to give the crude product. The crude product was purified by prep-HPLC (column: Phenomenex luna C18 150*40 mm*15 um; mobile phase: [water (0.1% TFA) -ACN]; B %: 47%-77%, 11 mins) to give (S)-methyl 3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoate (100 mg, 67.9 μmol, 6.26% yield, 93.1% purity) as light yellow oil.

### Step 2. Preparation of (S)-3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoic Acid

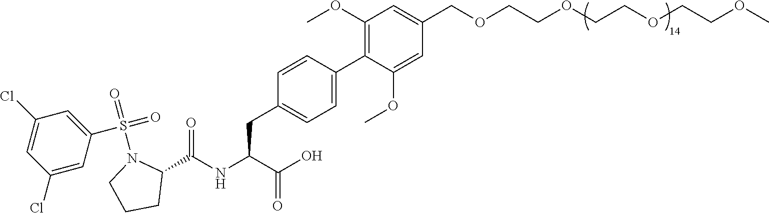

To a solution of (S)-methyl 3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoate (100 mg, 67.9 μmol, 1.00 eq) in THF (5.00 mL) was dropwise added LiOH·H₂O (11.4 mg, 271 μmol, 4.00 eq) in H₂O (1.00 mL). The mixture was stirred at 25° C. for 0.5 hr. The reaction mixture was diluted with water (20.0 mL) and adjusted pH to 3 with 2 M HCl. The mixture was extracted with ethyl acetate (20.0 mL*3). The aqueous phase was freeze-dried to give a residue. The residue was purified by prep-HPLC (column: Waters Xbridge C18 150*50 mm*10 um; mobile phase: [water (10 mM NH₄HCO₃)-ACN]; B %: 23%-53%, 11.5 mins) to give (S)-3-(4′-(2,5,8,11,14,17,20,23,26,29,32,35,38,41,44,47,50-heptadecaoxahenpentacontyl)-2′,6′-dimethoxy-[1,1′-biphenyl]-4-yl)-2-((S)-1-((3,5-dichlorophenyl)sulfonyl)pyrrolidine-2-carboxamido)propanoic acid, Comparator Example 14 (71.57 mg, 52.4 μmol, 77.1% yield, 99.3% purity) as colorless oil. ¹H NMR (400 MHz, CDCl₃): δ 7.73 (s, 2H), 7.60 (s, 1H), 7.41-7.33 (m, 1H), 7.22 (s, 3H), 6.64 (s, 2H), 4.80-4.68 (m, 1H), 4.59 (s, 2H), 4.12-4.10 (m, 1H), 3.71 (s, 9H), 3.68-3.62 (m, 59H), 3.57-3.55 (m, 2H), 3.44-3.40 (m, 1H), 3.39 (s, 3H), 3.25-3.19 (m, 1H), 3.13-3.05 (m, 1H), 2.12-2.03 (m, 2H), 1.63-1.55 (m, 3H) The desired product was confirmed by QTOF-MS with a mass of 1354.5353.

### Comparator Compound 15

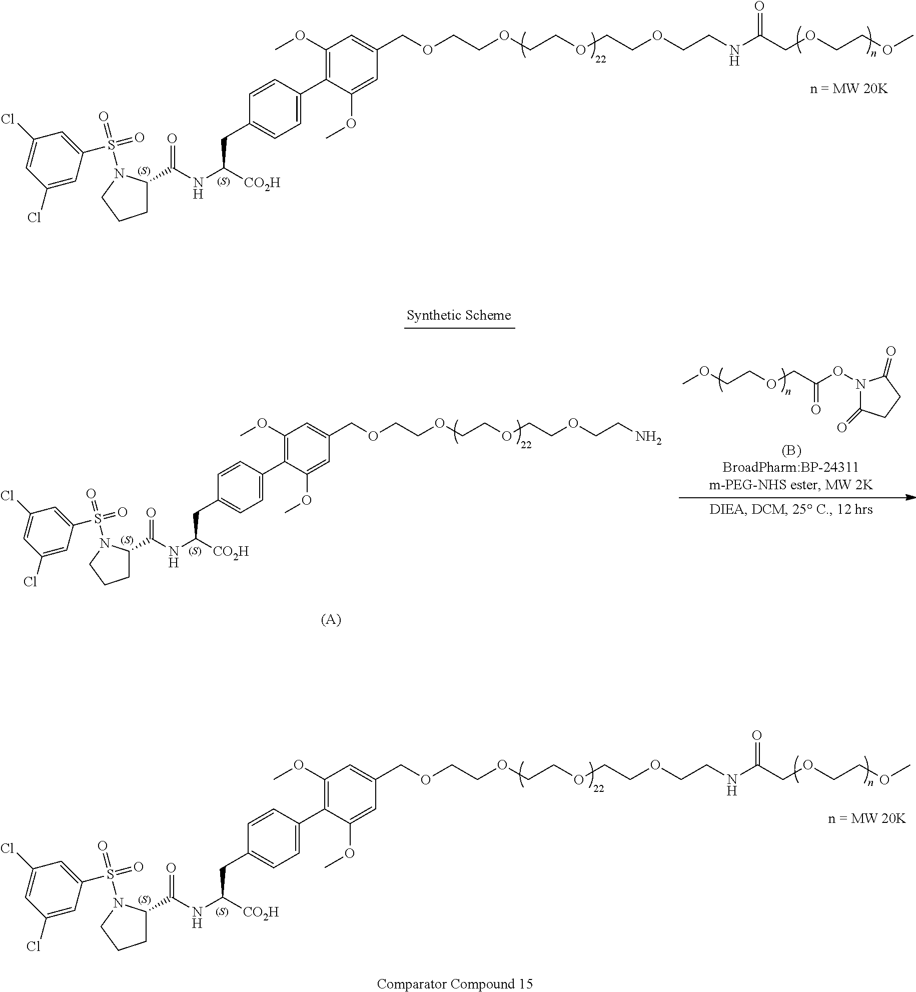

### Step 1. Preparation of (2S)-3-[4-[4-[2-[2-[2-[2-[2-[2-[2-[2-[2-[2-[2-[2-[2-[2- [2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxymethyl]-2,6-dimethoxy-phenyl]phenyl]-2-[[(2S)-1-(3,5-dichlorophenyl)sulfonylpyrrolidine-2-carbonyl]amino]propanoic Acid (Intermediate (A) from the Scheme Above)

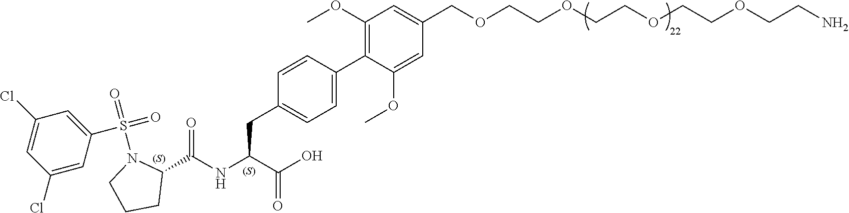

To a mixture of methyl (2S)-2-[[(2S)-1-(3,5-dichlorophenyl)sulfonylpyrrolidine-2-carbonyl]amino]-3-[4-[4-(hydroxymethyl)-2,6-dimethoxy-phenyl]phenyl]propanoate (from Example 2, Step7 above) (110 mg, 170 μmol) and N-Boc-mPEG24-OH (200 mg, 170 μmol) (from BroadPharm) was added InCl₃ (30.1 mg, 136 μmol, 8.71 μL). The reaction mixture was stirred at 120° C. for 12 hrs. LCMS showed the reaction was complete. The reaction was concentrated under vacuum to give the desired product (29.05 mg, 1.85% yield) as light yellow gum, which was purified by prep-HPLC (column: Phenomenex luna C18 150*40 mm*15 um; mobile phase: [water (0.1% TFA)-ACN]; B %: 30%-60%, 11 min) and prep-HPLC (column: Waters Xbridge 150*25 mm*5 um; mobile phase: [water (10 mM NH₄HCO₃)-ACN]; B %: 37%-67%, 9 min). ¹H NMR (400 MHz, CDCl₃): δ 7.73 (d, J=2.0 Hz, 2H), 7.61 (t, J=2.0 Hz, 1H), 7.25 (s, 2H), 7.19-7.16 (m, 2H), 7.10-7.07 (m 1H), 6.67 (s, 2H), 4.93-4.88 (m, 1H), 4.73 (s, 2H), 4.41-4.33 (m, 2H), 4.19-4.04 (m, 1H), 3.75-3.74 (m, 2H), 3.72 (s, 6H), 3.66-3.64 (m, 92H), 3.42-3.30 (m, 3H), 3.20-3.05 (m, 3H), 2.95-2.89 (m, 2H).

### Step 2

To a solution of Intermediate (A), above (40.0 mg, 20.6 μmol, 93.5% purity, 1.00 eq, TFA) and m-PEG-NHS ester, MW 20K (Intermediate (B) from the scheme above (500 mg, 1.00 eq) in DCM (10.0 mL) was added DIEA (10.7 mg, 82.7 μmol, 14.4 μL, 4.00 eq). The mixture was stirred at 25° C. for 12 hrs. LCMS showed that SM (RT=0.876 min) was retained and several new peaks (RT=0.699 min, 0.908 min, 0.939 min, 1.002 mins) were detected. The mixture was concentrated under vacuum to give crude product. The crude product was purified by prep-HPLC (column: Phenomenex luna C18 150*40 mm*15 um; mobile phase: [water (0.1% TFA)-ACN]; B %: 30%-60%, 10 min). From ¹H NMR LCMS, and HPLC the desired product, Comparator Compound 15 (37.65 mg, 97.1% purity) was obtained as off-white solid (see e.g., FIG. 4A-FIG. 4B).

δ 7.73 (d, J=1.2 Hz, 2H), 7.60 (br s, 1H), 7.25-7.13 (m, 4H), 6.71-6.63 (m, 2H), 5.00-4.85 (m, 1H), 4.72 (br s, 2H), 4.38-4.24 (m, 3H), 4.16-4.09 (m, 4H), 4.00 (s, 2H), 3.87-3.80 (m, 20H), 3.65 (s, 2282H), 3.49-3.43 (m, 16H), 3.38 (s, 3H), 3.15-3.00 (m, 4H), 2.01 (s, 42H), 1.26 (br t, J=7.2 Hz, 12H), 0.91-0.80 (m, 6H).

### Comparator Compound 16

### Preparation of (2S)-2-[[(2S)-1-(3,5-dichlorophenyl)sulfonylpyrrolidine-2-carbonyl]amino]-3-[4-[2,6-dimethoxy-4-[2-[2-[2-[2-[2-[2-[2-(2-thiomethyl-ethoxy)ethoxy]ethoxy]ethoxy]ethoxy]ethoxy]ethoxy]ethoxymethyl]phenyl]phenyl]propanoic Acid

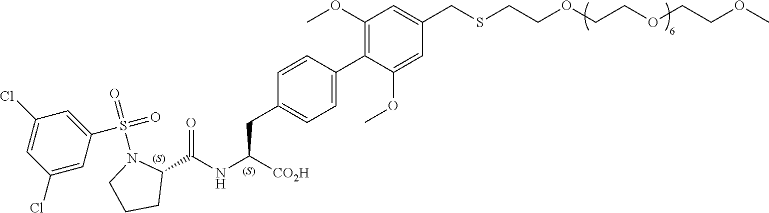

Comparator Example 16 was prepared according to the method of Example 4, substituting m-PEG8-thiol (BroadPharm) for m-PEG-thiol, MW 5K in Step 2 yielding a white solid.

¹H NMR (400 MHz, CDCl₃): δ 1.34-1.50 (m, 1H), 1.51-1.69 (m, 2H), 1.98-2.11 (m, 1H), 2.68 (d, J=5.84 Hz, 1H), 3.02-3.16 (m, 2H), 3.37 (s, 3H), 3.43 (dd, J=14.60, 5.26 Hz, 2H), 3.54 (dd, J=5.84 Hz and 3.50 Hz, 2H), 3.58-3.66 (brs, 28H), 3.67-3.71 (m, 3H), 3.73-3.82 (m, 3H), 4.11 (d, J=8.56 Hz, 2H), 4.79-4.92 (m, 1H), 6.60 (s, 2H), 7.07-7.12 (m, 2H), 7.14 (s, 1H), 7.16-7.21 (m, 2H), 7.21-7.31 (m, 2H), 7.57-7.61 (m, 1H), 7.70 (s, 2H), 9.95 (s, 1H). LC-MS analysis of the solid showed the desired product's mass: m/z 1020 (^{35Cl} M+H), m/z 1022 (^{37Cl} M+H), m/z 1042 (^{35Cl} M+Na) and m/z 1044 (^{37Cl} M+Na); Calcd Mass for C₄₆H₆₅C₁₂N₂O₁₅S₂:1021.04 (M+H).

### Comparator Compound 17

### Preparation of (S)-4-((1-carboxy-2-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)ethyl)carbamoyl)-3,5-dichlorobenzoic Acid

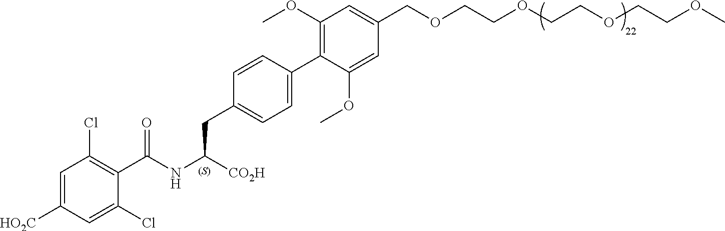

### Step 1. Preparation of (S)-methyl 3-(4-bromophenyl)-2-((tert-butoxycarbonyl)amino)propanoate

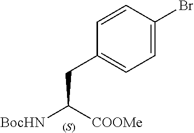

To the solution of (S)-methyl 2-amino-3-(4-bromophenyl)propanoate (45.0 g, 152 mmol, HCl) in DCM (250 mL) was added Boc₂O (50.0 g, 229 mmol, 52.6 mL) and TEA (54.1 g, 534 mmol, 74.4 mL). The mixture was stirred at 25° C. for 3 h. TLC showed the reaction was complete. The reaction mixture was washed with water (500 mL*3). The organic layer was dried by sodium sulfate, filtered and concentrated in vacuum to give the desired product (26.6 g, yield 48.6%) as white solid, which was recrystallized by Petroleum ether/Ethyl acetate=100/1 (300 mL) ¹H NMR (400 MHz, CDCl₃): δ 7.42 (d, J=8.4 Hz, 2H), 7.01 (d, J=8.2 Hz, 2H), 4.97-4.98 (m, 1H), 4.56-4.58 (m, 1H), 3.72-3.73 (m, 3H), 2.97-3.12 (m, 2H), 1.42-1.57 (m, 9H).

### Step 2. Preparation of (S)-methyl 2-((tert-butoxycarbonyl)amino)-3-(4-(4,4,5,5-tetramethyl-1,3,2-dioxaborolan-2-yl)phenyl)propanoate

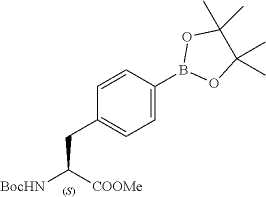

To the solution of (S)-methyl 3-(4-bromophenyl)-2-((tert-butoxycarbonyl)amino)propanoate from Step 1 above (26.6 g, 74.2 mmol) and 4,4,4′,4′,5,5,5′,5′-octamethyl-2,2′-bi(1,3,2-dioxaborolane) (28.2 g, 111 mmol) in dioxane (250 mL) was added Pd(dppf)Cl₂ (5.43 g, 7.43 mmol) and KOAc (21.8 g, 222 mmol). The mixture was stirred at 90° C. for 12 h under N₂ atmosphere. TLC and LCMS showed the reaction was complete. The reaction mixture was filtered and concentrated in vacuum to give the desired product (20.0 g, yield 63.9%) as yellow oil, which was purified by column chromatography (SiO₂, Petroleum ether/Ethyl acetate=100/1 to 10/1, Rt=0.50). ¹H NMR (400 MHz, CDCl₃): δ 7.74 (d, J=8.0 Hz, 2H), 7.13 (d, J=7.8 Hz, 2H), 4.96 (d, J=7.8 Hz, 1H), 4.56-4.61 (m, 1H), 3.70 (s, 3H), 3.05-3.15 (m, 2H), 2.05 (s, 1H), 1.42 (s, 9H), 1.34 (s, 12H), 1.24-1.27 (m, 3H).

### Step 3. Preparation of (S)-methyl 2-((tert-butoxycarbonyl)amino)-3-(4′-(hydroxymethyl)-2′,6′-dimethoxy-[1,1′-biphenyl]-4-yl)propanoate

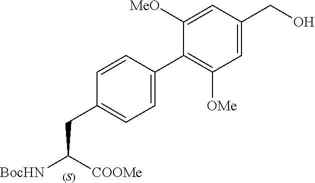

To a solution of (S)-methyl 2-((tert-butoxycarbonyl)amino)-3-(4-(4,4,5,5-tetramethyl-1,3,2-dioxaborolan-2-yl)phenyl)propanoate from Step 2 above (10.0 g, 23.7 mmol), (4-bromo-3,5-dimethoxyphenyl) methanol, from Example 9, step 6 (7.04 g, 28.4 mmol), Pd(dppf)Cl₂ (1.74 g, 2.37 mmol) and K₃PO₄ (15.1 g, 71.2 mmol) in dioxane (50.0 mL) and H₂O (10.0 mL) was stirred at 80° C. for 12 h. TLC and LCMS showed the reaction was complete. The reaction mixture was filtered. The filtrate was poured into water (50.0 mL) and extracted with ethyl acetate (50.0 mL*3). The combined organic layer was washed with brine (100 mL), dried over sodium sulfate, filtered and concentrated to give the desired product (6.27 g, yield 59.1%) as light yellow oil, which was purified by column chromatography (SiO₂, Petroleum ether/Ethyl acetate=20/1 to 1/1, Rt=0.20) and further purified by prep-HPLC (column: Phenomenex luna C18 250*50 mm*10 um; mobile phase: [water (0.1% TFA)-ACN]; B %: 30%-55%, 24 min). ¹H NMR (400 MHz, CDCl₃): δ 7.27-7.29 (m, 2H), 7.17 (d, J=8.0 Hz, 2H), 6.68 (s, 2H), 5.09 (d, J=7.8 Hz, 1H), 4.73 (s, 2H), 4.62-4.65 (m, 1H), 3.74 (s, 9H), 3.06-3.17 (m, 2H), 1.44 (s, 9H).

### Step 4. Preparation of (S)-methyl 2-amino-3-(4′-(hydroxymethyl)-2′,6′-dimethoxy-[1,1′-biphenyl]-4-yl)propanoate

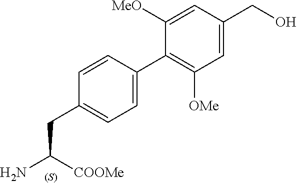

A solution of (S)-methyl 2-((tert-butoxycarbonyl)amino)-3-(4′-(hydroxymethyl)-2′,6′-dimethoxy -[1,1′-biphenyl]-4-yl)propanoate from Step 3 above (10.2 g, 23.1 mmol) in HCl/dioxane (4.00 M, 100 mL) was stirred at 25° C. for 0.5 h. TLC and LCMS showed the reaction was complete. The reaction mixture was concentrated under vacuum to give the desired product (8.17 g, crude, HCl) as white solid, which was directly used for next step without further purification.

### Step 5. Preparation of (S)-methyl 2-(4-bromo-2,6-dichlorobenzamido)-3-(4′-(hydroxymethyl)-2′,6′-dimethoxy-[1,1′-biphenyl]-4-yl)propanoate

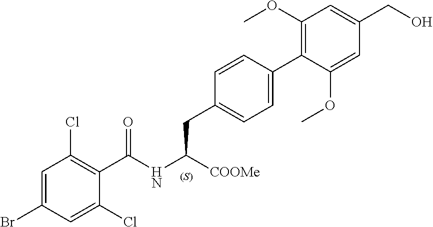

To a solution of 4-bromo-2,6-dichlorobenzoic acid (1.83 g, 6.78 mmol, 1.00 eq) in DMF (30.0 mL) was added DIEA (4.38 g, 33.9 mmol, 5.90 mL, 5.00 eq), HATU (3.87 g, 10.1 mmol, 1.50 eq) and HOBt (274 mg, 2.03 mmol, 0.300 eq). The mixture was stirred at 25° C. for 0.5 hr. Then (S)-methyl 2-amino-3-(4′-(hydroxymethyl)-2′,6′-dimethoxy-[1,1′-biphenyl]-4-yl)propanoate hydrochloride from Step 4 above (3.00 g, 6.78 mmol, 1.00 eq, HCl) was added. The mixture was stirred at 25° C. for 12 hrs. The reaction mixture was poured into water (50.0 mL) and extracted with ethyl acetate (30.0 mL*3). The combined organic layer was washed with brine (80.0 mL), dried over Na₂SO₄, filtered and concentrated. The crude product was purified by prep-HPLC (column: Phenomenex luna C18 (250 * 70 mm, 15 um); mobile phase: [water (0.1% TFA)-ACN]; B %: 40ACN %-70ACN %, 20 min) to give (S)-methyl 2-(4-bromo-2,6-dichlorobenzamido)-3-(4′-(hydroxymethyl)-2′,6′-dimethoxy-[1,1′-biphenyl]-4-yl)propanoate (2.87 g, 4.78 mmol, 70.4% yield, 99.4% purity) as white solid. ¹H NMR (400 MHz, CDCl₃): δ 7.48 (s, 2H), 7.27-7.21 (m, 4H), 6.67 (s, 2H), 6.33 (d, J=8.4 Hz, 1H), 5.27-5.17 (m, 1H), 4.73 (s, 2H), 3.80 (s, 3H), 3.74 (s, 6H), 3.36-3.20 (m, 2H).

### Step 6. Preparation of (S)-methyl 2-(4-bromo-2,6-dichlorobenzamido)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate

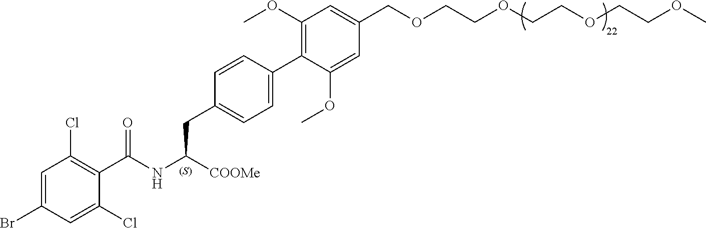

Twelve reactions were carried out in parallel.

The mixture of (S)-methyl 2-(4-bromo-2,6-dichlorobenzamido)-3-(4′-(hydroxymethyl)-2′,6′-dimethoxy-[1,1′-biphenyl]-4-yl)propanoate, from Step 5 above (110 mg, 183 μmol, 1.00 eq) and m-PEG24-alcohol (BroadPharm) (200 mg, 183 μmol, 1.00 eq) was added InCl₃ (32.4 mg, 146 μmol, 9.39 μL, 0.800 eq). The mixture was stirred at 120° C. for 12 hrs. The reaction was concentrated under vacuum to give the crude product. The crude product was purified by prep-HPLC (column: Phenomenex luna C18 150*40 mm*15 um; mobile phase: [water (0.1% TFA)-ACN]; B %: 44%-74%, 10 min) and further purified by prep-HPLC (column: Waters Xbridge C18 150 * 50 mm*10 um; mobile phase: [water (10 mM NH₄HCO₃)-ACN]; B %: 48%-78%, 10 min) to give (S)-methyl 2-(4-bromo-2,6-dichlorobenzamido)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate (150 mg, 51.5 μmol, 2.34% yield, 57.3% purity) as brown oil.

### Step 7. Preparation of (S)-methyl 3,5-dichloro-4-((3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)-1-methoxy-1-oxopropan-2-yl)carbamoyl)benzoate

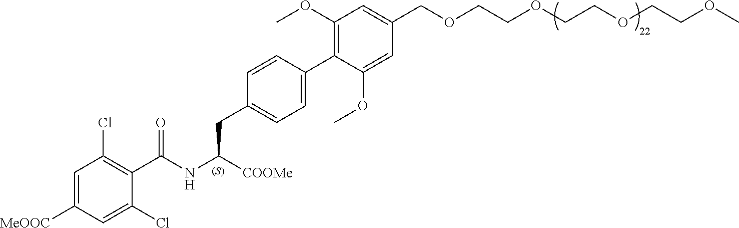

The mixture of (S)-methyl 2-(4-bromo-2,6-dichlorobenzamido)-3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)propanoate, from Step 6 above (150 mg, 51.5 μmol, 1.00 eq), TEA (20.8 mg, 206 μmol, 28.6 μL, 4.00 eq), Pd(dppf)Cl₂ (3.77 mg, 5.15 μmol, 0.10 eq) in MeOH (20.0 mL) was heated to 80° C. under CO atmosphere (50 psi) for 16 hrs. The reaction mixture was filtered and the filtrate was concentrated under vacuum to give (S)-methyl 3,5-dichloro-4-((3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)-1-methoxy-1-oxopropan-2-yl)carbamoyl)benzoate (80 mg, crude) as brown oil, which was used for next step directly without further purification.

### Step 8. Preparation of (S)-4-((1-carboxy-2-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)ethyl)carbamoyl)-3,5-dichlorobenzoic Acid

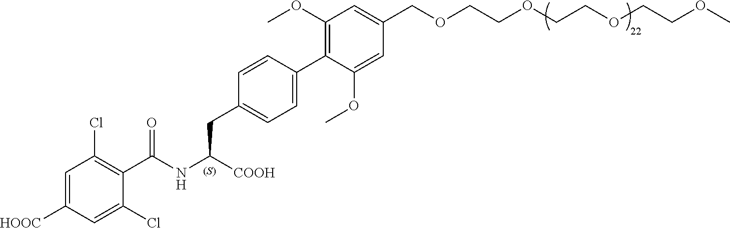

To a solution of (S)-methyl 3,5-dichloro-4-((3-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)-1-methoxy-1-oxopropan-2-yl)carbamoyl)benzoate, from Step 7 above (80 mg, 48.5 μmol, 1.00 eq) in THF (5.00 mL) was dropwise added LiOH·H₂O (8.15 mg, 194 μmol, 4.00 eq) in H₂O (1.00 mL). The mixture was stirred at 15° C. for 0.5 hr. The mixture was poured into water (20.0 mL) and then extracted with ethyl acetate (20.0 mL*3). The aqueous phase was freeze-dried to get a residue. The residue was purified by prep-HPLC (column: Phenomenex luna C18 150*40 mm*15 um; mobile phase: [water (0.1% TFA)-ACN]; B %: 23%-53%, 10 min) and further purified by prep-HPLC (column: Waters Xbridge 150*25 mm*5 um; mobile phase: [water (10 mM NH₄HCO₃)-ACN]; B %: 8%-38%, 9 min) to give (S)-4-((1-carboxy-2-(2′,6′-dimethoxy-4′-(2,5,8,11,14,17,20,23,26,29,32,35,38,41,44,47,50,53,56,59,62,65,68,71,74-pentacosaoxapentaheptacontyl)-[1,1′-biphenyl]-4-yl)ethyl)carbamoyl)-3,5-dichlorobenzoic acid, Comparator Example 17, (50.72 mg, 30.7 μmol, 53.2% yield, 98.3% purity) as white solid. ¹H NMR (400 MHz, CDCl₃): δ 7.73-7.31 (m, 6H), 6.61 (br s, 2H), 5.20-4.86 (m, 1H), 4.57 (br s, 2H), 3.64 (br s, 101H), 3.38 (s, 6H).

MS: Q-Tof exact mass 1617.7269; 1640.7137 (M+Na)+

### Comparator Compound 18

### Preparation of 4-[(4-bromo-3,5-dimethoxy-phenyl)methoxymethyl]-1-[2-[2-[2-[2-[2-[2-[2-[2-[2-[2-[2-[2-[2-[2-[2-[2-[2-[2-[2-[2-[2-[2- [2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]triazole

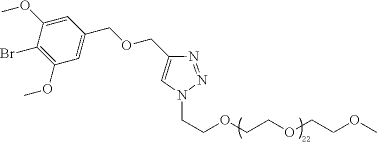

### Step 1. Preparation of 2-bromo-1,3-dimethoxy-5-((prop-2-yn-1-yloxy)methyl)benzene

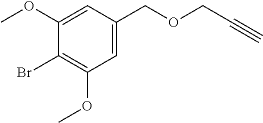 [SMILES: C#CCOCc1cc(OC)c(Br)c(OC)c1]

To a solution of NaH (971 mg, 24.2 mmol, 60.0% purity) in THF (25.0 mL) was added (4-bromo-3,5-dimethoxyphenyl)methanol (5.00 g, 20.2 mmol) at 0° C. The mixture was stirred at 25° C. for 0.5 h. Then 3-bromoprop-1-yne (2.89 g, 24.2 mmol, 2.09 mL) was drop-wise added and the mixture was stirred at 25° C. for 16 h. TLC and LCMS showed the reaction was completed. The reaction mixture was poured into ice water (100 mL) and extracted with ethyl acetate (50.0 mL*3). The organic layers were dried by sodium sulfate, filtered and concentrated in vacuum to give the desired product (4.60 g, yield 78.9%) as light yellow solid, which was purified by column chromatography (SiO₂, Petroleum ether/Ethyl acetate=200/1 to 50/1)¹H NMR (400 MHz, CDCl₃): δ. 6.59 (s, 2H), 4.58 (s, 2H), 4.21 (d, J=2.4 Hz, 2H), 3.91 (s, 6H), 2.50 (t, J=2.4 Hz, 1H).

### Step 2. Preparation of 4-[(4-bromo-3,5-dimethoxy-phenyl)methoxymethyl]-1-[2-[2-[2-[2-[2-[2-[2-[2-[2-[2-[2-[2-[2-[2-[2-[2-[2-[2-[2-[2- [2-[2-[2-(2-methoxyethoxy)ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethoxy]ethyl]triazole

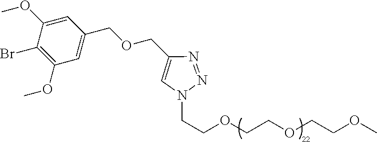

To a solution of 2-bromo-1,3-dimethoxy-5-((prop-2-yn-1-yloxy)methyl)benzene (21.1 mg, 71.0 μmol) in THF (5.00 mL) and H₂O (1.00 mL) was added m-PEG24-azide (80.0 mg, 69.6 μmol), CuSO₄ (11.3 mg, 71.0 μmol, 10.9 μL) and SODIUM ASCORBATE (14.0 mg, 71.0 μmol). The mixture was stirred at 100° C. for 12 h. LCMS showed the reaction was completed. The reaction mixture was concentrated to 3.00 mL under vacuum to give the desired product (32.4 mg, yield 32.2%) as an off-white gum, which was purified by prep-HPLC (column: Xtimate C18 10u 250 mm*80 mm; mobile phase: [water (0.05% ammonia hydroxide v/v)-ACN]; B %: 33%-53%, 10 min). ¹H NMR (400 MHz, CDCl₃): δ 7.77 (s, 1H), 6.60 (s, 2H), 4.69 (s, 2H), 4.57 (s, 2H), 4.55 (t, J=5.2 Hz, 2H), 3.90 (s, 6H), 3.89-3.85 (m, 2H), 3.67-3.61 (m, 92H), 3.38 (s, 3H).

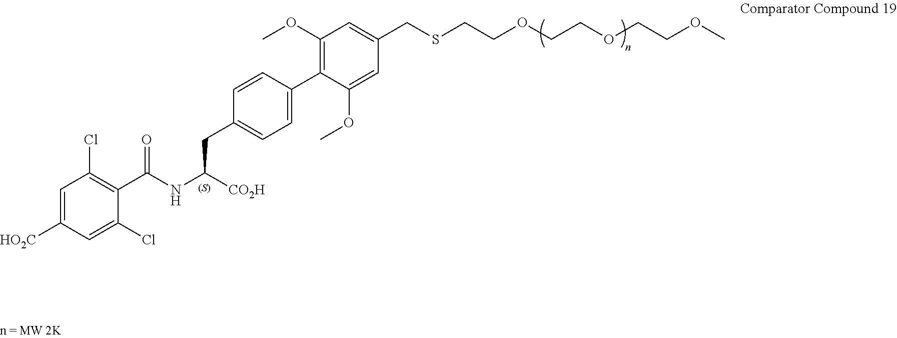

### Preparation of (S)-4-((1-carboxy-2-(2′,6′-dimethoxy-4′-2K MW PEG-thiomethyl)-[1,1′-biphenyl]-4-yl)ethyl)carbamoyl)-3,5-dichlorobenzoic Acid

The method for the synthesis of Example 11 is as described for Example 9 above, substituting m-PEG-thiol, MW 2K (BroadPharm Catalogue #23720) for m-PEG-thiol, MW 20K in Step 9, yielding a white solid.

¹H NMR (400 MHz, CDCl₃): δ 2.69 (t, J=6.81 Hz, 2H), 3.24 (dd, J=14.21 and 7.20 Hz, 2H), 3.40 (s, 3H), 3.42-3.51 (m, 2H), 3.55-3.60 (m, 2H), 3.61-3.69 (brs, 170H), 3.72 (s, 3H), 3.79 (s, 2H), 3.83 (d, J=4.67 Hz, 1H), 5.22 (dd, J=12.85 and 7.01 Hz, 1H), 6.62 (s, 2H), 6.75 (d, J=8.17 Hz, 1H), 7.24 (brs, 2H), 7.28-7.33 (d, J=8.17 Hz, 2H), 7.92 (s, 1H), 8.16 (brs, 2H). LC-MS analysis of the solid showed the desired product's mass: m/z 530 (^{35Cl} M+H-SPEG-m), m/z 532 (^{37Cl} M+H-SPEG-m); Calcd Mass for the Product: 2531.36 (Scaffold+m-PEG-S).

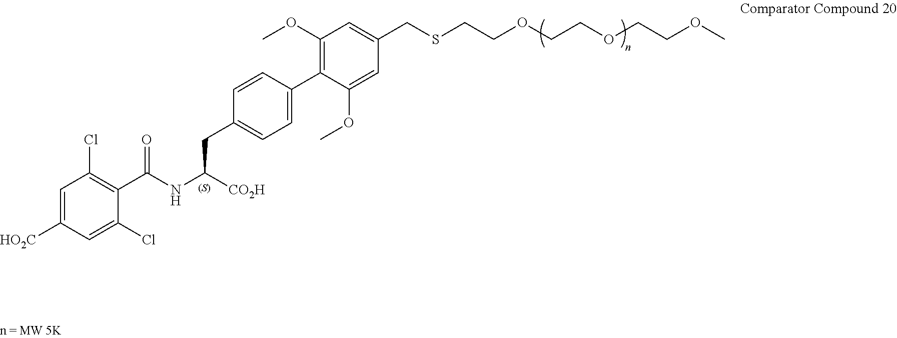

### Preparation of (S)-4-((1-carboxy-2-(2′,6′-dimethoxy-4′-5K MW PEG-thiomethyl)-[1,1′-biphenyl]-4-yl)ethyl)carbamoyl)-3,5-dichlorobenzoic Acid

The method for the synthesis of Example 12 is as described for Example 9 above, substituting m-PEG-thiol, MW 5K (BroadPharm Catalogue #23721) for m-PEG-thiol, MW 20K in Step 9, yielding a white solid.

¹H NMR (400 MHz, CDCl₃): δ 2.65-2.72 (m, 2H), 3.25 (dd, J=14.21 and 6.81 Hz, 2H), 3.38 (s, 3H), 3.44-3.50 (m, 2H), 3.53-3.58 (m, 2H), 3.65 (brs, 444H), 3.72 (s, 3H), 3.78-3.87 (m, 2H), 5.19 (dd, J=13.43 and 6.81 Hz, 1H), 6.62 (s, 2H), 6.75 (d, J=7.79 Hz, 1H), 7.24 (d, J=8.17 Hz, 2H), 7.31 (d, J=8.17 Hz, 2H), 7.93 (s, 1H). LC-MS analysis of the solid showed the desired product's mass: m/z 530 (^{35Cl} M+H—S-PEG-m), m/z 532 (^{37Cl} M+H—S-PEG-m); Calcd Mass for the Product: 5531.36 (Scaffold+m-PEG-S).

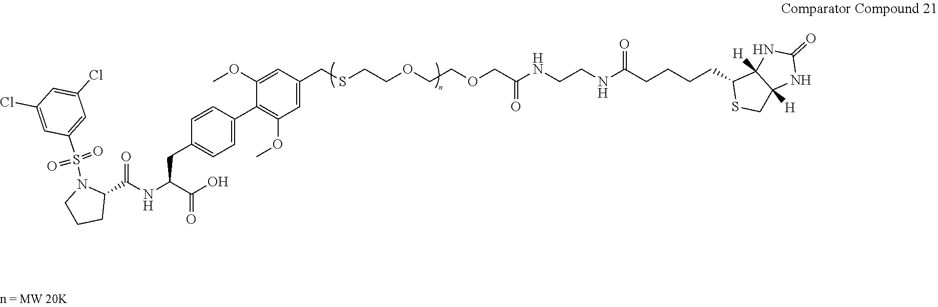

### Preparation of (S)-2-((S)-1-((3,5-dichlorophenyl)sulfonyl)pyrrolidine-2-carboxamido)-3-(4′-(10,15-dioxo-19-((3aR,4R,6aS)-2-oxohexahydro-1H-thieno[3,4-d]imidazol-4-yl)-5,8-dioxa-2-thia-11,14-diazanonadecyl)-2′,6′-dimethoxy-[1,1′-biphenyl]-4-yl)propanoic Acid

### Step 1. Preparation of N-(2-(2-(2-(2-mercaptoethoxy)ethoxy)acetamido)ethyl)(HS-PEG-CH₂COOH-20000)-5-((3aS,4S,6aR)-2-oxohexahydro-1H-thieno[3,4-d]imidazol-4-yl)pentanamide

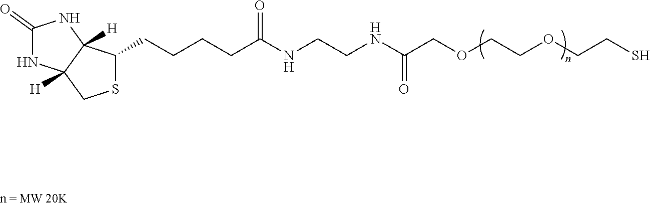

To a solution of a mixture of N-(2-aminoethyl)biotinamide (1.43 mg, 0.0050 mmol) and 20K thiol-PEG-CH₂COOH (100.0 mg, 0.0050 mmol), EDCl (1.92 mg, 0.010 mmol), and HOBt (0.77 mg, 0.0050 mmol) in anhydrous DMF (2.0 mL) was added DIEA (1.7 uL, 0.010 mmol and the reaction mixture was stirred at room temperature overnight. The mixture was concentrated and purified by reverse-phase preparative HPLC and lyophilization afforded the desired product as a colorless powder (72.8 mg). LC-MS analysis of the solid showed the desire product's mass: m/z 309 (M+Na-COCH₂-PEG-thiol), m/z 287 (M+NH-COCH₂-PEG-thiol); Calcd mass for the Product: 20268.39. The product was used as such for the next step.

### Step 2. Preparation of methyl (S)-2-((S)-1-((3,5-dichlorophenyl)sulfonyl)pyrrolidine-2-carboxamido)-3-(4′-(10,15-dioxo-19-((3aR,4R,6aS)-2-oxohexahydro-1H-thieno[3,4-d]imidazol-4-yl)-5,8-dioxa-2-thia-11,14-diazanonadecyl)-2′,6′-dimethoxy-[1,1′-biphenyl]-4-yl)propanoate

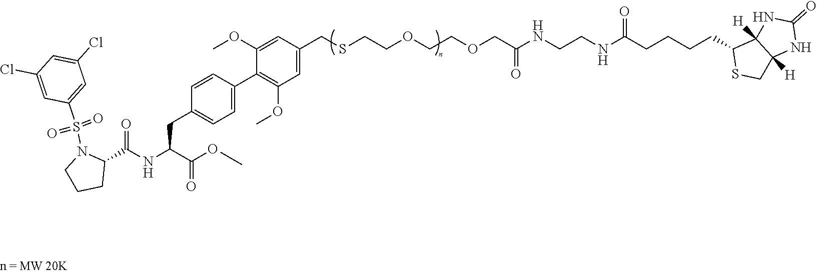

To a solution of N-(2-(2-(2-(2-mercaptoethoxy)ethoxy)acetamido)ethyl)(HS-PEG-CH₂COOH-20000)-5-((3aS,4S,6aR)-2-oxohexahydro-1H-thieno[3,4-d]imidazol-4-yl)pentanamide (72.8 mg, 0.0036 mmol) and methyl (S)-3-(4′-(bromomethyl)-2′,6′-dimethoxy-[1,1′-biphenyl]-4-yl)-2-((S)-1-((3,5-dichlorophenyl) sulfonyl)pyrrolidine-2-carboxamido)propanoate (4.0 mg; 0.0056 mmol) in anhydrous DMF (2.0 mL) was added anhydrous K₂CO₃ (8.0 mg, 0.058 mmol) containing a small crystal of KI and the reaction mixture was heated at 35° C. under nitrogen atmosphere overnight. The reaction mixture was evaporated in-vacuo. The residue was dissolved in acetonitrile, filtered through a 0.45 uM syringe filter and evaporated in-vacuo to afford a colorless solid (0.0898 g). LC-MS analysis of the solid showed the desired product's mass: m/z 633 (^{35Cl} M+H—S-PEG-CH₂—CONH-Biotinamide) and m/z 635 (^{37Cl} M+H—S-PEG-CH₂—CONH-Biotinamide). The crude product was used as such for the saponification with LiOH·H₂O.

### Step 3. Preparation of (S)-2-((S)-1-((3,5-dichlorophenyl)sulfonyl)pyrrolidine-2-carboxamido)-3-(4′-(10,15-dioxo-19-((3aR,4R,6aS)-2-oxohexahydro-1H-thieno[3,4-d]imidazol-4-yl)-5,8-dioxa-2-thia-11,14-diazanonadecyl)-2′,6′-dimethoxy-[1,1′-biphenyl]-4-yl)propanoic Acid

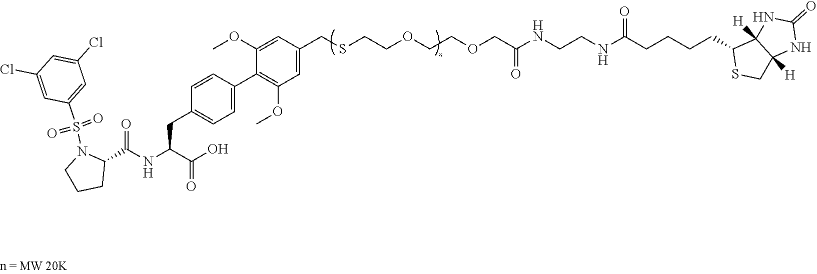

To a solution of the crude methyl (S)-2-((S)-1-((3,5-dichlorophenyl)sulfonyl)pyrrolidine-2-carboxamido)-3-(4′-(10,15-dioxo-19-((3aR,4R,6aS)-2-oxohexahydro-1H-thieno[3,4-d]imidazol-4-yl)-5,8-dioxa-2-thia-11,14-diazanonadecyl)-2′,6′-dimethoxy-[1,1′-biphenyl]-4-yl)propanoate in a mixture of acetonitrile/water (4.0 mL) was added LiOH·H₂O (0.007 g, 0.1668 mmol) and the reaction mixture was stirred at room temperature overnight. The crude product was purified by reverse-phase preparative HPLC (acetonitrile/water, 0.05% TFA) to afford the desired product as a colorless glassy solid after lyophilization (12.7 mg). LC-MS analysis of the solid showed the desired product's mass: m/z 619 (^{35Cl} M+H—S-PEG-CH₂CONH-Biotinamide) and m/z 621 (^{37Cl} M+H-S-PEG-CH₂CONH-Biotinamide). Calcd Mass for the Product: 20987.91 (Scaffold-Thiol-PEG-CH₂CONH-Biotinamide).

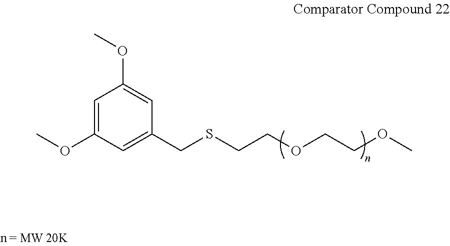

Comparator Compound 22 was synthesized from commercially available 3,5-di-methoxy benzyl bromide and 20 KD PEG mercaptan (BroadPharm) under basic conditions. Calcd Mass for the Product: 20150.18

Methods for the synthesis of Comparator Compounds 1-8 are referenced in WO 2018/085552.

Methods for the synthesis of BOP are referenced in Org. Biomol. Chem., 2014, 12, 965-978.

Firategrast is commercially available from multiple vendors.

### Exemplary Embodiment 2: VLA-4 Inhibiting Potency and Mobilization

The following exemplary embodiment describes the VLA-4 (a4b1) inhibitor compounds that were tested for their ability to inhibit the binding of soluble VCAM-1 to human G2 acute lymphoblastic leukemia (ALL) cells and block ligand binding to integrin a4b7 and HSPC mobilization data.

sVCAM Flow Cytometry Cell-Based Assay.

VLA-4 (a4b1) inhibitor compounds were tested for their ability to inhibit the binding of soluble VCAM-1 to human G2 acute lymphoblastic leukemia (ALL) cells. Briefly, G2 ALL cells are pre-incubated with increasing concentrations (0.1 to 1000 nM) of compounds for 30 minutes. Soluble VCAM/Fc chimera protein (R & D systems) is then added to the mixture and the cells incubated for an additional 30 minutes. Afterwards, cells are washed and VCAM-1 is detected using a PE-conjugated secondary polyclonal antibody. In each experiment, an aliquot of cells are stained with isotype control antibodies to serve as a negative control. The percentage of VCAM-1 binding cells was then determined by flow cytometry. Antibodies used: Jackson Immunuoresearch Cat #: 709-116-098 (PE-labeled donkey anti-human IgG) and Jackson Immunuoresearch Cat #: 017-110-006 (PE-labeled donkey IgG, negative control).

TABLE 3
Inhibition of sVCAM-1 binding to human G2 ALL cells.

| VLA-4 inhibitor | SVCAM-1; IC₅₀ (nM) |
|---|---|
| Example 1 | 0.373 |
| Example 2 | 0.833 |
| Example 3 | 1.281 |
| Example 4 | 0.527 |
| Example 5 | 0.788 |
| Example 6 | 0.796 |
| Example 7 | 1.356 |
| Example 8 | 0.379 |
| Example 9 | 1.280 |
| Example 10 | 1.786 |
| Example 11 | 0.803 |
| Example 15 | 1.270 |
| Comparator Compounds | |
| C1 | 4.980 |
| C2 | 3.832 |
| C3 | 1.351 |
| C4 | 1.693 |
| C5 | 3.589 |
| C6 | 0.445 |
| C7 | 0.194 |
| C8 | 0.199 |
| C9 | 0.579 |
| C10 | 0.552 |
| C11 | >1000 |
| C12 | 0.573 |
| C13 | 0.673 |
| C14 | 0.702 |
| C15 | 7.108 |
| C16 | 0.155 |
| C17 | 1.490 |
| C18 | >1000 |
| C19 | 2.61 |
| C20 | 5.94 |
| C21 | 74.8 |
| C22 | >1000 |
| Firategrast | 230.0 |
| BOP | 47.1 |
| RO0270608 | 98.7 |
| TR-14035 | 247 |
| Carotegrast | 118.3 |

a4b7 Solid Phase Receptor Assay (SPRA)

The potency of compounds in blocking ligand binding to integrin a4b7 was determined by modification of previously described methods (Henderson et al, Nature Medicine, 2013). Briefly, purified human VCAM1 (R & D Systems) diluted to 5 μg/ml in TBS+ buffer (25 mM Tris pH 7.4, 137 mM NaCl, 2.7 mM KCl, 1 mM CaCl₂), 1 mM MgCl₂, 1 mM MnCl₂) was added to wells of a 96-well transparent microtiter plate and incubated overnight at 4° C. Wells were washed 3 times with TBS+ and blocking buffer (TBS+ with 1% bovine serum albumin), the plate was incubated for 1 hr at 37° C., and then washed 3× with TBS+ buffer. Recombinant human a4b7 (R & D Systems) was diluted to 1 μg/ml in TBS+/0.1% bovine serum albumin. Test compounds were diluted into the integrin solution and added to the washed ligand-coated plate according to a standard template with each sample repeated in triplicate. After incubation for 2 hr at room temperature, the plate was washed 3× with 150 μl of TBS+ buffer. To each well, biotinylated anti-A4 antibody (R & D Systems) at 1 μg/ml in TBS+/0.1% BSA was added and the plate covered and incubated for 1 hr at room temperature. After washing the plate 3× with TBS+ buffer, streptavidin-conjugated horseradish peroxidase (R & D Systems) diluted in TBS+ blocking buffer was added to the wells and the plate incubated for 20 min at room temperature. The plate was washed 3× with TBS+ buffer followed by addition of 50 μl of TMB substrate (Sigma T4444). After incubation for 20 min at room temperature, plates were stopped with TMB stop solution (Sigma S5689) by colorimetric detection at 450 nm wavelength using a Tecan Safire II plate reader. Concentration-response curves were constructed by non-linear regression (best fit) analysis, and IC₅₀ values were calculated for each compound. BIO5192, a selective a4b1 inhibitor, was included as a negative control.

TABLE 4
a4b7 SPRA Assay

| Examples | a4b7 SPRA; IC₅₀ (nM) |
|---|---|
| Example 1 | 5.6 |
| Example 2 | 5.4 |
| Example 8 | 4.8 |
| Example 9 | 1.6 |
| Example 10 | 3.9 |
| C1 | 1.1 |
| C6 | 3.9 |
| C17 | 1.5 |
| BIO5192 | >10000 |

### HSPC Mobilization Experiments

Mice. DBA/2J, C₅₇BL/6J (CD45.2) and congenic B6.SJL-Ptprc^{a} Pep3^{b}/BoyJ (CD45.1) mice were purchased from the Jackson Laboratory (Bar Harbor, ME, USA). F1-hybrid mice (CD45.1/2) were obtained through breeding CD45.2 and CD45.1 mice. Animals were housed at the Washington University Medical School vivarium under SPF conditions. All experiments were performed in accordance with the guidelines of the Washington University Animal Studies Committee and the institutional animal care and use committee (IACUC), in agreement with AAALAC guidelines.

HSPC mobilization. All VLA-4 inhibitor compounds were solubilized from the solid powder to a 50 mg/mL stock solution in DMSO or, for the claimed examples with long PEG groups attached, straight saline. For those that dissolve in straight saline, a 0.2 mL dose, diluted with saline from the stock solution to provide the target dose (in mg/kg), was injected s.c. to each test mouse. For compounds not soluble in saline, the 50 mg/mL DMSO stock solution was formulated as in the following example:

For a 3 mg/kg dosing target (assume a 25 g mouse injected with 0.2 mL drug mixture):

$$1. VLA - 4 i :$$

$$( 0.025 kg / mouse ) ( 3 mg / kg ) = 0.0 \; 75 mg / mouse$$

$$( 0.075 mg ) / ( 0.2 mL ) = 0.3 \; 75 mg / mL$$

2. Make 1.2 mL of 0.375 mg/mL VLA4i:

$$VLA 4 i : ( x ) ( 50 mg / mL ) = ( 1200 μL ) ( 0.375 mg / mL )$$

$$x = 9 μL of 50 mg / mL VLA 4 i$$

3. Add 10 mM bicarbonate buffer (pH 8), vortex V(bicarbonate) buffer: (1200 μL)(0.495)=594 μL
4. Bring to volume with saline (0.9% w/v NaCl), vortex V(saline): (1200 μL)−(9 μL+594 μL)=597 μL
5. Add 5-10 μL increments of 1 M NaOH if cloudy.

When doing combination studies with the VLA4 inhibitor plus a CXCR4 inhibitor and/or a CXCL2 agonist, the other agents were dosed as follows:

The CXCR4 inhibitor AMD3100 (Mozobil®, Genzyme, Cambridge, MA, USA) suspension was prepared in PBS and administered s.c. at a dose of 5 mg/kg.

Recombinant human CXCL2 (Groβt; R & D systems) was reconstituted in sterile Ca⁺²/Mg⁺²-free phosphate buffered saline (PBS) and injected subcutaneously (s.c.) at a dose of 2.5 mg/kg.

Colony forming unit (CFU) assay. Peripheral blood (PB) was drawn from the facial vein without anaesthesia into K/EDTA anti-coagulated tubes (Sarstedt AG & Co, NOmbrecht, Germany) at various time points post dosing of the test compounds. Red blood cells were removed from aliquots of blood using hypotonic lysis (Ammonium-Chloride-Potassium, ACK buffer, 5-10 min at RT) and samples were mixed with 2 mL mouse methylcellulose complete media, supplemented with a cocktail of recombinant cytokines (HS0007; R & D Systems, Minneapolis, USA). Cultures were plated in duplicate in 35 mm dishes and placed in a humidified chamber with 5% CO₂ at 37° C. After 7 d of culture, colonies containing at least 50 cells were counted using an inverted microscope in a blinded fashion.

Hematopoietic stem/progenitor cell (HSPC) mobilization data for disclosed Examples and Comparator Compounds was measured by colony forming units (CFU's: No. CFU's (×10³)/mL) is described below and shown in TABLE 5-TABLE 20, and FIG. 11-FIG. 26. Sufficient mobilization requires sustained mobilization of HSPC's into the peripheral blood for greater than 4-6 hours after administration of a single dose of drug. The data in the figures supports the claims that attaching certain PEG groups having a minimum length or greater, as defined in the generic claims depending on the nature of the core scaffold, is necessary to provide for such extended mobilization of HSPCs greater than 4 hours compared to analogues with PEG lengths below the defined amounts claimed or no PEG groups attached to the core.

TABLE 5
HSPC mobilization data for C1-C4.

| | sVCAM | No. CFU (×10³)/mL ± SD | | | |
|---|---|---|---|---|---|
| | IC₅₀ (nM) | Baseline | 0.25 hr | 2 hr | 4 hr |
| Vehicle | — | 0.91 ± 0.20 | 0.53 ± 0.19 | 0.29 ± 0.08 | 0.32 ± 0.08 |
| C1 | 4.90 | 0.91 ± 0.20 | 3.98 ± 1.09 | 0.86 ± 0.25 | 0.27 ± 0.20 |
| C2 | 3.832 | 0.91 ± 0.20 | 4.08 ± 1.66 | 2.87 ± 1.47 | 0.53 ± 0.30 |
| C3 | 1.351 | 0.91 ± 0.20 | 3.69 ± 0.52 | 1.58 ± 0.74 | 0.30 ± 0.11 |
| C4 | 1.693 | 0.91 ± 0.20 | 4.73 ± 1.31 | 1.47 ± 0.51 | 0.49 ± 0.12 |

TABLE 6
HSPC mobilization data for C5-C7.

| | sVCAM | No. CFU (×10³)/mL ± SD | | | |
|---|---|---|---|---|---|
| | IC₅₀ (nM) | Baseline | 0.25 hr | 2 hr | 4 hr |
| Vehicle | — | 0.83 ± 0.26 | 0.66 ± 0.35 | 0.18 ± 0.10 | 0.10 ± 0.06 |
| C5 | 3.589 | 0.83 ± 0.26 | 2.78 ± 0.48 | 1.38 ± 0.53 | 0.31 ± 0.09 |
| C8 | 0.199 | 0.83 ± 0.26 | 2.74 ± 0.69 | 2.10 ± 0.85 | 0.42 ± 0.15 |
| C6 | 0.445 | 0.83 ± 0.26 | 2.58 ± 0.65 | 2.12 ± 0.70 | 0.46 ± 0.20 |
| C7 | 0.194 | 0.83 ± 0.26 | 2.57 ± 0.62 | 2.52 ± 0.37 | 0.50 ± 0.18 |

FIG. 11, FIG. 12, TABLE 5, and TABLE 6 demonstrate HSPC mobilization after a single sub cutaneous (s.c.) injection of Comparator Compounds C1-C8. As can be seen, compared to vehicle, there is rapid increase in the number of CFUs mobilized into the peripheral blood that extends to varying degrees out to 2 hours, but has come down to baseline by 4 hours post dosing.

TABLE 7
HSPC mobilization data for Example 1, C1, and C8.

| | sVCAM | No. CFU (×10³)/mL ± SD | | | |
|---|---|---|---|---|---|
| | IC₅₀ (nM) | Baseline | 0.5 hr | 2 hr | 4 hr |
| Vehicle | — | 0.31 ± 0.14 | 0.10 ± 0.07 | 0.14 ± 0.08 | 0.06 ± 0.07 |
| C1 | 4.90 | 0.31 ± 0.14 | 0.96 ± 0.33 | 0.33 ± 0.14 | 0.08 ± 0.04 |
| C8 | 0.199 | 0.31 ± 0.14 | 0.85 ± 0.30 | 0.87 ± 0.30 | 0.27 ± 0.36 |
| Example 1 | 0.373 | 0.31 ± 0.14 | 1.22 ± 0.35 | 2.12 ± 1.04 | 2.30 ± 0.25 |

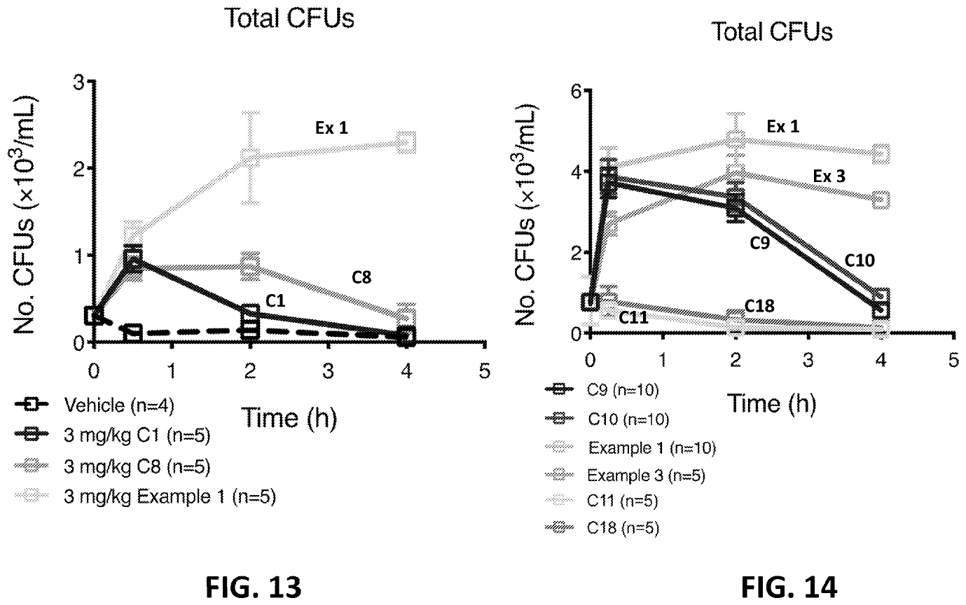

FIG. 13 and TABLE 7 compare claimed Example 1 to structurally related Comparator Compounds C1 and C8. As shown, Example 1 continues to provide significant HSPC mobilization at 4 hours, whereas the Comparator Compounds have returned to baseline. Furthermore, even at 2 hours post dose, Example 1 provides significantly more mobilization than the Comparator Compounds, which is then sustained at this level out at 4 hours, whereas the Comparator Compounds have now returned to baseline.

TABLE 8
HSPC mobilization data for Examples 1 and 3 and
Comparator Compounds C9, C10, C11, and C18.

| | sVCAM | No. CFU (×10³)/mL ± SD | | | |
|---|---|---|---|---|---|
| | IC₅₀ (nM) | Baseline | 0.25 hr | 2 hr | 4 hr |
| C9 | 0.579 | 0.77 ± 0.57 | 3.72 ± 1.18 | 3.09 ± 1.06 | 0.57 ± 0.28 |
| C10 | 0.552 | 0.77 ± 0.57 | 3.87 ± 1.33 | 3.37 ± 1.12 | 0.90 ± 0.52 |
| Example 1 | 0.373 | 0.77 ± 0.57 | 4.10 ± 1.53 | 4.79 ± 2.05 | 4.43 ± 0.66 |
| Example 3 | 1.281 | 0.77 ± 0.57 | 2.71 ± 0.63 | 3.97 ± 0.96 | 3.30 ± 0.22 |
| C11 | >1000 | 0.80 ± 0.59 | 0.56 ± 0.05 | 0.14 ± 0.10 | 0.10 ± 0.07 |
| C18 | >1000 | 0.80 ± 0.59 | 0.78 ± 0.38 | 0.33 ± 0.22 | 0.14 ± 0.06 |

FIG. 14 and TABLE 8 compare claimed Examples 1 and 3 to Comparator Compounds C9 and C10. Similarly to the data in FIG. 13, FIG. 14 and TABLE 8 demonstrate that Example 1 and Example 3 both rapidly mobilize HSPCs and that this mobilization is sustained and extended to the same degree out to 4 hours whereas the Comparator Compounds C9 and 10 are returning to baseline by 4 hours. Furthermore, as previously shown, Example 1 shows greater mobilization than these Comparator Compounds even at 2 hours. Importantly, FIG. 14 and TABLE 8 present data in support of the non-obvious nature of the generic structure claims of the present disclosure in that placing a long PEG group anywhere on the core structure of these VLA4 inhibitors will result in significant and extended HSPC mobilization beyond 4 hours. In this case, utilizing click chemistry to attach a long PEG group to the core structure via a triazole linker only provides extended HSPC mobilization if the triazole linker is separated from the core molecule by a short PEG chain, as represented by Example 3. If the triazole linker is attached closer to the core via a simple benzyl ether linkage, as in C₉ and C10, then extended mobilization is not afforded. Comparator Compounds C11 and C18 are negative controls, demonstrating in FIG. 14 and TABLE 8 that a long PEG group attached to a truncated portion of the core inhibitor structure does not mobilize HSPCs, and ruling out the possibility that a long PEG group by itself results in mobilization of HSPCs.

TABLE 9
HSPC mobilization data for Examples 1 and 2 and
Comparator Compounds C7, C12, C13, and C14.

| | sVCAM | No. CFU (×10³)/mL ± SD | | | |
|---|---|---|---|---|---|
| | IC₅₀ (nM) | Baseline | 2 hr | 4 hr | 6 hr |
| C7 | 0.194 | 0.48 ± 0.13 | 2.65 ± 1.39 | 0.44 ± 0.17 | 0.11 ± 0.08 |
| C12 | 0.573 | 0.48 ± 0.13 | 2.42 ± 1.06 | 1.19 ± 0.53 | 0.24 ± 0.15 |
| C13 | 0.673 | 0.48 ± 0.13 | 2.01 ± 0.75 | 0.41 ± 0.19 | 0.10 ± 0.04 |
| C14 | 0.702 | 0.48 ± 0.13 | 3.17 ± 1.17 | 0.50 ± 0.25 | 0.08 ± 0.11 |
| Example 1 | 0.373 | 0.48 ± 0.13 | 4.72 ± 1.19 | 5.00 ± 1.26 | 1.84 ± 0.63 |
| Example 2 | 0.833 | 0.48 ± 0.13 | 4.10 ± 1.40 | 4.98 ± 1.88 | 1.87 ± 0.58 |

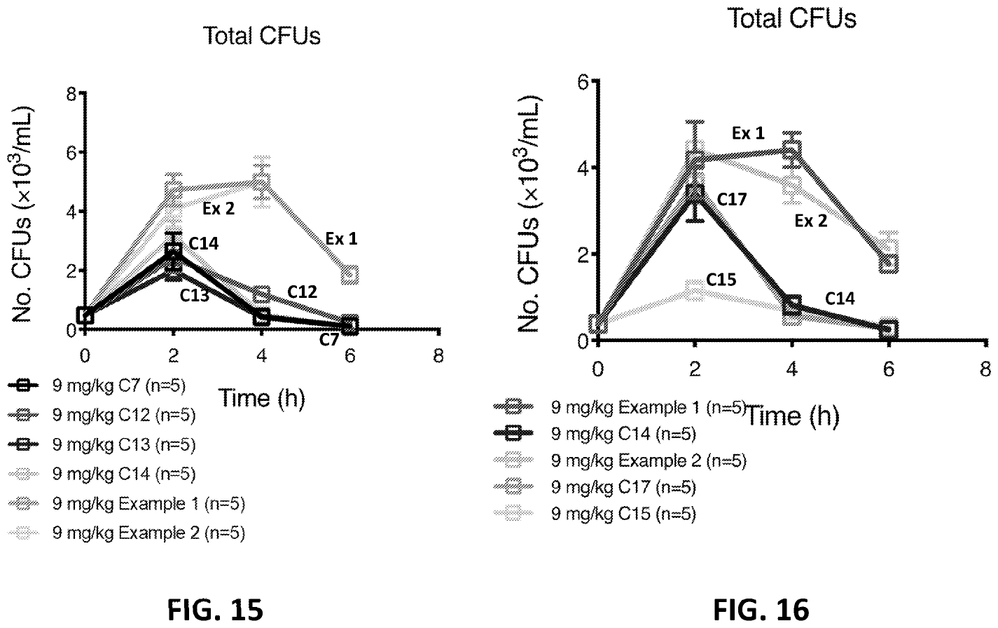

FIG. 15 and TABLE 9 provide additional HSPC mobilization data in support of the non-obvious nature of the generic structure claims of the present disclosure in that placing a PEG group of any length on the core structure of these VLA4 inhibitors will result in significant and extended HSPC mobilization beyond 4 hours. FIG. 15 and TABLE 9 demonstrate that, with all else structurally the same for the core molecule, a PEG group of at least 24 PEG units is required for the desired extended HSPC mobilization effect. Examples 1 and 2 have PEG units of 24 and 36 in length, respectively and, as in the previous figures, show superior mobilization of HSPCs at 2 hours and sustains the same degree of mobilization at 4 hours, while both still retaining mobilization out to 6 hours. In contrast, Comparator Compounds C7 (3 PEG units), C12 (8 PEG units), C13 (12 PEG units) and C14 (16 PEG units) are all returning to baseline by 4 hours and clearly at baseline by 6 hours.

TABLE 10
HSPC mobilization data for Examples 1 and 2
and Comparator Compounds C14, C15, and C17.

| | sVCAM | No. CFU (×10³)/mL ± SD | | | |
|---|---|---|---|---|---|
| | IC₅₀ (nM) | Baseline | 2 hr | 4 hr | 6 hr |
| Example 1 | 0.373 | 0.39 ± 0.16 | 4.18 ± 1.99 | 4.41 ± 0.89 | 1.78 ± 0.38 |
| C14 | 0.702 | 0.39 ± 0.16 | 3.40 ± 1.42 | 0.82 ± 0.47 | 0.25 ± 0.16 |
| Example 2 | 0.833 | 0.39 ± 0.16 | 4.42 ± 1.41 | 3.59 ± 0.93 | 2.12 ± 0.84 |
| C17 | 1.490 | 0.39 ± 0.16 | 3.67 ± 1.09 | 0.57 ± 0.21 | 0.28 ± 0.17 |
| C15 | 7.108 | 0.39 ± 0.16 | 1.15 ± 0.47 | 0.68 ± 0.36 | 0.33 ± 0.28 |

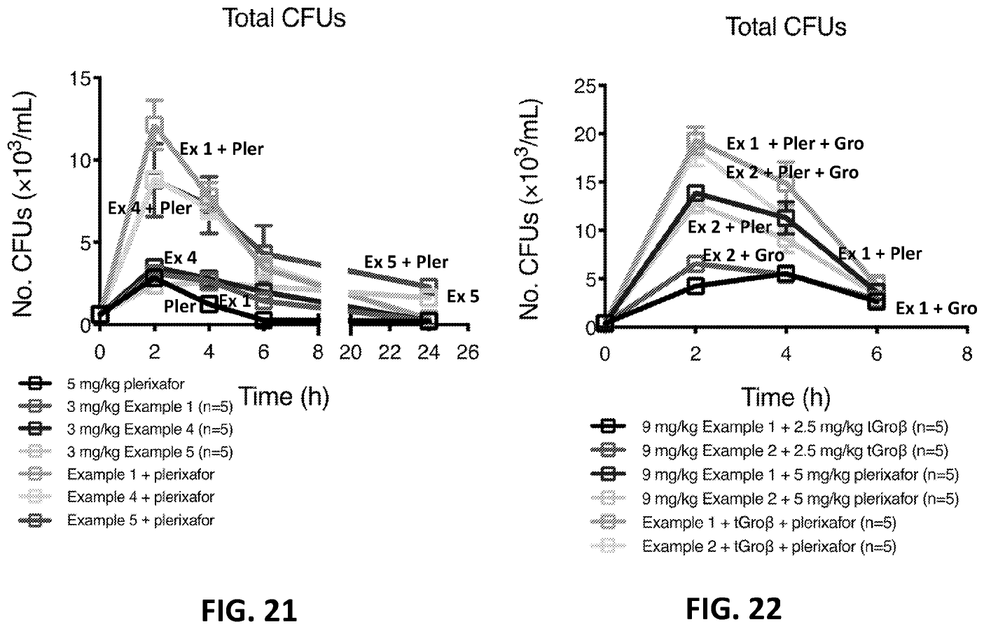

FIG. 16 and TABLE 10 demonstrate the non-obvious nature of the claimed Examples in regards to providing extended mobilization beyond 4 hours after administration of a single dose of drug compared to similar analogues as represented by Comparator Compounds C14, C15 and C17. In this mobilization study, claimed Examples 1 and 2, with PEG chain lengths of 24 and 36 PEG units respectively and attached to the “di-chlorophenyl sulfonamide core” (refer to TABLE 1 and TABLE 2 for full structural clarity) provide for such extended mobilization, with significant mobilization observed at 2 hours post dose and extended out to 6 hours. In contrast, and as also shown in FIG. 15 and TABLE 9, C14, the 16mer PEG version of this core scaffold, shows mobilization at 2 hours post dose but is returning to baseline at 4 hours and affords no mobilization effects at 6 hours. C15 provides another comparator example showing that not any long PEG configuration will provide extended mobilization. Insertion of an amide functionality into a long 20 KD PEG chain in C15 provides weak mobilization even at 2 hours and exhibits no extended mobilization and is 10× less potent in the VCAM assay for inhibition of VLA4 compared to Example 5, a 20 KD PEG analogue without this amide insertion. Example 5 also shows extensive mobilization which extends out to 8 hours and beyond (see e.g., FIG. 18 and FIG. 21). C17 has a 24 PEG unit chain attached to a “di-chlorobenzoic acid core” (refer to the compound structures section in TABLE 1 for full structural clarity), a core that also provides potent inhibition of VLA4. However, unlike Example 1, which also has a 24 PEG unit group attached to its core, C17 exhibits the mobilization profile of the other Comparator Compounds, namely good mobilization of HSPCs at 2 hours post dose, but returning to baseline by 4 hours and no mobilization at 6 hours. C17 lends to the non-obvious nature of the generic claims and examples disclosed herein, as a 24 PEG unit chain length attached to the core of Example 1 provides significant extended mobilization, whereas the same PEG unit length attached to a related VLA4 inhibitor core (as in C17) does not.

TABLE 11
HSPC mobilization data for Examples 1, 4, 6, 7,
and 9 and Comparator Compounds C12 and C16.

| | sVCAM | No. CFU (x10³)/mL ± SD | | | | |
|---|---|---|---|---|---|---|
| | IC₅₀ (nM) | Baseline | 2 hr | 4 hr | 6 hr | 24 hr |
| Vehicle | | 0.58 ± 0.14 | 0.20 ± 0.15 | 0.26 ± 0.12 | 0.19 ± 0.08 | 0.13 ± 0.07 |
| C12 | 0.573 | 0.58 ± 0.14 | 0.78 ± 0.17 | 0.58 ± 0.19 | 0.22 ± 0.10 | 0.14 ± 0.07 |
| Example 1 | 0.373 | 0.58 ± 0.14 | 3.14 ± 0.63 | 4.14 ± 1.31 | 2.20 ± 0.63 | 0.29 ± 0.11 |
| C16 | 0.155 | 0.58 ± 0.14 | 2.34 ± 0.55 | 1.14 ± 0.86 | 0.38 ± 0.24 | 0.14 ± 0.06 |
| Example 7 | 1.356 | 0.58 ± 0.14 | 2.53 ± 1.11 | 3.91 ± 1.44 | 2.96 ± 1.15 | 0.15 ± 0.05 |
| Example 4 | 0.527 | 0.58 ± 0.14 | 4.18 ± 0.93 | 4.74 ± 1.57 | 3.62 ± 1.03 | 0.30 ± 0.13 |
| Example 6 | 0.796 | 0.58 ± 0.14 | 2.78 ± 0.70 | 3.56 ± 0.93 | 2.22 ± 0.51 | 0.22 ± 0.08 |
| Example 9 | 1.280 | 0.58 ± 0.14 | 2.65 ± 0.41 | 3.55 ± 0.69 | 3.04 ± 0.88 | 2.02 ± 0.78 |

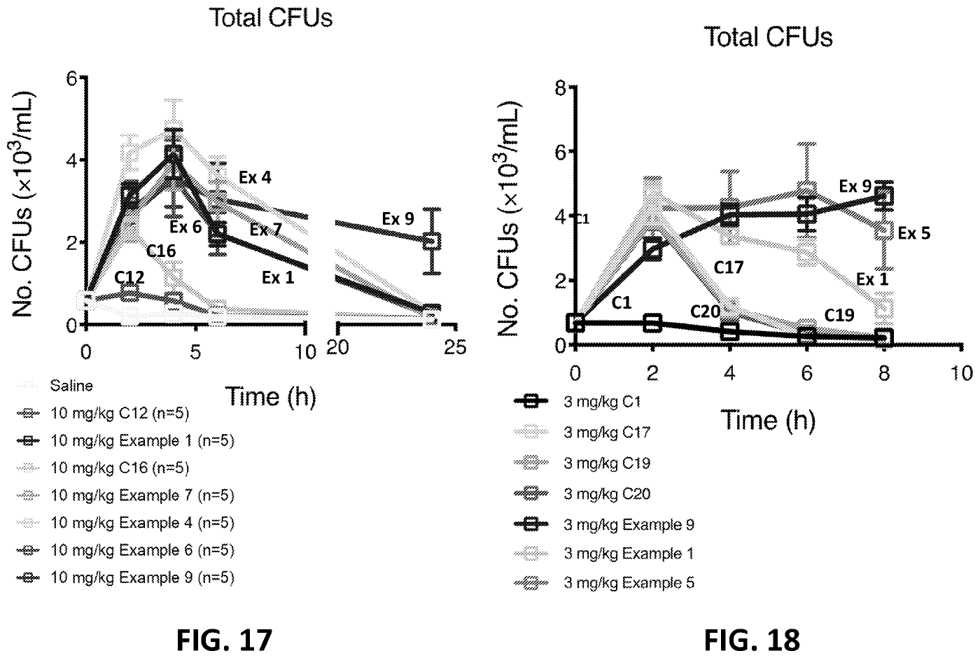

FIG. 17 and TABLE 11 supports the definition that X₂ in the generic claims may be sulfur or oxygen with equivalent results in regards to extended mobilization, with all other defined parameters being equal. As shown, Example 1 and Example 7 are the same, except for X₂ being oxygen in Example 1 and sulfur in Example 7, and both provide for extended HSPC mobilization into the peripheral blood out to 6 hours. Additionally, Comparator Compound C16, with a PEG chain of 8 PEG units and where X₂ is sulfur, and its oxygen counterpart C12, do not provide the same extended mobilization as the 24 PEG unit analogues Example 1 and 7, again supporting the claims that a minimum of 24 PEG units is required for extended mobilization when attached to the “di-chlorophenyl sulfonamide core”. Supporting the claims that PEG units longer than 24 also provide for extended mobilization, Example 4, with a PEG MW of 5 K and Example 6 with a 5K MW PEG thiol attached to the “di-chlorophenyl sulfonamide core” via a short PEG linker (refer to the compound structures section in TABLE 1 for full structural clarity) provide extended mobilization out to 6 hours. FIG. 17 and TABLE 11 also support the non-obvious nature of the claims of this disclosure, as demonstrated by Example 9. Example 9 has a 20K MW PEG unit chain attached to a “di-chlorobenzoic acid core” (refer to the compound structures section in TABLE 1 for full structural clarity) and provides for extended mobilization all the way out to 24 hours after a single dose. This is in contrast to C17 which has a 24 PEG unit attachment to this same core, but does not extend mobilization. This supports the non-obvious claims of this disclosure that a minimum number of PEG units required for extended mobilization for one VLA4 inhibitor core is not universally equivalent for a similar, but structurally different VLA4 inhibitor core.

TABLE 12
HSPC mobilization data for Examples 1, 5, and 9
and Comparator Compounds C1, C17, C19, and C20.

| | sVCAM | No. CFU (x10³)/mL ± SD | | | | |
|---|---|---|---|---|---|---|
| | IC₅₀ (nM) | Baseline | 2 hr | 4 hr | 6 hr | 8 hr |
| C1 | 4.90 | 0.69 ± 0.13 | 0.67 ± 0.12 | 0.42 ± 0.14 | 0.26 ± 0.07 | 0.22 ± 0.02 |
| C17 | 1.490 | 0.69 ± 0.13 | 4.32 ± 1.23 | 1.21 ± 0.40 | 0.26 ± 0.07 | 0.19 ± 0.09 |
| C19 | | 0.69 ± 0.13 | 3.94 ± 0.67 | 1.11 ± 0.28 | 0.51 ± 0.25 | 0.22 ± 0.06 |
| C20 | | 0.69 ± 0.13 | 3.90 ± 0.69 | 1.02 ± 0.56 | 0.32 ± 0.04 | 0.18 ± 0.05 |
| Example 9 | 1.280 | 0.69 ± 0.13 | 2.97 ± 0.57 | 4.03 ± 0.73 | 4.06 ± 1.17 | 4.62 ± 0.98 |
| Example 1 | 0.373 | 0.69 ± 0.13 | 4.72 ± 0.33 | 3.38 ± 0.21 | 2.87 ± 0.39 | 1.12 ± 0.48 |
| Example 5 | 0.788 | 0.69 ± 0.13 | 4.25 ± 0.94 | 4.26 ± 1.12 | 4.78 ± 1.45 | 3.55 ± 1.19 |

FIG. 18 and TABLE 12 provide additional support for the claims in this disclosure that PEG unit chains attached to a “di-chlorophenyl sulfonamide core” (refer to the compound structures section in TABLE 1 for full structural clarity) 24 PEG units or longer result in extended mobilization of HSPCs after a single injection out to 6 hours or longer. Example 1 (24 PEG units) extends mobilization out to 6 hours, whereas Example 5, which has a 20K MW PEG chain attached to this core, extends significant mobilization out to 8 hours (the last time point taken in this study). This demonstrates that attaching very long PEG units to this core does not diminish VLA4 inhibition potency or mobilization efficacy, but actually enhances the duration of the desired effect. FIG. 18 and TABLE 12 further support the non-obvious nature of the disclosed claims, as demonstrated by Example 9 and Comparator Compounds C1, C17, C19 and C20 (all belonging to the “di-chlorobenzoic acid core” (refer to the compound structures section in TABLE 1 and TABLE 2 for full structural clarity). Example 9, which has a 20K MW PEG chain attached to this core, significantly extends HSPC mobilization out to 8 hours in this study, and out to 24 hours as shown in FIG. 17. This is in contrast to C1, which has no PEG units attached to this core, C17, which has a 24 PEG unit chain attached to this core, C19, which has a 2K MW PEG chain attached to this core, and C20, which has a 5K MW PEG chain attached to this core. None of these Comparator Compounds extend mobilization of HSPCs beyond 2 hours. Importantly, neither the 2 or 5K MW PEG chains attached to this “di-chlorobenzoic acid core” extend mobilization. This is in contrast to Example 1 (24 PEG units) and Example 4 (a 5K MW PEG chain) attached to the “di-chlorophenyl sulfonamide core”, which do extend mobilization past 6 hours. This again supports the non-obvious nature of the disclosed claims in that you can't attach the same PEG chain lengths to any VLA4 inhibitor core and achieve the same extended mobilization. In this case a PEG chain of at least 20K MW is required for the “di-chlorobenzoic acid core” to achieve extended mobilization, as opposed to a 24 PEG unit chain minimum required for the “di-chlorophenyl sulfonamide core”.

TABLE 13
HSPC mobilization data for BOP or Firategrast
in combination with Groβt.

| | sVCAM | No. CFU (×10³)/mL ± SD | | |
|---|---|---|---|---|
| | IC₅₀ (nM) | Baseline | 0.5 hr | 2 hr |
| BOP ± Groβt | (BOP) | 0.15 ± 0.07 | 5.13 ± 3.07 | 2.05 ± 1.30 |
| | 47.0 | | | |
| Firategrast ± | (Firat.) | 0.15 ± 0.07 | 2.30 ± 0.85 | 1.19 ± 0.54 |
| Groβt | 230.0 | | | |

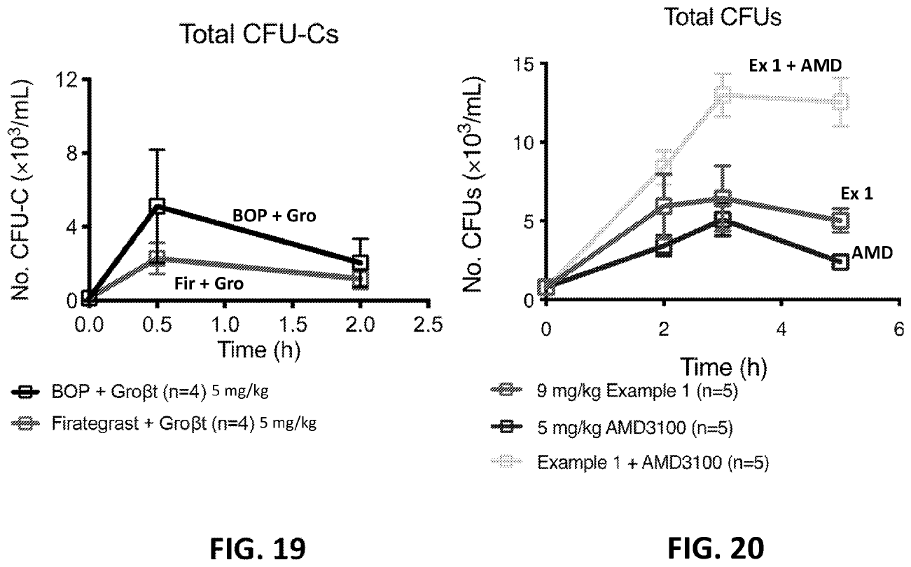

FIG. 19 and TABLE 13 demonstrate that two prior art compounds with analogous structures to the claimed examples disclosed herein, “BOP” and Firategrast (structures depicted in TABLE 2) show a rapid but transient mobilization of HSPCs but are returning to baseline by 2 hours. And this despite being dosed in combination with another mobilization agent, the CXCR2 agonist truncated Groβ(Groβt). This again demonstrates the novelty of the claims of this disclosure in providing extended HSPC mobilization after a single dose of a claimed example compared to compounds of similar structures that do not provide such properties.

TABLE 14
HSPC mobilization data for Example 1 alone or in combination with AMD3100.

| | sVCAM | No. CFU (×10³)/mL ± SD | | | |
|---|---|---|---|---|---|
| | IC₅₀ (nM) | Baseline | 2 hr | 3 hr | 5 hr |
| Example 1 | 0.373 | 0.79 ± 0.33 | 5.94 ± 4.52 | 6.42 ± 4.64 | 5.02 ± 1.67 |
| AMD3100 | | 0.79 ± 0.33 | 3.42 ± 1.42 | 5.08 ± 2.32 | 2.38 ± 0.73 |
| Example 1 ± | | 0.79 ± 0.33 | 8.38 ± 2.39 | 12.99 ± 3.05 | 12.54 ± 3.43 |
| AMD3100 | | | | | |

FIG. 20 and TABLE 14 show the synergistic effect on extended HSPC mobilization after a single dose of Example 1 in combination with a single dose of the CXCR4 inhibitor AMD3100 (Plerixafor). CXCR4 inhibitors are known to mobilize HSPCs into the peripheral blood.

TABLE 15
HSPC mobilization data for Examples 1, 4, and 5 alone or in combination with Plerixafor.

| | sVCAM | No. CFU (x10³)/mL ± SD | | | | |
|---|---|---|---|---|---|---|
| | IC₅₀ (nM) | Baseline | 2 hr | 4 hr | 6 hr | 24 hr |
| Plerixafor | | 0.62 ± 0.19 | 2.84 ± 0.24 | 1.25 ± 0.30 | 0.28 ± 0.12 | 0.22 ± 0.08 |
| Example 1 | 0.373 | 0.62 ± 0.19 | 3.12 ± 0.76 | 2.65 ± 1.15 | 1.43 ± 0.56 | 0.22 ± 0.10 |
| Example 4 | 0.527 | 0.62 ± 0.19 | 3.50 ± 0.51 | 2.79 ± 0.91 | 2.00 ± 0.84 | 0.24 ± 0.06 |
| Example 5 | 0.788 | 0.62 ± 0.19 | 2.42 ± 1.07 | 2.57 ± 1.00 | 2.34 ± 1.29 | 1.62 ± 0.75 |
| Example 1 + | | 0.62 ± 0.19 | 12.13 ± 3.35 | 7.79 ± 1.94 | 3.41 ± 1.37 | 0.37 ± 0.13 |
| Plerixafor | | | | | | |
| Example 4 + | | 0.62 ± 0.19 | 8.83 ± 0.62 | 7.04 ± 1.38 | 3.65 ± 0.31 | 0.19 ± 0.13 |
| Plerixafor | | | | | | |
| Example 5 + | | 0.62 ± 0.19 | 8.77 ± 2.22 | 7.26 ± 1.72 | 4.32 ± 1.69 | 2.29 ± 0.40 |
| Plerixafor | | | | | | |

FIG. 21 and TABLE 15 show the synergistic effects on extended HSPC mobilization after a single dose of Examples 1, 4 and 5, each in combination with a single dose of the CXCR4 inhibitor AMD3100 (Plerixafor). Note that the very long PEG chain (MW 20K) of Example 5 provides for additional extended mobilization above baseline out to 24 hours, even after the effects of AMD3100 have worn off (in effect, mobilization being driven by Example 5 alone). Further note that despite having such a long PEG group covalently attached to the core VLA4 inhibitor, VAM inhibition remains sub nM.

TABLE 16
HSPC mobilization data for Examples 1 and 2
in combination with Plerixafor and/or tGroβ.

| sVCAM | No. CFU (×10³)/mL ± SD | | | |
|---|---|---|---|---|
| IC₅₀ (nM) | Baseline | 2 hr | 4 hr | 6 hr |

| | | | | |
|---|---|---|---|---|
| Example 1 ± | 0.39 ± 0.22 | 4.26 ± 0.65 | 5.55 ± 0.67 | 2.64 ± 0.43 |
| tGroβ | | | | |
| Example 2 ± | 0.39 ± 0.22 | 6.54 ± 0.97 | 5.44 ± 0.79 | 2.91 ± 0.84 |
| tGroβ | | | | |
| Example 1 ± | 0.39 ± 0.22 | 13.86 ± 0.71 | 11.30 ± 3.71 | 3.65 ± 1.09 |
| Plerixafor | | | | |
| Example 2 ± | 0.39 ± 0.22 | 12.72 ± 2.00 | 9.14 ± 3.19 | 3.36 ± 1.16 |
| Plerixafor | | | | |
| Example 1 ± | 0.39 ± 0.22 | 19.29 ± 3.17 | 14.84 ± 4.97 | 4.42 ± 2.09 |
| Plerixafor ± | | | | |
| tGroβ | | | | |
| Example 2 ± | 0.39 ± 0.22 | 18.36 ± 3.79 | 11.29 ± 2.20 | 3.73 ± 1.47 |
| Plerixafor ± | | | | |
| tGroβ | | | | |

FIG. 22 and TABLE 16 show the combination effects on HSPC mobilization with Examples 1 and 2 combined with either the CXCR4 inhibitor plerixafor (AMD3100), with the CXCR2 agonist truncated Groβ (tGroβ), or where all 3 are used in combination. As shown, a high degree of extended mobilization occurs with Examples 1 and 2 when dosed in the triple combination regimen, with all groups still retaining significant mobilization out to 6 hours.

TABLE 17
HSPC mobilization data for Example 1 and Comparator Compound
C17 alone or in combination with Plerixafor and tGroβ.

| | sVCAM | No. CFU (×10³)/mL ± SD | | | |
|---|---|---|---|---|---|
| | IC₅₀ (nM) | Baseline | 2 hr | 4 hr | 6 hr |
| C17 | 1.490 | 0.61 ± 0.08 | 4.77 ± 1.21 | 0.81 ± 0.26 | 0.26 ± 0.13 |
| C4 | 1.693 | 0.61 ± 0.08 | 2.91 ± 0.83 | 0.53 ± 0.16 | 0.28 ± 0.09 |
| Example 1 | 0.373 | 0.61 ± 0.08 | 5.69 ± 2.57 | 4.82 ± 1.07 | 2.97 ± 0.65 |
| C12 | 0.573 | 0.61 ± 0.08 | 4.02 ± 1.99 | 0.90 ± 0.61 | 0.25 ± 0.08 |
| C17 ± | | 0.61 ± 0.08 | 20.61 ± 3.78 | 8.14 ± 4.80 | 1.66 ± 0.57 |
| Plerixafor ± | | | | | |
| tGroβ | | | | | |
| Example 1 ± | | 0.61 ± 0.08 | 29.01 ± 2.15 | 23.52 ± 3.17 | 12.32 ± 2.28 |
| Plerixafor ± | | | | | |
| tGroβ | | | | | |

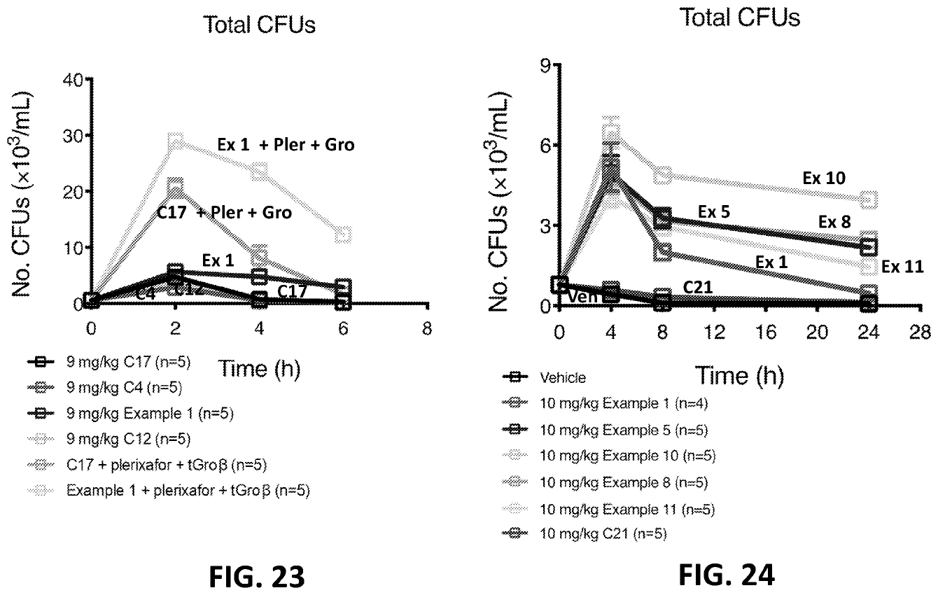

FIG. 23 and TABLE 17 compares Example 1 with Comparator Compounds C4, C12 and C17. Example 1 has a 24 PEG unit chain attached to the “di-chlorophenyl sulfonamide core” and shows extended HSPC mobilization out to 6 hours, in contrast to C12 which has an 8 PEG unit chain attached to the “di-chlorophenyl sulfonamide core” and does not extend mobilization past 2 hours. C4, a 4 PEG unit chain attached to the “di-chlorobenzoic acid core” and C17, a 24 PEG unit chain attached to the “di-chlorobenzoic acid core”, do not extend mobilization past 2 hours. This result with C17 compared to Example 1 reinforces the data shown in FIG. 15 and FIG. 17, highlighting the fact that a 24 PEG unit length on one VLA4 inhibitor core (the “di-chlorophenyl sulfonamide core” of Example 1) extends mobilization out to 6 hours, wherein the same 24 PEG unit chain attached to a different VLA4 inhibitor core (the “di-chlorobenzoic acid core” of C17) does not extend mobilization past 2 hours. Even with the triple combination regimen, C17 plus plerixafor and truncated Groβ (Groβt), is inferior to Example 1 plus plerixafor and truncated Groβ (Groβt), and does not extend mobilization out to 6 hours compared to Example 1.

TABLE 18
HSPC mobilization data for Examples 1, 5,
8, 10, and 11 and Comparator Compound C21.

| | sVCAM | No. CFU (×10³)/mL ± SD | | | |
|---|---|---|---|---|---|
| | IC₅₀ (nM) | Baseline | 4 hr | 8 hr | 24 hr |
| Vehicle | | 0.79 ± 0.24 | 0.42 ± 0.05 | 0.11 ± 0.03 | 0.08 ± 0.03 |
| Example 1 | 0.373 | 0.79 ± 0.24 | 5.18 ± 1.99 | 2.00 ± 0.66 | 0.46 ± 0.30 |
| Example 5 | 0.788 | 0.79 ± 0.24 | 4.94 ± 1.48 | 3.28 ± 0.79 | 2.18 ± 0.48 |
| Example 10 | 1.190 | 0.79 ± 0.24 | 6.47 ± 1.28 | 4.87 ± 0.61 | 3.95 ± 0.58 |
| Example 8 | 0.498 | 0.79 ± 0.24 | 4.79 ± 1.37 | 3.16 ± 0.61 | 2.45 ± 0.65 |
| Example 11 | 0.803 | 0.79 ± 0.24 | 4.08 ± 0.88 | 2.94 ± 0.67 | 1.46 ± 0.54 |
| C21 | 74.870 | 0.79 ± 0.24 | 0.58 ± 0.15 | 0.33 ± 0.11 | 0.12 ± 0.04 |

FIG. 24 and TABLE 18 show the extended mobilization effects of Example 8 (a 40 KD PEG attached to the “di-chlorophenyl sulfonamide core”) and Example 10 (a 40 KD PEG attached to the “di-chlorobenzoic acid core”), along with the previously shown Examples 1 and 5. It is noteworthy that even with a 40 KD PEG attached, VCAM potency remains unchanged compared to these cores attached to smaller PEG groups and mobilization magnitude and extension out to 24 hours and beyond are superb. These 20 and 40 KD PEG Examples 5, 8, 9, and 10 provide the opportunity for long term efficacy after a single injection for indications requiring a more chronic dosing regimen, such as multiple sclerosis, inflammatory bowel diseases, GvHD, inflammatory neurological diseases, multiple myeloma, AML, and others. Furthermore, a surprising result is shown with Example 11, a bis-“di-chlorophenyl sulfonamide core” linked together with a 10 KD PEG linker (refer to the compound structure for Example 11 in TABLE 1 for full structural clarity). As seen with other comparator compounds, usually any additions to the terminal end of the PEG chain attached to the VLA4 inhibitor core results in diminished VCAM potency and little to no HSPC mobilization. Comparator Compound C21 (a 20 KD PEG chain with the “di-chlorophenyl sulfonamide core” with a biotin derivative attached to the terminal end of the PEG chain) shown in FIG. 24 has significantly reduced VCAM potency and provides no HSPC mobilization. However, the bis Example 11 provides sub nM VCAM potency and extends HSPC mobilization out to 24 hours. This highlights the surprising nature of the Examples that support the nonobviousness of the claims of this invention.

TABLE 19
HSPC mobilization data for Examples 9 and 12 and Comparator Compound 20.

| | sVCAM | No. CFU (x10³)/mL ± SD | | | | | |
|---|---|---|---|---|---|---|---|
| | IC₅₀ (nM) | Baseline | 2 hr | 4 hr | 8 hr | 12 hr | 24 hr |
| Vehicle | | 0.65 ± 0.27 | 0.34 ± 0.09 | 0.18 ± 0.11 | 0.11 ± 0.05 | 0.15 ± 0.09 | |
| Example 9 | 1.215 | 0.65 ± 0.27 | 2.54 ± 0.83 | | 4.33 ± 1.27 | 3.46 ± 0.36 | 2.54 ± 0.41 |
| Example 12 | | 0.65 ± 0.27 | 2.37 ± 0.23 | | 4.00 ± 0.58 | 1.36 ± 0.39 | 0.33 ± 0.07 |
| C20 | 5.949 | 0.65 ± 0.27 | 2.73 ± 1.05 | | 0.66 ± 0.18 | 0.22 ± 0.09 | 0.19 ± 0.11 |

FIG. 25 and TABLE 19 show the extended mobilization effects of Example 12 out to 8 hours and still at 12 hours post dose. Example 12 is a 10 KD PEG group attached to the “di-chlorobenzoic acid core” and demonstrates the minimum PEG length needed to achieve extended mobilization after a single s.c. injection when attached to this core. This is further supported by similar extended mobilization of Example 9, the 20 KD PEG attached to the “di-chlorobenzoic acid core”, but lacking such robust extended mobilization at 8 hours (and back to baseline at 12 hours) by Comparator Compound C20, the 5 KD PEG chain attached to the “di-chlorobenzoic acid core”. Robust mobilization still at 24 hours post dose for Example 9 is exemplified as in previous Figures as providing extended mobilization beyond 24 hours for examples with PEG groups 20 KD or greater. FIG. 25, together with FIG. 18, supports the claims herein for the “di-chlorobenzoic acid core” needing a PEG group of at least ˜10 KD PEG length to achieve extended mobilization.

TABLE 20
HSPC mobilization data for Example 9, Comparator Compound
1, and prior art small molecule VLA-4 inhibitors Firategrast,
RO0270608, TR-14035, and Carotegrast.

| | sVCAM | No. CFU (×10³)/mL ± SD | | | |
|---|---|---|---|---|---|
| | IC₅₀ (nM) | Baseline | 1 hr | 4 hr | 8 hr |
| Example 9 | 1.280 | 0.47 ± 0.12 | 1.19 ± 0.46 | 2.75 ± 1.06 | 3.21 ± 1.19 |
| C1 | 4.9 | 0.47 ± 0.12 | 2.16 ± 1.22 | 0.26 ± 0.18 | 0.13 ± 0.10 |
| Firategrast | 230 | 0.47 ± 0.12 | 0.34 ± 0.12 | 0.24 ± 0.14 | 0.24 ± 0.14 |
| RO0270608 | 98.7 | 0.47 ± 0.12 | 0.64 ± 0.41 | 0.19 ± 0.06 | 0.11 ± 0.05 |
| TR-14035 | 247 | 0.47 ± 0.12 | 0.54 ± 0.15 | 0.18 ± 0.13 | 0.10 ± 0.05 |
| Carotegrast | 118.3 | 0.47 ± 0.12 | 0.58 ± 0.19 | 0.18 ± 0.08 | 0.19 ± 0.09 |

FIG. 26 and TABLE 20 show the extended mobilization effects of claimed Example 9 out to 8 hours, compared to Comparator Compound 1, which has returned to baseline by 4 hours, and a lack of any appreciable HSC mobilization by TR-14035 and similar prior art small molecule VLA4 inhibitors that have been in human clinical trials: Firategrast, R₀₀₂₇₀₆₀₈, and Carotegrast.

What is claimed is:

1. A composition comprising a VLA-4 inhibiting agent of formula: 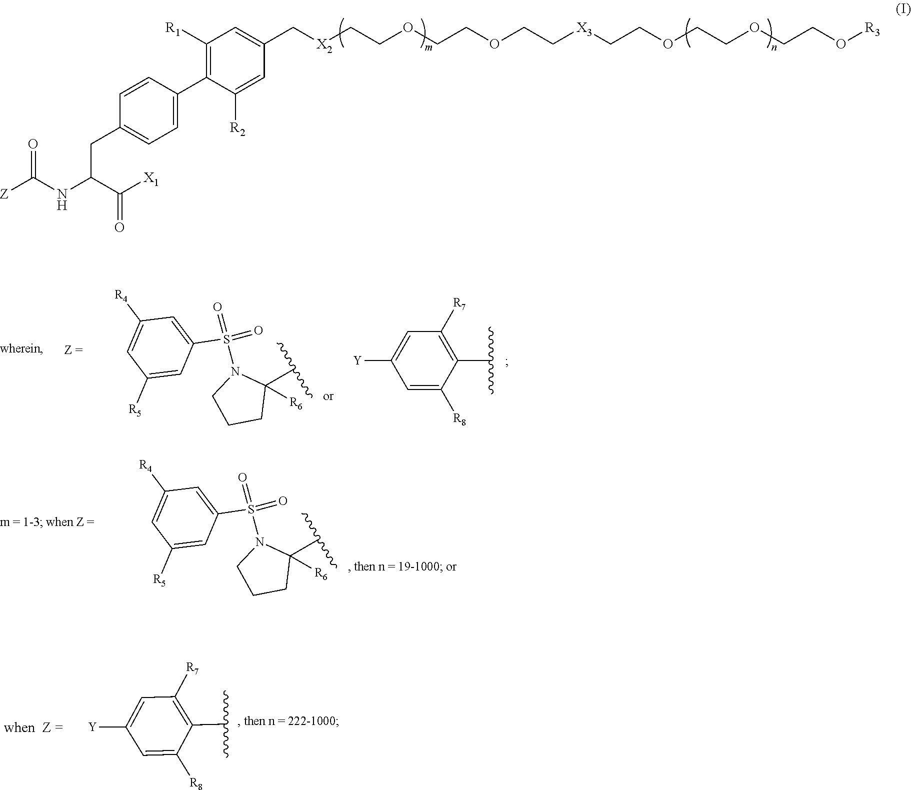
R₁ and R₂ are each independently hydroxyl, alkoxy_{(C≤8)} or substituted alkoxy_{(C≤8)};
R₃ is hydrogen, alkyl_{(C≤8)}, substituted alkyl_{(C≤6)}, haloalkyl, aryl, substituted aryl, —CH₂—CH₂—SO₂—alkyl_{(C≤8)}, —CH₂—CH₂—N(R₉)(R₁₀), wherein R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)}; or
wherein R₃ may represent a biomarker tag for in vitro or in vivo utility, an antibody targeting a specific protein or receptor, or another entity, which when attached to formula (I) enhances biological effects of combined entities, all attached to formula (I) at R₃;
if m=1, and n=19-32, and X₃ is oxygen, then R₃ can also be: 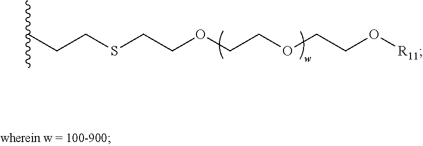
and/or R₁₁ is defined as above for R₃;
and/or, if X₃ is oxygen, m=1 and n=19-1000, then R₃ can also be: 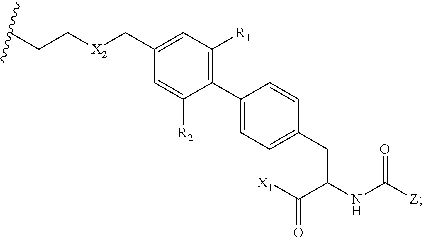
wherein R₁, R₂, X₁, and Z are defined above;
R₄ and R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, or a substituent convertible in vivo to hydroxy; or a pharmaceutically acceptable salt thereof;
R₆ is hydrogen, alkyl_{(C≤6)}, or substituted alkyl_{(C≤6)};
R₇ and R₈ are each independently hydrogen, halo, haloalkyl_{(C≤8)};
Y is hydrogen, cyano, halo, haloalkyl, hydroxy, or —C(O)X₄, wherein:
X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof;
X₂ is oxygen or sulfur;
X₃ is oxygen, sulfur, —NH(C═O)—, —(C═O)NH—, —N(R₁₂)—;
wherein R₁₂ is alkyl_{(C≤6)}, substituted alkyl_{(C≤6)}; or
a pharmaceutically acceptable salt, solvate, polymorph, tautomer, prodrug, analog, or stereoisomer thereof or optionally substituted analog thereof or bis-derivative thereof.

2. A composition comprising a VLA-4 inhibiting agent of formula: 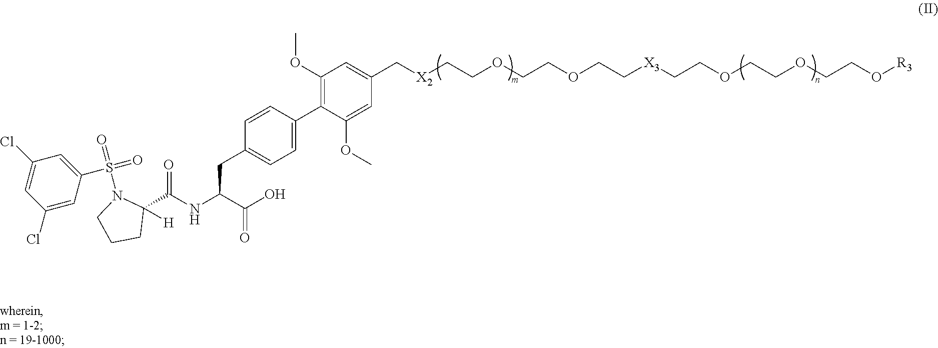
R₃ is hydrogen, alkyl_{(C≤6)}, substituted alkyl_{(C≤6)}, haloalkyl, aryl, substituted aryl, —CH₂—CH₂—SO₂—alkyl_{(C≤6)}, —CH₂—CH₂—N(R₉)(R₁₀), wherein R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)};
or wherein R₃ may represent a biomarker tag for in vitro or in vivo utility, an antibody targeting a specific protein or receptor, or another entity, which when attached to formula (II) enhances biological effects of combined entities, all attached to formula (II) at R₃; or
if m=1, and n=19-32, and X₃ is oxygen, then R₃ can also be: 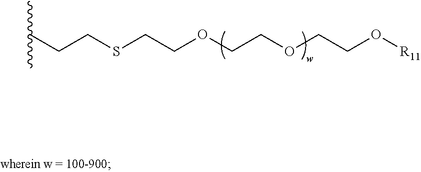
and/or R₁₁ is defined as above for R₃;
and/or, if X₃ is oxygen, m=1 and n=19-1000, then R₃ can also be: 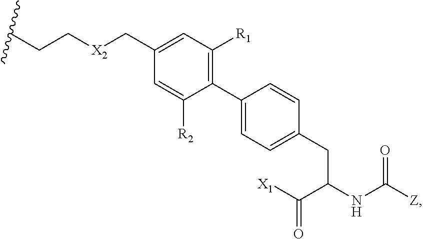
wherein R₁, R₂, X₁, and Z are defined above;
X₂ is oxygen or sulfur; or
X₃ is oxygen, sulfur, —NH(C═O)—, —(C═O)NH—, —N(R₁₂)—, wherein R₁₂ is alkyl_{(C≤6)}, substituted alkyl_{(C≤6)};
or a pharmaceutically acceptable salt, solvate, polymorph, tautomer, prodrug, analog, or stereoisomer thereof or optionally substituted analog thereof or bis-derivative thereof.

3. A composition comprising a VLA-4 inhibiting agent of formula: 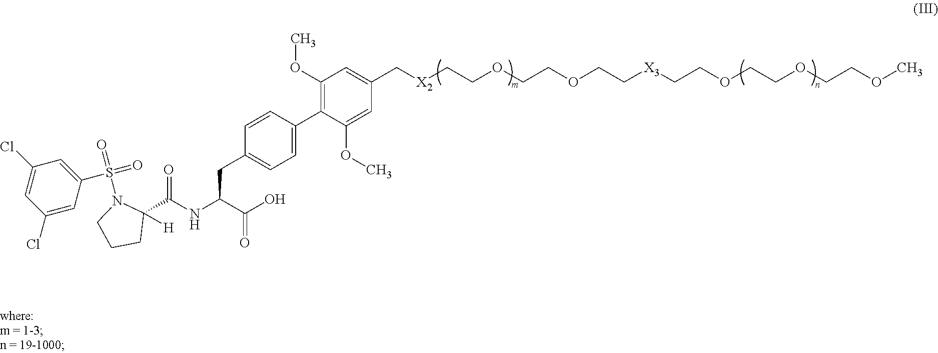
X₂ is oxygen or sulfur; or
X₃ is oxygen, sulfur, —NH(C═O)—, —(C═O)NH—, —N(R₁₂)—, wherein R₁₂ is alkyl_{(C≤6)}, substituted alkyl_{(C≤6)};
or a pharmaceutically acceptable salt, solvate, polymorph, tautomer, prodrug, analog, or stereoisomer thereof or optionally substituted analog thereof or bis-derivative thereof.

4. A composition comprising a VLA-4 inhibiting agent of formula: 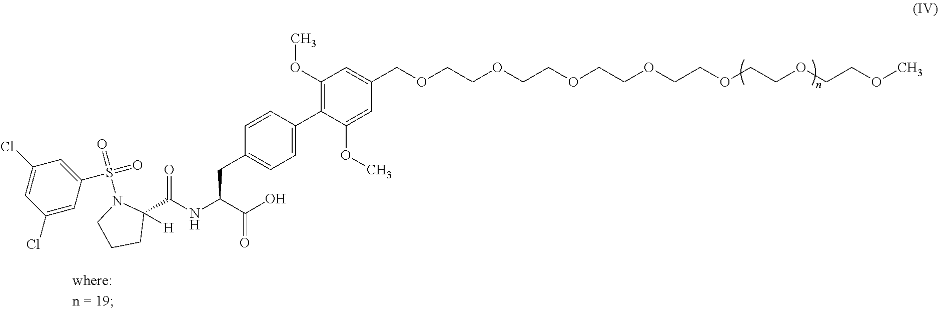
or a pharmaceutically acceptable salt, solvate, polymorph, tautomer, prodrug, analog, or stereoisomer thereof or optionally substituted analog thereof or bis-derivative thereof.

5. A composition comprising a VLA-4 inhibiting agent of formula: 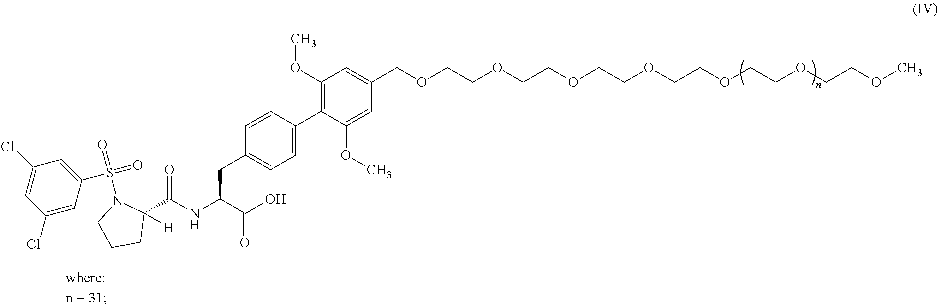
or a pharmaceutically acceptable salt, solvate, polymorph, tautomer, prodrug, analog, or stereoisomer thereof or optionally substituted analog thereof or bis-derivative thereof.

6. A composition comprising a VLA-4 inhibiting agent of formula: 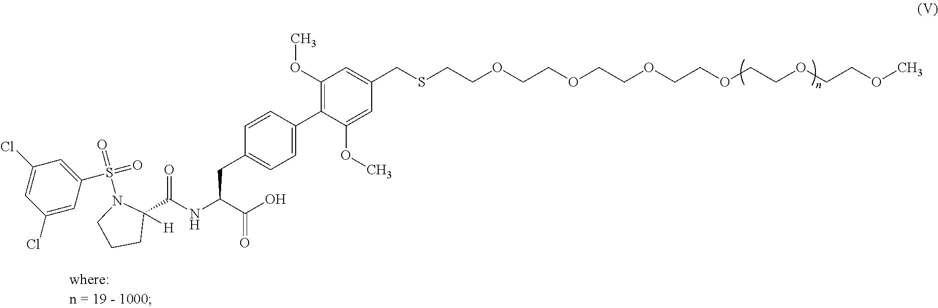
or a pharmaceutically acceptable salt, solvate, polymorph, tautomer, prodrug, analog, or stereoisomer thereof or optionally substituted analog thereof or bis-derivative thereof.

7. A composition comprising a VLA-4 inhibiting agent of formula: 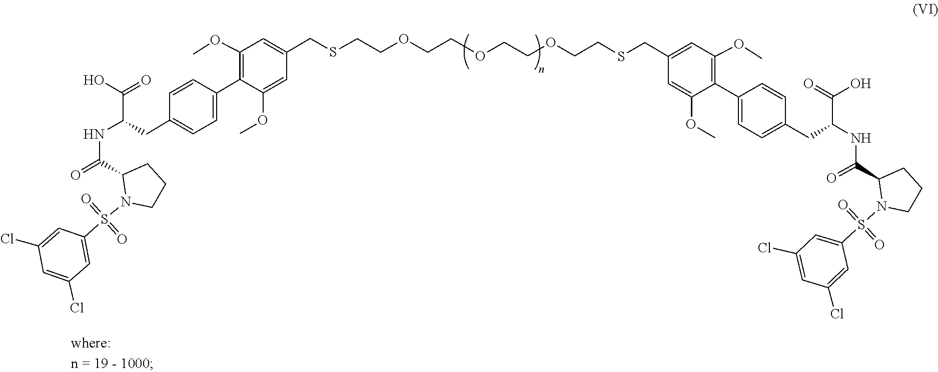
or a pharmaceutically acceptable salt, solvate, polymorph, tautomer, prodrug, analog, or stereoisomer thereof or optionally substituted analog thereof.

8. A composition comprising a VLA-4 inhibiting agent of formula: 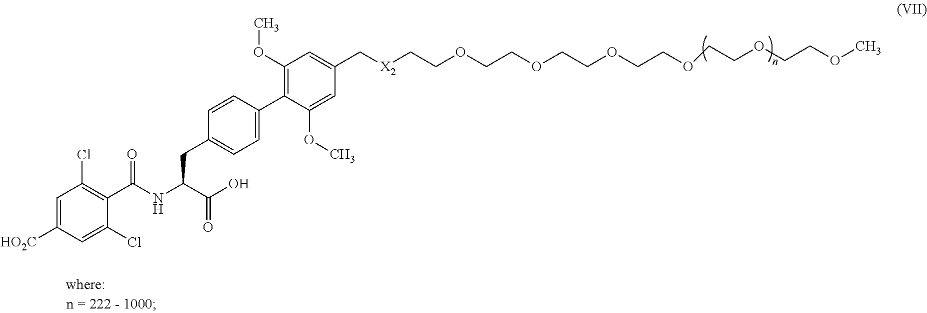
X₂ is oxygen or sulfur;
or a pharmaceutically acceptable salt, solvate, polymorph, tautomer, prodrug, analog, or stereoisomer thereof or optionally substituted analog thereof or bis-derivative thereof.

9. A composition comprising a VLA-4 inhibiting agent of formula: 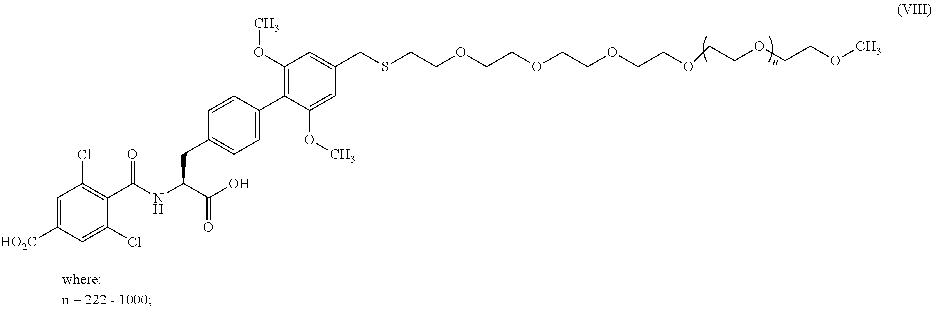
or a pharmaceutically acceptable salt, solvate, polymorph, tautomer, prodrug, analog, or stereoisomer thereof or optionally substituted analog thereof or bis-derivative thereof.

10. A composition comprising a VLA-4 inhibiting agent of formula: 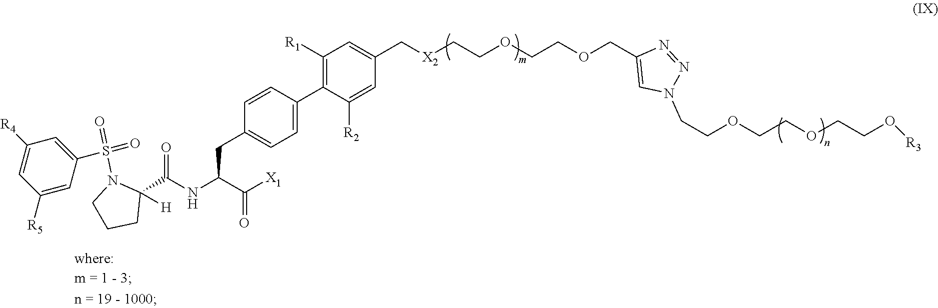
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy; or a pharmaceutically acceptable salt thereof;
X₂ is oxygen or sulfur;
R₁ and R₂ are each independently hydroxyl, alkoxy(cs) or substituted alkoxy(cs);
R₃ is hydrogen, alkyl_{(C≤6)}, substituted alkyl_{(C≤6)}, haloalkyl, aryl, substituted aryl, —CH₂—CH₂—SO₂—alkyl_{(C≤6)}, —CH₂—CH₂—N(R₉)(R₁₀), wherein R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)};
or wherein R₃ may represent a biomarker tag for in vitro or in vivo utility, an antibody targeting a specific protein or receptor, or another entity, which when attached to formula (IX) enhances biological effects of combined entities, all attached to formula (IX) at R₃; or
if m=1, and n=19-32, then R₃ can also be: 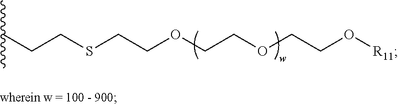
and R₁ is defined as above for R₃; or
R₄ and R₅ are each independently hydrogen, alkyl_{(C≤8)}, alkoxy_{(C≤8)}, halo, haloalkyl_{(C≤8)}, substituted haloalkyl_{(C≤8)}, or —C(O)X₅, wherein: X₅ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, or a substituent convertible in vivo to hydroxy; or a pharmaceutically acceptable salt thereof;
or a pharmaceutically acceptable salt, solvate, polymorph, tautomer, prodrug, analog, or stereoisomer thereof or optionally substituted analog thereof or bis-derivative thereof.

11. A composition comprising a VLA-4 inhibiting agent of formula: 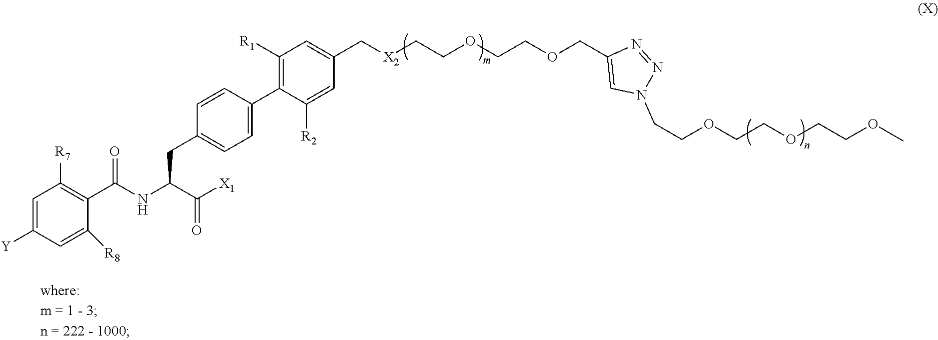
X₁, X₂, R₁, R₂, and R₃ are as defined above,
R₇ and R₆ are each independently hydrogen, halo, haloalkyl_{(C≤8)};
Y is hydrogen, cyano, halo, haloalkyl, hydroxy, or —C(O)X₄;
wherein, X₄ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenyl-amino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; or a pharmaceutically acceptable salt thereof;
or a pharmaceutically acceptable salt, solvate, polymorph, tautomer, prodrug, analog, or stereoisomer thereof or optionally substituted analog thereof or bis-derivative thereof.

12. A composition comprising a VLA-4 inhibiting agent of formula: 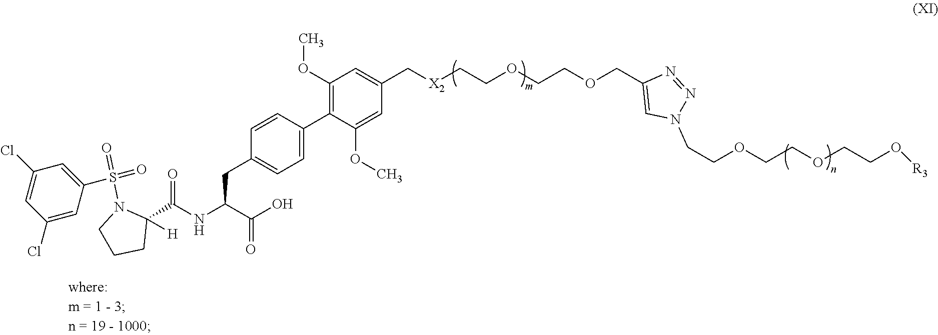
X₂ is oxygen or sulfur;
R₃ is hydrogen, alkyl_{(C≤6)}, substituted alkyl_{(C≤6)}, haloalkyl, aryl, substituted aryl, —CH₂—CH₂—SO₂—alkyl_{(C≤6)}, —CH₂—CH₂—N(R₉)(R₁₀), wherein R₉ and R₁₀ are each independently hydrogen, alkyl_{(C≤6)}, substituted alkyl_{(C≤6)}; —(CH₂)_{g}—CH₂—CO₂R₉, wherein g is 0 or 1, and wherein R₉ is hydrogen, alkyl_{(C≤6)}, substituted alkyl_{(C≤6)};
or wherein R₃ may represent a biomarker tag for in vitro or in vivo utility, an antibody targeting a specific protein or receptor, or another entity, which when attached to formula (XI) enhances biological effects of combined entities, all attached to formula (XI) at R₃;
if m=1, and n=19-32, then R₃ can also be: 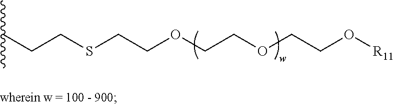
and R₁₁ is defined as above for R₃;
or a pharmaceutically acceptable salt, solvate, polymorph, tautomer, prodrug, analog, or stereoisomer thereof or optionally substituted analog thereof or bis-derivative thereof.

13. A composition comprising a VLA-4 inhibiting agent of formula: 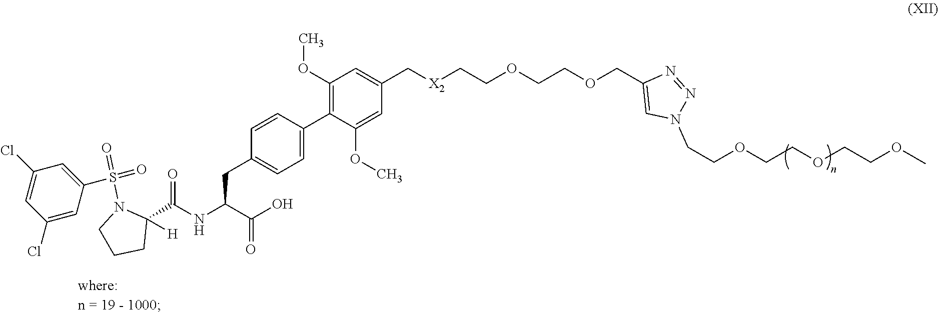
X₂ is oxygen or sulfur;
or a pharmaceutically acceptable salt, solvate, polymorph, tautomer, prodrug, analog, or stereoisomer thereof or optionally substituted analog thereof or bis-derivative thereof.

14. A composition comprising a VLA-4 inhibiting agent of formula: 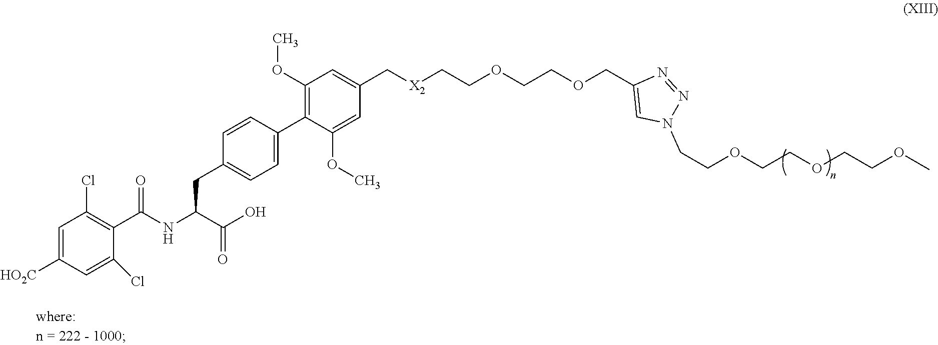
X₂ is oxygen or sulfur;
or a pharmaceutically acceptable salt, solvate, polymorph, tautomer, prodrug, analog, or stereoisomer thereof or optionally substituted analog thereof or bis-derivative thereof.

15. A composition comprising a formula selected from any one of the formulas:

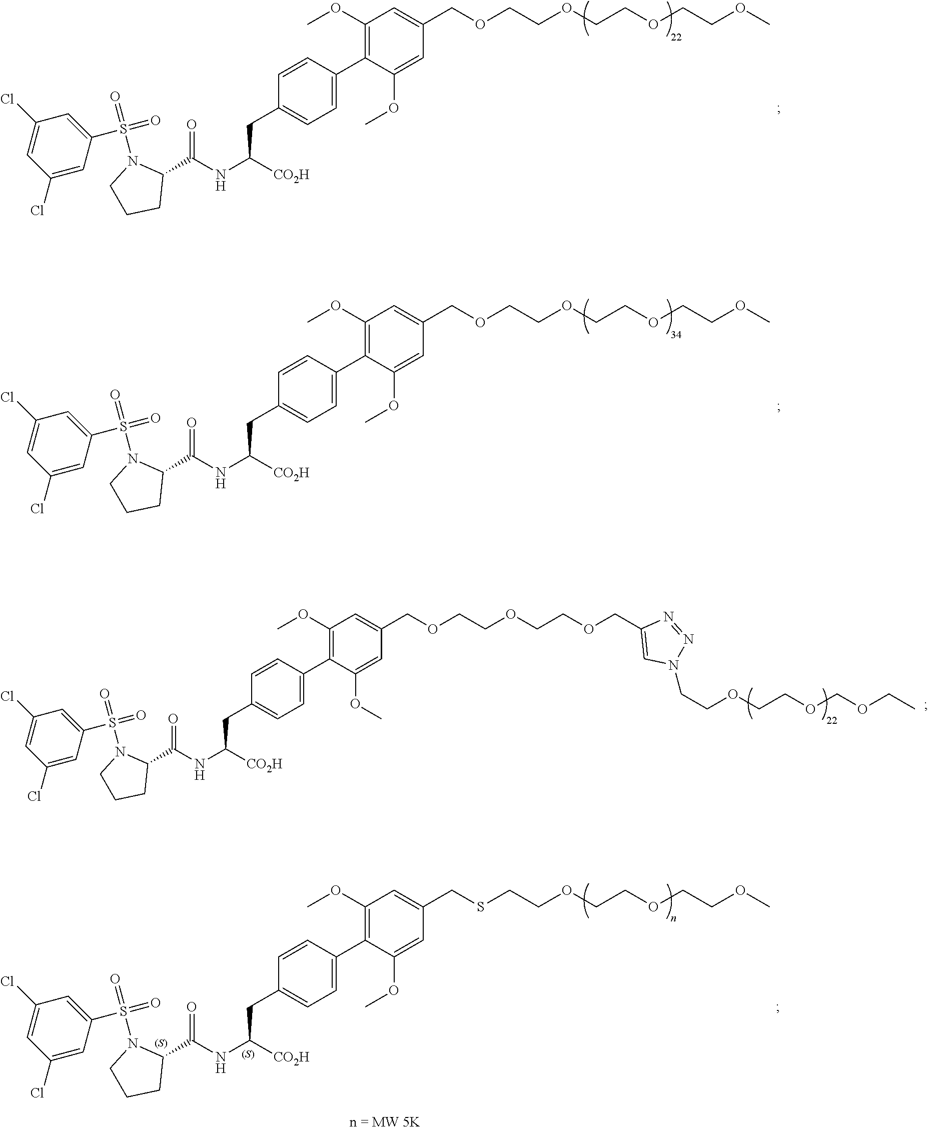

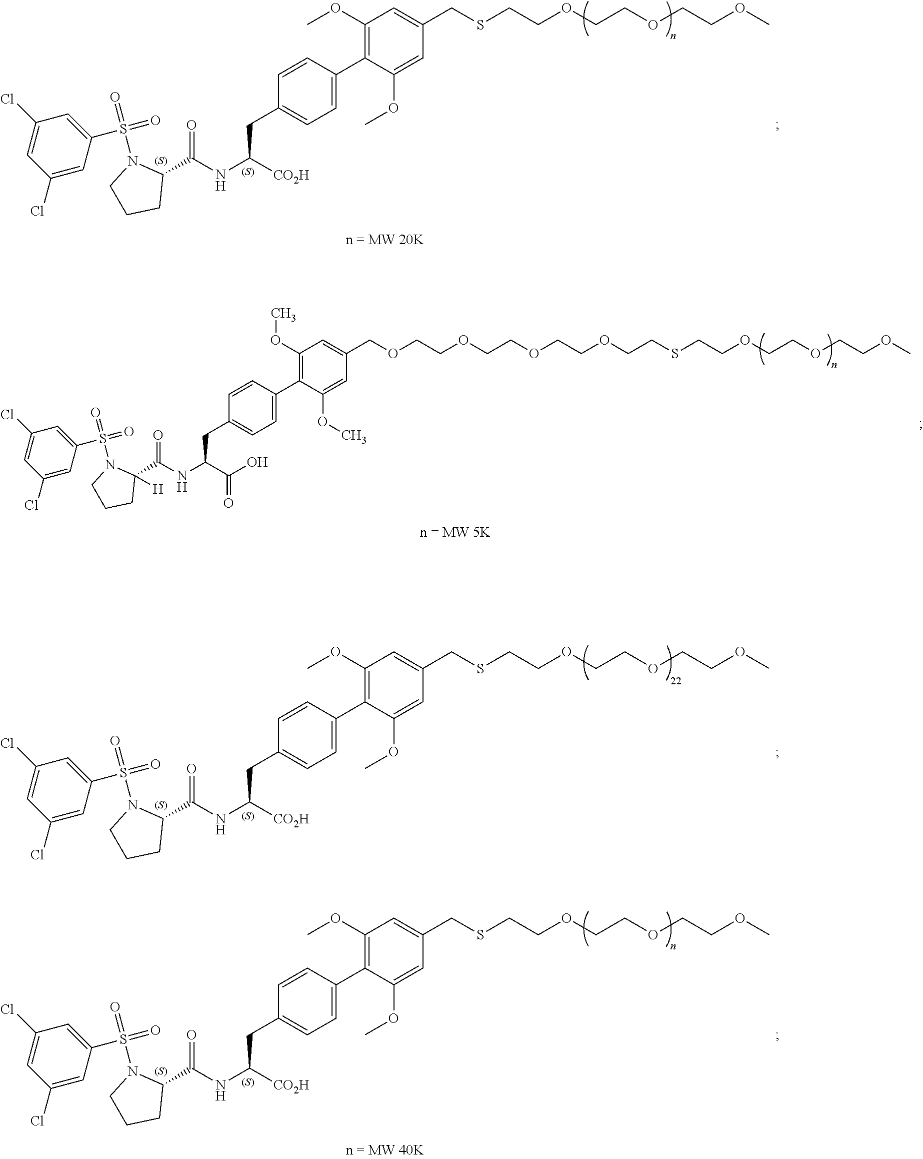

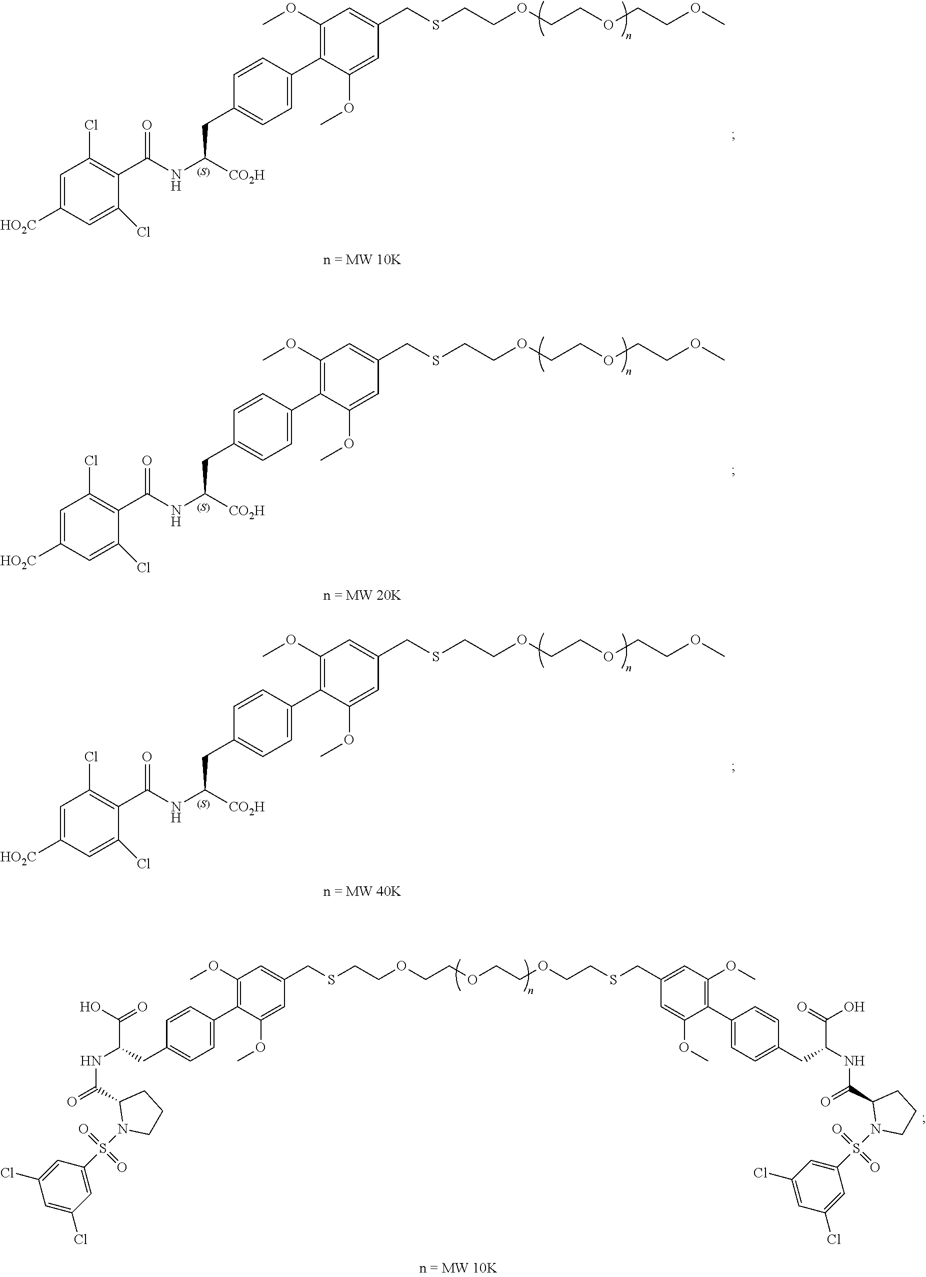

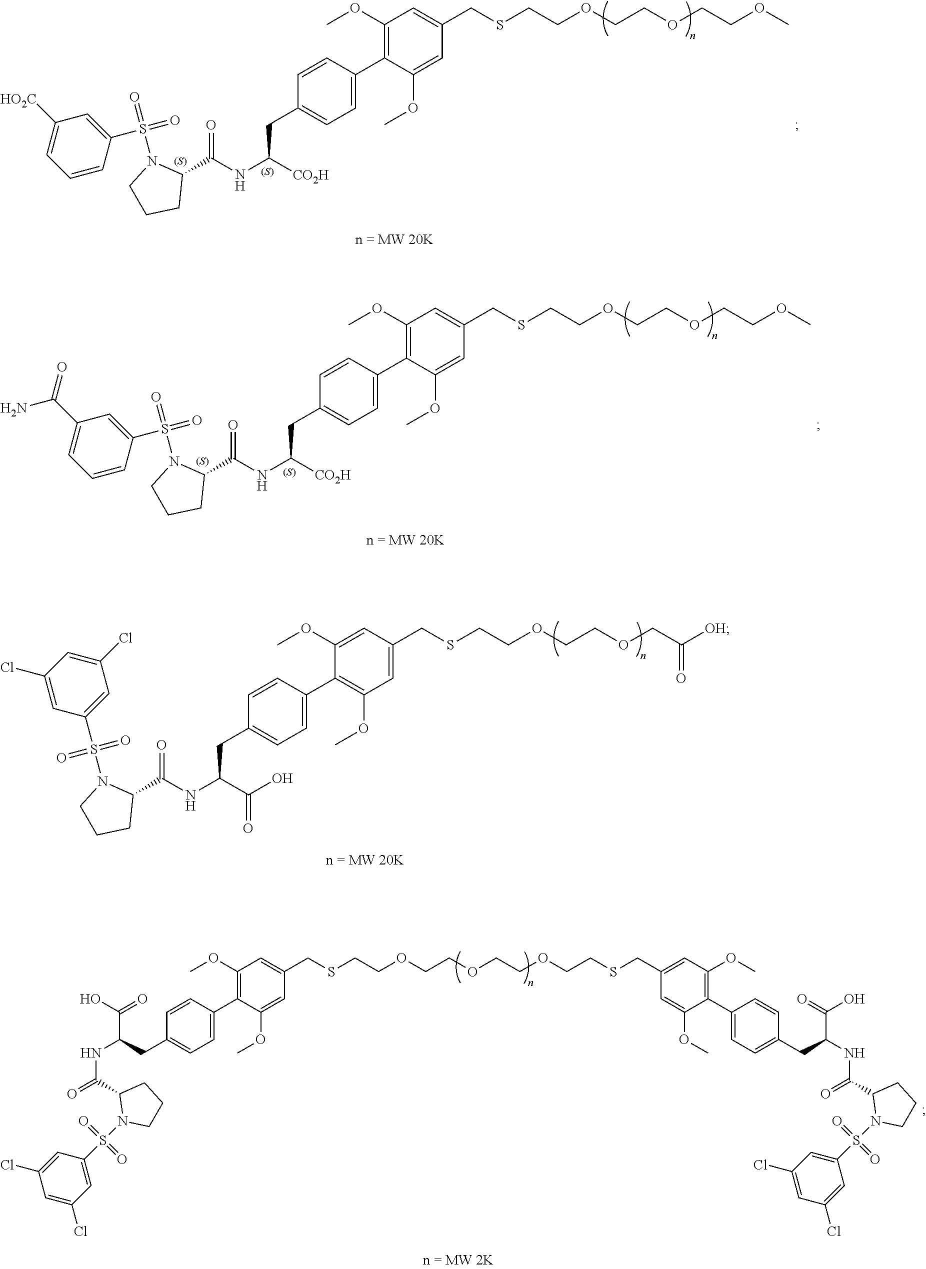

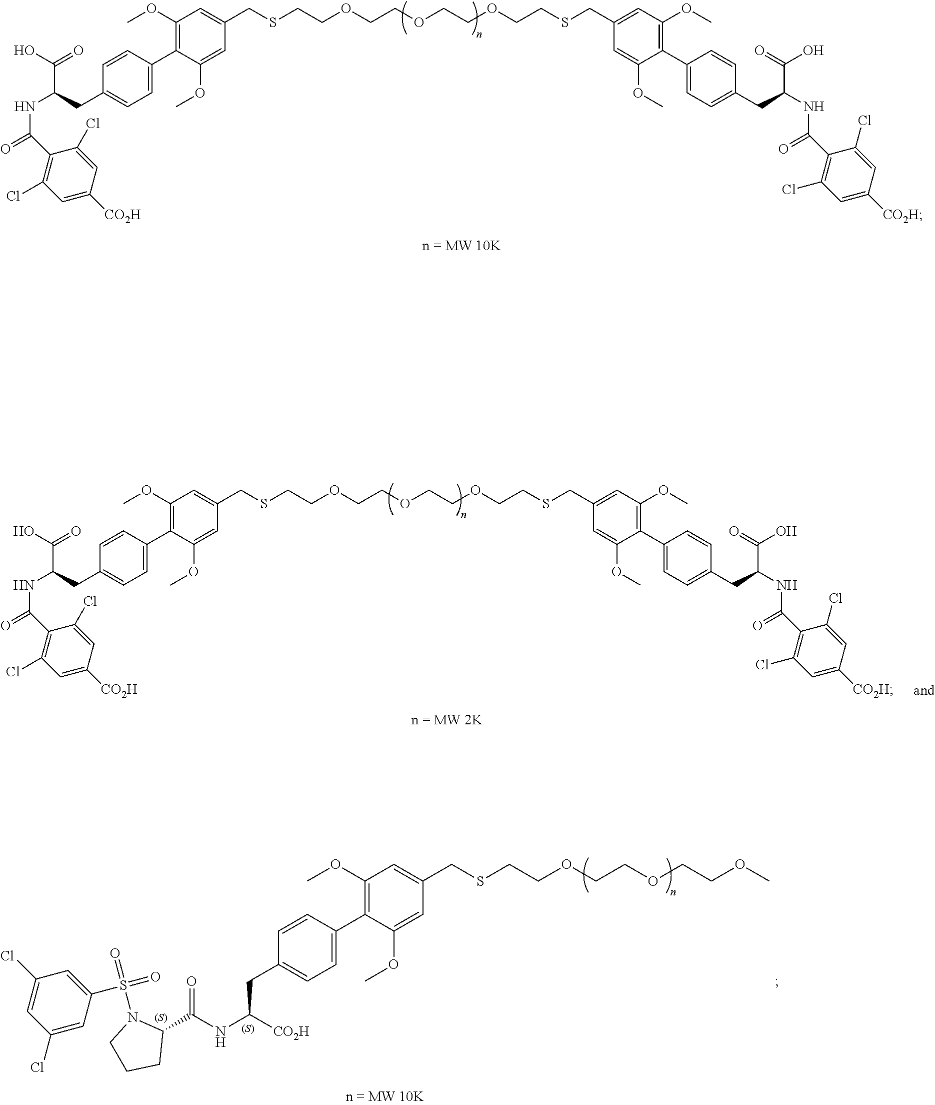

16. A pharmaceutical composition comprising:
a) the composition of any one of claims 1 to 15;
b) an excipient; and/or
c) saline.

17. The pharmaceutical composition of claim 16, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

18. The pharmaceutical composition of claim 16 or 17, wherein the pharmaceutical composition is formulated for oral, subcutaneous, intravenous, or intraperitoneal administration.

19. A composition comprising:
a VLA-4 inhibitor of any one of claims 1 to 15; and
one or more agents which interact with one or more chemokine receptors.

20. The composition of claim 19, wherein the one or more agents which interact with a chemokine receptor is an agent which interacts with a C—X—C chemokine receptor.

21. The composition of claim 20, wherein the one or more agents is a CXCR4 inhibitor or CXCR2 agonist.

22. The composition of claim 21, wherein the one or more agents is a CXCR4 inhibitor selected from, but not limited to AMD3100 (plerixafor), BL-8040 (Motixafortide), AMD3465, CTCE-0214, CTCE-9908, CP-1221 (linear peptides, cyclic peptides, natural amino-acids, unnatural amino acids, or peptidomimetic compounds), T140 or analogs, 4F-benzoyl-TN24003, KRH-1120, KRH-1636, KRH-2731, polyphemusin analogue, ALX40-4C, or combinations thereof.

23. The composition of claim 21, wherein the one or more agents is a CXCR2 agonist selected from Groβ or a derivative of Groβ.

24. The composition of claim 23, wherein the derivative of Groβ is a truncated Groβ.

25. The composition of claim 24, wherein the truncated Groβ is SB-251353.

26. The composition of claim 19, wherein the composition further comprises an inhibitor of integrin α9β1, G-CSF, a derivative of G-CSF, or a combination thereof.

27. A composition comprising:
a VLA-4 inhibitor compound; and
an agent which interacts with one or more chemokines.

28. The composition of claim 27, wherein the agent which interacts with a chemokine is selected from an agent which interacts with a C—X—C chemokine or a C—X—C chemokine receptor.

29. The composition of claim 28, wherein the agent is a CXCR4 inhibitor.

30. The composition of claim 28, wherein the agent is a CXCR2 agonist.

31. The composition of claim 29, wherein the CXCR4 inhibitor is one or more of, but not limited to AMD3100 (plerixafor), BL-8040 (Motixafortide), AMD3465, CTCE-0214, CTCE-9908, CP-1221 (e.g., linear peptides, cyclic peptides, natural amino-acids, unnatural amino acids, peptidomimetic compounds), T140 or analogs, 4F-benzoyl-TN24003, KRH-1120, KRH-1636, KRH-2731, polyphemusin analogue, ALX40-4C, or combinations thereof.

32. The composition of claim 30, wherein the CXCR2 agonist is Groβ or a derivative of Groβ.

33. The composition of claim 32, wherein the derivative of Groβ is a truncated Groβ.

34. The composition of claim 33, wherein the truncated Groβ is SB-251353.

35. The pharmaceutical composition of any one of claims 1 to 18, wherein the pharmaceutical composition is formulated as a unit dose.

36. A pharmaceutical composition comprising the composition of any one of claims 19 to 34, wherein the pharmaceutical composition of the combination of a VLA4 inhibitor and an agent which interacts with one or more chemokines of any one of the preceding claims is formulated and administered as a unit dose or formulated and administered independently of each other.

37. A method of treating a patient or a donor to enhance the mobilization and/or collection of a sufficient quantity of hematopoietic stem/progenitor cells into the peripheral blood of the patient or the donor comprising administering to the patient or donor the composition or pharmaceutical composition of any one of claims 1 to 36 in an amount sufficient to mobilize and/or collect a sufficient quantity of hematopoietic stem/progenitor cells into the peripheral blood.

38. The method of claim 37, wherein the amount sufficient to mobilize and/or collect a sufficient quantity of hematopoietic stem/progenitor cells is an amount that results in multilineage engraftment in a recipient.

39. The method of claim 37, wherein the amount sufficient to mobilize and/or collect a sufficient quantity of hematopoietic stem/progenitor cells is an amount that results in neutrophil and platelet engraftment.

40. The method of claim 37, wherein the amount sufficient to mobilize and/or collect a sufficient quantity of hematopoietic stem/progenitor cells is an amount sufficient for use in gene editing or genetic engineering.

41. The method of claim 37, wherein the amount sufficient to mobilize and/or collect a sufficient quantity of hematopoietic stem/progenitor cells is an amount sufficient to be therapeutically effective in a subject having a disease, disorder, or condition that is treatable with hematopoietic stem/progenitor cells.

42. The method of claim 41, wherein the disease, disorder, or condition is associated with impaired production of hematopoietic progenitor and/or stem cells resulting from a high dose of chemotherapy, radiotherapy, another therapeutic agent, such as for treating blood cancers or a genetic abnormality.

43. The method of claim 41, wherein the disease, disorder, or condition is associated with a blood cancer or a genetic abnormality; a blood borne disease (e.g., sickle cell disease); or a hematopoietic malignancy (e.g., leukemia, lymphoma, or myeloma, such as multiple myeloma or acute myeloid leukemia).

44. The method of claim 37, wherein the amount sufficient to mobilize and/or collect a sufficient quantity of hematopoietic stem/progenitor cells into the peripheral blood of a human donor is at least about 2 million CD34+ stem cells per kilogram recipient body weight.

45. A method of treating a patient, comprising
collecting hematopoietic stem/progenitor cells from a patient or the donor, resulting in collected hematopoietic stem/progenitor cells; and
infusing the collected hematopoietic stem/progenitor cells of claim 37, wherein the patient may have impaired production of hematopoietic progenitor and/or stem cells resulting from a high dose of chemotherapy, radiotherapy, another therapeutic agent, such as for treating blood cancers or a genetic abnormality.

46. The method of claim 37, further comprising:
collecting hematopoietic stem/progenitor cells from the patient, resulting in collected hematopoietic stem/progenitor cells;
gene editing the collected hematopoietic stem/progenitor cells of the patient, wherein the gene editing corrects a mutation causing a blood borne disease, resulting in gene edited hematopoietic stem/progenitor cells; and
infusing the gene edited hematopoietic stem/progenitor cells into the patient to attenuate or treat the cause or pathology of the blood borne disease.

47. The method of claim 46, wherein the blood borne disease is sickle cell disease.

48. A method of treating and/or preventing a disease, disorder, or condition in a patient in need thereof, comprising administering to the patient a composition or pharmaceutical composition of any one of claims 1 to 36 in an amount sufficient to treat and/or prevent the disease, disorder, or condition.

49. The method of claim 48, wherein a compound or composition of any one of claims 1 to 36 increases effectiveness of an anti-cancer therapy.

50. The method of claim 49, wherein the anti-cancer therapy is used to treat a patient who have or are at risk for a hematopoietic malignancy (e.g., lymphoma, myeloma, leukemia), wherein the compositions of any one of the preceding claims are administered or employed prior to, during, or subsequent to an anti-cancer therapy (e.g., chemotherapeutic agents, radiotherapy).

51. The method of claim 50, wherein the hematopoietic malignancy is multiple myeloma or acute myeloid leukemia.

52. The method of claim 49, wherein a compound or composition of any one of claims 1 to 36 is administered or employed in combination with bi-specific antibodies or other immuno-oncology agents for treating patients with a leukemia, lymphoma, or myeloma.

53. The method of claim 49, wherein a compound or composition of any one of claims 1 to 36 is administered or employed in combination with bi-specific antibodies or other immuno-oncology agents for treating a patient having multiple myeloma or acute myeloid leukemia.

54. A method of treating a disease, disorder, or condition associated with cell adhesion-mediated inflammatory pathways with a pharmaceutical composition of claim 16.

55. The method of claim 54 wherein the disease, disorder, or condition is, but not limited to, asthma, multiple sclerosis, rheumatoid arthritis, atherosclerosis, inflammatory bowel disease, Crohn's disease, ulcerative colitis, graft vs host disease, neuroinflammation, neurodegeneration, or spinal cord injury.

56. A method of binding inhibition of an integrin comprising contacting the integrin with a composition of any one of claims 1 to 15.

57. The method of claim 56, wherein the integrin is VLA4 (a4b1) or a4b7.

58. The method of claim 56, wherein the integrin is VLA4 (a4b1).

59. The method of claim 56, wherein the integrin is a4b7.

60. The method of any one of claims 56 to 59, wherein the method is performed in vitro.

61. The method of any one of claims 56 to 59, wherein the method is performed ex vivo or in vivo.

62. The method of any one of claims 56 to 59 or 61, wherein the binding inhibition is sufficient to treat or prevent a disease, disorder, or condition in a patient or to enhance or extend mobilization and/or collection of sufficient amounts of hematopoietic stem/progenitor cells into the peripheral blood of a subject.

63. The method of any one of claims 56 to 59 or 61, wherein the binding inhibition in combination with one or more agents which interact with one or more chemokine receptors is sufficient to treat or prevent a disease, disorder, or condition in a patient or to enhance or extend mobilization and/or collection of sufficient amounts of hematopoietic stem/progenitor cells into the peripheral blood of a subject.

64. A pharmaceutical composition comprising a composition of any one of claims 1 to 15 which provides for significant and extended mobilization of hematopoietic stem/progenitor cells into the peripheral blood of a patient or donor lasting greater than 4 hours after a single administered dose.

65. A pharmaceutical composition comprising a composition of any one of claims 1 to 15 comprising a PEG MW equal to or greater than 20 KD which provides for significant and extended mobilization of hematopoietic stem/progenitor cells into the peripheral blood of a patient or donor lasting greater than 24 hours after a single administered dose.
